(19) United States
(12) Patent Application Publication
(10) Pub. No.: US 2024/0033341 A1
(43) Pub. Date: Feb. 1, 2024

(54) HIV VACCINE IMMUNOGENS

(71) Applicants: California Institute of Technology, Pasadena, CA (US); THE ROCKEFELLER UNIVERSITY, NEW YORK, NY (US)

(72) Inventors: Harry Gristick, Pasadena, CA (US); Pamela J. Bjorkman, Altadena, CA (US); Harald Hartweger, New York, NY (US); Michel C. Nussenzweig, New York, NY (US)

(21) Appl. No.: 18/340,008

(22) Filed: Jun. 22, 2023

Related U.S. Application Data
(60) Provisional application No. 63/354,993, filed on Jun. 23, 2022.
(60) Provisional application No. 63/356,133, filed on Jun. 28, 2022.

(51) Int. Cl. A61K 39/21; A61P 37/04

(52) U.S. Cl. CPC A61K 39/21; A61P 37/04; A61K 2039/545

(57) ABSTRACT

Provided herein are HIV immunogens and uses thereof for generating an immune response in a subject. This disclosure further provides a method for treating or preventing a human immunodeficiency type I (HIV-I) infection in a subject using the disclosed HIV immunogens and/or antibodies generated by any of the methods disclosed herein.

### RELATED APPLICATIONS

The present application claims priority to U.S. Provisional Application No. 63/354,993, filed Jun. 23, 2022; and U.S. Provisional Application No. 63/356,133, filed Jun. 28, 2022. The entire contents of these applications are hereby expressly incorporated by reference in their entireties.

### STATEMENT REGARDING FEDERALLY SPONSORED R&D

This invention was made with government support under Grant No. AI100148 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-365858-US_Sequence_Listing, created Jun. 22, 2023, which is 265 kilobytes in size. The information in the electronic format of the Sequence Listing is incorporated herein by reference in its entirety.

### BACKGROUND

### Field

The present disclosure relates generally to the field of human immunodeficiency virus. More specifically, disclosed herein include immunogenic polypeptides capable of stimulating an immune response to HIV.

### Description of the Related Art

Passive transfer of broadly neutralizing anti-HIV-1 antibodies (bNAbs) protects against infection, and therefore eliciting bNAbs (e.g., IOMA antibody) by vaccination is a major goal of HIV-1 vaccine efforts. bNAbs that target the CD4-binding site (CD4bs) on HIV-1 Env are among the most broadly active, but to date, responses elicited against this epitope in vaccinated animals have lacked potency and breadth. There is a need for immunogens capable of eliciting bNAbs. Provided herein are polypeptides and methods for eliciting development of bNAbs. Also provided herein are vaccination regimens using the disclosed polypeptides.

### SUMMARY

Disclosed herein include isolated polypeptides. The isolated polypeptide can, for example, comprise an amino acid sequence that is at least 90% identical to the sequence of SEQ ID NO: 119, where the isolated polypeptide comprises at least an amino acid mutation in a position corresponding to D279, V430, D460, T461, T462, D463, or N464 of SEQ ID NO: 119. The mutation can be, for example, an amino acid substitution. In some embodiments, the isolated polypeptide comprises a D279N substitution and/or a V430P substitution. In some embodiments, the isolated polypeptide further comprises at least one of a D460N substitution, a T461S substitution, a T462Q substitution, a D463R substitution, and an N464E substitution. In some embodiments, the isolated polypeptide comprises the sequence of SEQ ID NO: 118. The isolated polypeptide can comprise an amino acid sequence that is at least 90% identical to the sequence of SEQ ID NO: 118 or comprising the sequence of SEQ ID NO: 118. In some embodiments, the isolated polypeptide consists of the sequence of SEQ ID NO: 118.

In some embodiments, the isolated polypeptide further comprises at least one of a D460N substitution, a T461A substitution, a T462L substitution, a D463R substitution, and an N464P substitution. In some embodiments, the isolated polypeptide comprises the sequence of SEQ ID NO: 117. The isolated polypeptide can comprise an amino acid sequence that is at least 90% identical to the sequence of SEQ ID NO: 117 or comprising the sequence of SEQ ID NO: 117. In some embodiments, the isolated polypeptide consists of the sequence of SEQ ID NO: 117.

In some embodiments, the isolated polypeptide binds to a neutralizing antibody with an affinity of about 30 μM or less. In some embodiments, the isolated polypeptide binds to a neutralizing antibody with an affinity of about 30 μM. In some embodiments, the isolated polypeptide binds to a neutralizing antibody with an affinity of about 0.5 μM. In some embodiments, the neutralizing antibody has specificity for a CD4 binding site of an HIV Env protein. In some embodiments, the neutralizing antibody comprises a heavy chain comprising an amino acid sequence selected from SEQ ID NOs: 1, 153, 155, 157, and 159 (ii) an amino acid sequence having at least 90% sequence identity to an amino acid sequence selected from SEQ ID NOs: 1, 153, 155, 157, and 159, or (iii) an amino acid sequence having one, two or three mismatches relative to an amino acid sequence selected from SEQ ID NOs: 1, 153, 155, 157, and 159. In some embodiments, the neutralizing antibody comprises a light chain comprising an amino acid sequence selected from SEQ ID NOs: 12, 154, 156, 158, and 160, (ii) an amino acid sequence having at least 90% sequence identity to an amino acid sequence selected from SEQ ID NOs: 12, 154, 156, 158, and 160, or (iii) an amino acid sequence having one, two or three mismatches relative to an amino acid sequence selected from SEQ ID NOs: 12, 154, 156, 158, and 160.

Disclosed herein include isolated polypeptides. In some embodiments, the isolated polypeptide comprises an amino acid sequence that is at least 90% identical to the sequence of SEQ ID NO: 122, wherein the isolated polypeptide comprises at least an amino acid mutation in a position corresponding to D279, V430, D460, T461, T462, D463, and N464 of SEQ ID NO: 122.

In some embodiments, the mutation is an amino acid substitution. In some embodiments, the isolated polypeptide comprises a D279N substitution and/or a V430P substitution. In some embodiments, the isolated polypeptide further comprises at least one of a D460N substitution, a T461S substitution, a T462Q substitution, a D463R substitution, and an N464E substitution. In some embodiments, the isolated polypeptide comprises the sequence of SEQ ID NO: 121. Disclosed herein include isolated polypeptides. In some embodiments, the isolated polypeptide comprises an amino acid sequence that is at least 90% identical to the sequence of SEQ ID NO: 121 or comprising the sequence of SEQ ID NO: 121. In some embodiments, the isolated polypeptide consists of the sequence of SEQ ID NO: 121.

In some embodiments, the isolated polypeptide further comprises at least one of a D460N substitution, a T461A substitution, a T462L substitution, a D463R substitution, and an N464P substitution. In some embodiments, the isolated polypeptide comprises the sequence of SEQ ID NO: 120. Disclosed herein include isolated polypeptides. In some embodiments, the isolated polypeptide comprises an amino acid sequence that is at least 90% identical to the sequence of SEQ ID NO: 120 or comprising the sequence of SEQ ID NO: 120. In some embodiments, the isolated polypeptide consists of the sequence of SEQ ID NO: 120.

Disclosed herein include isolated polypeptides. In some embodiments, the isolated polypeptide comprises an amino acid sequence that is at least 90% identical to the sequence of SEQ ID NO: 131 or comprising the sequence of SEQ ID NO: 131. In some embodiments, the isolated polypeptide consists of the sequence of SEQ ID NO: 131.

Disclosed herein include isolated polypeptides. In some embodiments, the isolated polypeptide comprises an amino acid sequence that is at least 90% identical to the sequence of SEQ ID NO: 130 or comprising the sequence of SEQ ID NO: 130. In some embodiments, the isolated polypeptide consists of the sequence of SEQ ID NO: 130.

Disclosed herein include isolated polypeptides. In some embodiments, the isolated polypeptide comprises an amino acid sequence that is at least 90% identical to the sequence of SEQ ID NO: 129 or comprising the sequence of SEQ ID NO: 129. In some embodiments, the isolated polypeptide consists of the sequence of SEQ ID NO: 129.

Also disclosed herein are nucleic acid molecules. In some embodiments, the nucleic acid molecule encodes any of the polypeptides described herein. There are provided vectors comprising any of the nucleic acid molecules disclosed herein. Also disclosed herein are host cells, comprising any of the nucleic acids disclosed herein. Disclosed herein are protein complexes. In some embodiments, the protein complex comprises at least one polypeptide of the disclosure. Also disclosed herein are virus-like particles. In some embodiments, the virus-like particles comprise at least one polypeptide disclosed herein.

Disclosed herein include immunogenic composition for stimulating an immune response in a subject in need thereof. In some embodiments, the immunogenic composition comprises any of: a polypeptide, nucleic acid molecule, host cell, protein complex, and/or virus-like particle disclosed herein; and (ii) a pharmaceutically acceptable carrier.

Disclosed herein include vaccine compositions. In some embodiments, the vaccine composition comprises a carrier associated with a plurality of human immunodeficiency virus (HIV) immunogens. The carrier can be a monovalent or a multivalent carrier.

In some embodiments, the plurality of HIV immunogens are displayed on the surface of the carrier. In some embodiments, the plurality of HIV immunogens are partially embedded in the carrier. In some embodiments, the plurality of HIV immunogens are covalently attached to the carrier. In some embodiments, the plurality of HIV immunogens are conjugated to the carrier. In some embodiments, the plurality of HIV immunogens are attached to the carrier through click chemistry. In some embodiments, the plurality of HIV immunogens are non-covalently attached to the carrier.

In some embodiments, the carrier is selected from: nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the carrier comprises a nanoparticle selected from: lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof. In some embodiments, the carrier comprises a virus-like particle (VLP). In some embodiments, the virus-like particle is Ap205 VLP. In some embodiments, the carrier comprises a self-assembling nanoparticle. In some embodiments, the self-assembling nanoparticle is an i301 nanoparticle or a variant thereof, or a mi3 nanoparticle or a variant thereof. The vaccine composition can comprise a plurality of particle-forming proteins. In some embodiments, one or more of the plurality of particle-forming proteins comprise a 2-dehydro-3-deoxy-phosphogluconate (KDPG) aldolase or a variant thereof.

In some embodiments, an HIV immunogen of the plurality of HIV immunogens is attached to a particle-forming protein of the plurality of particle-forming proteins. In some embodiments, the HIV immunogen of the plurality of HIV immunogens is attached to the particle-forming protein of the plurality of particle-forming proteins through a Spytag/SpyCatcher binding pair. In some embodiments, the HIV immunogen of the plurality of HIV immunogens comprises a Spytag at the C-terminus of the coronavirus antigen and the particle-forming protein of a plurality of particle-forming proteins comprises a SpyCatcher at the N-terminus of the particle-forming protein. In some embodiments, the HIV immunogen of the plurality of HIV immunogens comprises an Env protein or portion thereof, and the Env protein or portion thereof comprises a Spytag at the C-terminus of the Env protein or portion thereof and the particle-forming protein of a plurality of particle-forming proteins comprises a SpyCatcher at the N-terminus of the particle-forming protein.

The vaccine composition can comprise an adjuvant. In some embodiments, the adjuvant is selected from: saponin/MPLA nanoparticles (SMNP), aluminum hydroxide, alhydrogel, AddaVax, MF59, ASO3, Freund's adjuvant, Montanide ISA51, CpG, Poly I:C, glucopyranosyl lipid A, flagellin, resiquimod, and a combination thereof.

In some embodiments, the plurality of HIV immunogens comprise any of the isolated polypeptides disclosed herein. In some embodiments, the plurality of HIV immunogens comprise an amino acid sequence of any of SEQ ID NOs 117-125 and 129-131 or variants thereof. In some embodiments, the plurality of HIV immunogens comprise an isolated polypeptide comprising an amino acid sequence of any of SEQ ID NOs: 119, 122, and 131 or variants thereof. In some embodiments, the plurality of HIV immunogens comprise an isolated polypeptide comprising an amino acid sequence of any of SEQ ID NOs: 118, 121, and 130 or variants thereof. In some embodiments, the plurality of HIV immunogens comprise an isolated polypeptide comprising an amino acid sequence of any of SEQ ID NOs: 117, 120, and 129 or variants thereof.

In some embodiments, the plurality of HIV immunogens comprise three, four, five, six, seven, or eight HIV immunogens. In some embodiments, each of the three, four, five, six, seven, or eight of HIV immunogens is derived from an HIV variant different from the other. In some embodiments, at least one of the plurality of HIV immunogens has a sequence identity of at least 65%, 70%, 75%, 80%, 85%, 90%, 95%, 98%, or 99% with another one of the plurality of HIV immunogens. In some embodiments, each of the plurality of HIV immunogens have a sequence identity of at least 65%, 70%, 75%, 80%, 85%, 90%, 95%, 98%, or 99% with one another. In some embodiments, the plurality of HIV immunogens each comprise an Env protein or a portion thereof, the Env proteins or portions thereof having a sequence identity of at least 65%, 70%, 75%, 80%, 85%, 90%, 95%, 98%, or 99% with one another. In some embodiments, the HIV variant is selected from: X2278, X1632, Troll, CNE55, CE0217, CE1176, BJOX2000, and 398F1.

In some embodiments, the plurality of HIV immunogens each comprise (1) an amino acid sequence having at least 65%, 70%, 75%, 80%, 85%, 90%, 95%, 98%, or 99% sequence identity to an amino acid sequence selected from SEQ ID NOs: 132-139; or (2) an amino acid sequence selected from SEQ ID NOs: 132-139. In some embodiments, each of the plurality of HIV immunogens comprises (1) an amino acid sequence having at least 65%, 70%, 75%, 80%, 85%, 90%, 95%, 98%, or 99% sequence identity to an amino acid sequence selected from SEQ ID NOs: 132-139; or (2) an amino acid sequence selected from SEQ ID NOs: 132-139. In some embodiments, each of the three, four, five, six, seven, or eight HIV immunogens comprise (1) an amino acid sequence having at least 65%, 70%, 75%, 80%, 85%, 90%, 95%, 98%, or 99% sequence identity to an amino acid sequence selected from SEQ ID NOs: 132-139; or (2) an amino acid sequence selected from SEQ ID NOs: 132-139.

Disclosed herein include kits. In some embodiments, the kit comprises any of the immunogenic compositions of or the vaccine compositions described herein.

Disclosed herein include methods of stimulating an immune response in a subject in need thereof. In some embodiments, the method comprises: administering to the subject one or more vaccine compositions disclosed herein, thereby stimulating an immune response in the subject.

Disclosed herein include methods for treating or preventing an HIV infection in a subject in need thereof. In some embodiments, the method comprises: administering to the subject one or more vaccine compositions disclosed herein, thereby treating or preventing the HIV infection in the subject.

Disclosed herein include methods of treating or preventing a disease or disorder caused by an HIV infection in a subject in need thereof. In some embodiments, the method comprises: administering to the subject one or more of vaccine compositions disclosed herein, thereby treating or preventing the disease or disorder caused by the HIV infection in the subject.

In some embodiments, the one or more vaccine compositions are administered to the subject two or more times. In some embodiments, administering the one or more vaccine compositions induces a polyclonal serum response in the subject. In some embodiments, administering the one or more vaccine compositions induces broadly neutralizing responses in the subject against one or more HIV variants. In some embodiments, administering the one or more vaccine compositions boosts a neutralizing antibody response in the subject.

In some embodiments, administering the one or more vaccine compositions comprises administering to the subject a first vaccine composition and administering to the subject a second vaccine composition. In some embodiments, the first vaccine composition comprises the vaccine composition comprising an isolated polypeptide comprising an amino acid sequence of any of SEQ ID NOs: 117, 120, and 129 or variants thereof. In some embodiments, the second vaccine composition comprises the vaccine composition comprising an isolated polypeptide comprising an amino acid sequence of any of SEQ ID NOs: 118, 121, and 130 or variants thereof. In some embodiments, administration of the second vaccine composition to the subject occurs about two, three, four, or five weeks after administration of the first vaccine composition to the subject.

In some embodiments, administering the one or more vaccine compositions further comprises administering a third, fourth, and fifth vaccine composition. In some embodiments, the third vaccine composition comprises the vaccine composition comprising an isolated polypeptide comprising an amino acid sequence of any of SEQ ID NOs: 119, 122, and 131 or variants thereof. In some embodiments, the fourth and fifth vaccine compositions comprise the vaccine composition comprising an isolated polypeptide comprising an amino acid sequence of any of SEQ ID NOs: 132-139. In some embodiments, administration of the third vaccine composition to the subject occurs about two, three, four, or five weeks after administration of the second vaccine composition to the subject. In some embodiments, administration of the fourth vaccine composition to the subject occurs about two, three, four, or five weeks after administration of the third vaccine composition to the subject. In some embodiments, administration of the fifth vaccine composition to the subject occurs about two, three, four, or five weeks after administration of the fourth vaccine composition to the subject.

In some embodiments, the neutralizing antibody response in the subject is characterized by at least a 2-fold increase in neutralizing titer following administration of the second vaccine composition as determined by a pseudo-virus neutralization assay. In some embodiments, as compared to the subject prior to or after administration of the first vaccine composition to the subject.

In some embodiments, administration of the first and second vaccine compositions results in at least a 2-fold increase in the number of antibodies from the serum of the subject capable of specifically binding to a CD4 binding site epitope of an Env protein. In some embodiments, as compared to the subject prior to or after administration of the first vaccine composition.

In some embodiments, administration of the first and second vaccine compositions results in at least a 2-fold increase in the number of antibodies from the serum of the subject capable of binding to one or more of a polypeptide each selected from IGT1, IGT2, and variants thereof. In some embodiments, administration of the first and second vaccine compositions results in at least a 2-fold increase in the number of antibodies from the serum of the subject capable of binding to one or more of a polypeptide each comprising an amino acid sequence selected from SEQ ID NOs: 117-118 and 120-121. In some embodiments, as compared to the subject prior to or after administration of the first vaccine composition.

In some embodiments, the neutralizing antibody response in the subject is characterized by neutralization of two or more pseudo-viruses comprising an Env protein or portion thereof, each of an HIV variant different from one another, by the sera of the subject, following administration of the fifth vaccine composition. In some embodiments, neutralization is defined as having a percent neutralization of about 40% or more at a serum dilution of about 1:100, as measured by a pseudo-virus neutralization assay.

In some embodiments, administration of the first, second, third, fourth, and fifth vaccine compositions results in at least a 0.5-fold increase in the number of antibodies from the serum of the subject capable of specifically binding to a CD4 binding site epitope of an Env protein. In some embodiments, as compared to the subject prior to or after administration of the first vaccine composition.

In some embodiments, administration of the first, second, third, fourth, and fifth vaccine compositions results in at least a 0.5-fold increase in the number of antibodies from the serum of the subject capable of binding to one or more of a polypeptide each selected from IGT1, IGT2, 426c, and variants thereof. In some embodiments, administration of the first, second, third, fourth, and fifth vaccine compositions results in at least a 0.5-fold increase in the number of antibodies from the serum of the subject capable of binding to one or more of a polypeptide each comprising an amino acid sequence selected from SEQ ID NOs: 117-125. In some embodiments, as compared to the subject prior to or after administration of the first vaccine composition.

In some embodiments, administration of the first, second, third, fourth, and fifth vaccine compositions results in at least a 0.5-fold increase in the number of antibodies from the serum of the subject capable of binding one or more of a polypeptide each comprising an HIV Env protein selected from BG505 Env protein, AMCO11 Env protein, B41 Env protein, CH119 Env protein, CE0217 Env protein, CNE8 Env protein, CNE8 N276A Env protein, CNE20 Env protein, CNE20 N276A Env protein, and variants thereof. In some embodiments, as compared to the subject prior to or after administration of the first vaccine composition.

In some embodiments, administration of the first, second, third, fourth, and fifth vaccine compositions results in at least a 0.5-fold increase in the number of antibodies from the serum of the subject capable of binding one or more of a polypeptide each comprising an amino acid sequence selected from SEQ ID NOs: 140-148. In some embodiments, as compared to the subject prior to or after administration of the first vaccine composition.

In some embodiments, administering the first vaccine composition is a prime and administration of the second vaccine composition is a boost. In some embodiments, administering the first vaccine composition is a prime and administration of the second, third, fourth and fifth vaccine compositions are each a boost. In some embodiments, the administration comprises intravenous, intraperitoneal or subcutaneous administration. In some embodiments, the subject is a mammal. In some embodiments, the mammal is a mouse, a rat, a rabbit, or a primate. In some embodiments, the primate is a rhesus macaque, a cynomolgus macaque, a pigtail macaque, an ape, or a human.

Disclosed herein are antibodies or fragments thereof. In some embodiments, the antibody or fragment thereof has specificity to a CD4 binding site of an HIV Env protein and comprises: (a) a heavy chain variable region (VH) CDR1 comprising an amino acid sequence selected from SEQ ID NOs: 203-210 or a variant thereof having a single substitution, deletion or insertion from any one of SEQ ID NOs: 203-210; (b) a VH CDR2 comprising an amino acid sequence selected from SEQ ID NOs: 213-220 or a variant thereof having a single substitution, deletion or insertion from any one of SEQ ID NOs: 213-220; (c) a VH CDR3 comprising an amino acid sequence selected from SEQ ID NOs: 222-229 or a variant thereof having a single substitution, deletion or insertion from any one of SEQ ID NOs: 222-229; (d) a light chain variable region (VL) CDR1 comprising an amino acid sequence selected from SEQ ID NOs: 230 and 232-238 or a variant thereof having a single substitution, deletion or insertion from any one of SEQ ID NOs: 230 and 232-238; (e) a VL CDR2 comprising an amino acid sequence selected from SEQ ID NOs: 239 and 241-245, or a variant thereof having a single substitution, deletion or insertion from any one of SEQ ID NOs: 239 and 241-245; and (f) a VL CDR3 comprising an amino acid sequence selected from SEQ ID NOs: 248-254 or a variant thereof having a single substitution, deletion or insertion from any one of SEQ ID NOs: 248-254.

The antibody or fragment thereof can comprise a heavy chain variable region comprising (i) an amino acid sequence selected from SEQ ID NOs: 3-11, (ii) an amino acid sequence having at least 90% sequence identity to an amino acid sequence selected from SEQ ID NOs: SEQ ID NOs: 3-11, or (iii) an amino acid sequence having one, two or three mismatches relative to an amino acid sequence selected from SEQ ID NOs: 3-11. The antibody or fragment thereof can comprise a light chain variable region comprising (i) an amino acid sequence selected from SEQ ID NOs: 14-22, (ii) an amino acid sequence having at least 90% sequence identity to an amino acid sequence selected from SEQ ID NOs: 14-22, or (iii) an amino acid sequence having one, two or three mismatches relative to an amino acid sequence selected from SEQ ID NOs: 14-22.

In some embodiments, the antibody or fragment thereof comprises an Fc domain. In some embodiments, the antibody or fragment thereof is a single-chain variable fragment (scFv), a single-domain antibody, an immunoglobulin molecule, a monoclonal antibody, a chimeric antibody, a CDR-grafted antibody, a humanized antibody, a Fab fragment, a Fab′ fragment, a F(ab′)₂ fragment, an Fv fragment, a disulfide linked Fv, an scFv, a single domain antibody, a diabody, a multispecific antibody, a dual specific antibody, an anti-idiotypic antibody, a bispecific antibody, or a functionally active epitope-binding fragment thereof.

In some embodiments, the antibody or fragment thereof inhibits infectivity of a virus comprising an HIV Env protein with an IC50 less than 100 μg/mL, less than 10 μg/mL, less than 1 μg/mL or less than 0.1 μg/mL. In some embodiments, as measured by a pseudo-virus neutralization assay. In some embodiments, the antibody or fragment thereof inhibits infectivity of two or more viruses each comprising a different HIV Env protein. In some embodiments, as measured by a pseudo-virus neutralization assay. In some embodiments, the antibody or fragment thereof inhibits infectivity of at least one, at least two, or all of the two or more viruses with an IC50 less than 100 μg/mL, less than 10 μg/mL, less than 1 μg/mL, or less than 0.1 μg/mL. In some embodiments, the antibody or fragment thereof inhibits infectivity of at least one, at least two, or all of the two or more viruses with an IC50 of about 0.01 μg/mL to about 100 μg/mL, about 0.01 μg/mL to about 10 μg/mL, or about 0.01 μg/mL to about 1 μg/mL.

In some embodiments, the HIV Env protein comprises an Env protein of an HIV variant selected from 426c, 426 N276A, CNE20, CNE20 N276A, JRCSF, YU2, PVO.4, Q23.17, Q842.D12, BG505/T332N, ZM214M.PL15, WITO4160.33, and 25710.

Also disclosed herein are polynucleotides encoding one or more of the antibody or fragment thereof described herein. Disclosed herein include isolated cells. In some embodiments, the isolated cell comprises the polynucleotide encoding one or more of the antibody or fragment thereof described herein. Disclosed herein include compositions. In some embodiments, the composition comprises any of the antibody or fragment thereof disclosed herein and a pharmaceutically acceptable carrier, polynucleotides encoding one or more of the antibody or fragment thereof described herein, and/or a polynucleotide encoding one or more of the antibody or fragment thereof described herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A-FIG. 1F show non-limiting exemplary data related to the design and characterization of IOMA-iGL targeting immunogens. FIG. 1A shows an overview of strategy to engineer and test immunogens designed to elicit IOMA-like antibodies. FIG. 1B displays residues selected from the unmutated starting protein 426c.TM4 gp120 that were mutated in yeast display library (left), FACS summary (top, right), and SPR data for highest affinity immunogen selected from each library as gp120 (bottom, right, 1^{st} panel) and SOSIP (bottom, right, 2^{nd} panel) are shown. Same analysis for Library 1 is shown in FIG. 1C, and for Library 2 in FIG. 1D. Representative sensorgrams are shown, with the 1:1 binding model fits shown in black. IgG was immobilized to the CM5 chip and gp120 at varying concentrations (dilutions in various shading) was flowed over the chip surface (IGT2 gp120: 4.9 nM-5,000 nM; IGT1 gp120: 2.3 nM-150,000 nM; 426c.TM4 gp120: 7,000 nM-609,000 nM; IGT2 SOSIP: 31 nM-2,000 nM; IGT1 SOSIP: 78 nM-10,000 nM; 426c degly2 SOSIP: 313 nM-40,000 nM). FIG. 1E displays ELISA data demonstrating binding of CD4bs IgGs to various Env proteins. Bars indicate mean and 95% confidence interval. FIG. 1F shows a representative negative stain EM micrographs of unconjugated SpyCatcher003-mi3 nanoparticles (left) and IGT2-SpyTag SOSIP conjugated to SpyCatcher003-mi3 nanoparticles (right). Scale bar is 50 nm.

FIG. 2A-FIG. 2H display non-limiting exemplary data showing that sequential immunization with IOMA-targeting immunogens elicits heterologous neutralizing serum responses in IOMA iGL transgenic mice. FIG. 2A displays a schematic and timeline of immunization regimen for IOMA iGL knock-in mice. FIG. 2B-FIG. 2F show serum ELISA binding at the indicated time points for IGT2 and IGT2 KO (FIG. 2B), IGT1 and IGT1 KO (FIG. 2C), 426c D279N, 426c, and 426c KO (FIG. 2D), and to a panel of wild-type (wt) and N276A-versions of SOSIP-based Envs (FIG. 2E-FIG. 2F). FIG. 2G displays serum neutralization activity against a panel of 18 HIV pseudoviruses and a murine leukemia virus (MLV) control after terminal bleed. FIG. 2H displays 426c-binding serum IgG ELISA using serum samples isolated from mice at the end of the different sequential immunization regimens indicated underneath and detailed in FIG. 9A-FIG. 9B. Animal immunization studies were performed as 3 independent experiments. Each dot represents results from one mouse. Bars indicate mean and 95% confidence interval. AUC, area under the curve. n, number of animals. m8, mosaic8.

FIG. 3A-FIG. 3G display non-limiting exemplary data showing that monoclonal antibodies cloned from IOMA iGL transgenic mice neutralize heterologous HIV strains. FIG. 3A displays graphs showing the total number of V region (excluding CDR3) amino acid mutations in HC (top) and LC (bottom) of all antibody sequences (x-axis) vs. the number of mutations that are identical or chemically equivalent to mutations in IOMA for AA positions where IOMA and IOMA iGL differ (y-axis). Sequences derived from IOMAgl mice HP1, HP3 and ES30 from immunization group 1 (dark dots) and baseline human VH1-2*01 or VL2-23*02 sequences from peripheral blood of HIV-negative human donors (gray dot). The size of the dot is proportional to the number of sequences. Number of sequences (n), determination coefficient (Pearson, R²) and linear regression lines are indicated. Chemical equivalence classified in 7 groups as follows: (1) G=A=V=L=I; (2) S=T; (3) C=M; (4) D=N=E=Q; (5)_R=K=H; (6) F=Y=W; (7) P. FIG. 3B displays neutralization titers (IC₅₀s) of nine representative monoclonal antibodies isolated from IOMA iGL transgenic mice against a panel of 14 viruses and an MLV control. IC₅₀s for IOMA are shown on the far left. FIG. 3C is a 3D plot showing neutralization activity (coded by shading, some also noted with * or # as indicated), total number of amino acid mutations in both HC and LC V(D)Js (x-axis), and the number of mutations that are identical or chemically equivalent to mutations in the IOMA (y-axis) for all Env-binding monoclonal antibodies from IOMAgl mice HP1, HP3, and ES30 from immunization group 1. Chemical equivalence is as in FIG. 3A. For each antibody, a neutralization score was calculated (See, Example 1 below). Number of sequences (n) are indicated. FIG. 3D shows residues mutated from IOMA iGL shown as spheres mapped onto the crystal structure of mature IOMA (shown in cartoon representation) bound to BG505 gp120 (depicted in surface representation) (PDB 5T3Z). somatic hypermutations (SHMs) are depicted for mature IOMA (left panel) as well as two antibodies isolated from IOMAgl mice: the more potent IO-010 (middle panel) and weaker IO-040 (right panel). FIG. 3E displays total SHMs for mature IOMA (left panel) or SHMs found in the IOMA-gp120 interface (right panel) are colored according to their percentages of occurrence from green to magenta (left panel). Structures are depicted as in FIG. 3D. Shown in FIG. 3F are exemplary key mutations essential for IOMA binding to Env that were elicited in our immunization strategy are mapped onto antibody IO-010 and highlighted in each inset box. IO-010 depicted as in FIG. 3D. Each inset represents a different interaction between IOMA and gp120. FIG. 3G shows amino acid sequence alignment of IOMA V_{H} and V_{L} and monoclonal antibodies from FIG. 3B with IOMA iGL as a reference.

FIG. 4A-FIG. 4F display non-limiting exemplary data showing that sequential immunization with IOMA-targeting immunogens elicits CD4bs-specific responses and heterologous neutralizing serum responses in wildtype mice. FIG. 4A shows a schematic and timeline of immunization regimen for wild-type (WT) mice. FIG. 4B shows serum ELISA binding at the indicated time points to IGT1 or IGT1 CD4bs-KO (KO). FIG. 4C shows serum ELISA binding to anti-idiotypic monoclonal antibodies raised against IOMA iGL (left, 3D3) and IOMA iGL+mature IOMA (right, 3D7). Mean±SEM of 9 to 16 mice per time point are depicted. FIG. 3D-FIG. 3E show serum ELISA binding at the indicated time points to a panel of wt and N276A-versions of SOSIP-based Envs. FIG. 3F shows serum neutralization against a panel of 18 viruses and an MLV control at week 23 of wt mice. Animal immunization studies were performed as 3 independent experiments. Each dot represents results from one mouse. Bars indicate mean and 95% confidence interval. AUC, area under the curve.

FIG. 5A-FIG. 5C display non-limiting exemplary data showing that Prime-boost with IGT2-IGT1 elicits CD4bs-specific responses and potent autologous neutralization in rabbits and rhesus macaques. FIG. 5A shows a schematic and timeline of immunization regimen for rabbits and rhesus macaques. FIG. 5B shows serum ELISA binding to IGT1 and IGT1 KO for rabbits (top) and rhesus macaques (bottom). FIG. 5C shows serum neutralization ID₅₀s of IGT2 and IGT1 pseudoviruses for rabbits (top) and rhesus macaques (bottom). The dotted line at y=10² indicates the lowest dilution evaluated. Significance was demonstrated using a paired t test (p≤0.05).

FIG. 6A-FIG. 6F show non-limiting exemplary data related to the development and characterization of IGT1 and IGT2 immunogens. FIG. 6A shows amino acid alignment of IOMA and VRC01 to their respective germline V genes. FIG. 6B displays representative SPR sensorgrams demonstrating no detectable binding of IOMA iGL to previously described immunogens (eOD-GT8, 426c.TM4, BG505.v4.1-GT1). This experiment was performed to qualitatively evaluate binding of IGT2 and previously described CD4bs immunogens to IOMA iGL rather than to derive affinity or kinetic constants. FIG. 6C shows neutralization titers (IC₅₀s) of IOMA and IOMA iGL against a panel of 38 viruses and an MLV control. FIG. 6D displays a 2.07 A crystal structure of IOMA iGL Fab shown in two views. Shown in FIG. 6E is a structural overlay of IOMA iGL Fab and IOMA Fab from BG505-bound structure (PDB 5T3Z). FIG. 6F shows flow cytometric analysis of yeast cells expressing 426c.TM4 starting protein (left in each sheet), Library 1 (middle in each sheet), or Library 2 (right in each sheet) stained with IOMA iGL IgG/anti-IgG AF647 (x-axis) and anti-cMyc AF488 (y-axis). FIG. 6G displays representative size exclusion chromatography profiles and Coomassie-stained SDS-PAGE analysis for 426c.TM4 gp120, IGT1 gp120, and IGT2 gp120, 426c SOSIP, IGT1 SOSIP, and IGT2 SOSIP demonstrating that all of these proteins are monodispersed samples and that the selected mutations do not alter the stability or behavior of the immunogens compared to the starting proteins. FIG. 6H shows a coomassie-stained SDS-PAGE analysis for mi3, IGT2, IGT2-mi3, IGT1, and IGT1-mi3 under non-reducing and reducing conditions. FIG. 6I displays exemplary SPR sensorgrams demonstrating binding of IGT2 (dashed line) and IGT2-mi3 (solid line) to, from left to right, IOMA iGL IgG, VRC01 iGL IgG, 3BNC60 iGL IgG, and BG24 iGL IgG. IgG was immobilized to the CM5 chip and 1 μM SOSIP or 1 μM SOSIP-mi3 was flowed over the chip surface. FIG. 6J shows representative ELISA binding curves measuring binding of 426c.TM4 gp120, IGT1 gp120, and IGT2 gp120 to the same iGL IgGs as in FIG. 6I. Dots indicate mean and error bars indicate 95% confidence interval.

FIG. 7A-FIG. 7E show non-limiting exemplary cartoons and data related to the targeting strategy and characterization of IOMAgl mice. FIG. 7A shows that in Igh^{IOMAiGL} mice Ighd4-1 to Ighj4 are replaced by a self-excising Neomycin cassette followed by the mouse Ighv9-4 promoter, a leader sequence (L) followed by the iGL version of the IOMA HC VDJ sequence and a Ighj1 splice donor sequence. FIG. 7B shows that in Igk^{IOMAiGL} mice Igkj1 to Igkj5 are replaced by a self-excising Neomycin cassette followed by a mouse Igkv3-12 promoter, a leader sequence followed by the iGL version of the IOMA lambda LC VDJ sequence and a Igkj5 splice donor sequence. DTA, diphtheria toxin A. FIG. 7C shows flow cytometric analysis of B cell development in the bone marrow of control (C57BL/6J) or IOMAgl (Igh^{IOMAiGL/IOMAiGL} Igk^{IOMAiGL/IOMAiGL}) mice. FIG. 7D shows absolute cell number quantification from FIG. 7C. FIG. 7E displays geometric mean fluorescence intensity (gMFI) of IgD in mature recirculating B cells from the bone marrow. Shown in FIG. 7F is flow cytometric analysis of peripheral B cell development in the spleens of control (C57BL/6J) or IOMAgl mice. FIG. 7G displays absolute cell number quantification from FIG. 7F. FIG. 7H shows data related to gMFI of IgD in marginal zone and follicular B cell. MZ, marginal zone B cells; MZP, marginal zone precursors; FOB, follicular B cells. Data from 1 of 2 independent experiments, each dot represents a data from 1 mouse. Bars represent mean±SEM. Statistical analysis used unpaired t test.

FIG. 8A-FIG. 8X display non-limiting exemplary data related to serum neutralization from immunized mice. Neutralization curves of serum isolated from IOMA iGL transgenic mice (FIG. 8A-FIG. 8M) or C57BL/6J wildtype mice (FIG. 8N-FIG. 8X) against the following HIV strains or control MuLV: (FIG. 8A, FIG. 8N) CNE8, (FIG. 8B, FIG. 8O) CNE8 N276A, (FIG. 8C, FIG. 8P) CNE20, (FIG. 8D, FIG. 8Q) CNE20 N276A, (FIG. 8E, FIG. 8R) PVO.4, (FIG. 8F, FIG. 8U) Q23.17, (FIG. 8G, FIG. 8T) WITO4160.33, (FIG. 8H) YU2, (FIG. 8I) JRCSF, (FIG. 8J, FIG. 8V) 6535.5, (K) 3415_V1_C1, (FIG. 8L) CAAN5342.A2, (FIG. 8M, FIG. 8X) MuLV, (FIG. 8S) Q842.D12 and (FIG. 8W) BG505. Naïve serum was also tested against the same strains when available. Note that sera which showed neutralization activity of <40% as listed in Table 3 are presented in FIG. 2G as white rectangles; several of these sera neutralized strains above background including ET33 against PVO.4; ET34 against CNE20 N276A and Q23.17; HP1 against CNE8 N276A, CNE20, and WITO4160.33; HP2 against Q23.17; HP3 against Q23.17 and PVO.4; HP4 against CNE8 N276A, CNE20 N276A, and PVO.4.

FIG. 9A-FIG. 9B show non-limiting schematics and data related to screening immunization regimens to determine the optimal boosting strategy. FIG. 9A shows schematics and timelines of immunization strategies to determine the optimal regimen to elicit IOMA-like bNAbs. FIG. 9B shows serum ELISA binding to 426c degly2 represented as AUC using serum samples isolated from mice at the end of the regimen. m8, mosaic8.

FIG. 10A-FIG. 10C display non-limiting data and cartoons related to cell sorting strategies and sorting controls. FIG. 10A shows representative full gating of cell sorts for single cell Bait++BaitKO⁻ B cell cloning and 10× Genomics next generation VDJ sequencing of bulk-sorted GC B cells from splenic and mesenteric lymph nodes. Baits used were 426c degly2 D279N or CNE8 N276A with 426c degly2 D279N-CD4bs KO, the former is shown. FIG. 10B shows flow cytometry analysis of induction of germinal center response and wt SOSIP-binding cells by immunization regimen (group 1). Naïve IOMAgl mouse splenocytes and IOMA-expressing RAMOS cells served as negative and positive control, respectively. FIG. 10C displays the gene editing strategy to generate IOMA-expressing RAMOS cells. Simultaneous targeting of IgH, IgK and IgL loci with CRISPR/Cas9 to delete endogenous LCs and edit a promoterless tricistronic expression cassette into the IgH locus to express IOMA on the surface of RAMOS cells. A polycistronic mRNA was created using T2A and P2A sequences to induce ribosomal skipping.

FIG. 11A-FIG. 11B display amino acid alignments of selected IOMAgl mouse-derived antibodies. FIG. 11A shows V_{H} alignment of cloned antibodies IO-001 to IO-067 that were expressed and tested for Env binding. IOMA iGL and IOMA sequence at the bottom as reference. Mouse ID and population sorted are indicated. Differences to IOMA iGL are highlighted using chemically shading; dots indicate identical residues to IOMA iGL. Kabat numbering and percent identity of residues are indicated on top. Domains and residues of structural importance are annotated below. FIG. 11B is as above for FIG. 11A but corresponding V_{L} alignment.

FIG. 12A-FIG. 12E depict data showing next generation single cell VDJ analysis determines the extent of mutations in germinal centers of IOMAgl mice. FIG. 12A displays clonal analysis of paired HC and LC sequences from splenic and mesenteric lymph node germinal center B cells of IOMAgl mouse HP3. FIG. 12B show isotype distribution among these cells. FIG. 12C displays the frequency distribution of the number of amino acid mutations to IOMA iGL in the HC sequences of these cells. FIG. 12D shows the frequency distribution of the number of amino acid mutations to IOMA iGL in the LC sequences of these cells. FIG. 12E shows the frequency distribution of the number of amino acid mutations to IOMA iGL in the paired HC and LC sequences of these cells.

FIG. 13A-FIG. 13B display non-limiting exemplary data showing monoclonal antibodies cloned from IOMA iGL transgenic mice bind to heterologous Envs. FIG. 13A shows the area under the curve (AUC) of ELISA binding curves of selected monoclonal antibodies isolated from IOMA iGL knock-in mice to BG505, CE0217, CNE20 and CNE20 N276A SOSIPs. FIG. 13B shows an exemplary comparison of the occurrence frequency of key mutations among IOMA-like antibody sequences selected for cloning and VRC01-class antibody sequences from reference 37 at different time points throughout the respective sequential immunization regimen. Mutations essential for IOMA-class antibody binding to gp120 are listed first, while mutations essential for VRC01-class antibody binding to gp120 are listed second in brackets. Values for each residue represent the percentage of antibodies containing one of the essential mutations at that position.

FIG. 14 depicts an alignment generated in Clustal Omega between the HIV HXB2CG Env protein and the 426c.TM4 gp120 immunogen sequences. In some embodiments, DNA and protein sequence numbering across HIV variants is relative to the HXB2CG strain. Exemplary residues modified to generate the HIV immunogens disclosed herein are indicated by an arrow.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include isolated polypeptides. In some embodiments, the isolated polypeptide comprises an amino acid sequence that is at least 90% identical to the sequence of SEQ ID NO: 119, wherein the isolated polypeptide comprises at least an amino acid mutation in a position corresponding to D279, V430, D460, T461, T462, D463, or N464 of SEQ ID NO: 119. The isolated polypeptide can comprise an amino acid sequence that is at least 90% identical to the sequence of SEQ ID NO: 118 or comprising the sequence of SEQ ID NO: 118. The isolated polypeptide can comprise an amino acid sequence that is at least 90% identical to the sequence of SEQ ID NO: 117 or comprising the sequence of SEQ ID NO: 117.

In some embodiments, the isolated polypeptide comprises an amino acid sequence that is at least 90% identical to the sequence of SEQ ID NO: 122, wherein the isolated polypeptide comprises at least an amino acid mutation in a position corresponding to D279, V430, D460, T461, T462, D463, and N464 of SEQ ID NO: 122. In some embodiments, the isolated polypeptide comprises an amino acid sequence that is at least 90% identical to the sequence of SEQ ID NO: 121 or comprising the sequence of SEQ ID NO: 121. In some embodiments, the isolated polypeptide comprises an amino acid sequence that is at least 90% identical to the sequence of SEQ ID NO: 120 or comprising the sequence of SEQ ID NO: 120.

In some embodiments, the isolated polypeptide comprises an amino acid sequence that is at least 90% identical to the sequence of SEQ ID NO: 131 or comprising the sequence of SEQ ID NO: 131. In some embodiments, the isolated polypeptide comprises an amino acid sequence that is at least 90% identical to the sequence of SEQ ID NO: 130 or comprising the sequence of SEQ ID NO: 130. In some embodiments, the isolated polypeptide comprises an amino acid sequence that is at least 90% identical to the sequence of SEQ ID NO: 129 or comprising the sequence of SEQ ID NO: 129.

Also disclosed herein are nucleic acid molecules. In some embodiments, the nucleic acid molecule encodes any of the polypeptides described herein. There are provided vectors comprising any of the nucleic acid molecules disclosed herein. Also disclosed herein are host cells, comprising any of the nucleic acids disclosed herein. Disclosed herein are protein complexes. In some embodiments, the protein complex comprises at least one polypeptide of the disclosure. Also disclosed herein are virus-like particles. In some embodiments, the virus-like particles comprise at least one polypeptide disclosed herein.

Disclosed herein include immunogenic composition for stimulating an immune response in a subject in need thereof. In some embodiments, the immunogenic composition comprises any of: a polypeptide, nucleic acid molecule, host cell, protein complex, and/or virus-like particle disclosed herein; and (ii) a pharmaceutically acceptable carrier.

Disclosed herein include vaccine compositions. In some embodiments, the vaccine composition comprises a carrier associated with a plurality of human immunodeficiency virus (HIV) immunogens.

Disclosed herein include kits. In some embodiments, the kit comprises any of the immunogenic compositions of or the vaccine compositions described herein.

Disclosed herein include methods of stimulating an immune response in a subject in need thereof. In some embodiments, the method comprises: administering to the subject one or more vaccine compositions disclosed herein, thereby stimulating an immune response in the subject.

Disclosed herein include methods for treating or preventing an HIV infection in a subject in need thereof. In some embodiments, the method comprises: administering to the subject one or more vaccine compositions disclosed herein, thereby treating or preventing the HIV infection in the subject.

Disclosed herein include methods of treating or preventing a disease or disorder caused by an HIV infection in a subject in need thereof. In some embodiments, the method comprises: administering to the subject one or more of vaccine compositions disclosed herein, thereby treating or preventing the disease or disorder caused by the HIV infection in the subject.

Disclosed herein are antibodies or fragments thereof. In some embodiments, the antibody or fragment thereof has specificity to a CD4 binding site of an HIV Env protein and comprises: (a) a heavy chain variable region (VH) CDR1 comprising an amino acid sequence selected from SEQ ID NOs: 203-210 or a variant thereof having a single substitution, deletion or insertion from any one of SEQ ID NOs: 203-210; (b) a VH CDR2 comprising an amino acid sequence selected from SEQ ID NOs: 213-220 or a variant thereof having a single substitution, deletion or insertion from any one of SEQ ID NOs: 213-220; (c) a VH CDR3 comprising an amino acid sequence selected from SEQ ID NOs: 222-229 or a variant thereof having a single substitution, deletion or insertion from any one of SEQ ID NOs: 222-229; (d) a light chain variable region (VL) CDR1 comprising an amino acid sequence selected from SEQ ID NOs: 230 and 232-238 or a variant thereof having a single substitution, deletion or insertion from any one of SEQ ID NOs: 230 and 232-238; (e) a VL CDR2 comprising an amino acid sequence selected from SEQ ID NOs: 239 and 241-245, or a variant thereof having a single substitution, deletion or insertion from any one of SEQ ID NOs: 239 and 241-245; and (f) a VL CDR3 comprising an amino acid sequence selected from SEQ ID NOs: 248-254 or a variant thereof having a single substitution, deletion or insertion from any one of SEQ ID NOs: 248-254.

Also disclosed herein are polynucleotides encoding one or more of the antibody or fragment thereof described herein. Disclosed herein include isolated cells. In some embodiments, the isolated cell comprises the polynucleotide encoding one or more of the antibody or fragment thereof described herein. Disclosed herein include compositions. In some embodiments, the composition comprises any of the antibody or fragment thereof disclosed herein and a pharmaceutically acceptable carrier, polynucleotides encoding one or more of the antibody or fragment thereof described herein, and/or a polynucleotide encoding one or more of the antibody or fragment thereof described herein.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

“Glycosylation site” as used herein can refer to an amino acid sequence on the surface of a polypeptide, such as a protein, which accommodates the attachment of a glycan. An N-linked glycosylation site is triplet sequence of NXSIT in which N is asparagine, X is any residues except praline, SIT means serine or threonine. A glycan is a polysaccharide or oligosaccharide. Glycan may also be used to refer to the carbohydrate portion of a glycoconjugate, such as a glycoprotein, glycolipid, or a proteoglycan.

The term “antigen”, as used herein, can refer to an entity (e.g., a protein or portion thereof) bound by an antibody or receptor. The terms “immunogen” and “Immunogenic polypeptide”, as used herein may be used interchangeably and can refer to an entity (e.g., a protein or portion thereof) that induces antibody production or binds to a receptor. Where an entity discussed herein is both immunogenic and antigenic, reference to it as either an immunogen or antigen is made according to its intended utility. Administration of an immunogenic polypeptide can lead to protective immunity against a pathogen of interest. In some examples, an immunogenic polypeptide is an antigen that is resurfaced to focus immunogenicity to a target epitope. An “immunogenic gp120 polypeptide” is gp120 molecule, a resurfaced gp120 molecule, or a portion thereof capable of inducing an immune response in a mammal, such as a mammal with or without an HIV infection. Administration of an immunogenic gp120 polypeptide that induces an immune response can lead to protective immunity against HIV.

“Immune response” as used herein can refer to a response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response and results in the production of specific antibodies.

As used herein, the term “Isolated” shall be given its ordinary meaning and shall also refer to an “isolated” biological component (such as a protein, for example, a disclosed antigen or nucleic acid encoding such an antigen) has been substantially separated or purified away from other biological components in which the component naturally occurs, such as other chromosomal and extrachromosomal DNA, RNA, and proteins. Proteins, peptides, and nucleic acids that have been “isolated” include proteins purified by standard purification methods. The term also embraces proteins or peptides prepared by recombinant expression in a host cell as well as chemically synthesized proteins, peptides, and nucleic acid molecules. Isolated (or purified) does not require absolute purity, and can include protein, peptide, or nucleic acid molecules that are at least 50% isolated, such as at least 75%, 80%, 90%, 95%, 98%, 99%, or even 99.9% isolated.

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; Pearson et al., Meth. Mol. Bio. 24:307-31, 1994; and Altschul et al., J. Mol. Biol. 215:403-10, 1990 (the content of each of these references is incorporated herein in its entirety).

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to one of skill in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The term “substantially identical” as used herein in the context of two or more sequences refers to a specified percentage of amino acid residues or nucleotides that are identical or functionally equivalent, such as about, at least or at least about 65% identity, optionally, about, at least or at least about 70%, 75%, 80%, 85%, 90%, 95%, or 99% identity over a specified region or over the entire sequence.

As used herein, the term “variant” can refer to a polynucleotide or polypeptide or viral strain having a sequence substantially similar or identical to a reference (e.g., the parent) polynucleotide or polypeptide, or viral strain. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

As used herein, an “antibody” or “antigen-binding polypeptide” can refer to a polypeptide or a polypeptide complex that specifically recognizes and binds to an antigen (e.g., a spike protein receptor binding domain). An antibody can be a whole antibody and any antigen binding fragment or a single chain thereof. Thus, the term “antibody” includes any protein or peptide-containing molecule that comprises at least a portion of an immunoglobulin molecule having biological activity of binding to the antigen. Examples of such include, but are not limited to, a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, L-RNA aptamers (also known as spiegelmers), and diabodies. The term “antibody fragment” also includes any synthetic or genetically engineered protein that acts like an antibody by binding to a specific antigen to form a complex.

As used herein, a “single-chain variable fragment” or “scFv” refers to a fusion protein of the variable regions of the heavy (V_{H}) and light chains (V_{L}) of immunoglobulins. In some embodiments, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

As used herein, the term “antibody” encompasses various broad classes of polypeptides that can be distinguished biochemically. Those of skill in the art will appreciate that heavy chains are classified as gamma, mu, alpha, delta, or epsilon (γ, μ, α, δ, or ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight approximately 53,000-70,000 Daltons. The four chains are typically joined by disulfide bonds in a “Y” configuration wherein the light chains bracket the heavy chains starting at the mouth of the “Y” and continuing through the variable region.

Antibodies, antigen-binding polypeptides, fragments, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies. Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VL chains (i.e. VH CDR1, VH CDR2, VH CDR3, VL CDR1, VL CDR2 and VL CDR3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those of skill in the art can routinely determine which residues comprise a particular CDR given the variable region amino acid sequence of the antibody.

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

Kabat et al. also defined a numbering system for variable domain sequences that is applicable to any antibody. One of skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: VH CDR1 begins at approximately amino acid 31 (i.e., approximately 9 residues after the first cysteine residue), includes approximately 5-7 amino acids, and ends at the next tryptophan residue. VH CDR2 begins at the fifteenth residue after the end of VH CDR1, includes approximately 16-19 amino acids, and ends at the next arginine or lysine residue. VH CDR3 begins at approximately the thirty third amino acid residue after the end of VH CDR2; includes 3-25 amino acids; and ends at the sequence W-G-X-G, where X is any amino acid. VL CDR1 begins at approximately residue 24 (i.e., following a cysteine residue); includes approximately 10-17 residues; and ends at the next tryptophan residue. VL CDR2 begins at approximately the sixteenth residue after the end of VL CDR1 and includes approximately 7 residues. VL CDR3 begins at approximately the thirty third residue after the end of VL CDR2 (i.e., following a cysteine residue); includes approximately 7-11 residues and ends at the sequence F or W-G-X-G, where X is any amino acid.

Antibodies disclosed herein can be from any animal origin including vertebrates (e.g., birds, reptiles, amphibians, and mammals). In some embodiments, the antibodies are human, murine, donkey, rabbit, goat, guinea pig, camel, llama, horse, or chicken antibodies. In some embodiments, the variable region is condricthoid in origin (e.g., from sharks). In some embodiments, the antibody or fragment thereof is from a mammal.

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CHI domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CH2 regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In some embodiments, the target binding region or site is from a non-human source (e.g. mouse or primate) and the constant region is human.

As used herein, the term “binding” refers to a non-covalent interaction between macromolecules (e.g., between a protein and a nucleic acid or between a first protein and a second protein). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd. Binding interactions can also be characterized by an EC50. As used herein, “EC50” can refer to the concentration of an agent (e.g., an antibody or fragment thereof) which produces 50% of the maximal response possible for that agent. As described herein, binding interactions can be characterized by an EC50 of, or an EC50 less than 10 μg/mL, less than 1 μg/mL, less than 0.1 μg/mL, or less than 0.01 μg/mL.

The term “IC50,” as used herein, can refer to the half-maximal concentration of an antibody or an antigen-binding fragment thereof, which induces an inhibitory response (e.g., reduced infectivity, e.g. neutralization), either in an in vitro or an in vivo assay, which is 50% of the maximal response, i.e., halfway between the maximal response and the baseline. As used herein, “infectivity” shall have its ordinary meaning, and can also refer to the ability of a virus to enter or exit a cell. As described herein, the antibodies or fragments thereof provided herein can reduce, inhibit, block infectivity of a virus at an IC50 of, e.g., less than 10 μg/mL, less than 1 μg/mL, less than 0.1 μg/mL, or less than 0.01 μg/mL.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity (e.g., greater binding affinity) for a given epitope than antibody “B,” or antibody “A” may be said to bind to epitope “C” with a higher specificity (e.g., greater binding affinity) than it has for related epitope “D.”

A successful vaccine against HIV (e.g., HIV-1) would be the most effective way to contain the AIDS pandemic, which is responsible for >36 million deaths in total and 1-2 million new infections each year. Clinical trials of vaccine candidates have revealed disappointing outcomes, and as a result, there is no currently available protective vaccine against HIV-1, in part due to the large number of circulating HIV-1 (e.g., HIV) strains. For the last decade, a major focus of HIV-1 vaccine design has been on eliciting broadly neutralizing antibodies (bNAbs), which neutralize a majority of HIV-1 strains in vitro at low concentrations. Multiple studies have demonstrated that passively administered bNAbs can prevent HIV-1 or simian/human immunodeficiency virus (SHIV) infection, suggesting a vaccination regimen that elicits bNAbs at neutralizing concentrations would be protective.

The HIV-1 Envelope protein (Env), a trimeric membrane glycoprotein comprising gp120 and gp41 subunits that is found on the surface of the virus, is the sole antigenic target of neutralizing antibodies. An impediment to HIV-1 vaccine design is that most inferred germline (iGL) precursors of known bNAbs do not bind with detectable affinity to native Envs on circulating HIV-1 strains. As a result, potential Env immunogens must be modified to bind and select for bNAb precursors in vivo during immunization (e.g., a “germline-targeting” approach). This approach has been used to activate precursors of the VRC01-class of bNAbs that target the CD4 binding site (CD4bs) on gp120. Eliciting VRC01-class bNAbs that target the CD4bs would be desirable due to their breadth and potency. However, the VRC01-class of bNAbs may be difficult to elicit due to their requirement for rare short light chain complementarity region 3 (CDRL3) loops of 5 residues (present in only ˜1% of human antibodies) and many somatic hypermutations (SHMs), including a difficult-to achieve sequence of mutations to sterically accommodate the highly-conserved N276_{gp120} glycan.

Crystal structures of a natively glycosylated HIV-1 soluble Env trimer derived from the clade A BG505 strain (BG505 SOSIP.664) complexed with the CD4bs bNAb IOMA, revealed that this antibody exhibits distinct properties from VRC01-class bNAbs. In common with VRC01-class bNAbs, IOMA is derived from the VH1-2 immunoglobulin heavy chain (HC) gene segment, and it binds Env with a similar overall pose as other VH1-2-derived CD4bs bNAbs, but it is not as potent or broad as many of the VRC01-class antibodies. However, unlike VRC01-class bNAbs, IOMA includes a normal-length (8 residues) CDRL3 and is less mutated with 9.5% HC and 7% light chain (LC) nucleotide mutations to its iGL compared to VRC01 with 30% HC and 19% LC nucleotide mutations. In addition, IOMA accommodates the N276_{gp120} glycan, a roadblock for raising VRC01-class bNAbs, using a relatively easy-to-achieve mechanism involving a short helical CDRL1, and four amino acid changes (including a single mutated glycine) that each require single nucleotide substitutions. By contrast, the CDRL1s of VRC01-class bNAbs include either a 3-6 residue deletion or large numbers of SHMs that introduce multiple glycines and/or other insertions to create flexible CDRL1 loops. Thus, IOMA-like antibodies represent an easier pathway for vaccine induced maturation of CD4bs precursors to mature CD4bs bNAbs.

Provided herein are immunogens engineered to elicit IOMA and other CD4bs bNAbs. Using these immunogens, a sequential immunization strategy was devised that elicited broad heterologous serum neutralization in both IOMA iGL knock-in and wildtype (wt) mouse models. Notably, this was achieved using fewer than half of the immunizations in previous studies. Moreover, IOMA-like bNAbs elicited in knock-in mice were more potent than IOMA against some strains. Finally, the immunization regimen developed in knock-in mice also elicited CD4bs-specific responses in multiple wt animals including mice, rabbits, and rhesus macaques providing a rationale for using the IOMA-targeting immunogens described here as part of an effective HIV vaccine.

### Immunogens

This disclosure provides an immunogen and its variants for stimulating an immune response (e.g., against one or more HIV strains) in a subject in need thereof. In some embodiments, the immunogen includes a portion of the HIV envelope protein, e.g., gp120, which is located on the surface of the HIV. gp120 is the N-terminal segment of the HIV envelope protein gp160, anchored in the membrane bilayer at its carboxyl-terminal region. gp120 protrudes into the aqueous environment surrounding the virion, whereas its C-terminal counterpart, gp41, spans the membrane. The gp120 molecule consists of a polypeptide core of 60,000 daltons, which is extensively modified by N-linked glycosylation to increase the apparent molecular weight of the molecule to 120,000 daltons. The amino acid sequence of gp120 contains five relatively conserved domains interspersed with five hypervariable domains. The positions of the 18 cysteine residues in the gp120 primary sequence and the positions of 13 of the approximately 24 N-linked glycosylation sites in the gp120 sequence are common to all gp120 sequences.

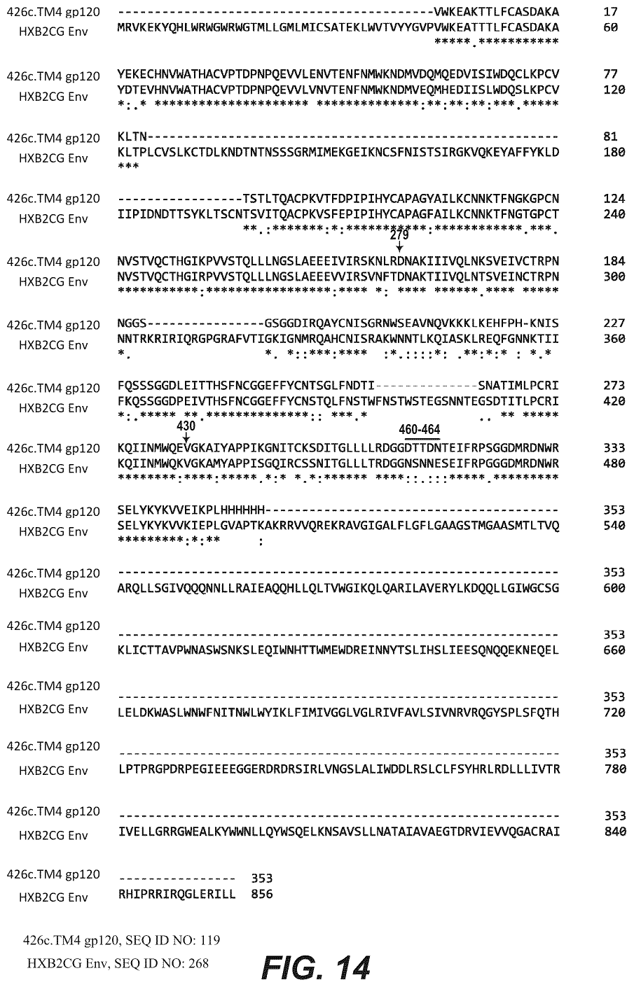

gp120 is an envelope protein from human immunodeficiency virus (HIV). The mature gp120 wild-type polypeptides have about 500 amino acids in the primary sequence. The gp120 is heavily N-glycosylated giving rise to an apparent molecular weight of 120 kD. The polypeptide is comprised of five conserved regions (C1-C5) and five regions of high variability (V1-V5). Exemplary sequences of wild-type gp160 polypeptides are shown on GENBANK®, for example, Accession Nos. AAB05604 and AAD12142, which are incorporated herein by reference in their entirety as available on Jun. 29, 2010. Exemplary sequences of gp120 polypeptides from HIV-1 DU156 are shown on GENBANK®, for example, Accession Nos. ABD83635, AAO50350, and AAT91997, which are incorporated herein by reference in their entirety as available on Sep. 27, 2010. Exemplary sequences of gp120 polypeptides from HIV-1 ZA012 are shown on GENBANK®, for example, Accession No. ACF75939, which is incorporated herein by reference in its entirety as available on Sep. 27, 2010. The numbering used in the gp120 derived HIV immunogens disclosed herein is relative to the HXB2CG numbering scheme as set forth in Korber B, Foley B T, Kuiken C, Pillai S K, Sodroski J G (1998).Numbering Positions in HIV Relative to HXB2CG. pp. III-102-111 in Human Retroviruses and AIDS 1998. Edited by: Korber B, Kuiken C L, Foley B, Hahn B, McCutchan F, Mellors J W, and Sodroski J. Published by: Theoretical Biology and Biophysics Group, Los Alamos National Laboratory, Los Alamos, NM (See, e.g, FIG. 14).

Disclosed herein include isolated polypeptides (e.g., HIV immunogens). In some embodiments, the isolated polypeptide comprises an amino acid sequence that is at least or at least about 90% identical (e.g., 90%, 91%, 92%, 93%, 94%, 95%, 96%, 97%, 98%, 99%, 100%, or a number or a range between any two of these values) to the sequence of SEQ ID NO: 119, wherein the isolated polypeptide comprises at least an amino acid mutation in a position corresponding to D279, V430, D460, T461, T462, D463, or N464 of SEQ ID NO: 119. The mutation can be a deletion, an insertion, or a substitution of one or more amino acids. The mutation can be an amino acid substitution. In some embodiments, the isolated polypeptide comprises a sequence having one, two, three, four, five, six, or seven mismatches relative to the amino acid sequence of SEQ ID NO: 119.

The isolated polypeptide can comprise a D279N substitution and/or a V430P substitution. In some embodiments, isolated polypeptide comprises at least one of a D460N substitution, a T461S substitution, a T462Q substitution, a D463R substitution, and an N464E substitution. The isolated polypeptide can comprise the sequence of SEQ ID NO: 118. The isolated polypeptide can comprise an amino acid sequence that is at least 90% identical (e.g., 90%, 91%, 92%, 93%, 94%, 95%, 96%, 97%, 98%, 99%, 100%, or a number or a range between any two of these values) to the sequence of SEQ ID NO: 118 or comprising the sequence of SEQ ID NO: 118. In some embodiments, the isolated polypeptide consists of the sequence of SEQ ID NO: 118.

In some embodiments, the isolated polypeptide comprises at least one of a D460N substitution, a T461A substitution, a T462L substitution, a D463R substitution, and an N464P substitution. The isolated polypeptide can comprise the sequence of SEQ ID NO: 117. The isolated polypeptide can comprise an amino acid sequence that is at least 90% identical (e.g., 90%, 91%, 92%, 93%, 94%, 95%, 96%, 97%, 98%, 99%, 100%, or a number or a range between any two of these values) to the sequence of SEQ ID NO: 117 or comprising the sequence of SEQ ID NO: 117. In some embodiments, the isolated polypeptide consists of the sequence of SEQ ID NO: 117.

The dissociation constant (K_{D}), e.g., affinity, of a binding protein can be determined, for example, by surface plasmon resonance. Generally, surface plasmon resonance analysis measures real-time binding interactions between ligand (a target antigen on a biosensor matrix) and analyte (a binding protein in solution) by surface plasmon resonance (SPR). Surface plasmon analysis can also be performed by immobilizing the analyte (binding protein on a biosensor matrix) and presenting the ligand (target antigen). The terms “K_{D},” and “affinity” can be used interchangeably, and as used herein refers to the dissociation constant of the interaction between a particular binding protein and a target antigen (e.g., between an immunogen disclosed herein and an antibody). In some embodiments, the isolated polypeptide binds to a neutralizing antibody with an affinity of about 30 μM or less. In some embodiments, the isolated polypeptide binds to a neutralizing antibody with an affinity of about 30 μM. In some embodiments, the isolated polypeptide binds to a neutralizing antibody with an affinity of about 0.5 μM.

In some embodiments, the neutralizing antibody has specificity for a CD4 binding site of an HIV Env protein. The neutralizing antibody can comprise a heavy chain comprising an amino acid sequence selected from SEQ ID NOs: 1, 153, 155, 157, and 159 (ii) an amino acid sequence having at least 90% sequence identity (e.g., 90%, 91%, 92%, 93%, 94%, 95%, 96%, 97%, 98%, 99%, 100%, or a number or a range between any two of these values) to an amino acid sequence selected from SEQ ID NOs: 1, 153, 155, 157, and 159, or (iii) an amino acid sequence having one, two or three mismatches relative to an amino acid sequence selected from SEQ ID NOs: 1, 153, 155, 157, and 159. The neutralizing antibody can comprise a light chain comprising an amino acid sequence selected from SEQ ID NOs: 12, 154, 156, 158, and 160, (ii) an amino acid sequence having at least 90% sequence identity (e.g., 90%, 91%, 92%, 93%, 94%, 95%, 96%, 97%, 98%, 99%, 100%, or a number or a range between any two of these values) to an amino acid sequence selected from SEQ ID NOs: 12, 154, 156, 158, and 160, or (iii) an amino acid sequence having one, two or three mismatches relative to an amino acid sequence selected from SEQ ID NOs: 12, 154, 156, 158, and 160.

In some embodiments, the HIV immunogens comprise the SOSIP mutations: “SOS” substitutions (A501Cgp120, T605Cgp41), “IP” (I559Pgp41), addition of the N-linked glycan sequence at residue 332gp120 (T332Ngp120), an enhanced gp120-gp41 cleavage site, and a stop codon after residue 664gp41. In some embodiments, SOSIP mutations can stabilize Env trimers relative to unmutated or wild-type sequences. In some embodiments, the isolated polypeptide comprises an amino acid sequence that is at least 90% identical (e.g., 90%, 91%, 92%, 93%, 94%, 95%, 96%, 97%, 98%, 99%, 100%, or a number or a range between any two of these values) to the sequence of SEQ ID NO: 122, wherein the isolated polypeptide comprises at least an amino acid mutation in a position corresponding to D279, V430, D460, T461, T462, D463, and N464 of SEQ ID NO: 122. The mutation can be a deletion, an insertion, or a substitution of one or more amino acids. The mutation can be an amino acid substitution. In some embodiments, the isolated polypeptide comprises a sequence having one, two, three, four, five, six, or seven mismatches relative to the amino acid sequence of SEQ ID NO: 122.

The isolated polypeptide can comprise a D279N substitution and/or a V430P substitution. In some embodiments, the isolated polypeptide comprises at least one of a D460N substitution, a T461S substitution, a T462Q substitution, a D463R substitution, and an N464E substitution. The isolated polypeptide can comprise the sequence of SEQ ID NO: 121. In some embodiments, the isolated polypeptide comprises an amino acid sequence that is at least 90% identical (e.g., 90%, 91%, 92%, 93%, 94%, 95%, 96%, 97%, 98%, 99%, 100%, or a number or a range between any two of these values) to the sequence of SEQ ID NO: 121 or comprising the sequence of SEQ ID NO: 121. In some embodiments, the isolated polypeptide consists of the sequence of SEQ ID NO: 121.

In some embodiments, the isolated polypeptide further comprises at least one of a D460N substitution, a T461A substitution, a T462L substitution, a D463R substitution, and an N464P substitution. The isolated polypeptide can comprise the sequence of SEQ ID NO: 120.In some embodiments, the isolated polypeptide comprises an amino acid sequence that is at least 90% identical (e.g., 90%, 91%, 92%, 93%, 94%, 95%, 96%, 97%, 98%, 99%, 100%, or a number or a range between any two of these values) to the sequence of SEQ ID NO: 120 or comprising the sequence of SEQ ID NO: 120. In some embodiments, the isolated polypeptide consists of the sequence of SEQ ID NO: 120.

In some embodiments, the HIV immunogens disclosed herein comprise a sequence (e.g., a tag, e.g., SpyTag) for attachment to a carrier (e.g., Spycatcher). In some embodiments, the isolated polypeptide comprises an amino acid sequence that is at least 90% identical (e.g., 90%, 91%, 92%, 93%, 94%, 95%, 96%, 97%, 98%, 99%, 100%, or a number or a range between any two of these values) to the sequence of SEQ ID NO: 131 or comprising the sequence of SEQ ID NO: 131. In some embodiments, the isolated polypeptide consists of the sequence of SEQ ID NO: 131. In some embodiments, the isolated polypeptide comprises an amino acid sequence that is at least 90% identical (e.g., 90%, 91%, 92%, 93%, 94%, 95%, 96%, 97%, 98%, 99%, 100%, or a number or a range between any two of these values) to the sequence of SEQ ID NO: 130 or comprising the sequence of SEQ ID NO: 130. In some embodiments, the isolated polypeptide consists of the sequence of SEQ ID NO: 130. In some embodiments, the isolated polypeptide comprises an amino acid sequence that is at least 90% identical (e.g., 90%, 91%, 92%, 93%, 94%, 95%, 96%, 97%, 98%, 99%, 100%, or a number or a range between any two of these values) to the sequence of SEQ ID NO: 129 or comprising the sequence of SEQ ID NO: 129. In some embodiments, the isolated polypeptide consists of the sequence of SEQ ID NO: 129.

Also disclosed herein are nucleic acid molecules. In some embodiments, the nucleic acid molecule encodes any of the polypeptides described herein. There are provided vectors comprising any of the nucleic acid molecules disclosed herein. Also disclosed herein are host cells, comprising any of the nucleic acids disclosed herein. Disclosed herein are protein complexes. In some embodiments, the protein complex comprises at least one polypeptide of the disclosure. Also disclosed herein are virus-like particles. In some embodiments, the virus-like particles comprise at least one polypeptide disclosed herein.

Nucleic Acids

Also disclosed herein are nucleic acid molecules. Another aspect of this disclosure features an isolated nucleic acid comprising a sequence that encodes the polypeptide or protein described above. A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

An “isolated nucleic acid” can refer to a nucleic acid the structure of which is not identical to that of any naturally occurring nucleic acid or to that of any fragment of a naturally occurring genomic nucleic acid. The term, therefore, covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), a restriction fragment, or a geneblock; and (d) a recombinant nucleotide sequence that is part of a hybrid gene, i.e., a gene encoding a fusion protein. The nucleic acid described above can be used to express the polypeptide, fusion protein, or antibody described herein. For this purpose, one can operatively link the nucleic acid to suitable regulatory sequences to generate an expression vector.

The nucleic acid and amino acid sequences disclosed herein are shown using standard letter abbreviations for nucleotide bases, and one or three letter code for amino acids. Only one strand of each nucleic acid sequence is shown, but the complementary strand is understood as included by any reference to the displayed strand of, e.g., a dsDNA.

This disclosure also includes vectors containing a coding sequence for the disclosed immunogen, host cells containing the vectors, and methods of making substantially pure immunogen comprising the steps of introducing the coding sequence for the immunogen into a host cell, and cultivating the host cell under appropriate conditions such that the immunogen is produced and secreted. The immunogen so produced may be harvested in conventional ways.

Therefore, the present disclosure also relates to methods of expressing the immunogen and biological equivalents disclosed herein, assays employing these gene products, and recombinant host cells which comprise DNA constructs which express these receptor proteins. The disclosed immunogens may be recombinantly expressed by molecular cloning the nucleic acid encoding the immunogens into an expression vector (such as pcDNA3.neo, pcDNA3.1, pCR2.1, pBlueBacHis2 or pLITMUS28) containing a suitable promoter and other appropriate transcription regulatory elements, and transferred into prokaryotic or eukaryotic host cells to produce the immunogens. Techniques for such manipulations can be found described in Sambrook et al., 1989, Molecular Cloning: A Laboratory Manual; Cold Spring Harbor Laboratory, Cold Spring Harbor, New York, and are well known and readily available to the artisan of ordinary skill in the art. Therefore, another aspect of the present disclosure includes host cells that have been engineered to contain and/or express DNA sequences encoding the immunogens. Such recombinant host cells can be cultured under suitable conditions to produce the disclosed immunogens or a biologically equivalent form. Recombinant host cells may be prokaryotic or eukaryotic, including but not limited to, bacteria such as E. coli, fungal cells such as yeast, mammalian cells including, but not limited to, cell lines of human, bovine, porcine, monkey and rodent origin, and insect cells including but not limited to Drosophila and silkworm derived cell lines. For instance, one insect expression system utilizes Spodoptera frugiperda (Sf21) insect cells (Invitrogen) in tandem with a baculovirus expression vector (pAcG2T, Pharmingen).

Host cells which can be suitable, include but are not limited to, L cells L-M(TK˜) (ATCC CCL 1.3), L cells L-M (ATCC CCL 1.2), Saos-2 (ATCC HTB-85), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171) and CPAE (ATCC CCL 209).

A variety of mammalian expression vectors may be used to express recombinant immunogens in mammalian cells. Expression vectors are defined herein as DNA sequences that are required for the transcription of cloned DNA and the translation of their mRNAs in an appropriate host. Such vectors can be used to express eukaryotic DNA in a variety of hosts such as bacteria, blue-green algae, plant cells, insect cells, and animal cells. Specifically designed vectors allow the shuttling of DNA between hosts such as bacteria-yeast or bacteria-animal cells. An appropriately constructed expression vector should contain: an origin of replication for autonomous replication in host cells, selectable markers, a limited number of useful restriction enzyme sites, a potential for high copy number, and active promoters. A promoter can be defined as a DNA sequence that directs RNA polymerase to bind to DNA and initiate RNA synthesis. A strong promoter is one which causes mRNAs to be initiated at high frequency. Expression vectors may include, but are not limited to, cloning vectors, modified cloning vectors, specifically designed plasmids or viruses. Commercially available mammalian expression vectors which may be suitable for immunogen expression, include but are not limited to, pIRES-hyg (Clontech), pIRES-puro (Clontech), pcDNA3.neo (Invitrogen), pcDNA3.1 (Invitrogen), pCI-neo (Promega), pLITMUS28, pLITMUS29, pLITMUS38 and pLITMUS39 (New England Bioloabs), pcDNAI, pcDNAiamp (Invitrogen), pcDNA3 (Invitrogen), pMClneo (Stratagene), pXTI (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and 1ZD35 (ATCC 37565).

Also, a variety of bacterial expression vectors may be used to express the disclosed immunogens in bacterial cells. Commercially available bacterial expression vectors that may be suitable for immunogen expression include, but are not limited to pCR2.1 (Invitrogen), pETl 1a (Novagen), lambda gtl 1 (Invitrogen), and pKK223-3 (Pharmacia).

In addition, a variety of fungal cell expression vectors may be used to express the immunogens in fungal cells. Commercially available fungal cell expression vectors which may be suitable for recombinant immunogen expression include but are not limited to pYES2 (Invitrogen) and Pichia expression vector (Invitrogen).

Also, a variety of insect cell expression vectors may be used to express a recombinant receptor in insect cells. Commercially available insect cell expression vectors which may be suitable for recombinant expression of the immunogens include but are not limited to pBlueBacIII and pBlueBacHts2 (Invitrogen), and pAcG2T (Pharmingen).

The expression vector may be introduced into host cells via any one of a number of techniques including but not limited to transformation, transfection, protoplast fusion, and electroporation. Transformation is meant to encompass a genetic change to the target cell resulting from incorporation of DNA. Transfection is meant to include any method known in the art for introducing the immunogens into the test cells. For example, transfection includes calcium phosphate or calcium chloride mediated transfection, lipofection, electroporation, as well as infection with, for example, a viral vector such as a recombinant retroviral vector containing the nucleotide sequence which encodes the immunogens, and combinations thereof. The expression vector-containing cells are individually analyzed to determine whether they produce the immunogens. Identification of immunogen expressing cells may be done by several means, including but not limited to immunological reactivity with specific bNAbs, labeled ligand binding and the presence of host cell-associated activity with respect to the immunogens.

Also within the scope of this disclosure is a host cell that contains the above-described nucleic acid. Examples include bacterial cells (e.g., E. coli cells), insect cells (e.g., using baculovirus expression vectors), yeast cells, or mammalian cells. See, e.g., Goeddel, (1990) Gene Expression Technology: Methods in Enzymology 185, Academic Press, San Diego, Calif. To produce a polypeptide of this disclosure, one can culture a host cell in a medium under conditions permitting expression of the polypeptide encoded by a nucleic acid of this disclosure, and purify the polypeptide from the cultured cell or the medium of the cell. Alternatively, the nucleic acid of this disclosure can be transcribed and translated in vitro, e.g., using T7 promoter regulatory sequences and T7 polymerase.

### Vaccine Compositions

Disclosed herein include immunogenic composition for stimulating an immune response in a subject in need thereof. In some embodiments, the immunogenic composition comprises any of: a polypeptide, nucleic acid molecule, host cell, protein complex, and/or virus-like particle disclosed herein; and (ii) a pharmaceutically acceptable carrier.

Disclosed herein include vaccine compositions. In some embodiments, the vaccine composition comprises a carrier associated with a plurality of human immunodeficiency virus (HIV) immunogens, comprising any of the isolated polypeptides described herein. The carrier can be a monovalent or a multivalent carrier.

Carriers

A carrier as used herein can be generally referred to a biocompatible molecular system having the capability of incorporating and transporting molecules (e.g., therapeutic agents such as HIV immunogens) to enhance their selectivity, bioavailability and efficiency. The carriers used in the methods, compositions, and systems herein described can be a biocompatible molecular system, either naturally occurring or synthetic, that can be functionalized or conjugated for coupling (e.g., covalently or non-covalently) to a plurality of protein antigens or immunogen polypeptides described herein. The carriers can comprise nanoparticles, nanotubes, nanowires, dendrimers, liposomes, ethosomes and aquasomes, polymersomes and niosomes, foams, hydrogels, cubosomes, quantum dots, exosomes, macrophages, and others identifiable to a person skilled in the art.

In some embodiments, the carrier used herein can be a nanosized carrier such as a nanoparticle. As used herein, the term “nanoparticle” can refer to a nanoscopic particle having a size measured in nanometers (nm). Size of the nanoparticles may be characterized by their maximal dimension. The term “maximal dimension” as used herein can refer to the maximal length of a straight line segment passing through the center of a nanoparticle and terminating at the periphery. In the case of nanospheres, the maximal dimension of a nanosphere corresponds to its diameter. The term “mean maximal dimension” can refer to an average or mean maximal dimension of the nanoparticles, and may be calculated by dividing the sum of the maximal dimension of each nanoparticle by the total number of nanoparticles. Accordingly, value of maximal dimension may be calculated for nanoparticles of any shape, such as nanoparticles having a regular shape such as a sphere, a hemispherical, a cube, a prism, or a diamond, or an irregular shape. The nanoparticles provided herein need not be spherical and can comprise, for example, a shape such as a cube, cylinder, tube, block, film, and/or sheet. In some embodiments, the maximal dimension of the nanoparticles is in the range from about 1 nm to about 5000 nm, such as between about 20 nm to about 1000 nm, about 20 nm to about 500 nm, about 50 nm to about 300 nm, or about 100 nm to about 200 nm.

The nanoparticle can be, but is not limited to, any one of lipid-based nanoparticles (nanoparticles where the majority of the material that makes up their structure are lipids, e.g., liposomes or lipid vesicles), polymeric nanoparticles, inorganic nanoparticles (e.g., magnetic, ceramic and metallic nanoparticles), surfactant-based emulsions, silica nanoparticles, virus-like particles (particles primarily made up of viral structural proteins that are not infectious or have low infectivity), peptide or protein-based particles (particles where the majority of the material that makes up their structure are peptides or proteins) and/or nanoparticles that are developed using a combination of nanomaterials such as lipid-polymer hybrid nanoparticles formed by polymer cores and lipid shells or nanolipoprotein particles formed by a membrane forming lipid arranged in a membrane lipid bilayer stabilized by a scaffold protein as will be understood by a person skilled in the art.

In some embodiments, a carrier is made up of a plurality of monomeric subunits which assemble with one another through covalent and/or non-covalent forces to form the carrier. In some embodiments, the carrier described herein is a protein nanoparticle comprising a plurality of particle-forming proteins, which are the monomeric subunit proteins that form the protein nanoparticle. Protein nanoparticles can be categorized into non-viral protein nanoparticles and viral-like particles. Examples of non-viral protein nanoparticles include but are not limited to ferritins, vaults, heat-shock proteins, chaperonins, lumazine synthase, encapsulins, and bacterial microcompartments. Viral-like particles can be derived from viruses including but not limited to adenovirus, cowpea mosaic virus, cowpea chlorotic mottle virus, brome mosaic virus, broad bean mottle virus, bacteriophage lambda (e.g., bacteriophage lambda procapsid), MS2 bacteriophage, Qβ bacteriophage, P22 phage capsid, and others identifiable to a person skilled in the art.

In some embodiments, the nanoparticles described herein comprise a virus-like particle (VLP). VLP refers to a non-replicating, viral shell, derived from any of several viruses. VLPs can be naturally occurring or synthesized through the individual expression of viral structural proteins, which can then self-assemble into the virus-like structure. VLPs are generally composed of one or more viral proteins, such as particle-forming proteins referred to as capsid, coat, shell, surface and/or envelope proteins, or particle-forming polypeptides derived from these proteins. In some embodiments, VLPs can form spontaneously upon recombinant expression of the protein in an appropriate expression system. VLPs can differ in morphology, size and number of subunits. Methods for producing particular VLPs are known in the art. The presence of VLPs following recombinant expression of viral proteins can be detected using conventional techniques also known in the art, such as by electron microscopy, biophysical characterization, and the like (See e.g., Baker et al. (1991) Biophys. J. 60:1445-1456; and Hagensee et al. (1994) J. Virol. 68:4503-4505). For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions. Any of a variety of VLPs known in the art can be used herein, including but not limited to, Aquifex aeolicus lumazine synthase, Thermotoga maritima encapsulin, Myxococcus xanthus encapsulin, bacteriophage Qbeta virus particle, Flock House Virus (FHV) particle, ORSAY virus particle, and infectious bursal disease virus (IBDV) particle. In some embodiments, the nanoparticle used herein can be a bacteriophage VLP, such as Ap205 VLP.

The nanoparticles described herein can comprise a self-assembling nanoparticle. A self-assembling nanoparticle typically refers to a ball-shape protein shell with a diameter of tens of nanometers and well-defined surface geometry that is formed by identical copies of a non-viral protein capable of automatically assembling into a nanoparticle with a similar appearance to VLPs. Examples of self-assembling nanoparticles include but are not limited to ferritin (FR) (e.g., Helicobacter pylori ferritin), which is conserved across species and forms a 24-mer, as well as B. stearothermophilus dihydrolipoyl acyltransferase (E2p), Aquifex aeolicus lumazine synthase (LuS), and Thermotoga maritima encapsulin, which all form 60-mers.

In some embodiments, the self-assembling nanoparticles comprise a plurality of particle-forming proteins of 2-keto-3-deoxy-phosphogluconate (KDPG) aldolase from the Entner-Doudoroff pathway of the hyperthermophilic bacterium Theremotoga maritima or a variant thereof. In some embodiments, mutations are introduced to the KDPG aldolase for improved particle yields, stability, and uniformity. For example, in some embodiments mutations can introduced to alter the interface between the wild-type protein trimer of KDPG aldolase. In some embodiments, the nanoparticle used herein is an i301 nanoparticle or a variant thereof. In some embodiments, the nanoparticle used herein is a mutated i301 nanoparticle (for example, mi3 nanoparticle). The self-assembling nanoparticles can form spontaneously upon recombinant expression of the protein in an appropriate expression system. Methods for nanoparticle production, detection, and characterization can be conducted using the same techniques developed for VLPs.

In some embodiments, the carriers and the related vaccine compositions, methods and kits disclosed herein can employ any of a variety of known nanoparticles, their conservatively modified variants in which some amino acid residues are substituted with a functionally equivalent residue, as well as variants with substantially identical sequences (e.g., at least 90%, 95%, or 99% identical).

In some embodiments, the carriers used herein are monovalent carriers which present a single HIV immunogen. The HIV immunogens presented on a monovalent carrier herein described have the same protein sequence. In some embodiments herein described, the carriers used herein are multivalent carriers. A multivalent nanoparticle presents a heterologous population of immunogens, comprising at least two immunogens of or derived from different HIV strains. Accordingly, the heterologous immunogens presented on a multivalent carrier herein described have different protein sequences.

The term “present” as used herein with reference to a compound (e.g., an antigen) or functional group indicates attachment performed to maintain the chemical reactivity of the compound or functional group attached. Accordingly, a functional group presented on a carrier is able to perform under the appropriate conditions the one or more chemical reactions that chemically characterize the functional group. A compound presented on a carrier is able to perform under the appropriate conditions the one or more chemical reactions that chemically characterize the compound. For example, where the compound is, or comprises, an HIV immunogen, the HIV immunogen presented by a carrier maintains the complex of reactions that are associated with the immunological activity characterizing the HIV immunogen. Accordingly, presentation of an HIV immunogen indicates an attachment such that the immunological activity associated to the HIV immunogen attached is maintained.

The HIV immunogens presented on the carrier herein described can be displayed on its surface. Alternatively, the HIV immunogens presented on the carrier herein described can be partially encapsulated or embedded such that at least an immunogenic portion of the HIV immunogen is exposed and accessible by a host cell receptor so as to induce an immune response.

Coupling of HIV Immunogens and Carriers

The HIV immunogens can be covalently or non-covalently attached to a carrier. The terms “attach”, “attached”, “couple” and “coupled” are used interchangeably to refer to a chemical association of one entity (e.g., a chemical moiety) with another. The attachment can be direct or indirect such that for example where a first entity is directly bound to a second entity or where a first entity is bound to a second entity via one or more intermediate entity. In some embodiments, the C-terminus of an HIV immunogens is attached to the N-terminus of a subunit forming the carrier.

In some embodiments, the attachment or coupling is covalent such that the attachment occurs in the context of the presence of a covalent bond between two entities. In some embodiments, the attachment or coupling is mediated by non-covalent interactions including but not limited to charge interactions, affinity interactions, metal coordination, hydrophobic interactions, hydrogen bonding interactions, van der Waals interactions, magnetic interactions, electrostatic interactions, dipole-dipole interactions, or combinations thereof. In some embodiments, encapsulation is a form of attachment. In some embodiments, the plurality of HIV immunogens are conjugated to the carrier.

The carrier herein described can, for example, be functionalized with a functional group or a reactive moiety that is presented for binding with a corresponding functional group or a corresponding reactive moiety of an HIV immunogen. Accordingly, the attachment between the HIV immunogen and the carrier can occur through the binding between the functional group pair or reactive moiety pair. Exemplary functional group pairs or reactive moiety pairs include but are not limited to avidin (e.g., streptavidin, NeutrAvidin, CaptAvidin) and biotin pair, Strep-Tactin and Strep-tag pair, a thiol and a thiol-reactive moiety (e.g., maleimide, haloacetamide, iodoacetamid, benzylic halides and bromomethylketones) pair, and an amine and an amine-reactive moiety (e.g., active esters such as succinimidyl, tetrafluorophenyl, Carbodiimide, isothiocyanates, sulfonyl chlorides, dichlorotriazines, acryl halides, acyl azides).

The HIV immunogen can be attached to the carrier via chemical and/or photoreactive crosslinkers (e.g., crosslinking reagents) that contain two reactive groups, thereby providing a means of covalently linking the antigen and the carrier. The reactive groups in a chemical crosslinking reagent typically belong to the classes of functional groups, including succinimidyl esters, maleimides and iodoacetamides and others identifiable to a skilled person. Additional examples of crosslinking and photoactivatable reagents are described, for example, in thermofisher.com/us/en/home/references/molecular-probes-the-handbook/crosslinking-and-photoactivatable-reagents.html, the content of which is incorporated by reference.

In some embodiments, the HIV immunogen can be attached to the carrier via a click chemistry moiety. The term “click chemistry,” as used herein, can refer to a chemical philosophy introduced by K. Barry Sharpless of The Scripps Research Institute, describing chemistry tailored to generate covalent bonds quickly and reliably by joining small units comprising reactive groups together. Click chemistry does not refer to a specific reaction, but to a concept including reactions that mimic reactions found in nature. In some embodiments, click chemistry reactions are modular, wide in scope, give high chemical yields, generate non-toxic byproducts, are stereospecific, exhibit a large thermodynamic driving force >84 kJ/mol to favor a reaction with a single reaction product, and/or can be carried out under physiological conditions. In particular, click chemistry reactions that can be carried out under physiological conditions and that do not produce toxic or otherwise harmful side products are suitable for the generation of hydrogels in situ. Reactive moieties that can partake in a click chemistry reaction are well known to those of skill in the art, and include, but are not limited to alkyne and azide, alkene and tetrazole or tetrazine, or diene and dithioester. Other suitable reactive click chemistry moieties suitable for use in the context of antigen binding are known to those of skill in the art.

In some embodiments, the HIV immunogen is attached to the carrier (e.g., particle-forming proteins of the carrier) through a Spy tag/SpyCatcher binding pair. The Spy tag/SpyCatcher binding pair refers to a protein ligation system that is based on the internal isopeptide bond of the CnaB2 domain of FbaB from Streptococcus pyogenes (see, e.g., Zakeri et al., Proc. Natl. Acad. Sci. USA. 2012; 109:E690-E697). CnaB2 is split and engineered into two complementary fragments, such that the first fragment (SpyCatcher) is able to bind and form a covalent isopeptide bond with the second fragment (SpyTag) through the side chains of a lysine in SpyCatcher and an aspartate in SpyTag. Carriers presenting a plurality of HIV immunogens can then be generated as a result of SpyTag/SpyCatcher mediated conjugation of the antigens to the carriers. The SpyTag/SpyCatcher binding system can in some embodiments provide improved stability and specificity of the interaction between the HIV immunogens and the particle-forming proteins of the carrier.

In some embodiments, the particle-forming protein of the carrier is a fusion protein containing amino acid sequences from at least two unrelated proteins that have been joined together, via peptide bond, to make a single protein. For example, the HIV immunogen can be fused to a SpyTag motif and the carrier subunit sequence can be fused to a SpyCatcher motif. Alternatively, the HIV immunogen can be fused to a SpyCatcher motif and the carrier subunit sequence can be fused to a SpyTag motif. The HIV immunogen of the plurality of HIV immunogens can comprise a SpyTag at the C-terminal of the HIV immunogen and the particle-forming protein of a plurality of particle-forming proteins comprises a SpyCatcher at the N-terminal of the particle-forming protein.

In some embodiments, the particle-forming protein can be a fusion protein containing a mi3 monomeric subunit protein at the C-terminal of the particle-forming protein and a SpyCatcher protein at the N-terminal of the particle-forming protein or a fusion protein containing a AP205-CP3 monomeric subunit protein at the C-terminal of the particle-forming protein and a SpyCatcher protein at the N-terminal of the particle forming protein such that the SpyCatcher proteins are presented or displayed for binding to the SpyTag of an HIV immunogen.

In some embodiments, the HIV immunogen of the plurality of HIV immunogens comprises an HIV immunogen, and the HIV immunogen comprises a Spy tag at the C-terminal of the HIV immunogen and the particle-forming protein of a plurality of particle-forming proteins comprises a SpyCatcher at the N-terminal of the particle-forming protein.

Multivalent and Monovalent Carriers

In some embodiments, the carrier is a monovalent carrier, comprising a plurality of HIV immunogens having the same sequence (e.g., IGT1 or IGT2). In some embodiments herein described, the carrier used herein can be a multivalent carrier and can comprise a plurality of HIV immunogens derived from a plurality of HIV variants (e.g., also referred to herein as HIV strains or HIV quasispecies), the plurality of HIV variants being different from one another. The plurality of HIV immunogens can comprise at least a first HIV immunogen of a first HIV variant and a second HIV immunogen of a second HIV variant that is different from the first HIV variant.

One of the hallmarks of HIV infection is the rapid development of a genetically complex population (quasispecies) from an initially limited number of infectious particles. Genetic diversity remains one of the major obstacles to eradication of HIV. The viral quasispecies can respond rapidly to selective pressures, such as that imposed by the immune system and antiretroviral therapy, and frustrates vaccine design efforts. Two unique features of retroviral replication are responsible for the unprecedented variation generated during infection. First, mutations are frequently introduced into the viral genome by the error prone viral reverse transcriptase and through the actions of host cellular factors, such as the APOBEC family of nucleic acid editing enzymes. Second, the HIV reverse transcriptase can utilize both copies of the co-packaged viral genome in a process termed retroviral recombination. When the co-packaged viral genomes are genetically different, retroviral recombination can lead to the shuffling of mutations between viral genomes in the quasispecies (e.g., strains or variants).

In some embodiments, the multivalent carrier comprises a plurality of HIV immunogens, the plurality of HIV immunogens comprising at least two, three, four, five, six, seven, or eight heterologous HIV immunogens, each of which is of or derived from an HIV strain or variant different from one another.

For example, the multivalent carrier can comprise a plurality of heterologous HIV immunogens, the plurality of HIV immunogens comprising at least a first HIV immunogen of a first HIV variant, a second HIV immunogen of a second HIV variant, and a third HIV immunogen of a third HIV variant, in which the first HIV variant, the second HIV variant and the third HIV variant are different from one another.

The multivalent carrier can comprise a plurality of heterologous HIV immunogens, the plurality of HIV immunogens comprising at least a first HIV immunogen of a first HIV variant, a second HIV immunogen of a second HIV variant, a third HIV immunogen of a third HIV variant, and a fourth HIV immunogen of a fourth HIV variant, in which the first HIV variant, the second HIV variant, the third HIV variant, and the fourth HIV variant are different from one another.

The multivalent carrier can comprise a plurality of heterologous HIV immunogens, the plurality of HIV immunogens comprising at least a first HIV immunogen of a first HIV variant, a second HIV immunogen of a second HIV variant, a third HIV immunogen of a third HIV variant, a fourth HIV immunogen of a fourth HIV variant, and a fifth HIV immunogen of a fifth HIV variant, in which the first HIV variant, the second HIV variant, the third HIV variant, the fourth HIV variant, and the fifth HIV variant are different from one another.

The multivalent carrier can comprise a plurality of heterologous HIV immunogens, the plurality of HIV immunogens comprising at least a first HIV immunogen of a first HIV variant, a second HIV immunogen of a second HIV variant, a third HIV immunogen of a third HIV variant, a fourth HIV immunogen of a fourth HIV variant, a fifth HIV immunogen of a fifth HIV variant, and a sixth HIV immunogen of a sixth HIV variant, in which the first HIV variant, the second HIV variant, the third HIV variant, the fourth HIV variant, the fifth HIV variant, and the sixth HIV variant are different from one another.

The multivalent carrier can comprise a plurality of heterologous HIV immunogens, the plurality of HIV immunogens comprising at least a first HIV immunogen of a first HIV variant, a second HIV immunogen of a second HIV variant, a third HIV immunogen of a third HIV variant, a fourth HIV immunogen of a fourth HIV variant, a fifth HIV immunogen of a fifth HIV variant, a sixth HIV immunogen of a sixth HIV variant, and a seventh HIV immunogen of a seventh HIV variant, in which the first HIV variant, the second HIV variant, the third HIV variant, the four HIV variant, the fifth HIV variant, the sixth HIV variant, and the seventh HIV variant are different from one another.

The multivalent carrier can comprise a plurality of heterologous HIV immunogens, the plurality of HIV immunogens comprising at least a first HIV immunogen of a first HIV variant, a second HIV immunogen of a second HIV variant, a third HIV immunogen of a third HIV variant, a fourth HIV immunogen of a fourth HIV variant, a fifth HIV immunogen of a fifth HIV variant, a sixth HIV immunogen of a sixth HIV variant, a seventh HIV immunogen of a seventh HIV variant, and an eighth HIV immunogen of an eight HIV variant, in which the first HIV variant, the second HIV variant, the third HIV variant, the fourth HIV variant, the fifth HIV variant, the sixth HIV variant, the seventh HIV variant, and the eighth HIV variant are different from one another.

In some embodiments, the carrier can comprise a plurality of HIV immunogens. The monovalent carriers described herein comprise HIV immunogens of the same sequence (e.g., IGT1 or IGT2). In some embodiments, the plurality of HIV immunogens comprise more than eight heterologous HIV immunogens, each of which is of or derived from an HIV variant different from one another (i.e. HIV variants of different taxonomic groups and/or antigenically divergent viruses).

The number of HIV immunogens presented by a carrier can be different in different embodiments. In some embodiments, the carrier herein described can present at least 2, 3, 4, 5, 6, 7, 8, 9, 10, 11, 12, 13, 14, 15, 16, 17, 18, 19, 20, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, HIV immunogens.

The total number of HIV immunogens presented by a carrier can be different in different embodiments. In some embodiments, the carrier can comprise a total number of HIV immunogens about, at least, at least about, at most, or at most about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, or a number or a range between any two of these values.

It should be understood that in some embodiments the total number of HIV immunogens presented by a nanoparticle is limited by the number of particle-forming subunits that make up the nanoparticle, such as the number of particle-forming lipids in lipid-based nanoparticles and the number of particle-forming proteins in protein-based nanoparticles. For example, encapsulin proteins from Thermotoga maritima form nanoparticles having 60-mers. Therefore, encapsulin-based nanoparticles (e.g., mi3 nanoparticle and i301 nanoparticle) can present a maximum of 60 protein antigens. In some embodiments, a particle-forming subunit of a carrier can be attached with more than one HIV immunogen.

The plurality of HIV immunogens attached to a multivalent carrier can be of a same protein type or corresponding proteins. HIV immunogens of a same protein type may or may not have identical amino acid sequences, but generally share some sequence homology. For example, the HIV Env proteins of different HIV strains are of a same protein type or corresponding proteins. As another example, envelope proteins from different HIV variants are considered the same protein type or corresponding proteins.

One or more of the plurality of HIV immunogens, or each of the plurality of HIV immunogens attached to a multivalent carrier, can have a sequence identity of about, at least, or at least about 65%, 70%, 75%, 80%, 85%, 90%, 95%, 98%, or 99% with one another. In some embodiments, the plurality of HIV immunogens each comprise an HIV Env protein or a portion thereof (e.g., CD4 binding site), the HIV Env proteins or portions thereof having a sequence identity of about, at least, or at least about, 65%, 70%, 75%, 80%, 85%, 90%, 95%, 98%, or 99% with one another. In some embodiments, the plurality of HIV immunogens each comprise an amino acid sequence having at least 65%, 70%, 75%, 80%, 85%, 90%, 95%, or 98% sequence identity to an amino acid sequence selected from SEQ ID NOs: 132-139. In some embodiments, the plurality of HIV immunogens each comprise an amino acid sequence selected from SEQ ID NOs: 132-139.

The number of attached HIV immunogens of different HIV strains can be the same or different. For example, the number of the first HIV immunogens of the first HIV variant and the number of the second HIV immunogens of the second HIV variant can be in a ratio from 1:50 to 50:1. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values.

In some embodiments, the number of the HIV immunogens of an HIV variant and the number of the HIV immunogens of another HIV variant can be in a ratio from 1:50 to 50:1. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values. In some embodiments, the ratio can be, be about, be at least, be at least about, be at most, be at most about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of the values.

In some embodiments, the monovalent carriers can comprise a plurality of HIV immunogens having the same amino acid sequence (e.g., IGT1 immunogen comprising SOSIP mutations and SpyTag sequence, SEQ ID NO: 130). The plurality of HIV immunogens can comprise any of the isolated polypeptides disclosed herein. The plurality of HIV immunogens can comprise an isolated polypeptide comprising or consisting of an amino acid sequence of any of SEQ ID NOs 117-125 and 129-131 or variants thereof. The plurality of HIV immunogens can comprise an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs 117-125 and 129-131. The plurality of HIV immunogens can comprise an isolated polypeptide comprising or consisting of an amino acid sequence of any of SEQ ID NOs: 119, 122, and 131 or variants thereof. The plurality of HIV immunogens can comprise an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs: 119, 122, and 131. The plurality of HIV immunogens can comprise an isolated polypeptide comprising or consisting of an amino acid sequence of any of SEQ ID NOs: 118, 121, and 130 or variants thereof. The plurality of HIV immunogens can comprise an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs: 118, 121, and 130. The plurality of HIV immunogens can comprise an isolated polypeptide comprising or consisting of an amino acid sequence of any of SEQ ID NOs: 117, 120, and 129 or variants thereof. The plurality of HIV immunogens can comprise an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs: 117, 120, and 129.

The carrier herein described (either the monovalent or multivalent carriers described herein or combinations thereof) can induce broadly protective anti-HIV responses by eliciting broadly neutralizing antibodies. Broadly neutralizing antibodies are antibodies that can neutralize HIV strains or variant not only the same as but also differ from the strains of HIV from which the HIV immunogens used to elicit the antibodies are derived. Broadly neutralizing response can also be referred to as heterologously neutralizing response. In some embodiments, the carriers herein described can elicit broadly neutralizing antibodies that neutralize one or more HIV variants (e.g., strains or quasispecies), and/or strain of the HIV virus from which the HIV immunogens are derived to produce the carriers.

The carriers herein described can be prepared using any standard molecular biology procedures known to the person skilled in the art as well as the protocols exemplified herein (see e.g., Example 1). In some embodiments, particle-forming subunits and/or the HIV immunogens can be produced by liquid-phase or solid-phase chemical protein synthetic methods known to those of skill in the art.

Production of the particle-forming subunits and/or the HIV immunogens can use recombinant DNA technology well known in the art. For example, a tagged HIV immunogen or an HIV immunogen functionalized with a protein tag can be synthesized using biosynthetic methods such as cell-based or cell-free methods known to the person skilled in the art. A tagged HIV immunogen can be produced using an expression vector comprising a nucleic acid molecule encoding the HIV immunogen. The nucleic acid molecule can be operably linked to appropriate regulatory elements including, but not limited to, a promoter, enhancer, transcription initiation site, termination site, and translation initiation site. The vector can also comprise a nucleic acid molecule encoding one or more protein tags (e.g., a poly(His) tag, SpyTag). In some embodiments, the vector can additionally include a nucleic acid molecule encoding a trimerization motif (e.g., a foldon trimerization domain from T4 fibritin or viral capsid protein SUP). The vector can also comprise a nucleic acid molecule encoding a signal peptide that directs the protein into the proper cellular pathway, such as a signal peptide for secretion of the expressed protein into supernatant medium. The vector may comprise one or more selectable marker genes such as gene providing ampicillin resistance or kanamycin resistance. Methods for the construction of nucleic acid constructs are well known. See, for example, Molecular Cloning: a Laboratory Manual, 3d edition, Sambrook et al. 2001 Cold Spring Harbor Laboratory Press, and Current Protocols in Molecular Biology, Ausubel et al. eds., John Wiley & Sons, 1994. Protein biosynthesis of tagged HIV immunogens can be performed by providing cell-based or cell-free protein translation systems with the expression vectors encoding the tagged HIV immunogens. Similarly, a tagged particle-forming protein can be produced using an expression vector comprising a nucleic acid molecule encoding a particle-forming subunit and a nucleic acid molecule encoding a protein tag (e.g., SpyCatcher). In an exemplary embodiment, the carriers are produced following the protocols described in Cohen A A et al, 2021, PLoS ONE 16(3): e0247963, the content of which is incorporated herein by reference.

In some embodiments, constructs expressing the carrier subunit and the HIV immunogens can be introduced together into a host or transformation-competent cell. Carriers can be generated as a result of conjugation of the expressed HIV immunogens to the self-assembled nanoparticles through a functional group pair or a reactive moiety pair described herein (e.g., SpyTag/SpyCatcher).

Carriers (e.g., nanoparticles with SpyCatcher) and HIV immunogens (e.g., SpyTagged protein antigens) can, for example, be prepared separately and then incubated under a condition (e.g., in a TBS buffer at room temperature) for a certain time period (e.g., about, at least, or at least about 1 hour, 2 hours, 5 hours, 10 hours, 12 hours, 15 hours) to allow for the conjugation of the carriers and the HIV immunogens. In some embodiments, the HIV immunogens are provided in an excess amount as compared to the particle-forming subunits of the carriers, such as 1-fold, 2-fold, 3-fold, 4-fold, 5-fold or greater than the particle-forming subunits.

Disclosed herein include kits. In some embodiments, the kit comprises any of the immunogenic compositions of or the vaccine compositions described herein.

### Methods for Stimulating Immune Response Using the Disclosed Immunogens

Disclosed herein include methods of stimulating an immune response in a subject in need thereof. In some embodiments, the method comprises: administering to the subject one or more vaccine compositions disclosed herein, thereby stimulating an immune response in the subject. Disclosed herein include methods for treating or preventing an HIV infection in a subject in need thereof. In some embodiments, the method comprises: administering to the subject one or more vaccine compositions disclosed herein, thereby treating or preventing the HIV infection in the subject. Disclosed herein include methods of treating or preventing a disease or disorder caused by an HIV infection in a subject in need thereof. In some embodiments, the method comprises: administering to the subject one or more vaccine compositions disclosed herein, thereby treating or preventing the disease or disorder caused by the HIV infection in the subject. Also provided herein is the use of one or more vaccine compositions disclosed herein for treating or preventing an HIV infection in a subject.

The immunogens, as disclosed herein, a nucleic acid molecule encoding the disclosed immunogen, the host cell, the protein complex, or the virus particle can be administered to a subject in order to generate an immune response to a pathogen, such as HIV. In another aspect, this disclosure provides a method of treating or preventing HIV infection in a subject in need thereof. The method includes administering to the subject a therapeutically effective amount of the immunogen, the nucleic acid, the host cell, the protein complex, or the virus particle described above, or a combination thereof. This disclosure also provides use of the immunogen, the nucleic acid, the host cell, the protein complex, or the virus particle described above, or a combination thereof in the preparation of a medicament to treat or prevent HIV infection in a subject.

In exemplary applications, compositions are administered to a subject suffering from HIV infection or at risk of becoming infected from HIV. In other applications, the immunogens disclosed herein can be administered prophylactically, for example, as part of an immunization regimen.

In some embodiments, administering the one or more vaccine compositions induces a polyclonal serum response in the subject. In some embodiments, administering the one or more vaccine compositions induces broadly neutralizing responses in the subject against one or more HIV variants. In some embodiments, administering the one or more vaccine compositions boosts a neutralizing antibody response in the subject. The one or more vaccine compositions can be administered to the subject two or more times.

Administering the one or more vaccine compositions can comprise administering to the subject a first vaccine composition and administering to the subject a second vaccine composition. The first vaccine composition can comprise a vaccine composition comprising an isolated polypeptide comprising or consisting of an amino acid sequence of any of SEQ ID NOs: 117, 120, and 129 or variants thereof. The first vaccine composition can comprise a vaccine composition comprising an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs: 117, 120, and 129.

In some embodiments, the first vaccine composition comprises a carrier associated with a plurality of HIV immunogens. The plurality of HIV immunogens can comprise an isolated polypeptide comprising or consisting of an amino acid sequence of any of SEQ ID NOs: 117, 120, and 129 or variants thereof. The plurality of HIV immunogens can comprise an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs: 117, 120, and 129.

The second vaccine composition can comprise a vaccine composition comprising an isolated polypeptide comprising or consisting of an amino acid sequence of any of SEQ ID NOs: 118, 121, and 130 or variants thereof. The second vaccine composition can comprise a vaccine composition comprising an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs: 118, 121, and 130.

In some embodiments, the second vaccine composition comprises a carrier associated with a plurality of HIV immunogens. The plurality of HIV immunogens can comprise an isolated polypeptide comprising or consisting of an amino acid sequence of any of SEQ ID NOs: 118, 121, and 130 or variants thereof. The plurality of HIV immunogens can comprise an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs: 118, 121, and 130.

In some embodiments, administration of the second vaccine composition to the subject occurs about two, three, four, or five weeks after administration of the first vaccine composition to the subject.

In some embodiments, administering the one or more vaccine compositions further comprises administering a third, fourth, and fifth vaccine composition. The third vaccine composition can comprise a vaccine composition comprising or consisting of an isolated polypeptide comprising an amino acid sequence of any of SEQ ID NOs: 119, 122, and 131 or variants thereof. The third vaccine composition can comprise a vaccine composition comprising an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs: 119, 122, and 131.

The third vaccine composition can comprise a carrier associated with a plurality of human immunodeficiency virus (HIV) immunogens. The plurality of HIV immunogens can comprise an isolated polypeptide comprising or consisting of an amino acid sequence of any of SEQ ID NOs: 119, 122, and 131 or variants thereof. The plurality of HIV immunogens can comprise an isolated polypeptide comprising an amino acid sequence that is at least 90% identical (e.g., 90%, 91%, 92%, 93%, 94%, 95%, 96%, 97%, 98%, 99%, 100%, or a number or a range between any two of these values) to any of SEQ ID NOs: 119, 122, and 131.

In some embodiments, the fourth and fifth vaccine compositions comprise a vaccine composition comprising one or more of an isolated polypeptide, each comprising or consisting of an amino acid sequence of any of SEQ ID NOs: 132-139 or a sequence that is at least 90% identical (e.g., 90%, 91%, 92%, 93%, 94%, 95%, 96%, 97%, 98%, 99%, 100%, or a number or a range between any two of these values) to any of SEQ ID NOs: 132-139.

In some embodiments, the fourth and fifth vaccine compositions comprise a multivalent carrier associated with a plurality of human immunodeficiency virus (HIV) immunogens. One or more of the plurality of HIV immunogens, or each of the plurality of HIV immunogens attached to a multivalent carrier, can have a sequence identity of about, at least, or at least about 65%, 70%, 75%, 80%, 85%, 90%, 95%, 98%, or 99% with one another. In some embodiments, the plurality of HIV immunogens each comprise an HIV Env protein or a portion thereof (e.g., CD4 binding site), the HIV Env proteins or portions thereof having a sequence identity of about, at least, or at least about, 65%, 70%, 75%, 80%, 85%, 90%, 95%, 98%, or 99% with one another. In some embodiments, the plurality of HIV immunogens each comprise an amino acid sequence having at least 65%, 70%, 75%, 80%, 85%, 90%, 95%, or 98% sequence identity to an amino acid sequence selected from SEQ ID NOs: 132-139. In some embodiments, the plurality of HIV immunogens each comprise an amino acid sequence selected from SEQ ID NOs: 132-139. The plurality of HIV immunogens can comprise three, four, five, six, seven, or eight HIV immunogens. Each of the three, four, five, six, seven, or eight of HIV immunogens can be derived from an HIV variant different from the other.

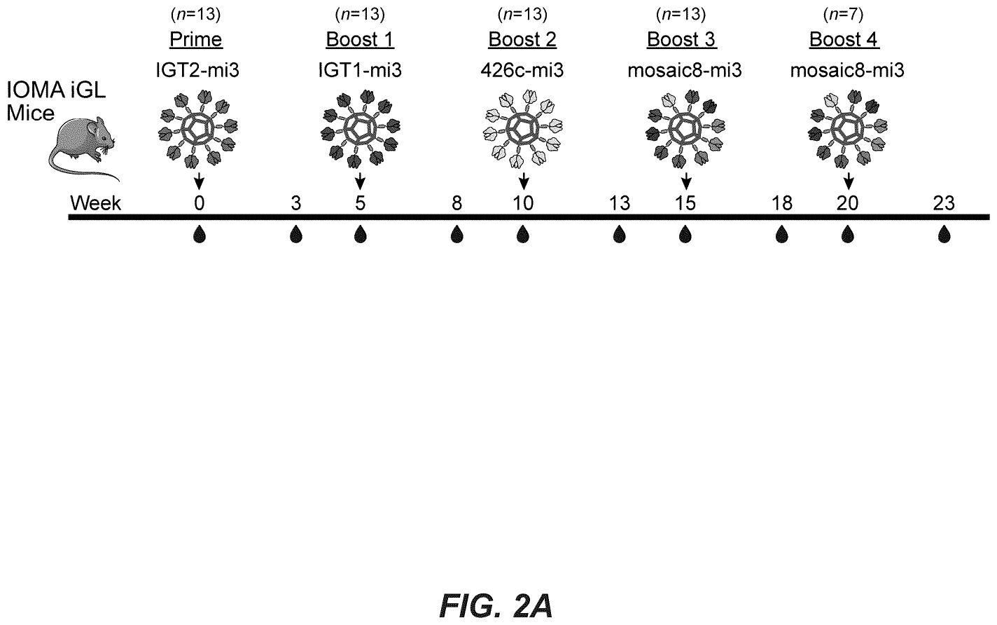

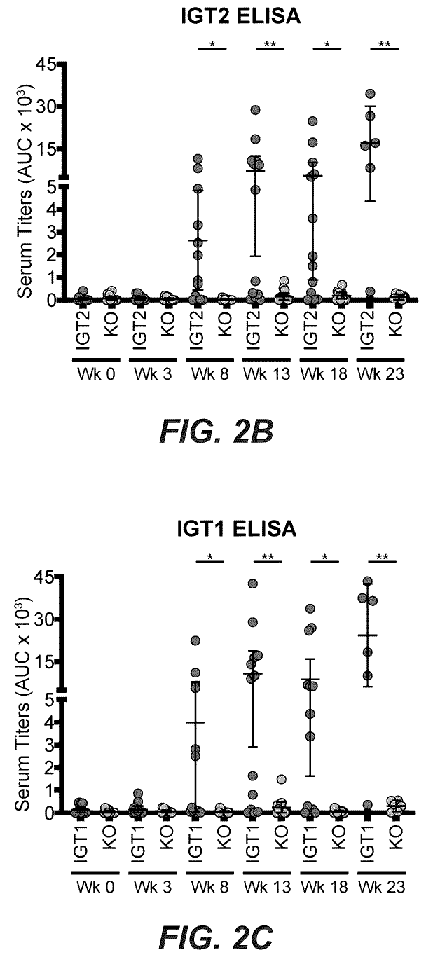

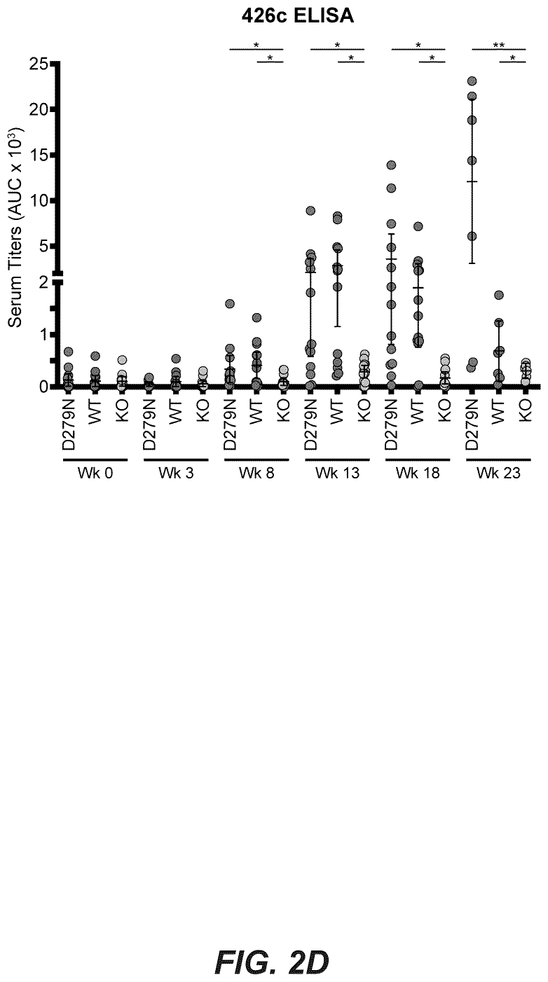

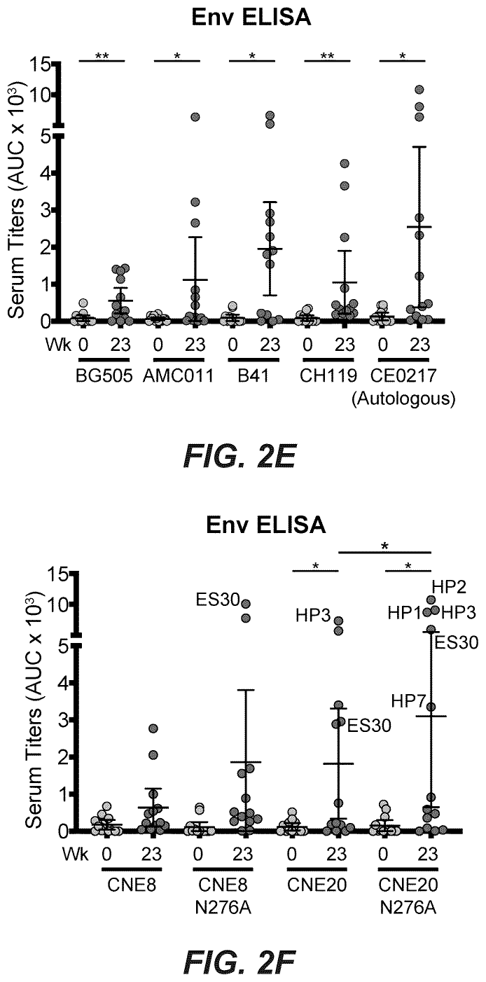

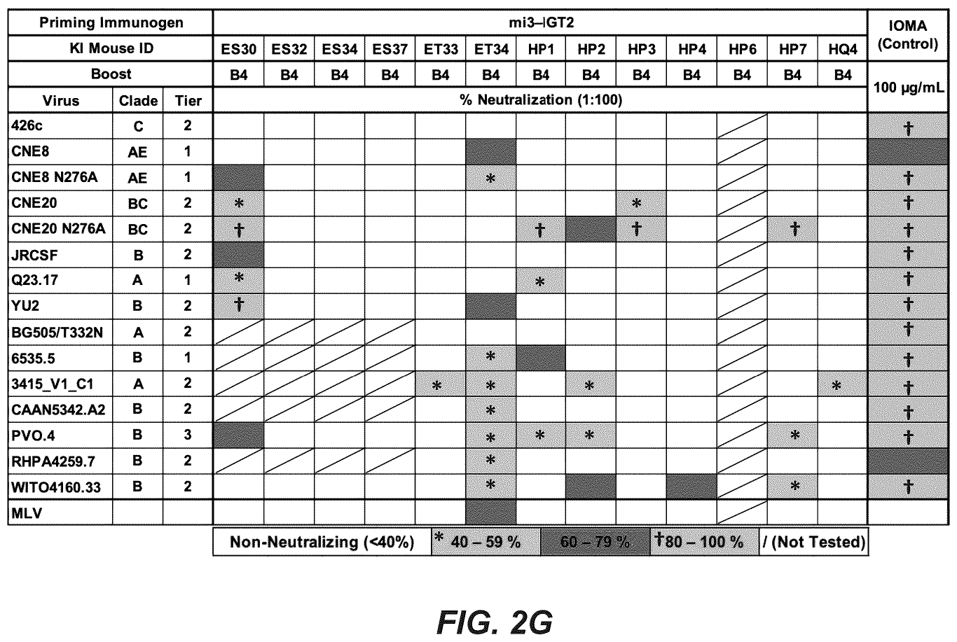

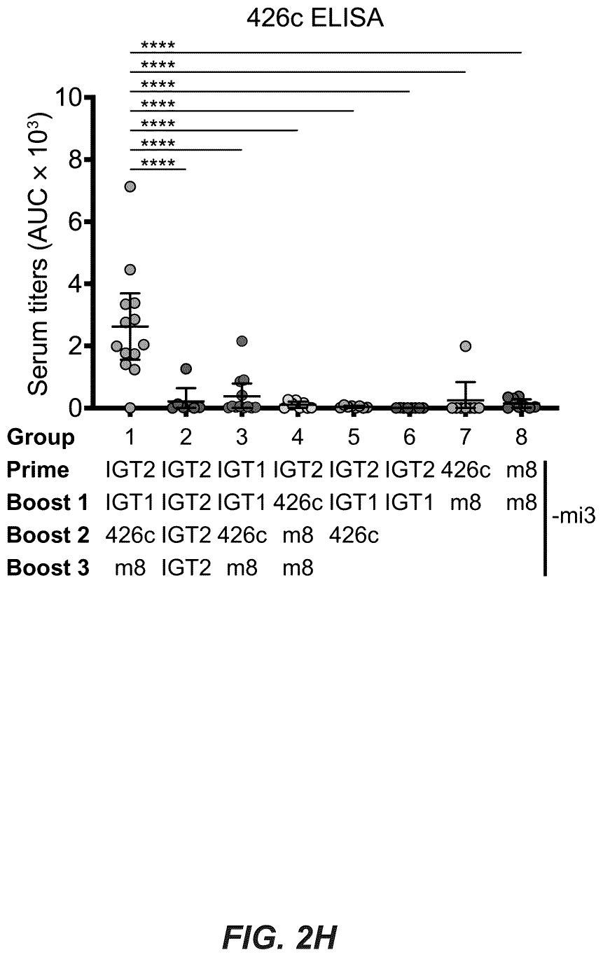

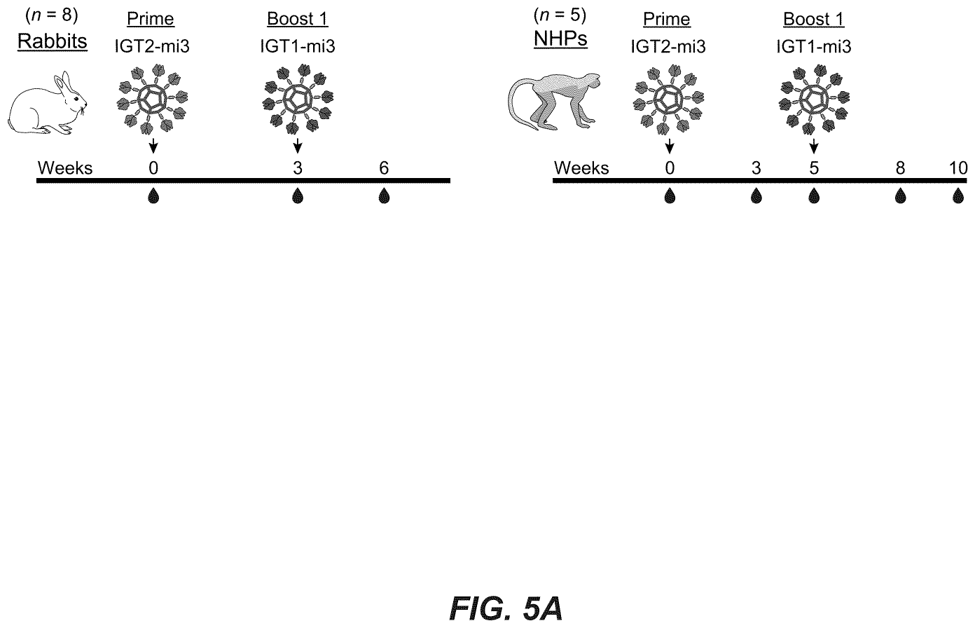

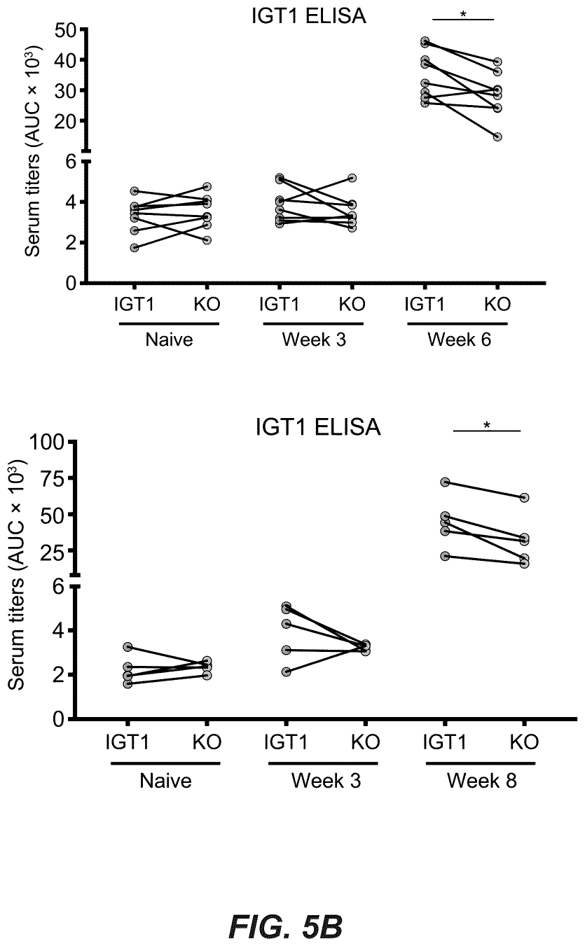

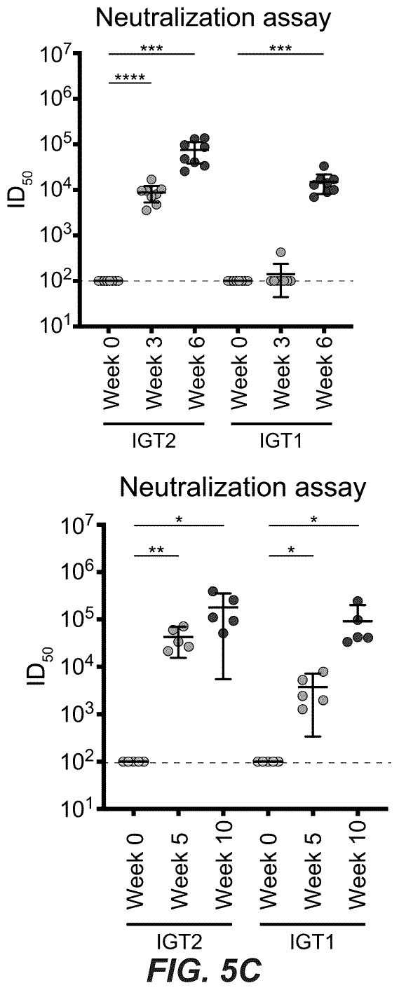

In some embodiments, administration of the third vaccine composition to the subject occurs about two, three, four, or five weeks after administration of the second vaccine composition to the subject. In some embodiments, administration of the fourth vaccine composition to the subject occurs about two, three, four, or five weeks after administration of the third vaccine composition to the subject. In some embodiments, administration of the fifth vaccine composition to the subject occurs about two, three, four, or five weeks after administration of the fourth vaccine composition to the subject. Exemplary immunization regimens are shown in, e.g., FIG. 2A and FIG. 5A.

The immunogen is administered in an amount sufficient to raise an immune response against the HIV virus. Administration induces a sufficient immune response to treat the infection, for example, to inhibit the infection and/or reduce the signs and/or symptoms of the infection. Amounts effective for this use will depend upon the severity of the disease, the general state of the subject's health, and the robustness of the subject's immune system. A therapeutically effective amount of the immunogen is that which provides either subjective relief of a symptom(s) or an objectively identifiable improvement as noted by the clinician or other qualified observers.

Therapeutically effective amount or effective amount refers to the amount of agents, such as nucleic acid vaccine or other therapeutic agents, that is sufficient to prevent, treat (including prophylaxis), reduce and/or ameliorate the symptoms and/or underlying causes of any of a disorder or disease, for example to prevent, inhibit, and/or treat HIV. In some embodiments, an “effective amount” is sufficient to reduce or eliminate a symptom of a disease, such as AIDS. For instance, this can be the amount necessary to inhibit viral replication or to measurably alter outward symptoms of the viral infection, such as an increase of T cell counts in the case of HIV-1 infection. In general, this amount will be sufficient to measurably inhibit virus (for example, HIV) replication or infectivity. When administered to a subject, a dosage will generally be used that will achieve target tissue concentrations (for example, in lymphocytes) that have been shown to achieve in vitro inhibition of viral replication.

An immunogen can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, P A, 1995) either locally or systemically, such as by intramuscular, subcutaneous, or intravenous injection, but even oral, nasal, or anal administration is contemplated. In one embodiment, the administration is by subcutaneous or intramuscular injection. To extend the time during which the disclosed immunogen is available to stimulate a response, the immunogen can be provided as an implant, an oily injection, or as a particulate system. The particulate system can be a microparticle, a microcapsule, a microsphere, a nanocapsule, or similar particle, (see, e.g., Banga, supra). A particulate carrier based on a synthetic polymer has been shown to act as an adjuvant to enhance the immune response, in addition to providing a controlled release. Aluminum salts can also be used as adjuvants to produce an immune response.

Optionally, one or more cytokines, such as interleukin (IL)-2, IL-6, IL-12, IL-15, RANTES, granulocyte-macrophage colony-stimulating factor (GM-CSP), tumor necrosis factor (TNF)-a, interferon (IFN)-a or IFN-y, one or more growth factors, such as GM-CSP or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oneal. 68(2): 122-38; Lotze et al., 2000, Cancer J Sci. Am. 6(Suppl 1):561-6; Cao et al., 1998, Stem Cells 16(Suppl 1 J.-251-60; Kuiper et al., 2000, Adv. Exp. Med. Biol. 465:381-90). These molecules can be administered systemically (or locally) to the host. In several examples, IL-2, RANTES, GM-CSP, TNF-a, IFN-y, G-CSF, LFA-3, CD72, B7-1, B7-2, B7-1 B.7-2, OX-40L, 41 BBL, and ICAM-1 are administered.

A pharmaceutical composition including an isolated immunogen is provided. In some embodiments, the immunogen is mixed with an adjuvant containing two or more of a stabilizing detergent, a micelle-forming agent, and an oil. Suitable stabilizing detergents, micelle-forming agents, and oils are detailed in U.S. Pat. Nos. 5,585,103; 5,709,860; 5,270,202; and 5,695,770. A stabilizing detergent is any detergent that allows the components of the emulsion to remain as a stable emulsion. Such detergents include polysorbate, (TWEEN) (Sorbitan-mono-9-octadecenoate-poly(oxy-1,2-ethanediyl; manufactured by ICI Americas, Wilmington, DD), TWEEN 40™, TWEEN 20™, TWEEN 60™ ZWITTERGENT™ 3-12, TEEPOL HB7™, and SPAN 85™.

These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, e.g., Schmolka, J. Am. Oil. Chem. Soc. 54: 110, 1977, and Hunter et al., J. Immunol 129: 1244, 1981, PLURONIC™ L62LF, LlOl, and L64, PEGlO00, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10%, or in an amount between 1.25 and 5%.

Controlled release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, P A, 1995. Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μmin diameter and are administered subcutaneously or intramuscularly (see Kreuter, Colloidal Drug Delivery Systems, J. Kreuter, ed., Marcel Dekker, Inc., New York, NY, pp. 219-342, 1994; Tice & Tabibi, Treatise on Controlled Drug Delivery, A. Kydonieus, ed., Marcel Dekker, Inc. New York, NY, pp. 315-339, 1992).

Polymers can be used for ion-controlled release. Various degradable and nondegradable polymeric matrices for use in controlled drug delivery are known in the art (Langer, Accounts Chem. Res. 26:53', 1993). For example, the block copolymer, poloxamer 407 exists as a viscous yet mobile liquid at low temperatures but forms a semisolid gel at body temperature. It has shown to be an effective vehicle for formulation and sustained delivery of recombinant interleukin-2 and urease (Johnston et ah, Pharm. Res. 9:425, 1992; and Pee, /. Parent. Sci. Tech. 44(2):58, 1990). Alternatively, hydroxyapatite has been used as a microcarrier for controlled release of proteins (Ijntema et ah, Int. J. Pharm. 112:215, 1994). In yet another aspect, liposomes are used for controlled release as well as drug targeting of the lipid-capsulated drug (Betageri et ah, Liposome Drug Delivery Systems, Technomic Publishing Co., Inc., Lancaster, P A, 1993). Numerous additional systems for controlled delivery of therapeutic proteins are known (e.g., U.S. Pat. Nos. 5,055,303; 5,188,837; 4,235,871; 4,501,728; 4,837,028; 4,957,735; and 5,019,369; 5,055,303; 5,514,670; 5,413,797; 5,268,164; 5,004,697; 4,902,505; 5,506,206; 5,271,961; 5,254,342; and 5,534,496).

In some embodiments, a pharmaceutical composition includes a nucleic acid encoding a disclosed immunogen (e.g., a nucleic acid comprising a modified mRNA). A therapeutically effective amount of the nucleic acid can be administered to a subject in order to generate an immune response. In one specific, non-limiting example, a therapeutically effective amount of a nucleic acid encoding a disclosed immunogen or immunogenic fragment thereof is administered to a subject to treat or prevent or inhibit HIV infection.

Optionally, one or more cytokines, such as IL-2, IL-6, IL-12, RANTES, GM-CSP, TNP-a, or IPN-y, one or more growth factors, such as GM-CSP or G-CSP, one or more costimulatory molecules, such as ICAM-1, LPA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oneal. 68(2): 122-38; Lotze et al., 2000, Cancer J Sci. Am. 6(Suppl 1):561-6; Cao et al., 1998, Stem Cells 16(Suppl 1):251-60; Kuiper et al., 2000, Adv. Exp. Med. Biol. 465:381-90). These molecules can be administered systemically to the host. It should be noted that these molecules can be co-administered via insertion of a nucleic acid encoding the molecules into a vector, for example, a recombinant pox vector (see, for example, U.S. Pat. No. 6,045,802). In various embodiments, the nucleic acid encoding the biological adjuvant can be cloned into the same vector as the disclosed immunogen coding sequence, or the nucleic acid can be cloned into one or more separate vectors for co-administration. In addition, nonspecific immunomodulating factors such as Bacillus Cahnette-Guerin (BCG) and levamisole can be co-administered. One approach to administration of nucleic acids is direct immunization with plasmid DNA, such as with a mammalian expression plasmid. As described above, the nucleotide sequence encoding the disclosed immunogen can be placed under the control of a promoter to increase expression of the molecule.

Immunization by nucleic acid constructs is well known in the art and taught, for example, in U.S. Pat. No. 5,643,578 (which describes methods of immunizing vertebrates by introducing DNA encoding a desired immunogen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil ATM (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In another approach to using nucleic acids for immunization, a disclosed immunogen can also be expressed by attenuated viral hosts or vectors or bacterial vectors. Recombinant vaccinia virus, adeno-associated virus (AAV), herpes virus, retrovirus, cytomegalovirus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991). In one embodiment, a nucleic acid encoding a disclosed immunogen is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites, including tissues in proximity to metastases. Dosages for injection are usually around 0.5 g/kg to about 50 mg/kg, and typically are about 0.005 mg/kg to about 5 mg/kg (see, e.g., U.S. Pat. No. 5,589,466).

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

It can be advantageous to administer the immunogenic compositions disclosed herein with other agents such as proteins, peptides, antibodies, and other antiviral agents, such as anti-HIV agents. Examples of such anti-HIV therapeutic agents include nucleoside reverse transcriptase inhibitors, such as abacavir, AZT, didanosine, emtricitabine, lamivudine, stavudine, tenofovir, zalcitabine, zidovudine, and the like, non-nucleoside reverse transcriptase inhibitors, such as delavirdine, efavirenz, nevirapine, protease inhibitors such as amprenavir, atazanavir, indinavir, lopinavir, nelfinavir, fosamprenavir, ritonavir, saquinavir, tipranavir, and the like, and fusion protein inhibitors such as enfuvirtide and the like. In certain embodiments, immunogenic compositions are administered concurrently with other anti-HIV therapeutic agents. In some examples, the disclosed immunogens are administered with T-helper cells, such as exogenous T-helper cells. Exemplary methods for producing and administering T-helper cells can be found in WO 03/020904, which is incorporated herein by reference. In certain embodiments, the immunogenic compositions are administered sequentially with other anti-HIV therapeutic agents, such as before or after the other agent. One of ordinary skill in the art would know that sequential administration can mean immediately following or after an appropriate period of time, such as hours, days, weeks, months, or even years later.

The disclosed immunogens or immunogenic fragments thereof and nucleic acids encoding these immunogens can be used in a multistep immunization regime. In some examples, the regime includes administering to a subject a therapeutically effective amount of a first immunogen or immunogenic fragments thereof as disclosed herein (the prime) and boosting the immunogenic response with one or more additional immunogens or immunogenic fragments thereof after an appropriate period of time. The method of eliciting such an immune reaction is what is known as “prime-boost.” In this method, the antibody response to the selected immunogenic surface is focused by giving the subject's immune system a chance to “see” the antigenic surface in multiple contexts. In other words, the use of multiple immunogens or immunogenic fragments thereof with an antigenic surface in common selects for antibodies that bind the immunogen's surface in common. The prime-boost regime can comprise any of the prime-boost regimens disclosed herein, e.g., as shown in FIG. 2A.

One can also use cocktails containing the disclosed immunogenic agents, for example, the immunogen, the nucleic acid encoding the immunogen, the host cell, the protein complex, or the virus particle described above, or a combination thereof to prime and then boost with trimers from a variety of different HIV immunogens or with trimers that are a mixture of multiple HIV immunogens. The prime can be administered as a single dose or multiple doses, for example, two doses, three doses, four doses, five doses, six doses or more can be administered to a subject over days, weeks or months. The boost can be administered as a single dose or multiple doses, for example, two to six doses or more can be administered to a subject over a day, a week or months. Multiple boosts can also be given, such as one to five, or more. Different dosages can be used in a series of sequential inoculations. For example, a relatively large dose in a primary inoculation and then a boost with relatively smaller doses. The immune response against the selected antigenic surface can be generated by one or more inoculations of a subject with an immunogenic composition disclosed herein.

Disclosed herein include methods of stimulating an immune response in a subject in need thereof. In some embodiments, the method comprises: administering to the subject one or more vaccine compositions disclosed herein, thereby stimulating an immune response in the subject. Disclosed herein include methods for treating or preventing an HIV infection in a subject in need thereof. In some embodiments, the method comprises: administering to the subject one or more vaccine compositions disclosed herein, thereby treating or preventing the HIV infection in the subject. Disclosed herein include methods of treating or preventing a disease or disorder caused by an HIV infection in a subject in need thereof. In some embodiments, the method comprises: administering to the subject one or more of vaccine compositions disclosed herein, thereby treating or preventing the disease or disorder caused by the HIV infection in the subject.

In some embodiments, administering the one or more vaccine compositions induces a polyclonal serum response in the subject. In some embodiments, administering the one or more vaccine compositions induces broadly neutralizing responses in the subject against one or more HIV variants. In some embodiments, administering the one or more vaccine compositions boosts a neutralizing antibody response in the subject.

The neutralizing antibody response in the subject can be characterized by at least a 2-fold increase in neutralizing titer following administration of the second vaccine composition as determined by a pseudo-virus neutralization assay. In some embodiments, as compared to the subject prior to or after administration of the first vaccine composition to the subject.

In some embodiments, administration of the first and second vaccine compositions results in at least a 2-fold increase in the number of antibodies from the serum of the subject capable of specifically binding to a CD4 binding site epitope of an Env protein. In some embodiments, as compared to the subject prior to or after administration of the first vaccine composition.

In some embodiments, administration of the first and second vaccine compositions results in at least a 2-fold increase in the number of antibodies from the serum of the subject capable of binding to one or more of a polypeptide each selected from IGT1, IGT2, and variants thereof. In some embodiments, administration of the first and second vaccine compositions results in at least a 2-fold increase in the number of antibodies from the serum of the subject capable of binding to one or more of a polypeptide each comprising an amino acid sequence selected from SEQ ID NOs: 117-118 and 120-121. In some embodiments, as compared to the subject prior to or after administration of the first vaccine composition.

The neutralizing antibody response in the subject can be characterized by neutralization of two or more pseudo-viruses comprising an Env protein or portion thereof, each of an HIV variant different from one another, by the sera of the subject, following administration of the fifth vaccine composition. Neutralization can be defined as having a percent neutralization of about 40% or more at a serum dilution of about 1:100, as measured by a pseudo-virus neutralization assay.

In some embodiments, administration of the first, second, third, fourth, and fifth vaccine compositions results in at least a 0.5-fold increase in the number of antibodies from the serum of the subject capable of specifically binding to a CD4 binding site epitope of an Env protein. In some embodiments, as compared to the subject prior to or after administration of the first vaccine composition.

In some embodiments, administration of the first, second, third, fourth, and fifth vaccine compositions results in at least a 0.5-fold increase in the number of antibodies from the serum of the subject capable of binding to one or more of a polypeptide each selected from IGT1, IGT2, 426c, and variants thereof. In some embodiments, administration of the first, second, third, fourth, and fifth vaccine compositions results in at least a 0.5-fold increase in the number of antibodies from the serum of the subject capable of binding to one or more of a polypeptide each comprising an amino acid sequence selected from SEQ ID NOs: 117-125. In some embodiments, as compared to the subject prior to or after administration of the first vaccine composition.

In some embodiments, administration of the first, second, third, fourth, and fifth vaccine compositions results in at least a 0.5-fold increase in the number of antibodies from the serum of the subject capable of binding one or more of a polypeptide each comprising an HIV Env protein selected from BG505 Env protein, AMCO11 Env protein, B41 Env protein, CH119 Env protein, CE0217 Env protein, CNE8 Env protein, CNE8 N276A Env protein, CNE20 Env protein, CNE20 N276A Env protein, and variants thereof. In some embodiments, as compared to the subject prior to or after administration of the first vaccine composition.

In some embodiments, administration of the first, second, third, fourth, and fifth vaccine compositions results in at least a 0.5-fold increase in the number of antibodies from the serum of the subject capable of binding one or more of a polypeptide each comprising an amino acid sequence selected from SEQ ID NOs: 140-148. In some embodiments, as compared to the subject prior to or after administration of the first vaccine composition.

Administering the first vaccine composition can be a prime and administration of the second vaccine composition can be a boost. Administering the first vaccine composition can be a prime and administration of the second, third, fourth and fifth vaccine compositions can be each a boost.

The administration can comprise intravenous, intraperitoneal or subcutaneous administration. The subject can be a mammal. The mammal can be a mouse, a rat, a rabbit, or a primate. The primate can be a rhesus macaque, a cynomolgus macaque, a pigtail macaque, an ape, or a human.

Pharmaceutical Compositions and Therapeutic Applications

Also provided herein include a vaccine composition comprising the carrier as herein described, in combination with one or more compatible and pharmaceutically acceptable carriers. A vaccine composition is a pharmaceutical composition that can elicit a prophylactic (e.g., to prevent or delay the onset of a disease, or to prevent the manifestation of clinical or subclinical symptoms thereof) or therapeutic (e.g., suppression or alleviation of symptoms) immune response in a subject.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, pharmaceutically acceptable carriers comprise a pharmaceutical acceptable salt. As used herein, a “pharmaceutical acceptable salt” includes a salt of an acid form of one of the components of the compositions herein described. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

The vaccine composition can further comprise appropriate adjuvants. Adjuvant refers to any immunomodulating substance capable of being combined with the protein antigens herein described to enhance, improve or otherwise modulate an immune response in a subject. The adjuvants can be covalently or non-covalently attached or coupled to the surface of the carrier via any of a variety approaches known in the art. Exemplary adjuvants that can be attached to the carrier include, but are not limited to, immunostimulatory peptides, oligonucleotide CpG motifs, immunostimulatory carbohydrates and polysaccharides, and immunostimulatory protein or peptide molecules (e.g. cytokines, chemokines, flagellin, and derivatives thereof), Freund's adjuvant, sapanin (e.g., Matrix M1), lecithin, aluminum hydroxide, monophosphoryl lipid A, interleukin-12, STING agonist, Advax, and AS01_{B}, STING agonist (e.g., bis-(3′,5′)-cyclic dimeric guanosine monophosphate (c-di-GMP or cdGMP)).

The vaccine composition can comprise an adjuvant. In some embodiments, the adjuvant is selected from: saponin/MPLA nanoparticles (SMNP), aluminum hydroxide, alhydrogel, AddaVax, MF59, AS03, Freund's adjuvant, Montanide ISA51, CpG, Poly J:C, glucopyranosyl lipid A, flagellin, resiquimod, and a combination thereof. As a skilled person will understand, both MF58® and AddaVax™ are squalene-based oil-in-water nano-emulsion.

The vaccine composition can be formulated for a variety of modes of administration. Techniques for formulation and administration can be found, for example, in “Remington's Pharmaceutical Sciences”, 18^{th} ed., 1990, Mack Publishing Co., Easton, Pa. In some embodiments, the vaccine compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the hydrogel composition. The pharmaceutical compositions can comprise one or more pharmaceutically-acceptable carriers.

Formulations useful in the methods of the present disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a pharmaceutically acceptable carrier to produce a single dosage form will vary depending upon the host being treated, the particular mode of administration. The amount of active ingredient, which can be combined with a pharmaceutically acceptable carrier to produce a single dosage form can generally be that amount of the carrier which produces a therapeutic effect or an immune response. Generally, out of one hundred percent, this amount can range from about 1% to about 99% of active ingredient, preferably from about 5% to about 70%, most preferably from about 10% to about 30%.

The vaccine composition can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Applications

The vaccine compositions disclosed herein can be employed in a variety of therapeutic or prophylactic applications to stimulate an immune response in a subject in need, to treat or prevent an HIV infection in a subject in need, and/or to treat or prevent a disease or disorder caused by an HIV infection in a subject in need.

As used herein, the term “treatment” or “treat” refers to an intervention made in response to a disease, disorder or physiological condition (e.g., an HIV infection) manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

The term “condition” as used herein indicates a physical status of the body of an individual (as a whole or as one or more of its parts), that does not conform to a standard physical status associated with a state of complete physical, mental and social well-being for the individual. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

The terms “subject”, “subject in need”, and “individual” as used herein refer to an animal and in particular higher animals and in particular vertebrates such as mammals and more particularly human beings. In some embodiments, the subject or individual has been exposed to HIV. The term “exposed” indicates the subject has come in contact with a person or an animal that is known to be infected with HIV. In some embodiments, a subject in need can be a healthy subject exposed to or at risk of being exposed to HIV. In some embodiments, subjects in need include those already suffering from the disease or disorder caused by an HIV infection or those diagnosed with an HIV infection.

Accordingly, the one or more vaccine compositions can be administered in advance of any symptom, for example, in advance of an HIV infection. The one or more vaccine compositions can also be administered at or after the onset of a symptom of disease or infection, for example, after development of a symptom of infection or after diagnosis of the infection.

The phrase “therapeutically effective amount” as used herein means that amount of carriers disclosed herein which is effective for producing some desired therapeutic effect and/or generating a desired response, such as reduce or eliminate a sign or symptom of a condition or disease, such as pneumonia, at a reasonable benefit/risk ratio. The therapeutically effective amount also varies depending on the structure and antigens of the carrier, the route of administration utilized, and the specific diseases or disorders to be treated as will be understood to a person skilled in the art. For example, if a given clinical treatment is considered effective when there is at least a 20% reduction in a measurable parameter associated with a disease or disorder, a therapeutically effective amount of the carriers for the treatment of that disease or disorder is the amount necessary to achieve at least a 20% reduction in that measurable parameter.

In some embodiments, a therapeutically effective amount is necessary to inhibit HIV replication or to measurably alleviate outward symptoms of the viral infection or inhibiting further development of the disease, condition, or disorder. In some embodiments, a therapeutically effective amount is an amount that prevents one or more signs or symptoms that can be caused by an HIV infection. In some embodiments, a therapeutically effective amount can be an amount that prevents one or more signs or symptoms of a particular disease or condition from developing, such as one or more signs or symptoms associated with HIV infections.

A therapeutically effective amount of the one or more vaccine compositions herein described can be estimated from data obtained from cell culture assays and further determined from data obtained in animal studies, followed up by human clinical trials. For example, toxicity and therapeutic efficacy of the one or more vaccine compositions described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

In some embodiments, the determination of a therapeutically effective amount of the one or more vaccine compositions can be measured by measuring the titer of antibodies produced against an HIV strain. Methods of determining antibody titers and methods of performing virus neutralization arrays are known to those skilled in the art as well as exemplified in the example section of the present disclosure.

Methods of stimulating an immune response in a subject in need are disclosed herein, comprising administering to the subject a pharmaceutically effective amount of the one or more vaccine compositions, thereby stimulating an immune response in the subject in need. In some embodiments, administering the one or more vaccine compositions induces neutralizing responses against HIV variants (e.g., strains) different from the first HIV variant and the second HIV variant. In some embodiments, administering the one or more vaccine compositions induces neutralizing responses against additional HIV strains different from the HIV strains from which the plurality of HIV immunogens are derived to produce the one or more vaccine compositions. In some embodiments, administering the one or more vaccine compositions induces neutralizing responses against the HIV strains from which the plurality of HIV immunogens are derived to produce the one or more vaccine compositions.

In some embodiments, a method for treating or preventing an HIV infection in a subject in need thereof is disclosed, the method comprising administering to the subject a pharmaceutically effective amount of the one or more vaccine compositions herein described, thereby treating or preventing the HIV infection in the subject. In some embodiments, administering the one or more vaccine compositions results in treating or preventing infection caused by an HIV variant different from the first HIV variant and the second HIV variant. In some embodiments, administering the one or more vaccine compositions results in treating or preventing infection caused by additional HIV variants different from the HIV variants from which the plurality of HIV immunogens are derived to produce the one or more vaccine compositions. In some embodiments, administering the one or more vaccine compositions results in treating or preventing infection caused by the HIV strains (e.g., variants) from which the plurality of HIV immunogens are derived to produce the one or more vaccine compositions.

In some embodiments, a method of treating or preventing a disease or disorder caused by an HIV infection in a subject in need thereof is disclosed, the method comprising administering to the subject a pharmaceutically effective amount of the one or more vaccine compositions herein described, thereby treating or preventing the disease or disorder caused by the HIV infection in the subject. In some embodiments, administering the one or more vaccine compositions results in treating or preventing the disease or disorder caused by an HIV variant different from the first HIV variant and the second HIV variant. In some embodiments, administering the one or more vaccine compositions results in treating or preventing the disease or disorder caused by additional HIV variants (e.g., strains or quasiospecies) different from the HIV strains from which the plurality of HIV immunogens are derived to produce the one or more vaccine compositions. In some embodiments, administering the one or more vaccine compositions results in treating or preventing the disease or disorder caused by the HIV strains from which the plurality of HIV immunogens are derived to produce the one or more vaccine compositions.

In some embodiments, the one or more vaccine compositions can be used for treating and preventing a broad spectrum of HIV infections or a disease and disorder caused by such infections (e.g., acquired immune deficiency syndrome or AIDS) by inducing broadly protective anti-HIV responses. For example, the one or more vaccine compositions herein described can elicit broadly neutralizing antibodies that neutralize one or more HIV variants, strains, or quasispecies that differ from the HIV variants, strains, or quasispecies from which the HIV immunogens are derived to produce the one or more vaccine compositions.

The one or more vaccine compositions herein described can be administered using techniques well known to those skilled in the art, such as injection, inhalation or insulation or by oral, parenteral or rectal administration. The one or more vaccine compositions can be administered by means including, but not limited to, traditional syringes and needleless injection devices. Suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections. For injection, the one or more vaccine compositions herein described can be formulated in aqueous solutions, preferably in physiologically compatible buffers such as Hanks' solution, Ringer's solution, or physiological saline buffer.

In some embodiments, the carriers and vaccine compositions thereof can be administered to a subject systematically. The wording “systemic administration” as used herein indicates any route of administration by which a vaccine composition is brought in contact with the body of the individual, so that the resulting composition location in the body is systemic (i.e. non limited to a specific tissue, organ or other body part where the vaccine is administered). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

The one or more vaccine compositions herein disclosed can be administered to a subject using a prime/boost protocol. In such protocol, a first vaccine composition is administered to the subject (prime) and then after a period of time, a second vaccine composition can be administered to the subject (boost). Administration of the second composition (boost composition) can occur days, weeks or months after administration of the first composition (prime composition). For example, the boost composition can be administered about three days, 1 week, 2 weeks, 3 weeks, 4 weeks, 5 weeks, 6 weeks, 7 weeks, 8 weeks, 12 weeks, 16 weeks, 20 weeks, 24 weeks, or 28 weeks, or a number or a range between any two of these values, after the prime composition is administered. In some embodiments, the boost composition can be administered about 4 weeks after administration of the prime composition.

Therefore, the one or more vaccine compositions can be administered to the subject in need two or more times. For example, the methods herein described can comprise administering to the subject a first vaccine composition, and after a period of time, administering to the subject a second vaccine composition.

The prime vaccine composition and the boost vaccine composition can be, but need not be, the same composition. In some embodiments, the prime vaccine composition and the boost vaccine composition can contain the same or different HIV immunogens attached to the carrier. In some embodiments, the prime vaccine composition and the boost vaccine composition can contain the same HIV immunogens attached to the carrier, but with the carrier in different pharmaceutically effective amounts. In some embodiments, the prime vaccine composition and the boost vaccine composition can contain different adjuvants. In some embodiments, the prime vaccine composition comprises a monovalent carrier disclosed herein.

The carriers and the vaccine compositions thereof can be used to protect a subject against infection by heterologous HIV strains. In other words, a vaccine composition made using HIV immunogens and the methods disclosed herein are capable of protecting an individual against infection by two or more HIV variants. In some embodiments, the carriers and the vaccine composition thereof can protect an individual against infection by an antigenically divergent HIV variant.

### Antibodies

The present disclosure provides specific antibodies or fragments thereof with high affinity and specificity to the Env protein of HIV, in particular, the CD4 binding site (CD4bs) of the Env protein. In some embodiments, the CD4bs-specific antibodies herein described are broadly neutralizing and potent. In some embodiments, the antibodies herein described can be used for treating a patient in need thereof, who is suffering from, e.g., an HIV infection. There are provided, in some embodiments, compositions comprising one or more of the antibody or fragment thereof as described herein.

In some embodiments, the antibodies or fragments thereof disclosed herein contain CDR regions defined in SEQ ID NOs: 203-254 or variants thereof having one, two or three mismatches (e.g., a single substitution, deletion or insertion) in any one of SEQ ID NOs: 203-254.

Disclosed herein include antibodies or fragments thereof. In some embodiments, the antibody or fragment thereof has specificity to a CD4 binding site of an HIV Env protein and comprises: (a) a heavy chain variable region (VH) CDR1 comprising an amino acid sequence selected from SEQ ID NOs: 203-210 or a variant thereof having a single substitution, deletion or insertion from any one of SEQ ID NOs: 203-210; (b) a VH CDR2 comprising an amino acid sequence selected from SEQ ID NOs: 213-220 or a variant thereof having a single substitution, deletion or insertion from any one of SEQ ID NOs: 213-220; (c) a VH CDR3 comprising an amino acid sequence selected from SEQ ID NOs: 222-229 or a variant thereof having a single substitution, deletion or insertion from any one of SEQ ID NOs: 222-229; (d) a light chain variable region (VL) CDR1 comprising an amino acid sequence selected from SEQ ID NOs: 230 and 232-238 or a variant thereof having a single substitution, deletion or insertion from any one of SEQ ID NOs: 230 and 232-238; (e) a VL CDR2 comprising an amino acid sequence selected from SEQ ID NOs: 239 and 241-245, or a variant thereof having a single substitution, deletion or insertion from any one of SEQ ID NOs: 239 and 241-245; and (f) a VL CDR3 comprising an amino acid sequence selected from SEQ ID NOs: 248-254 or a variant thereof having a single substitution, deletion or insertion from any one of SEQ ID NOs: 248-254.

The antibody or fragment thereof can comprise a heavy chain variable region comprising (i) an amino acid sequence selected from SEQ ID NOs: 3-11, (ii) an amino acid sequence having at least 90% sequence identity (e.g., 90%, 91%, 92%, 93%, 94%, 95%, 96%, 97%, 98%, 99%, 100%, or a number or a range between any two of these values) to an amino acid sequence selected from SEQ ID NOs: SEQ ID NOs: 3-11, or (iii) an amino acid sequence having one, two or three mismatches relative to an amino acid sequence selected from SEQ ID NOs: 3-11. The antibody or fragment thereof can comprise a light chain variable region comprising (i) an amino acid sequence selected from SEQ ID NOs: 14-22, (ii) an amino acid sequence having at least 90% sequence identity (e.g., 90%, 91%, 92%, 93%, 94%, 95%, 96%, 97%, 98%, 99%, 100%, or a number or a range between any two of these values) to an amino acid sequence selected from SEQ ID NOs: 14-22, or (iii) an amino acid sequence having one, two or three mismatches relative to an amino acid sequence selected from SEQ ID NOs: 14-22. The antibody or fragment thereof can comprise a heavy chain variable region comprising (i) an amino acid sequence selected from SEQ ID NOs: 3-11 and 27-76, (ii) an amino acid sequence having at least 90% sequence identity (e.g., 90%, 91%, 92%, 93%, 94%, 95%, 96%, 97%, 98%, 99%, 100%, or a number or a range between any two of these values) to an amino acid sequence selected from SEQ ID NOs: SEQ ID NOs: 3-11 and 27-76, or (iii) an amino acid sequence having one, two or three mismatches relative to an amino acid sequence selected from SEQ ID NOs: 3-11 and 27-76. The antibody or fragment thereof can comprise a light chain variable region comprising (i) an amino acid sequence selected from SEQ ID NOs: 14-22 and 77-116, (ii) an amino acid sequence having at least 90% sequence identity (e.g., 90%, 91%, 92%, 93%, 94%, 95%, 96%, 97%, 98%, 99%, 100%, or a number or a range between any two of these values) to an amino acid sequence selected from SEQ ID NOs: 14-22 and 77-116, or (iii) an amino acid sequence having one, two or three mismatches relative to an amino acid sequence selected from SEQ ID NOs: 14-22 and 77-116.

The antibody or fragment thereof can comprise an Fc domain. The antibody or fragment thereof can be a single-chain variable fragment (scFv), a single-domain antibody, an immunoglobulin molecule, a monoclonal antibody, a chimeric antibody, a CDR-grafted antibody, a humanized antibody, a Fab fragment, a Fab′ fragment, a F(ab′)₂ fragment, an Fv fragment, a disulfide linked Fv, an scFv, a single domain antibody, a diabody, a multispecific antibody, a dual specific antibody, an anti-idiotypic antibody, a bispecific antibody, or a functionally active epitope-binding fragment thereof.

The antibody or fragment thereof can be a single-chain variable fragment (scFv) or a single-domain antibody. Single-chain variable fragment can be formed by linking the heavy and light chain fragments of the Fv region via an amino acid bridge (e.g., a disulfide linkage), resulting in a single-chain fusion peptide. The heavy and light chain fragments of the Fv region can be selected from any of the heavy and light chain fragments and variants thereof described herein (e.g., SEQ ID NOs: 3-11 and 14-22 or variants thereof). Examples of techniques which can be used to produce scFvs and antibodies include those described in U.S. Pat. Nos. 4,946,778 and 5,258,498; Huston et al., Methods in Enzymology 203:46-88 (1991); Shu et al., Proc. Natl. Sci. USA 90:1995-1999 (1993); and Skerra et al., Science 240:1038-1040 (1988). In some embodiments, the antibody or fragment thereof is a single-domain antibody comprising a heavy chain variable region or a variant thereof described herein.

In some embodiments, the antibodies or fragments thereof do not elicit an undesirable (e.g., deleterious) immune response in a subject to be treated, e.g., in a human. In some embodiments, antibodies, fragments, variants, or derivatives thereof of the disclosure are modified to reduce their immunogenicity using techniques recognized in the art. For example, antibodies can be humanized, primatized, deimmunized, or chimeric antibodies.

In some embodiments, the antibody or fragment thereof specifically binds to two or more different HIV Env proteins. Binding of the antibodies and fragments thereof as disclosed herein can be assessed by any assay known in the art, including, but not limited to, precipitation assay, agglutination assay, ELISA, surface plasma resonance, western blot, and FACS. In some embodiments, binding can be assessed by an optofluidic system (e.g., Berkeley Light Beacon® Optofluidic System). As described herein, the optofluidic technology can comprise distributing cells within a sample into individual compartments using microfluidic devices, and detecting a signal associated with the subset of cells with the property of interest. The term “enzyme linked immunosorbent assay” (ELISA) as used herein can refer to an antibody-based assay in which detection of the antigen of interest is accomplished via an enzymatic reaction producing a detectable signal. An ELISA can be run as a competitive or non-competitive format.

The antibody or fragment thereof can inhibit infectivity of a virus comprising an HIV Env protein with an IC50 less than 100 μg/mL, less than 10 μg/mL, less than 1 μg/mL or less than 0.1 μg/mL. In some embodiments, as measured by a pseudo-virus neutralization assay. The antibody or fragment thereof can inhibit infectivity of two or more viruses each comprising a different HIV Env protein. In some embodiments, as measured by a pseudo-virus neutralization assay. The antibody or fragment thereof inhibits infectivity of at least one, at least two, or all of the two or more viruses with an IC50 less than 100 μg/mL, less than 10 μg/mL, less than 1 μg/mL, or less than 0.1 μg/mL.

The antibody or fragment thereof inhibits infectivity of at least one, at least two, or all of the two or more viruses with an IC50 of about 0.01 μg/mL to about 100 μg/mL (e.g., 0.01, 0.05, 0.1, 0.5, 1, 1.5, 2, 3, 4, 5, 6, 7, 8, 9, 10, 50, 100 μg/mL or a number or a range between any two of these values), about 0.01 μg/mL to about 10 μg/mL (e.g., 0.01, 0.05, 0.1, 0.5, 1, 1.5, 2, 3, 4, 5, 6, 7, 8, 9, 10, μg/mL or a number or a range between any two of these values), or about 0.01 μg/mL to about 1 μg/mL (e.g., 0.01, 0.05, 0.1, 0.5, 1, μg/mL or a number or a range between any two of these values).

The HIV Env protein can comprise an Env protein of an HIV variant selected from 426c, 426 N276A, CNE20, CNE20 N276A, JRCSF, YU2, PVO.4, Q23.17, Q842.D12, BG505/T332N, ZM214M.PL15, WIT04160.33, and 25710.

In some embodiments, the antibodies and fragments thereof comprise potent and/or broad neutralization activities against, e.g., one or more HIV strains or variants. “Potency” as used herein can refer to a measure of how effective an antibody or fragment thereof is at producing the desired response (e.g., inhibiting infectivity) and can be expressed in terms of the concentration (e.g., IC50) which produces a particular level of the response attainable. Broadly neutralizing antibodies can be antibodies that can neutralize two or more HIV strains. Broadly neutralizing response can also be referred to as heterologous neutralizing response. In some embodiments, the methods described herein can elicit broadly neutralizing antibodies that neutralize one or more HIV strains that differ from the HIV strain from which the HIV immunogens are derived to produce the nanoparticles.

The term “neutralizing,” as used herein, in relation to the antibodies of the disclosure refers to antibodies that are capable of preventing, reducing or inhibiting infection of a cell by the virus, by neutralizing, inhibiting, or reducing its biological effect and/or reducing the infectious titer of the virus, regardless of the mechanism by which neutralization is achieved. Neutralization can, e.g., be achieved by inhibiting the attachment or adhesion of the virus to the cell surface, or by inhibition of the fusion of viral and cellular membranes following attachment of the virus to the target cell, and the like. Neutralization potencies can be determined by any method known in the art. In some embodiments, reduced infectivity and IC50 values can be determined by, e.g., a pseudovirus neutralization assay.

There are also provided polynucleotides encoding one or more of the antibody or fragment thereof provided herein. Disclosed herein include isolated cells comprising any of the polynucleotides provided herein. There are provided compositions comprising any of the polynucleotides and/or an isolated cells provided herein.

The present disclosure provides isolated polynucleotides or nucleic acid molecules encoding the antibodies, fragments, variants or derivatives thereof of the disclosure. The polynucleotides of the present disclosure can encode the heavy and light chain variable regions of the antibodies, fragments, variants or derivatives thereof on the same polynucleotide molecule or on separate polynucleotide molecules. In some embodiments, the polynucleotides of the present disclosure can encode portions of the heavy and light chain variable regions of the antibodies (e.g., the CDR regions), fragments, variants or derivatives thereof on the same polynucleotide molecule or on separate polynucleotide molecules.

Methods of making antibodies are well known in the art and described herein. For example, polynucleotides encoding desired antibodies can be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA can be placed into expression vectors, which are then transfected into antibody-producing cells including prokaryotic or eukaryotic host cells such as E. coli cells, simian COS cells, Chinese Hamster Ovary (CHO) cells or myeloma cells that do not otherwise produce immunoglobulins. The isolated DNA can be used to clone constant and variable region sequences for the manufacture antibodies as described in Newman et al., U.S. Pat. No. 5,658,570 which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As described herein, transformed cells expressing the desired antibody can be grown up in relatively large quantities to provide clinical and commercial supplies of the immunoglobulin.

In some embodiments, mutations can be introduced in the nucleotide sequence encoding an antibody of the present disclosure using standard techniques known to those of skill in the art, including, but not limited to, site-directed mutagenesis and PCR-mediated mutagenesis which result in amino acid substitutions.

In some embodiments, the antibodies, fragments, variants, or derivatives thereof can further comprise a chemical moiety not naturally associated with an antibody. For example, the antibody or fragment thereof can comprise a flexible linker or can be modified to add a functional moiety such as a detectable label. The antibodies, fragments, variants, or derivatives thereof can be modified, i.e., by the covalent or non-covalent attachment of a chemical moiety to the antibody such that the attachment does not interfere or prevent the antibody from binding to the epitope. The chemical moiety can be conjugated to an antibody using any technique known in the art.

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

In some embodiments, the prepared antibodies do not elicit a deleterious immune response in the subject to be treated, e.g., in a human. In some embodiments, antigen-binding polypeptides, variants, or derivatives thereof of the disclosure are modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. USA 57:6851-6855 (1984); Morrison et al., Adv. Immunol. 44:65-92 (1988); Verhoeyen et al., Science 239:1534-1536 (1988); Padlan, Molec. Immun. 25:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T-cell epitopes (see, e.g., International Application Publication Nos.: WO/9852976 A1 and WO/0034317 A2). For example, variable heavy chain and variable light chain sequences from the starting antibody are analyzed and a human T-cell epitope “map” from each V region showing the location of epitopes in relation to complementarity-determining regions (CDRs) and other key residues within the sequence is created. Individual T-cell epitopes from the T-cell epitope map are analyzed in order to identify alternative amino acid substitutions with a low risk of altering activity of the final antibody. A range of alternative variable heavy and variable light sequences are designed comprising combinations of amino acid substitutions and these sequences are subsequently incorporated into a range of binding polypeptides. Typically, between 12 and 24 variant antibodies are generated and tested for binding and/or function. Complete heavy and light chain genes comprising modified variable and human constant regions are then cloned into expression vectors and the subsequent plasmids introduced into cell lines for the production of whole antibody. The antibodies are then compared in appropriate biochemical and biological assays, and the optimal variant is identified.

The binding specificity of the antibodies or fragments thereof of the present disclosure can be determined by in vitro assays such as immunoprecipitation, radioimmunoassay (RIA) or enzyme-linked immunoabsorbent assay (ELISA).

Alternatively, techniques described for the production of single-chain units (U.S. Pat. No. 4,694,778; Bird, Science 242:423-442 (1988); Huston et al., Proc. Natl. Acad. Sci. USA 55:5879-5883 (1988); and Ward et al., Nature 334:544-554 (1989)) can be adapted to produce single-chain units of the present disclosure. Single-chain units are formed by linking the heavy and light chain fragments of the Fv region via an amino acid bridge, resulting in a single-chain fusion peptide. Techniques for the assembly of functional Fv fragments in E. coli may also be used (Skerra et al., Science 242: 1038-1041 (1988)).

Examples of techniques which can be used to produce single-chain Fvs (scFvs) and antibodies include those described in U.S. Pat. Nos. 4,946,778 and 5,258,498; Huston et al., Methods in Enzymology 203:46-88 (1991); Shu et al., Proc. Natl. Sci. USA 90:1995-1999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816397, which are incorporated herein by reference in their entireties.

Humanized antibodies are antibody molecules derived from a non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen-binding. These framework substitutions are identified by methods well known in the art, e.g., by modeling of the interactions of the CDR and framework residues to identify framework residues important for antigen-binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Natl. Sci. USA 91:969-973 (1994)), and chain shuffling (U.S. Pat. No. 5,565,332, which is incorporated by reference in its entirety).

Completely human antibodies can be particularly desirable for therapeutic treatment of human patients. Human antibodies can be made by a variety of methods known in the art including phage display methods using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies can be engaged to provide human antibodies directed against a selected antigen using technology similar to that described above.

Completely human antibodies which recognize a selected epitope can also be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 72:899-903 (1988). See also, U.S. Pat. No. 5,565,332, which is incorporated by reference in its entirety.)

Additionally, using routine recombinant DNA techniques, one or more of the CDRs of the antibodies of the present disclosure, may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present disclosure and within the skill of the art.

In addition, techniques developed for the production of “chimeric antibodies” (Morrison et al., Proc. Natl. Acad. Sci. USA:851-855 (1984); Neuberger et al., Nature 372:604-608 (1984); Takeda et al., Nature 314:452-454 (1985)) by splicing genes from a mouse antibody molecule, of appropriate antigen specificity, together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

Alternatively, antibody-producing cell lines may be selected and cultured using techniques well known to the skilled artisan. Such techniques are described in a variety of laboratory manuals and primary publications. In this respect, techniques suitable for use in the disclosure as described below are described in Current Protocols in Immunology, Coligan et al., Eds., Green Publishing Associates and Wiley-Interscience, John Wiley and Sons, New York (1991) which is herein incorporated by reference in its entirety, including supplements.

Additionally, standard techniques known to those of skill in the art can be used to introduce mutations in the nucleotide sequence encoding an antibody of the present disclosure, including, but not limited to, site-directed mutagenesis and PCR-mediated mutagenesis which result in amino acid substitutions. Preferably, the variants (including derivatives) encode fewer than 50 amino acid substitutions, fewer than 40 amino acid substitutions, fewer than 30 amino acid substitutions, fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the reference variable heavy chain region, CDR-H1, CDR-H2, CDR-H3, variable light chain region, CDR-L1, CDR-L2, and/or CDR-L3. In some embodiments, one or more mutations are introduced randomly along all or part of the coding sequence, such as by saturation mutagenesis, and the resultant mutants can be screened for biological activity to identify mutants that retain activity.

### Kits

The HIV immunogens and compositions as described herein can be provided as components of a kit.

Kits can include carriers or vaccines of the present disclosure as well components for making such carriers and vaccines. As such, kits can include, for example, primers, nucleic acid molecules, expression vectors, nucleic acid constructs encoding protein antigens and/or particle-forming subunits described herein, cells, buffers, substrates, reagents, administration means (e.g., syringes), and instructions for using any of said components. Kits can also include pre-formed carriers and HIV immunogens herein described. It should be appreciated that a kit may comprise more than one container comprising any of the aforementioned, or related, components. For example, certain parts of the kit may require refrigeration, whereas other parts can be stored at room temperature. Thus, as used herein, a kit can comprise components sold in separate containers by one or more entity, with the intention that the components contained therein be used together.

### EXAMPLES

Some aspects of the embodiments discussed above are disclosed in further detail in the following examples, which are not in any way intended to limit the scope of the present disclosure.

### Example 1

### CD4-Binding Site Immunogens Elicit Heterologous Anti-HIV-1 Neutralizing Antibodies in Transgenic and Wildtype Animals

Described in this example include methods for making HIV immunogens capable of inducing a broadly neutralizing response in a subject. Also disclosed herein are compositions comprising the isolated immunogens, methods for immunization of a subject, and one or more antibodies capable of neutralizing one or more HIV variants.

Passive transfer of broadly neutralizing anti-HIV-1 antibodies (bNAbs) protects against infection, and therefore eliciting bNAbs (e.g., IOMA antibody) by vaccination is a major goal of HIV-1 vaccine efforts. bNAbs that target the CD4-binding site (CD4bs) on HIV-1 Env are among the most broadly active, but to date, responses elicited against this epitope in vaccinated animals have lacked potency and breadth. There is a need for CD4bs bNAbs resembling the antibody IOMA that can be easier to elicit than other CD4bs antibodies that exhibit higher somatic mutation rates, a difficult-to-achieve mechanism to accommodate HIV Env's N276_{gp120} N-glycan, and rare 5-residue light chain complementarity determining region 3s (CDRL3s). Ad described herein, IOMA germline-targeting Env immunogens were developed and a sequential immunization regimen was evaluated in transgenic mice expressing germline-reverted IOMA. These mice developed CD4bs epitope-specific responses with heterologous neutralization, and cloned antibodies overcame neutralization roadblocks including accommodating the N276_{gp120} glycan, with some neutralizing selected HIV-1 strains more potently than IOMA. The immunization regimen also elicited CD4bs-specific responses in mice containing polyclonal antibody repertoires as well as rabbits and rhesus macaques. Thus, germline-targeting of IOMA-class antibody precursors represents a vaccine strategy to induce CD4bs bNAbs.

### Design of IOMA-Targeting Immunogens

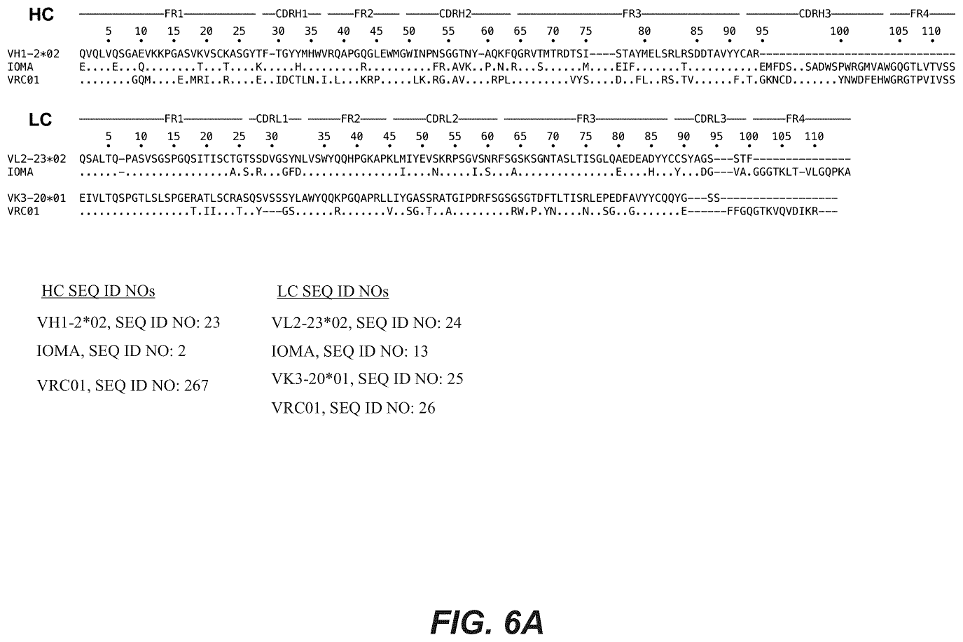

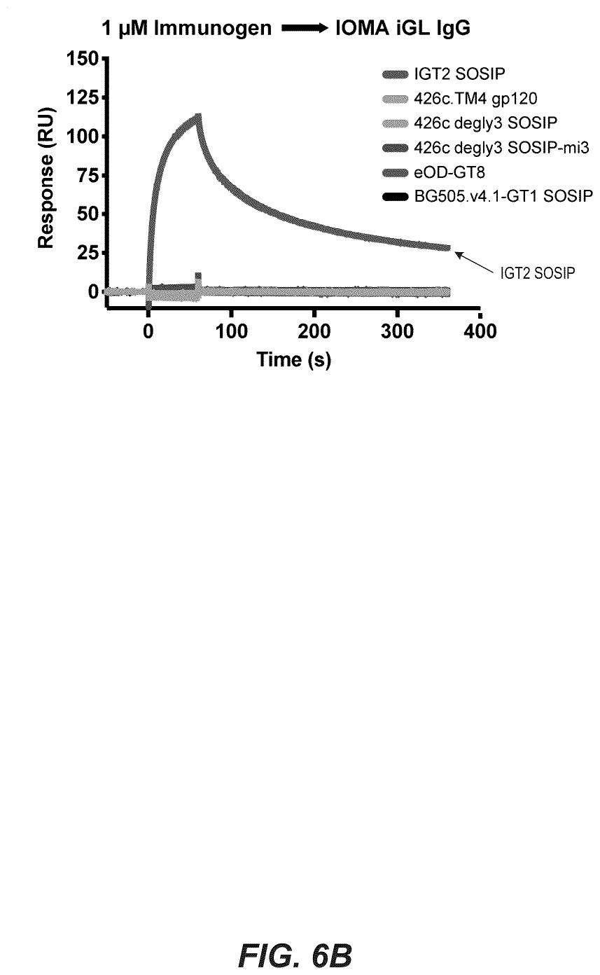

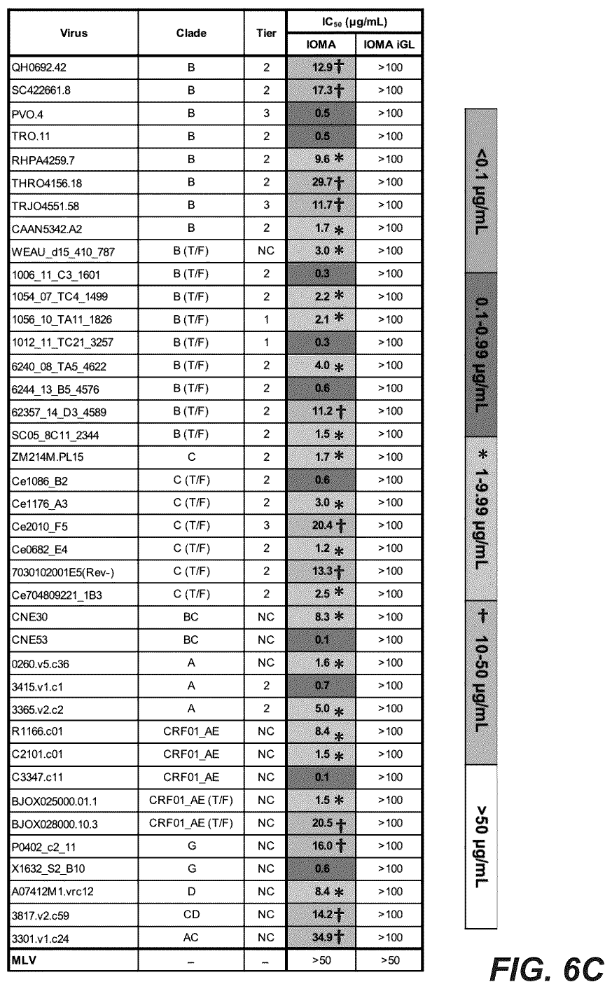

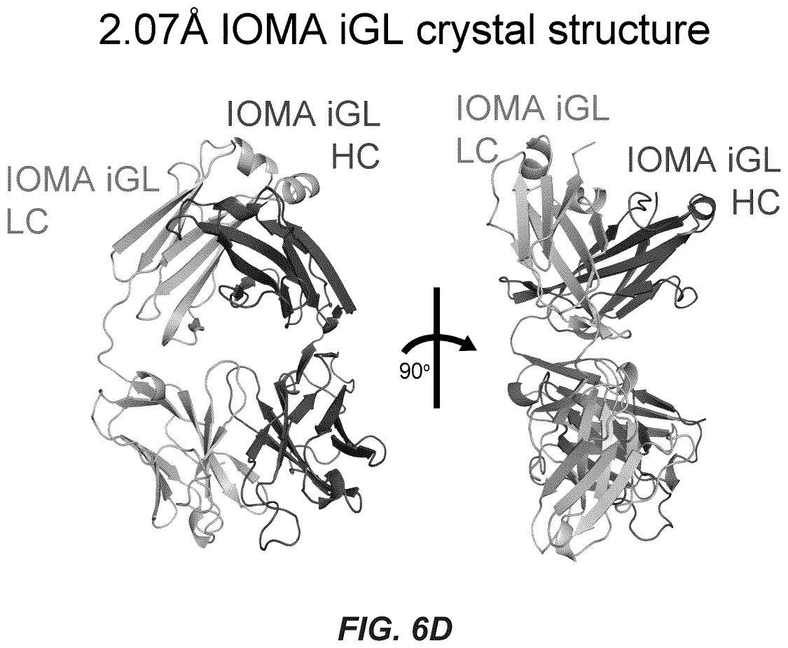

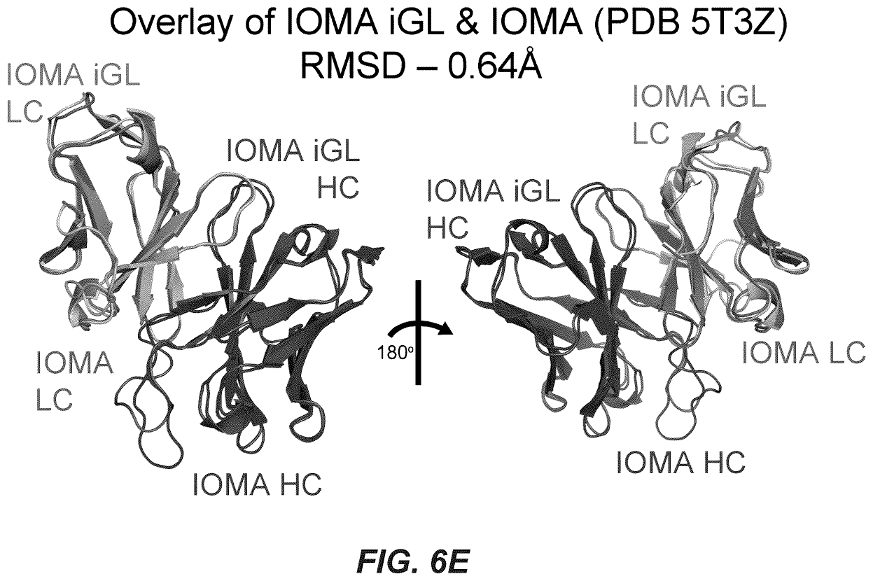

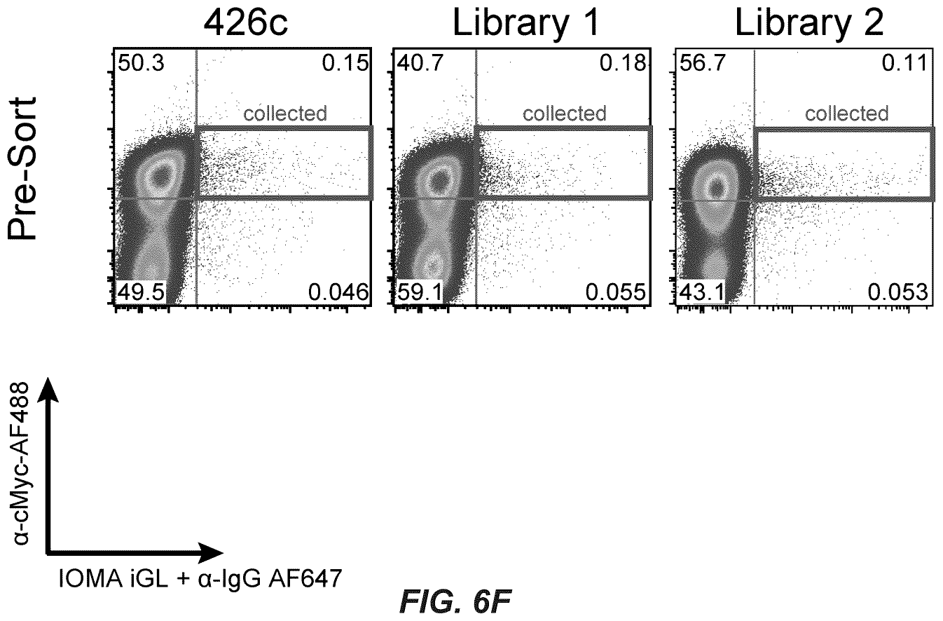

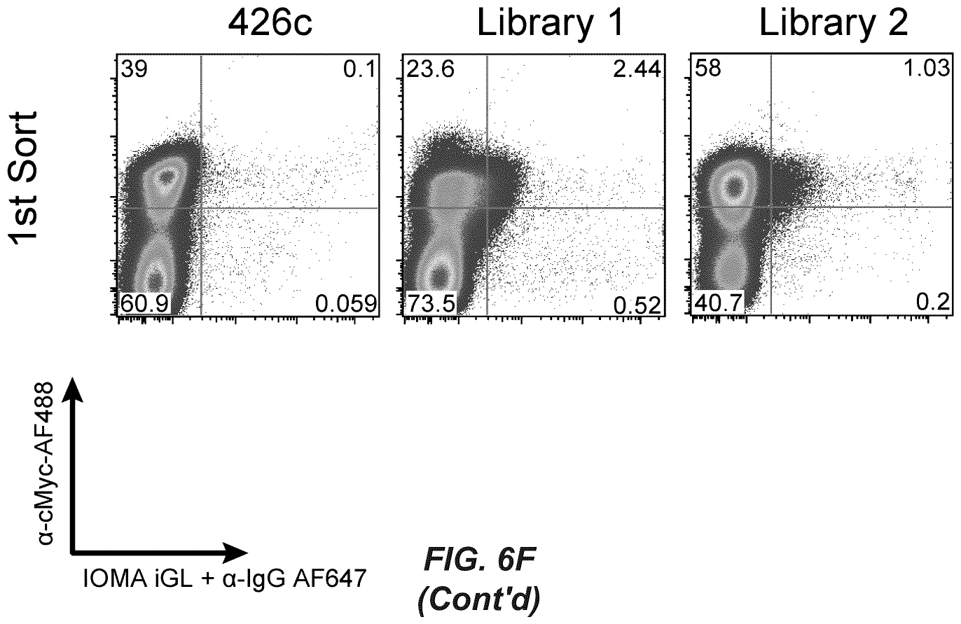

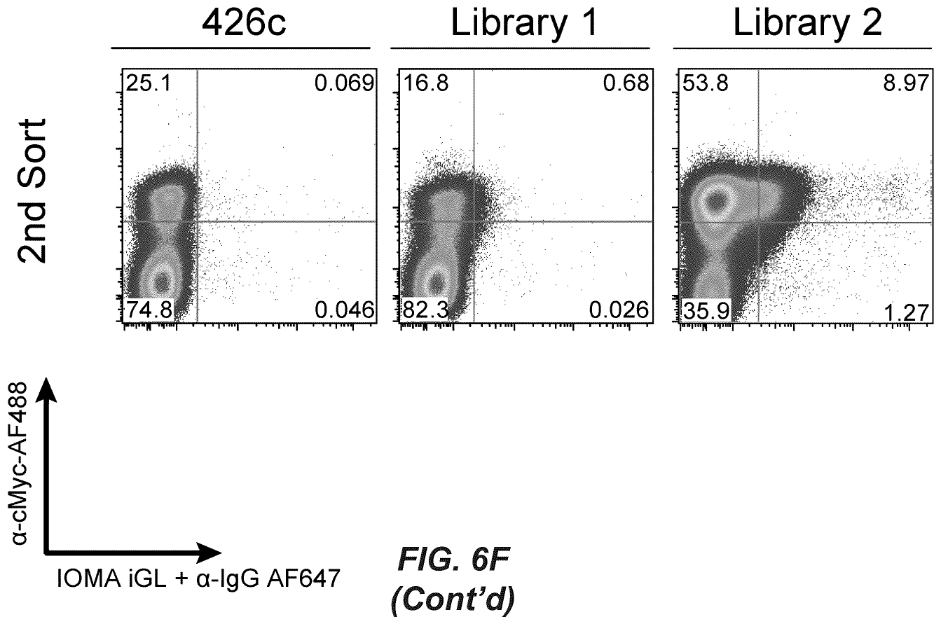

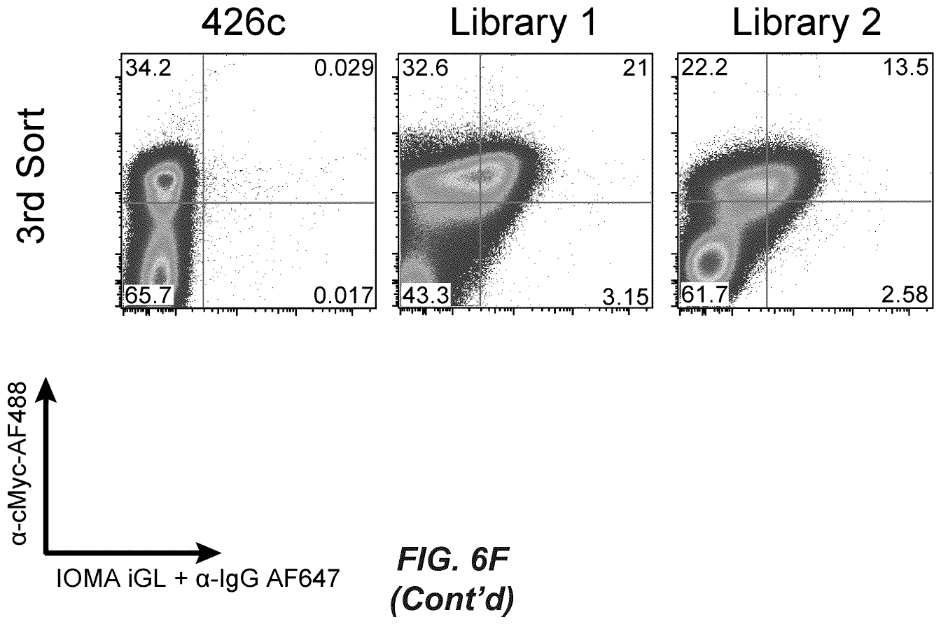

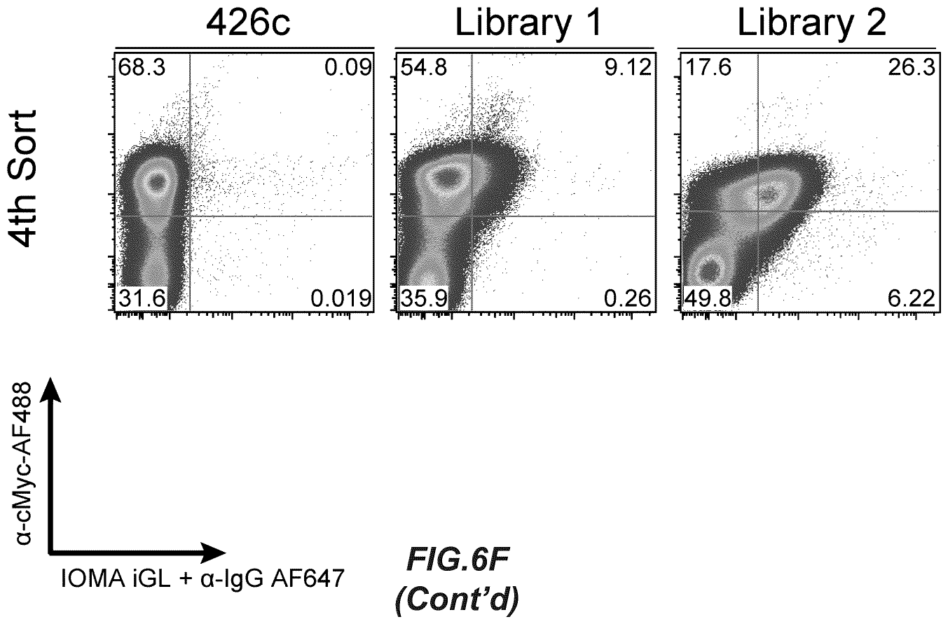

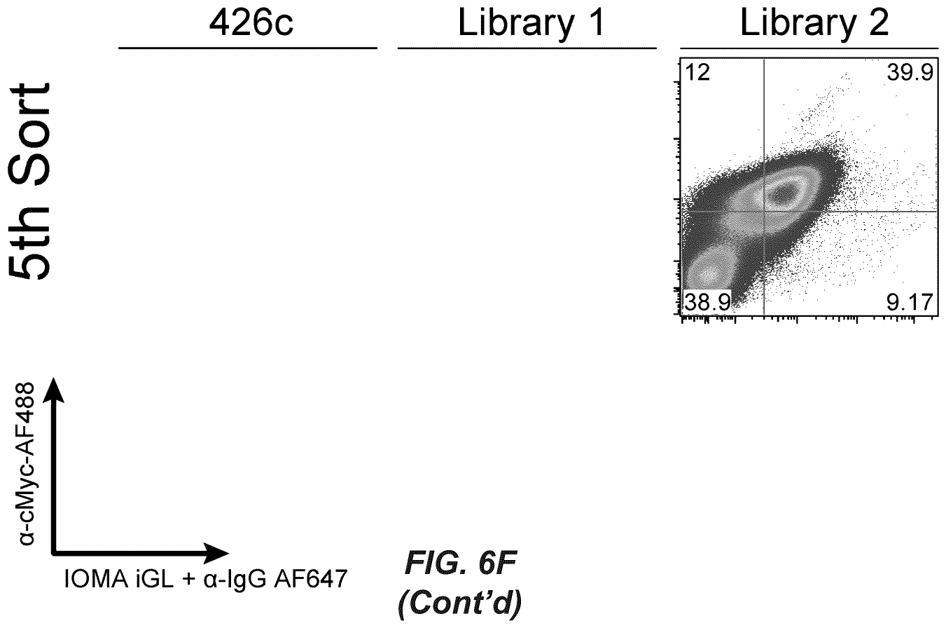

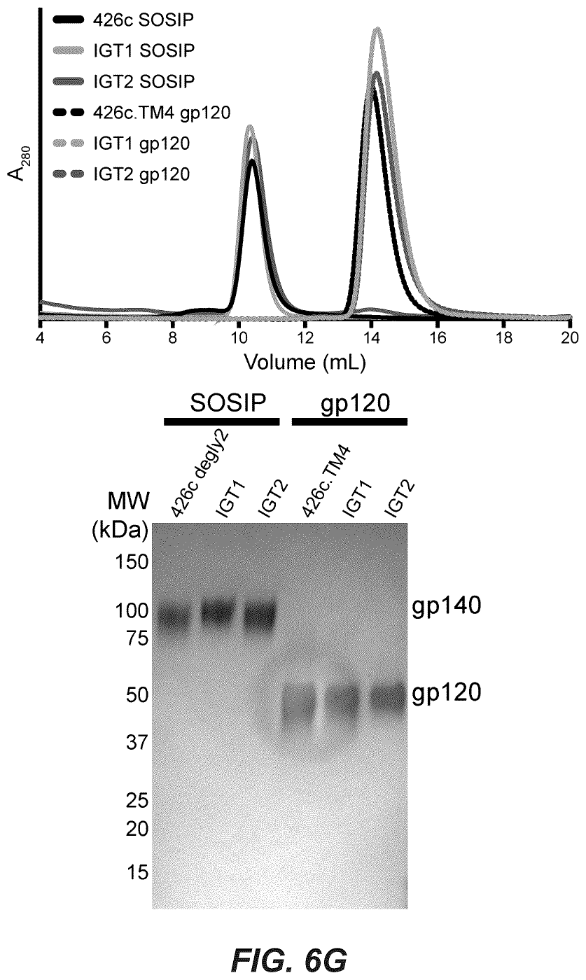

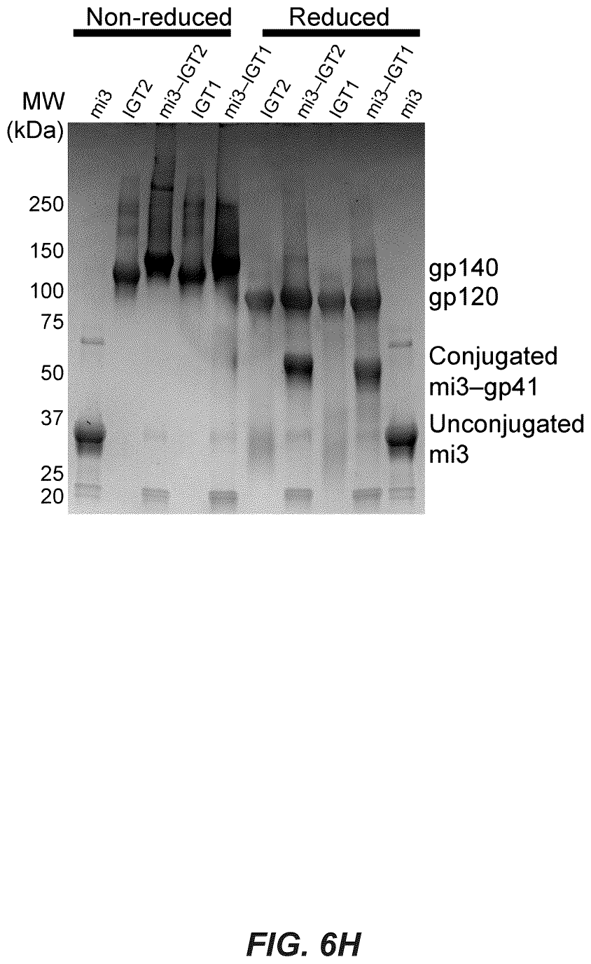

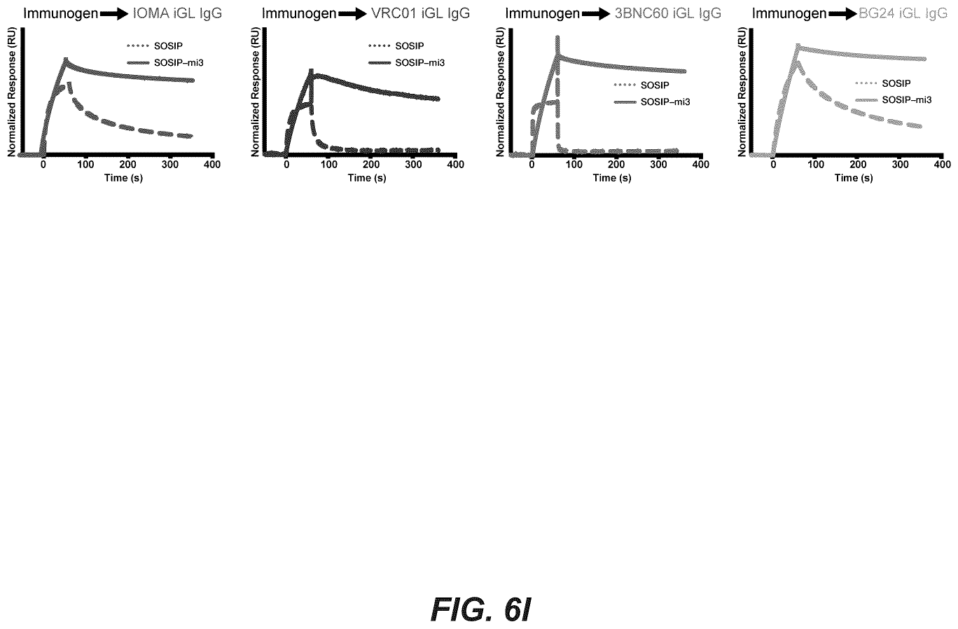

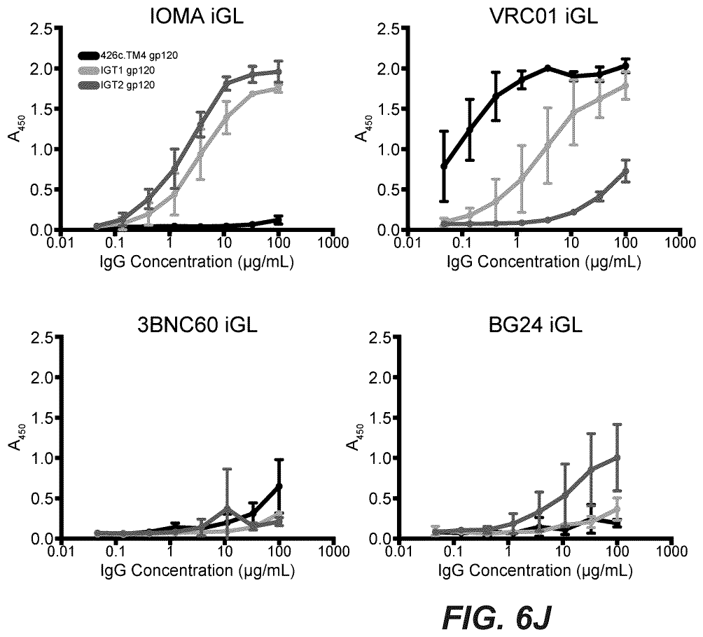

To create the IOMA iGL antibody, the HC and LC sequences of mature IOMA were reverted to their presumptive germline sequences. The IOMA iGL HC sequence was based on human IGHV1-2*02, IGHD3-22*01 and IGHJ6*02 and contained 22 amino acid changes compared to the HC of IOMA, all within the V gene. The CDRH3 was unaltered due to, in some embodiments, uncertainty with respect to D gene alignment and potential P and N nucleotides—the IOMA iGL HC sequence maintains one gp120-contacting residue (W100F; Kabat numbering) found in mature IOMA. The sequence of the IOMA iGL LC was derived from human IGLV2-23*02 and IGLJ2*01 containing 16 amino acid changes compared with mature IOMA, including 3 SHMs in CDRL3. Of the 3 mutations in CDRL3, two non-contact amino acids (V96, A97; Kabat numbering) at the V-J junction were left as in mature IOMA (FIG. 6A, Table 1).

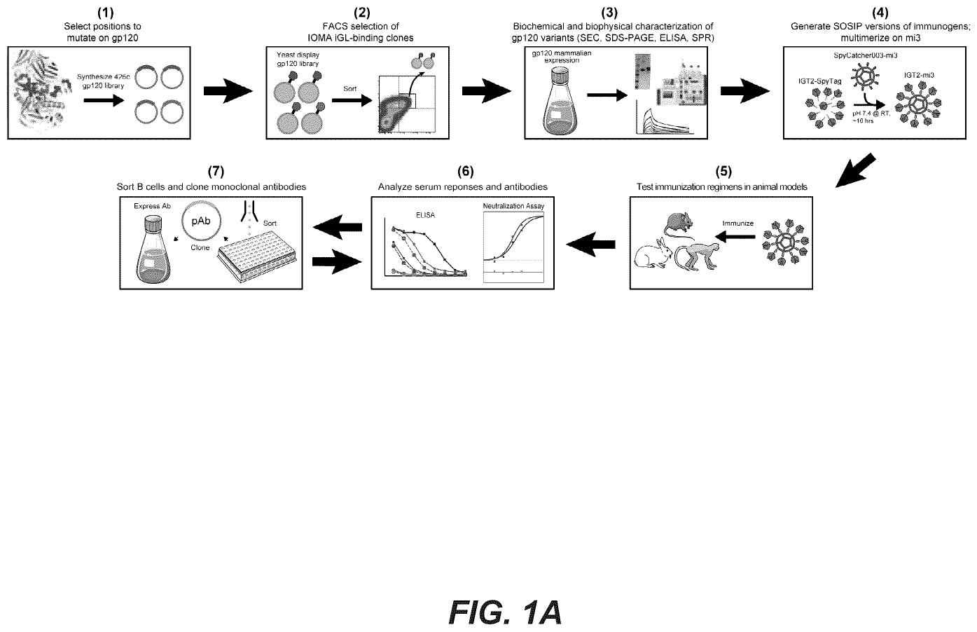

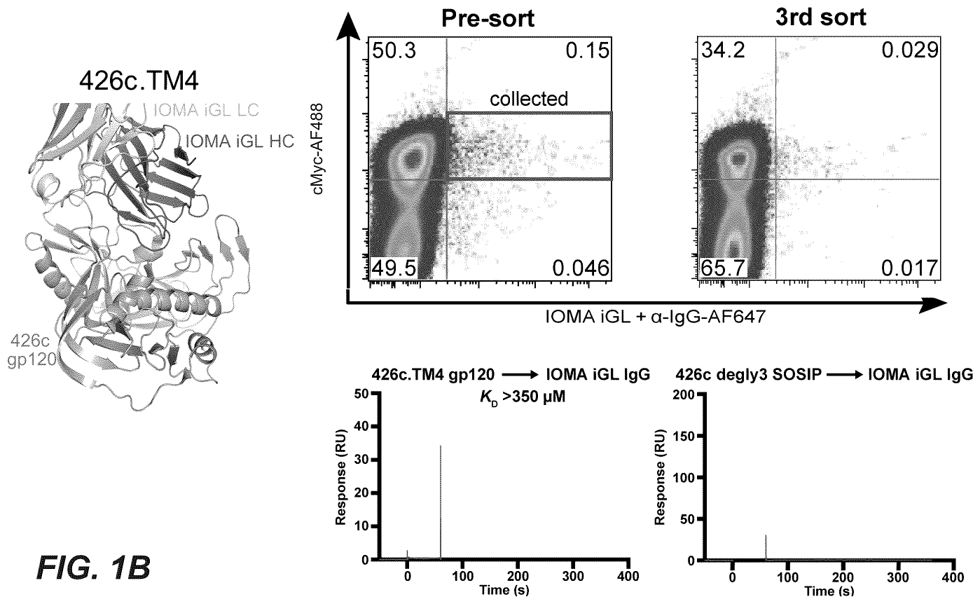

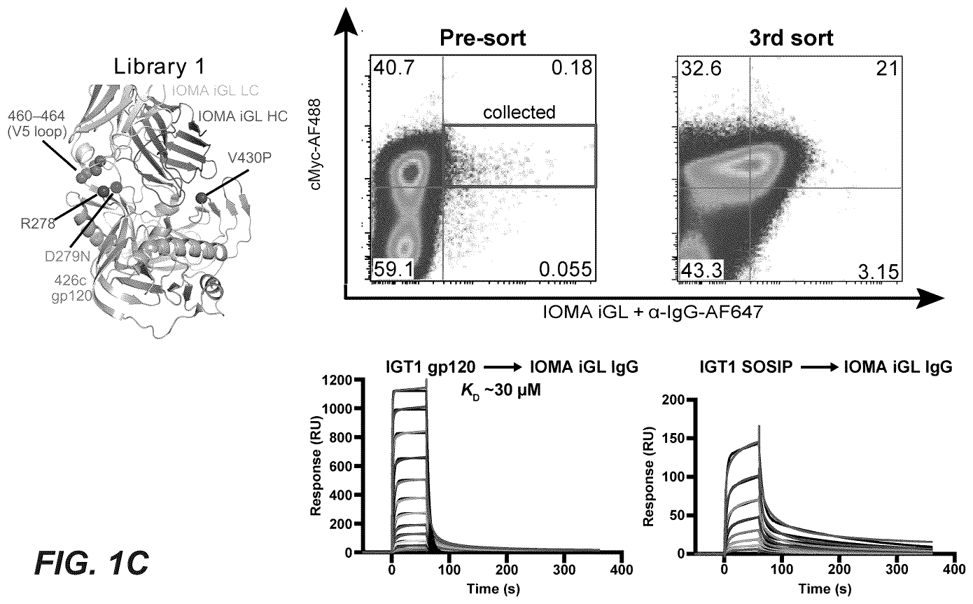

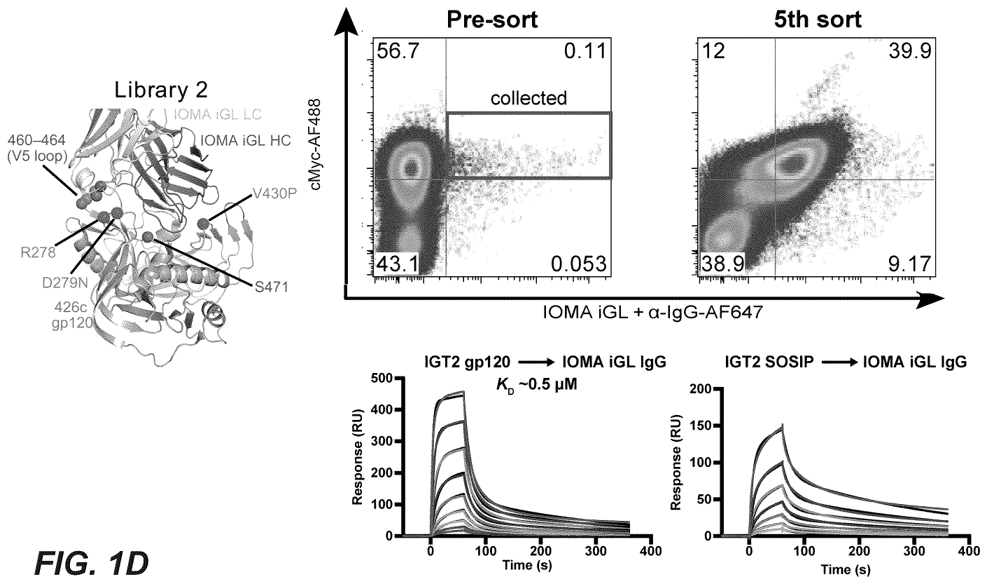

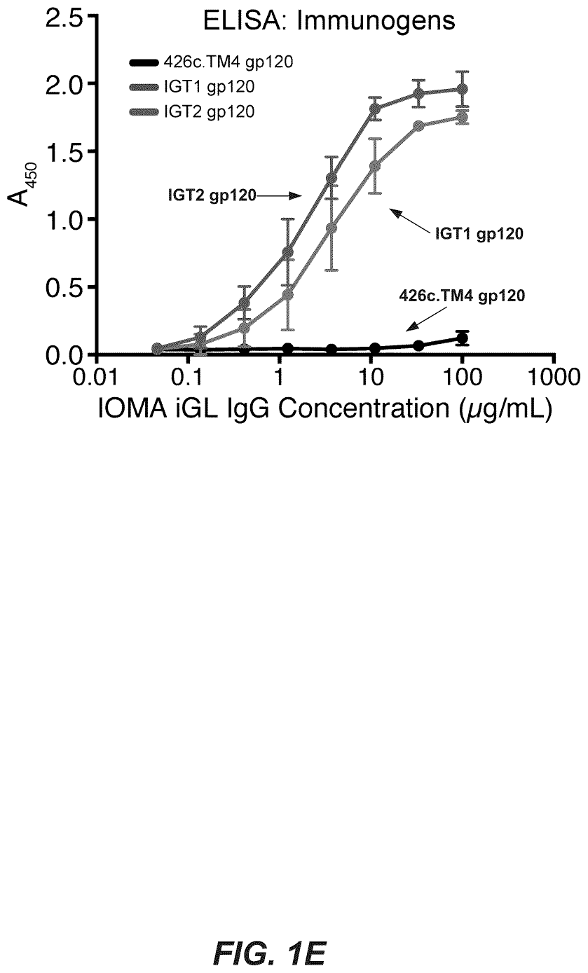

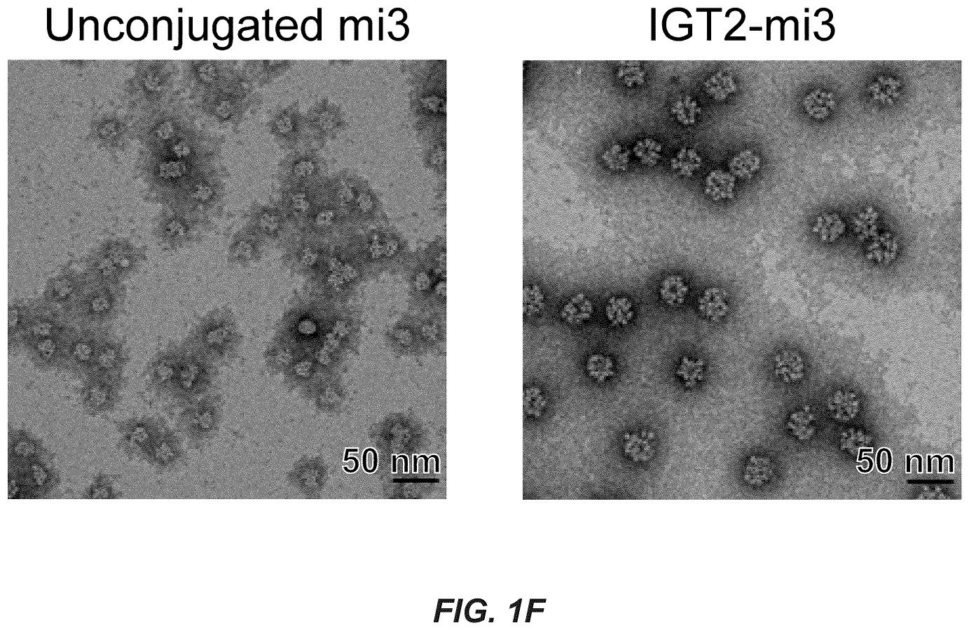

No currently available germline-targeting CD4bs immunogens bind IOMA iGL with detectable affinity (FIG. 6B), and IOMA iGL does not neutralize primary HIV-1 strains (FIG. 6C). in vitro selection methods were therefore used to identify potential IOMA-targeting immunogens (FIG. 1A). S. cerevisiae yeast display was chosen for selecting an IOMA-targeting immunogen for two reasons: (i) Yeast libraries can contain up to 1×10⁹ variants and therefore allow screening a large number of immunogen constructs, a necessity from starting with no detectable binding of IOMA iGL to any CD4bs-targeting immunogens, and (ii) S. cerevisiae attach different forms of N-linked glycans to glycoproteins than mammalian cells; e.g., yeast can add up to 50 mannoses to Man₈₋₉GlcNAc₂. Such glycan differences may be an advantage because N-glycosylated immunogens selected in a yeast library to bind IOMA iGL and increasingly mature forms of IOMA might stimulate an antibody maturation pathway that is relatively insensitive to the form of N-glycan at any potential N-linked glycosylation site (PNGS) on HIV-1 Env. Promiscuous glycan recognition is desired because Env trimers on viruses exhibit heterogeneous glycosylation at single PNGSs, even within one HIV-1 strain. Using yeast display for immunogen selection, promiscuous N-glycan accommodation was achieved through recognition of a glycan's core pentasaccharide, a common feature of both complex-type and high-mannose glycans, which were observed as being recognized at some N-glycan sites in structures of antibody Fab-Env complexes.

Yeast display libraries were produced using variants of the 426c.NLGS.TM4ΔV1-3 monomeric gp120 immunogen (hereafter referred to as 426c.TM4 gp120), a modified clade C gp120 that was designed to engage VRC01-class precursor antibodies. A gp120-based immunogen was used to start instead of the engineered outer domain immunogens (eODs) previously used to select for VRC01-class bNAb precursors because, unlike certain other CD4bs bNAbs, IOMA contacts the inner domain of gp120, which is absent in the eOD constructs. To aid in determining which immunogen residues should be varied to achieve IOMA iGL binding, a 2.07 Å crystal structure of IOMA iGL Fab was solved (FIG. 6D, Table 2), which was nearly identical (root mean square deviation, RMSD, of 0.64 Å for 209 Ca atoms) to the mature IOMA Fab structure complexed with BG505 Env trimer (FIG. 6E). Based on modeling the IOMA iGL Fab structure (FIG. 1B-FIG. 1C, FIG. 6D) into the mature IOMA Fab-Env structure, 7 positions were varied in 426c.TM4 gp120. A library with ˜10⁸ variants was produced using degenerate codons so that all possible amino acids were incorporated at the selected positions (See, methods below). R278_{gp120} was varied because this position might select for IOMA iGL's unique CDRL1 conformation. In addition, a D279N_{gp120} substitution was introduced because IOMA is ˜2-3-fold more potent against HIV-1 viruses that have an N at this position. Next, V430_{gp120} was varied to increase the interaction with the HC of IOMA iGL. Lastly, residues 460_{gp120}-464_{gp120} were varied in the V5-loop of 426c gp120 to accommodate and select for IOMA iGL's normal length CDRL3.

Following three rounds of fluorescence-activated cell sorting (FACS) using one fluorophore for IOMA iGL and another against a C-terminal Myc tag to monitor gp120 expression, there was a >100-fold enrichment for gp120 variants that bound IOMA iGL (FIG. 6F), demonstrated by increased staining for IOMA iGL compared to the starting 426c.TM4 gp120 (FIG. 1B-FIG. 1C, FIG. 6F). Two clones (from ˜100 sequenced after the third sort) accounted for 50% of the sequences, suggesting that IOMA iGL-binding activity was enriched. IGT1, the best variant identified by the initial yeast display library, had an affinity of ˜30 μM for IOMA iGL, as determined by a surface plasmon resonance (SPR)-based binding assay (FIG. 1C, bottom right panel). IGT1 was then used as a guide to construct a second yeast library to select for an immunogen with higher affinity to IOMA iGL (FIG. 1D). Based on their selection in IGT1, residues R278_{gp120}, N279_{gp120}, and P430_{gp120} were maintained, while amino acids R/N/K/S were allowed to be sampled at position 460. In addition, residues 461-464 and 471 were allowed to be fully degenerate and sample all possible amino acids. Following 7 rounds of sorting, multiple clones were selected including IGT2, which bound to IOMA iGL with a 0.5 μM affinity (FIG. 1D, bottom right panel and FIG. 6F, Library 2).

IOMA-targeting mutations selected by yeast display were transferred onto a 426c soluble native-like Env trimer (a SOSIP.664 construct) to hide potentially immunodominant off-target epitopes within the Env trimer core that are exposed in a monomeric gp120 protein. The SOSIP versions of IGT1 and IGT2 were well behaved in size-exclusion chromatography and SDS-PAGE (FIG. 6G-FIG. 6H). IGT1 and IGT2 SOSIPs bound to IOMA iGL IgG with higher apparent affinities than IGT1 and IGT2 gp120s due to avidity effects (FIG. 1C-FIG. 1D). IGT1 and IGT2 SOSIP- and gp120-based immunogens were also evaluated for binding to a panel of VRC01-class iGL antibodies (VRC01, 3BNC60, BG24). IGT2 bound all the iGLs tested, making it the only reported immunogen that binds to iGLs from both IOMA- and VRC01-class CD4bs bNAbs (FIG. 1E, FIG. 6I-FIG. 6J). Finally, using the SpyCatcher-SpyTag system (See, e.g., US Patent publication US20220168414A1, which is hereby incorporated by reference in its entirety), SpyTagged SOSIP-based immunogens were covalently linked to the designed 60-mer nanoparticle SpyCatcher003-mi3 (FIG. 1A, FIG. 1F), thereby enhancing antigenicity and immunogenicity through avidity effects from multimerization (FIG. 6I), while also reducing the exposure of undesired epitopes at the base of soluble Env trimers. Efficient covalent coupling of the immunogens to SpyCatcher003-mi3 was demonstrated by SDS-PAGE (FIG. 6H), and negative stain electron microscopy (EM) showed that these nanoparticles were densely conjugated and uniform in size and shape (FIG. 6F).

### Sequential Immunization of Transgenic IOMA iGL Knock-In Mice Elicits Broad Heterologous Neutralizing Serum Responses

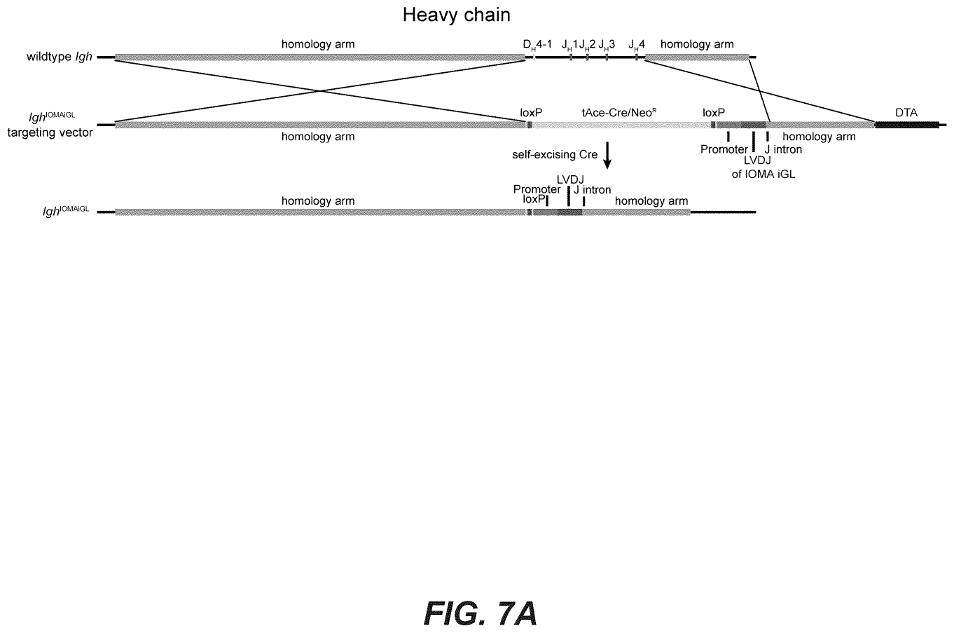

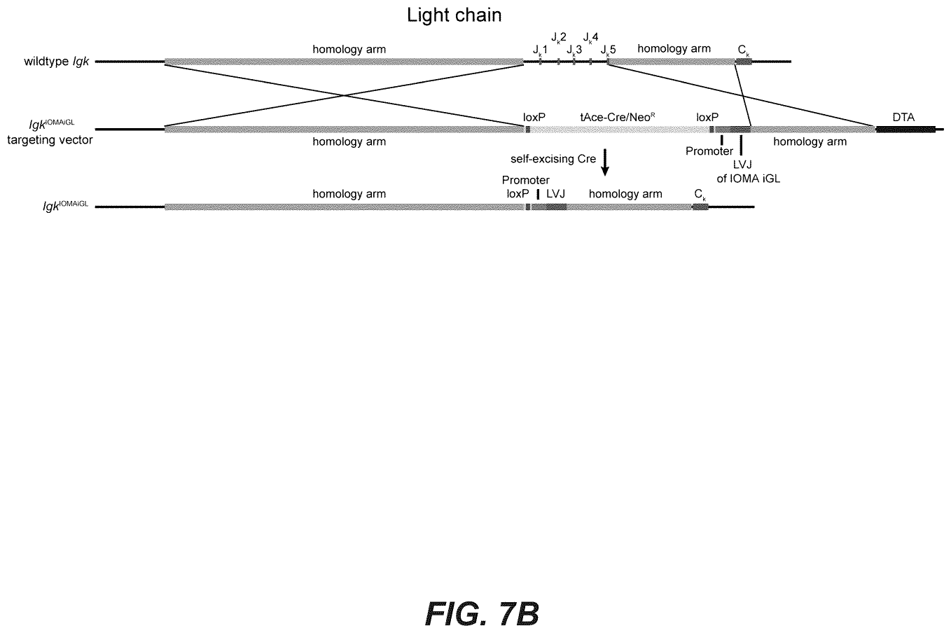

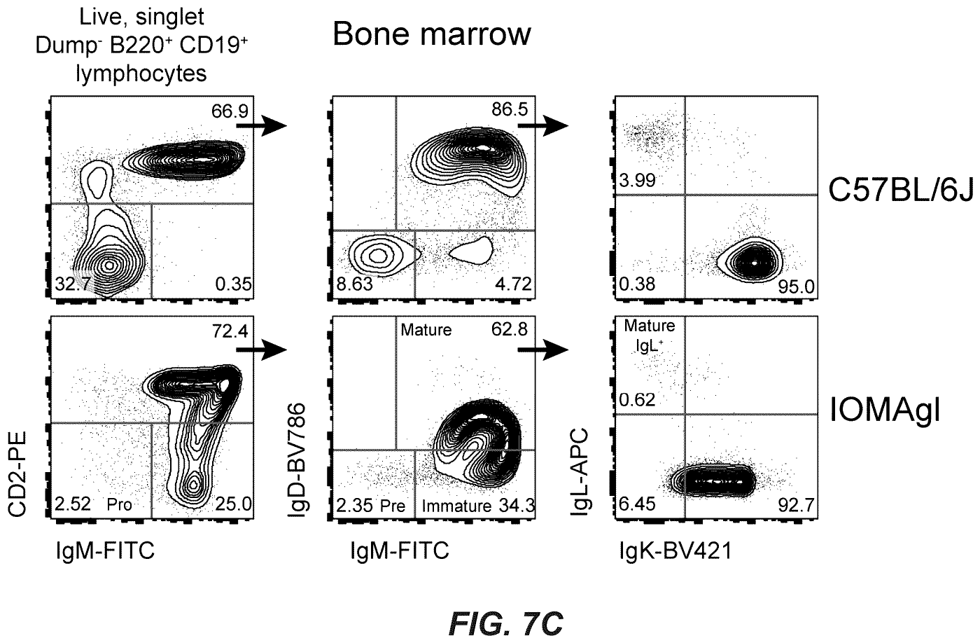

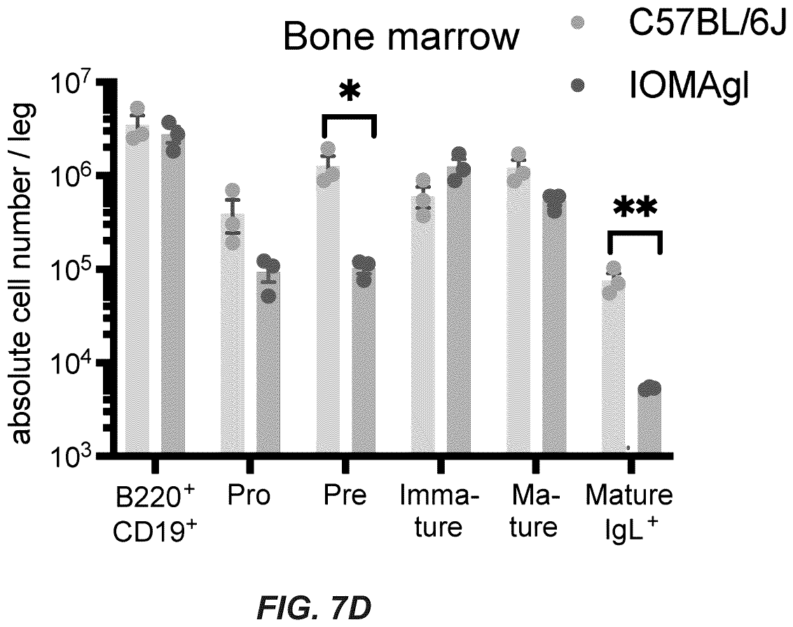

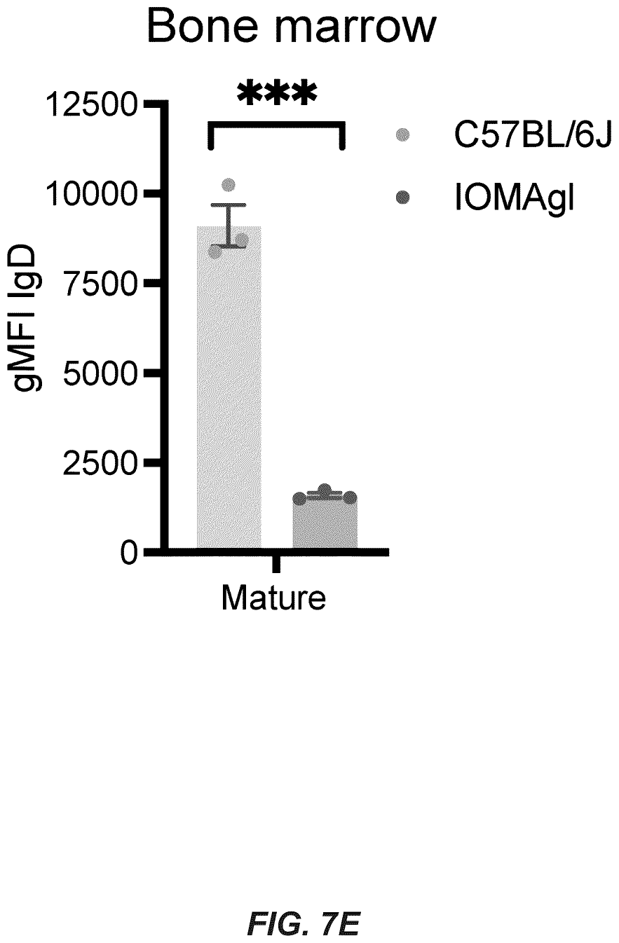

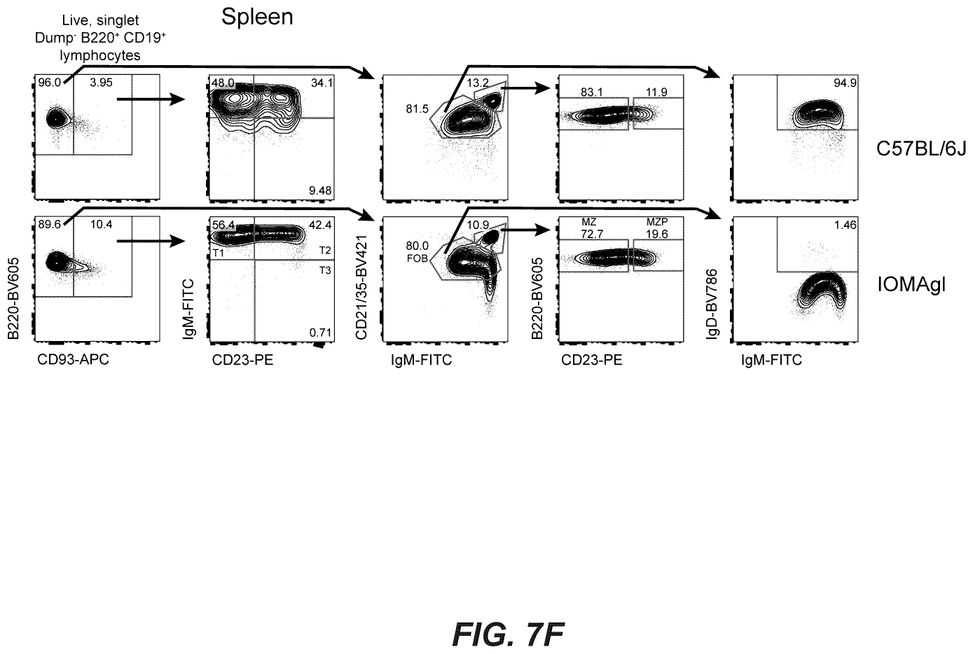

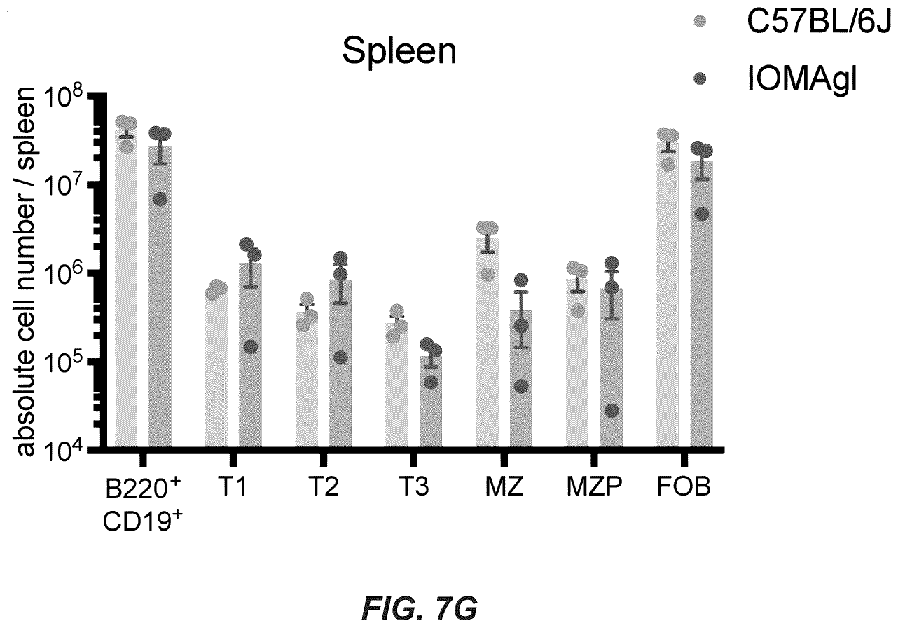

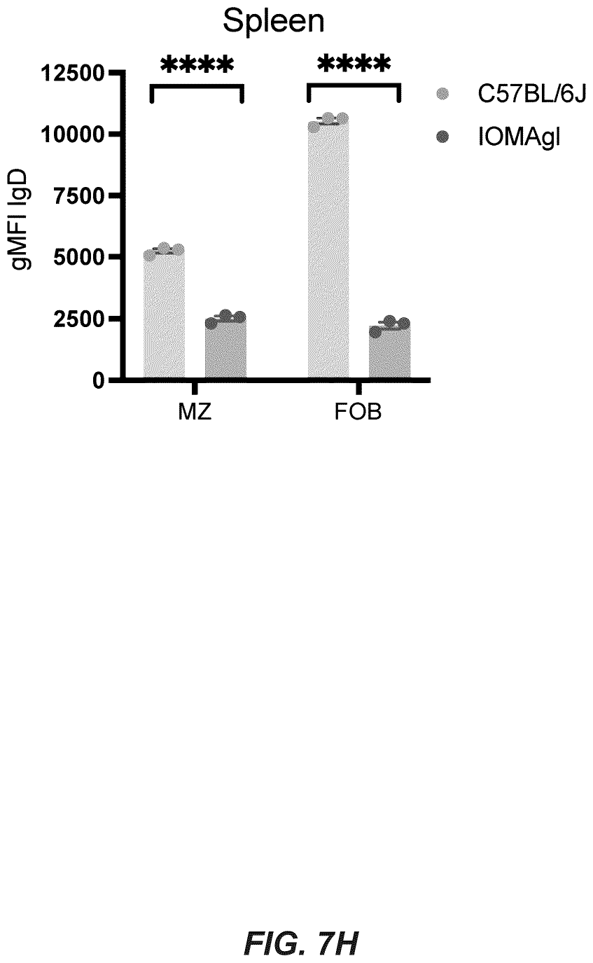

To evaluate whether the immunogens induced IOMA-like antibody responses, transgenic mouse models were generated expressing the full, rearranged IOMA iGL VH or VL genes in the mouse Igh (IghIOMAiGL) and Igk loci (IgkIOMAiGL) (FIG. 7A-FIG. 7B). Mice homozygous for both chains, termed IOMAgl mice, showed overall normal B cell development with reduced numbers of pre-B cells and late upregulation of CD2 (suggesting accelerated B cell development due to the already rearranged VDJ and VJ genes), a preference for the IOMA iGL Igκ as seen by a reduction of mouse Igλ-expressing cells, and a reduction in IgD expression indicative of low autoreactivity (FIG. 7C-FIG. 7H). Total B cell numbers in IOMAgl mice were grossly normal, making them suitable to test IOMA germline-targeting immunogens (FIG. 7D, FIG. 7G).

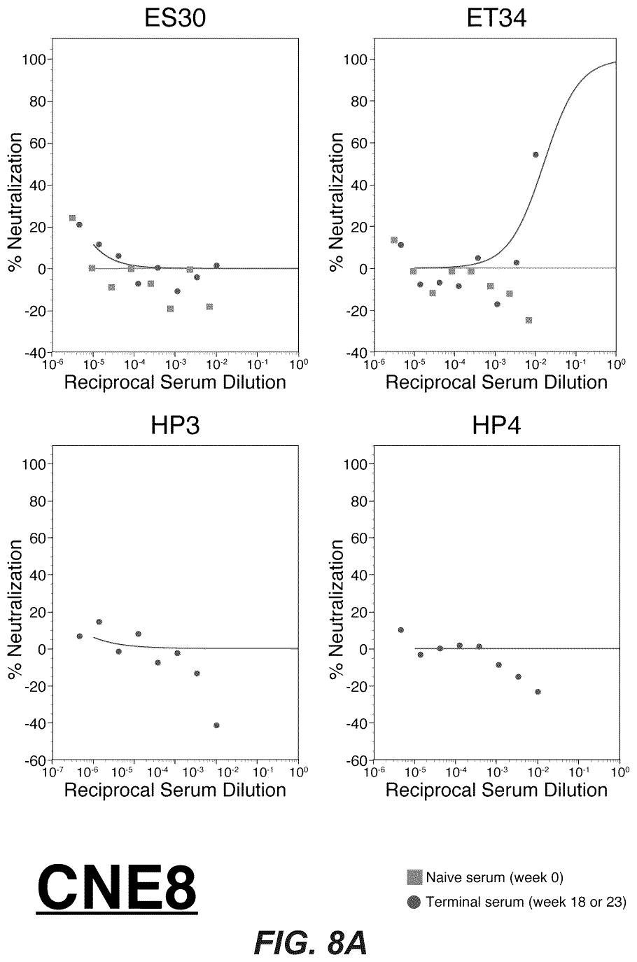

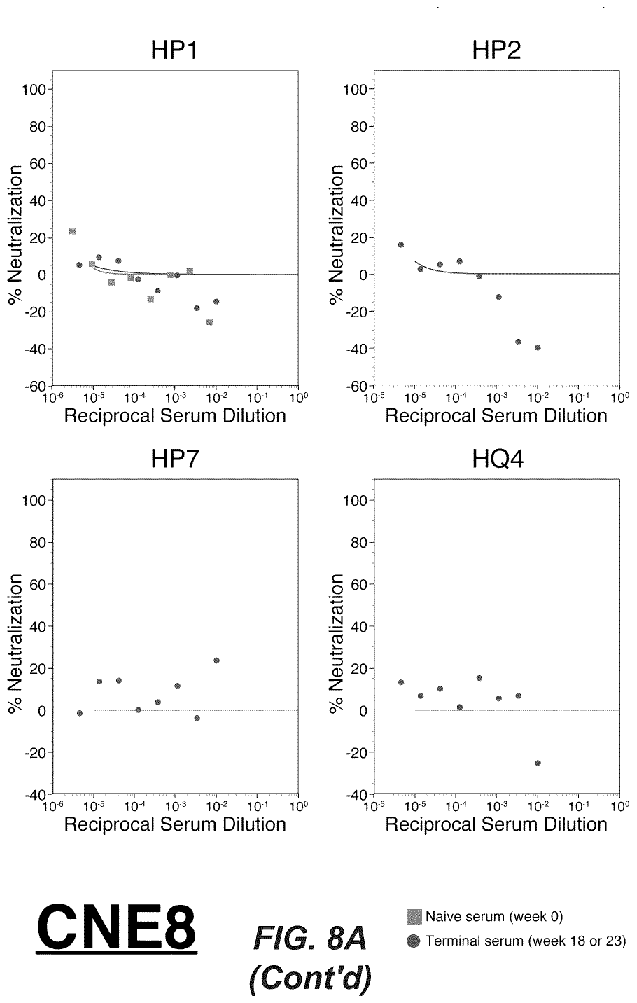

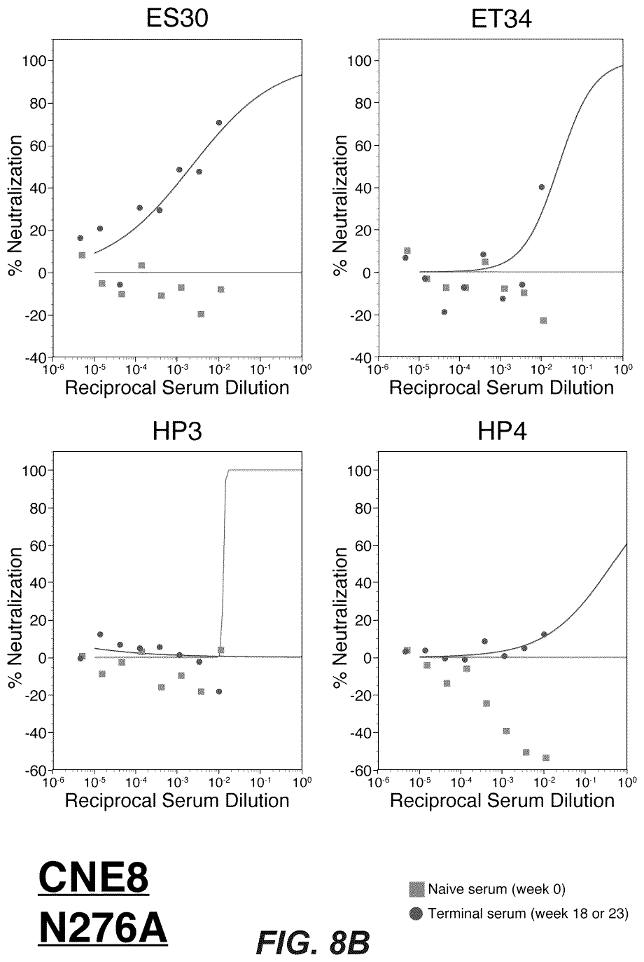

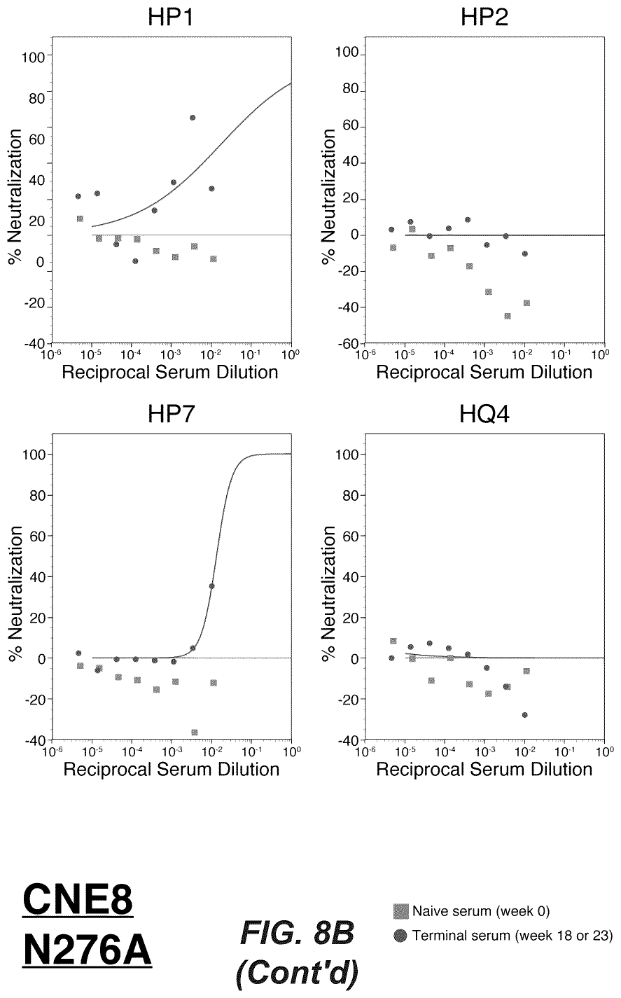

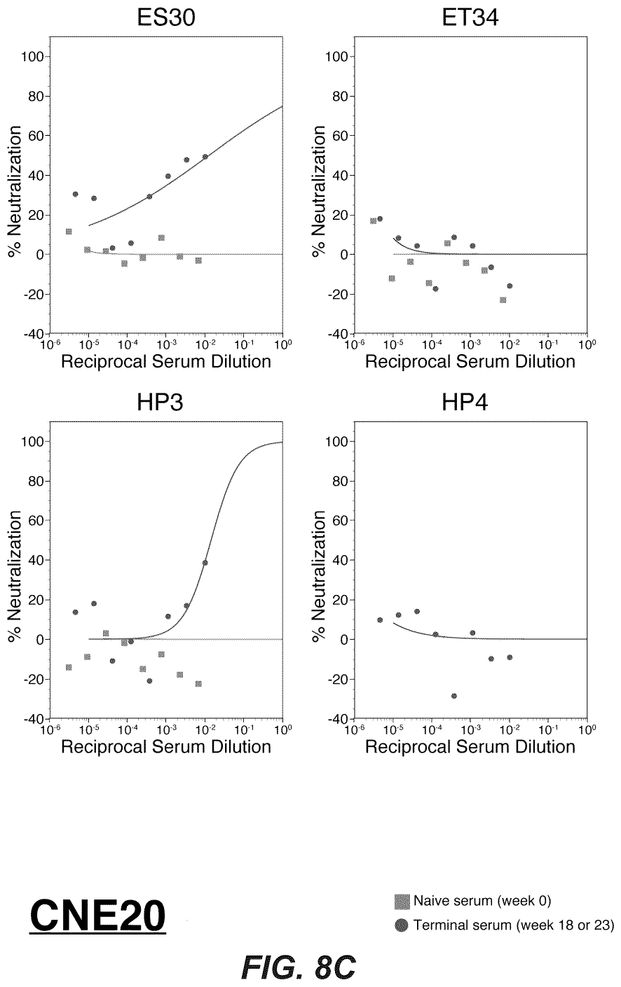

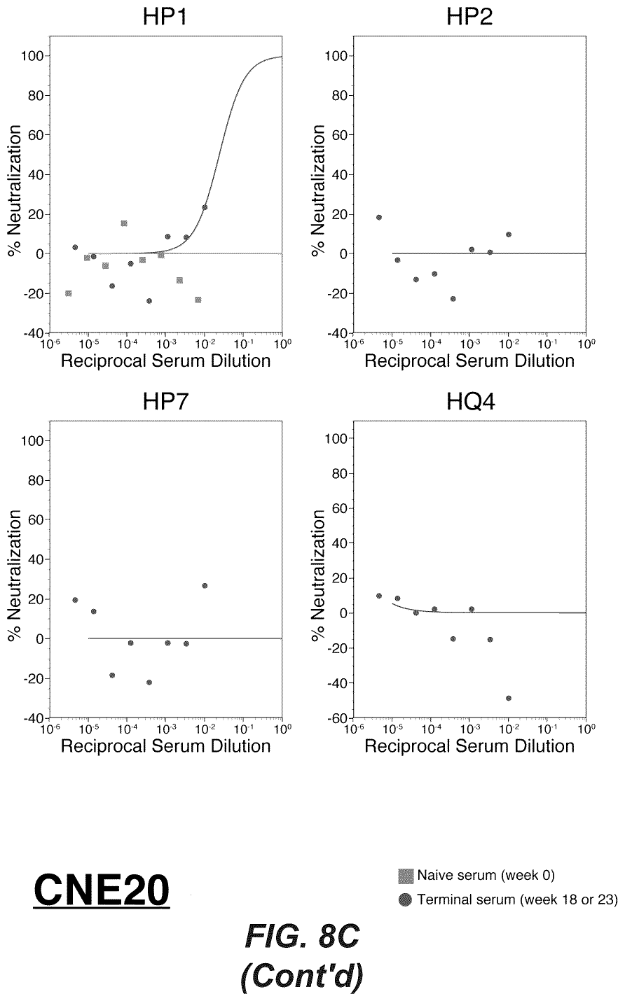

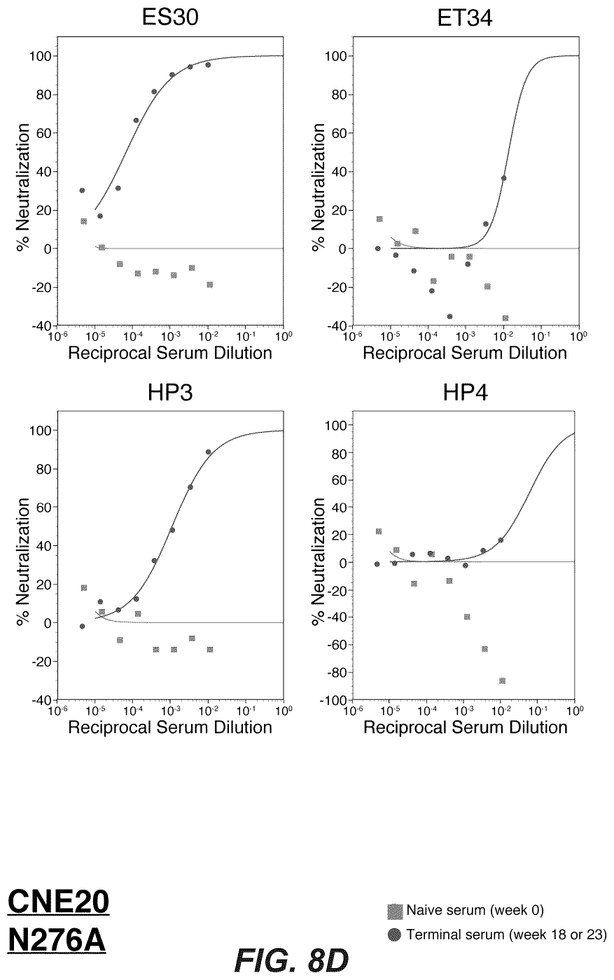

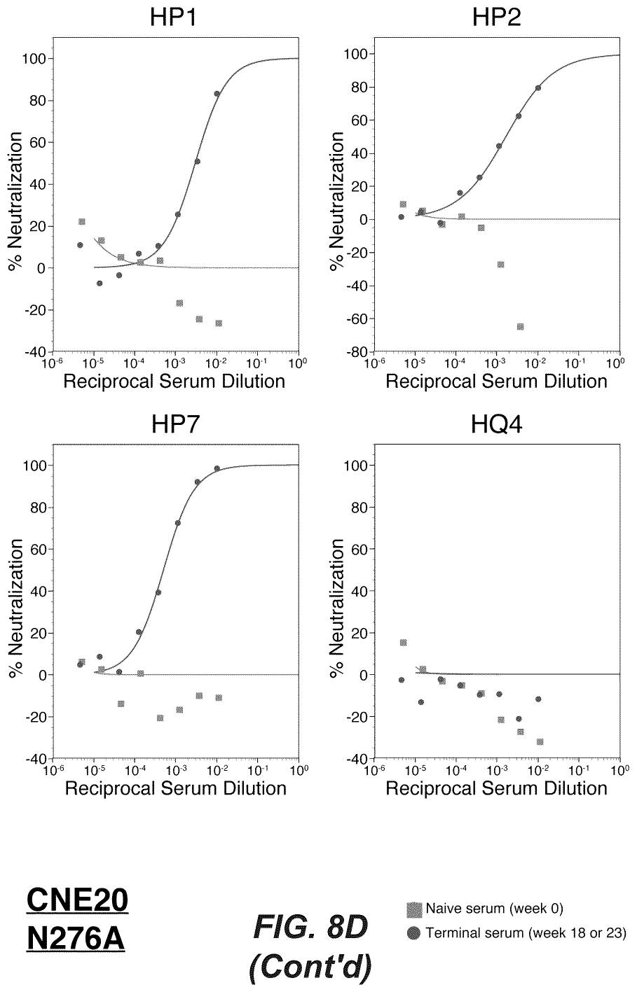

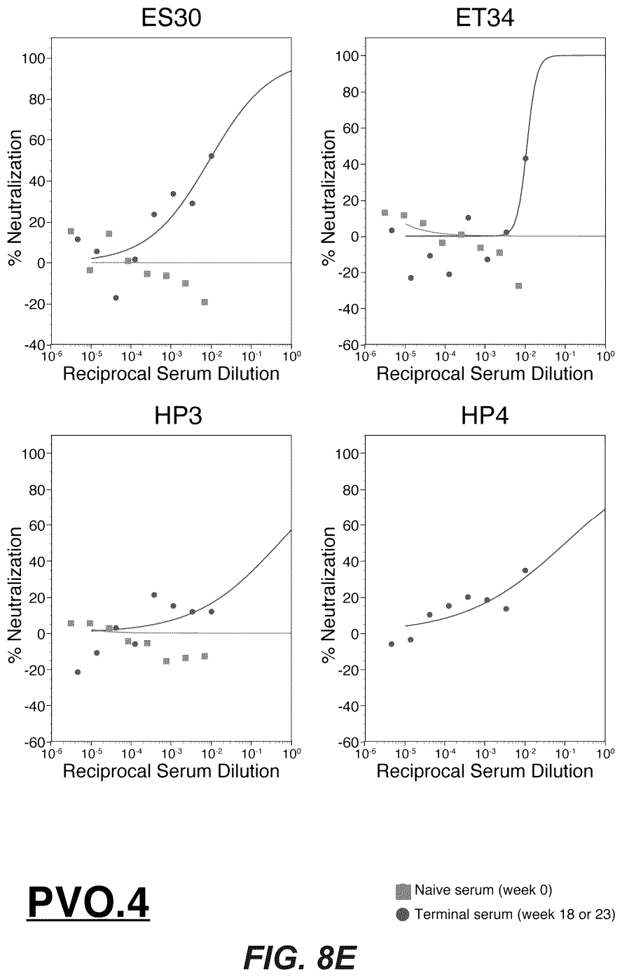

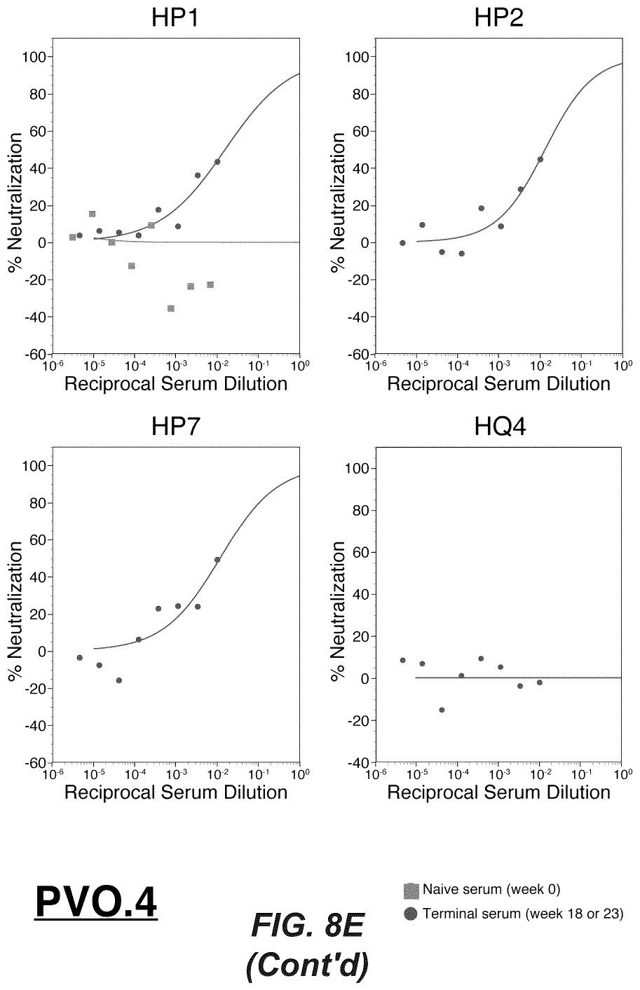

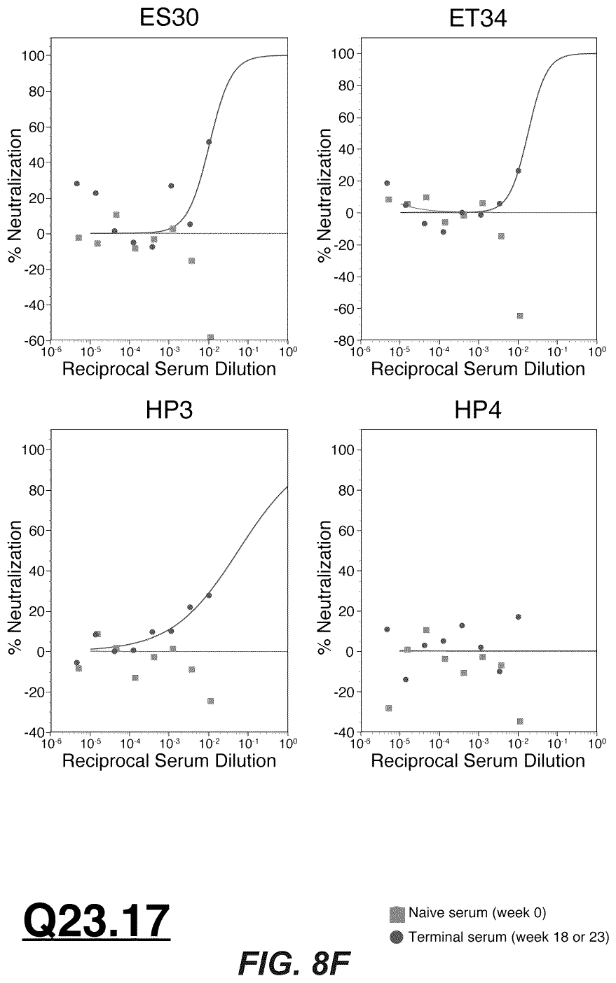

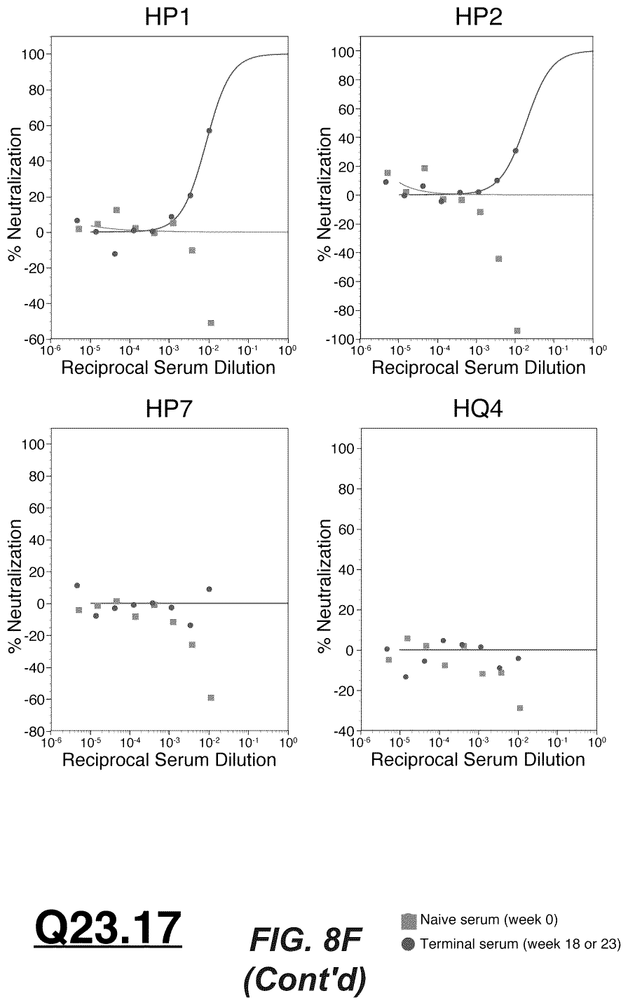

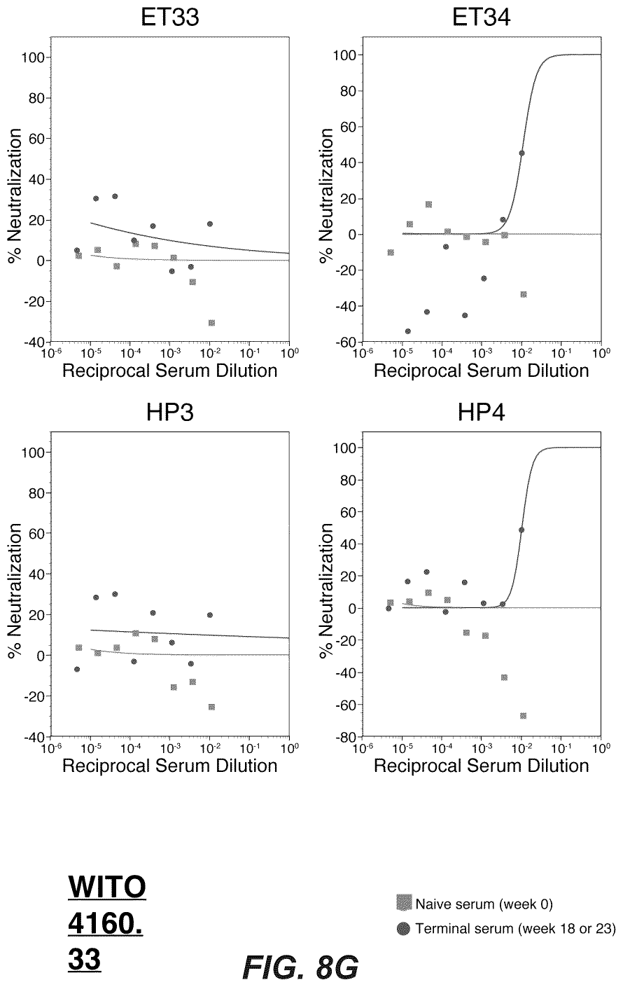

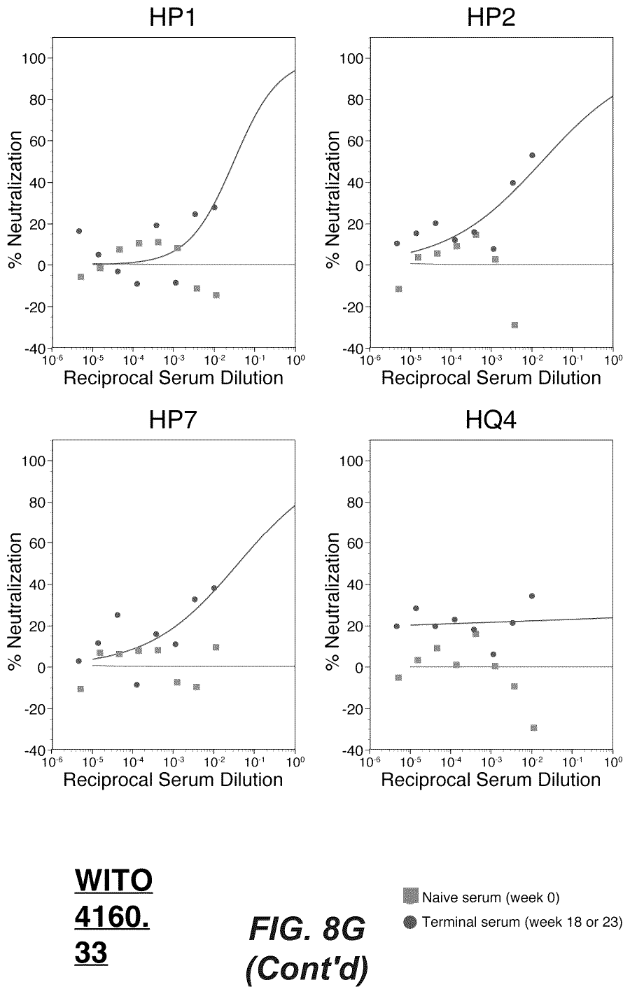

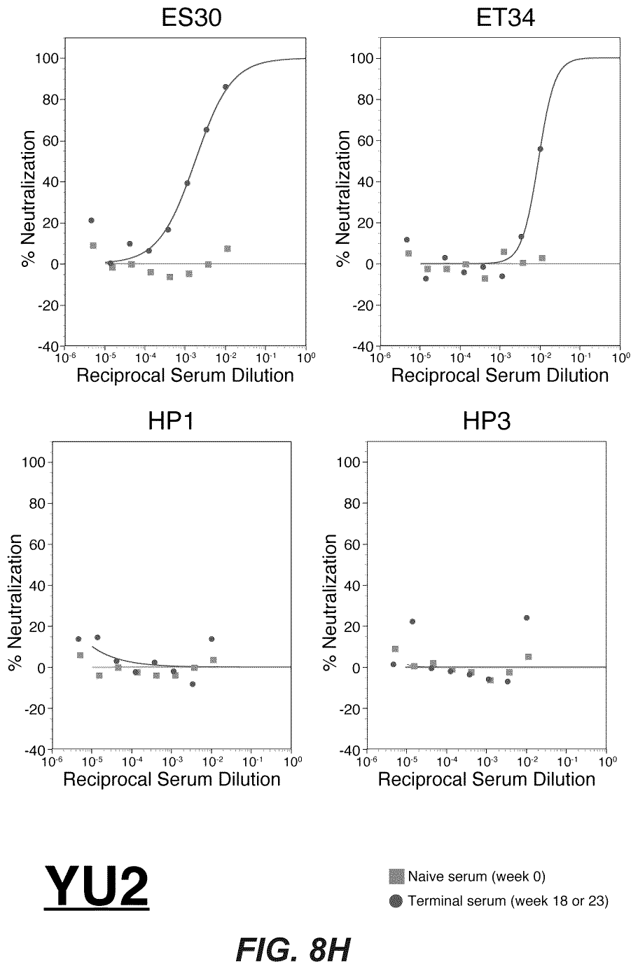

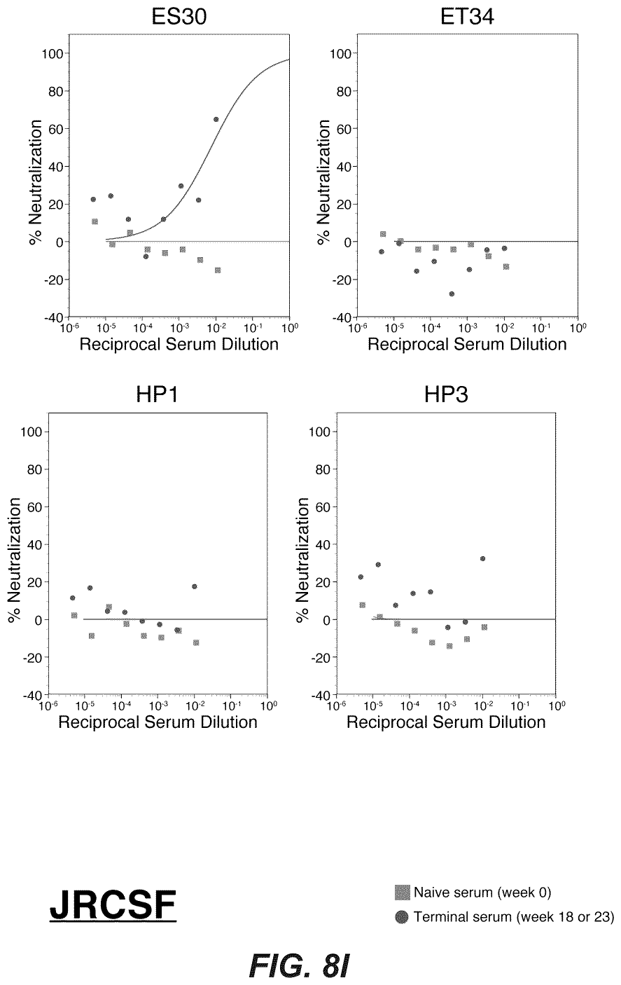

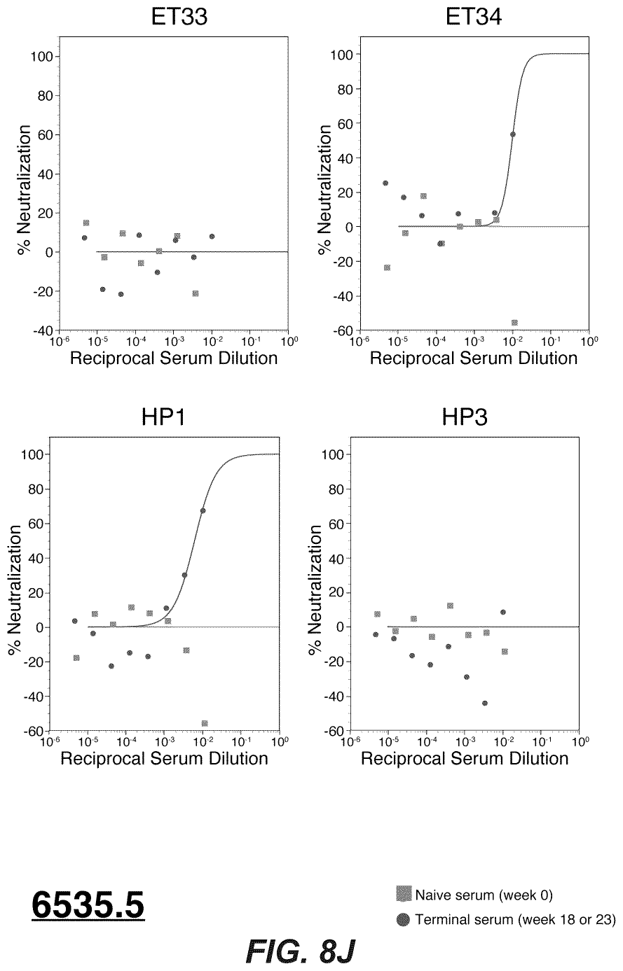

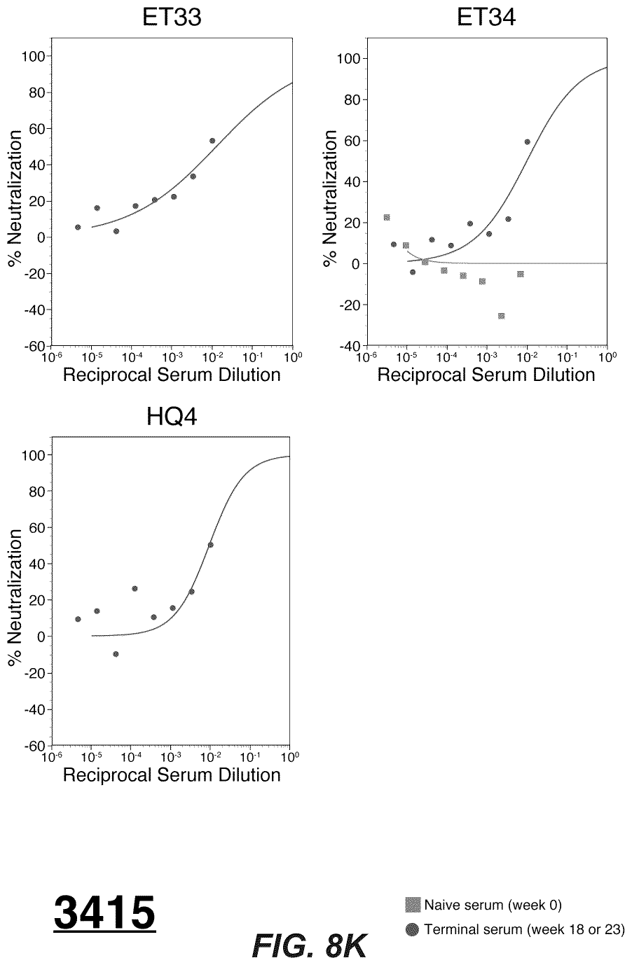

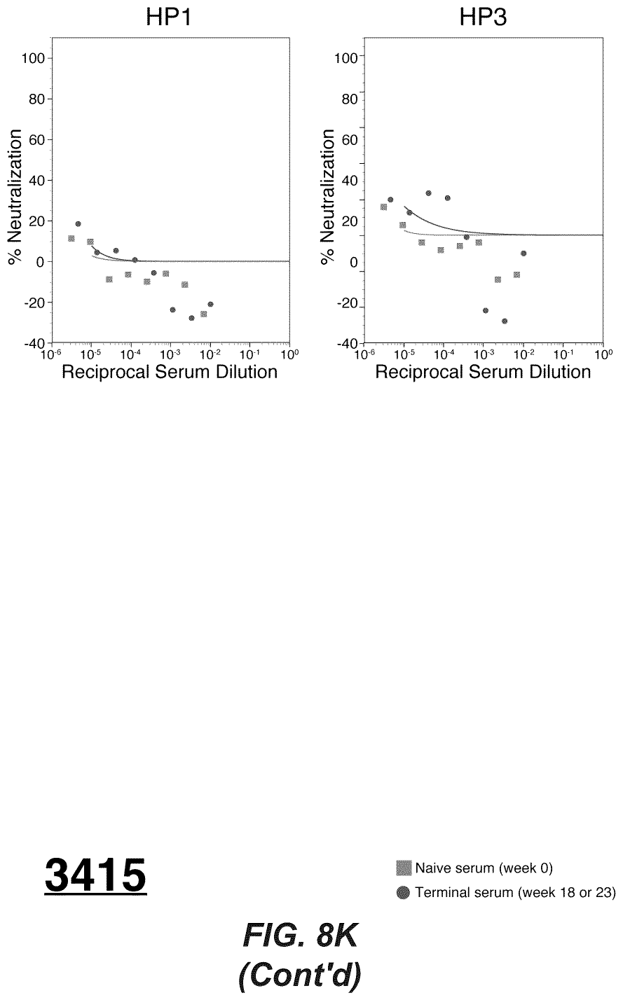

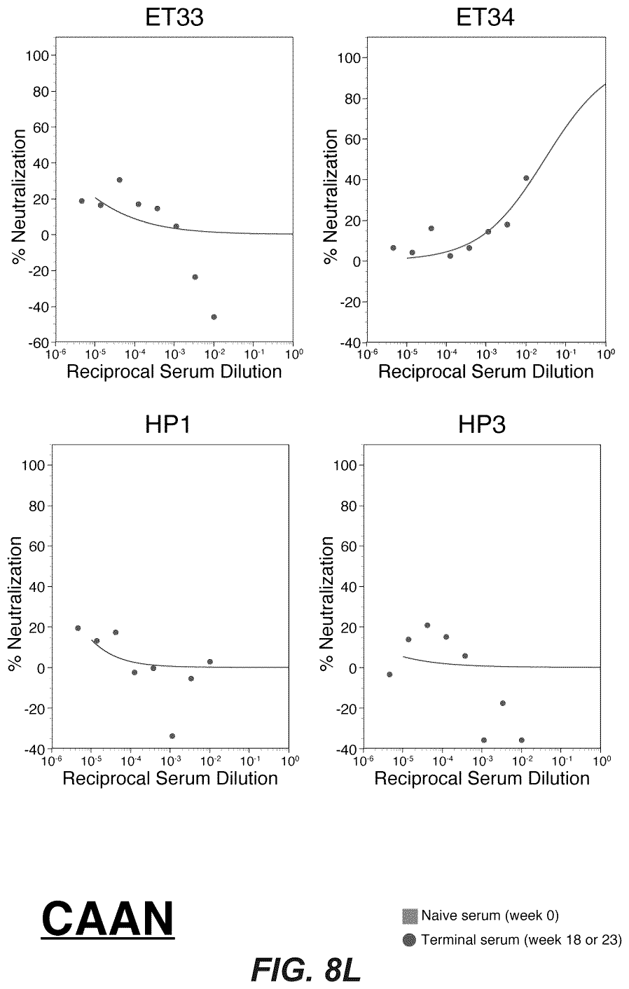

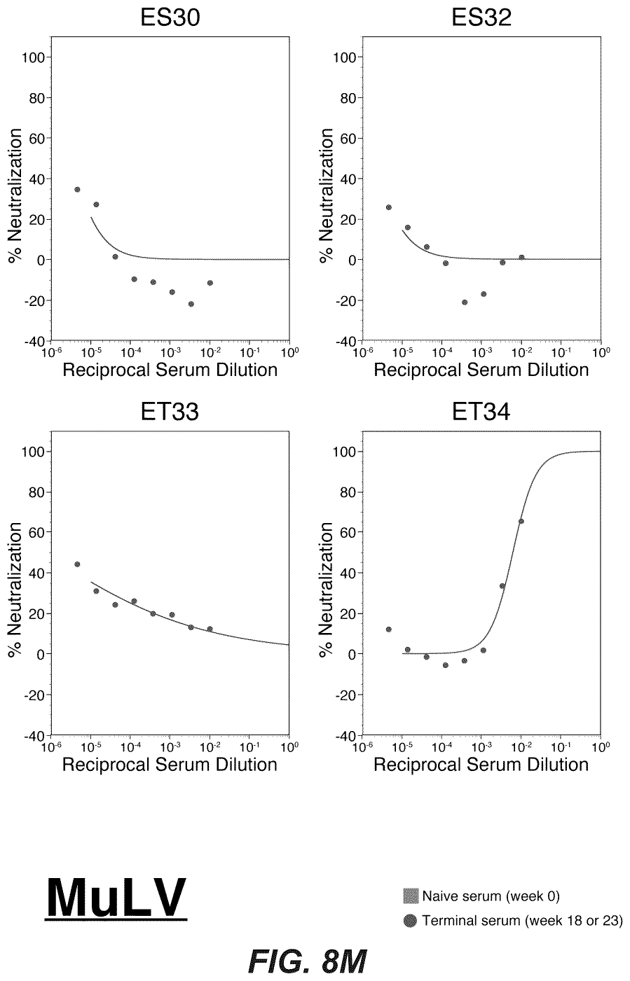

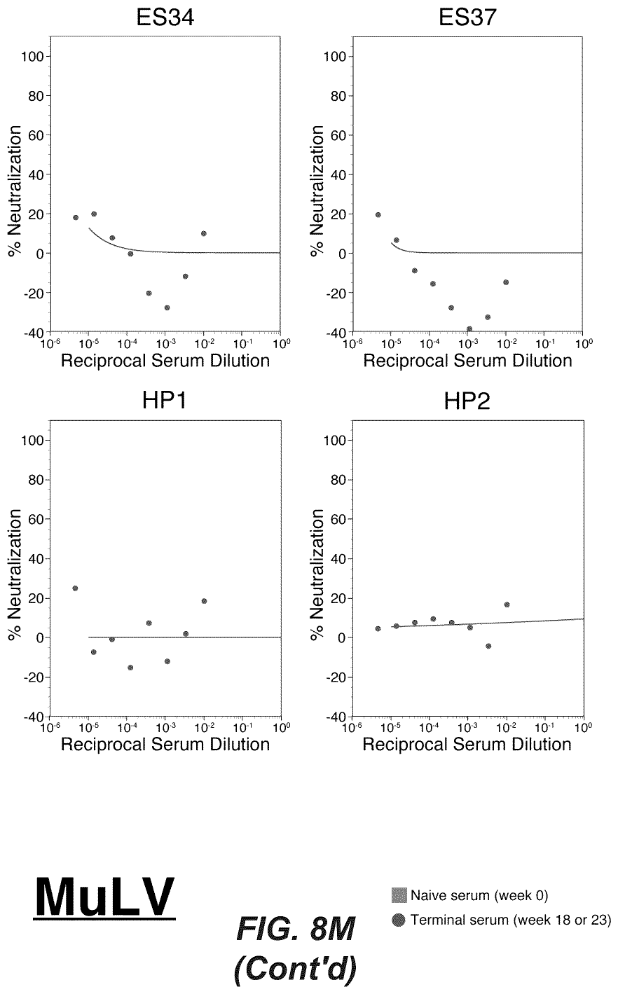

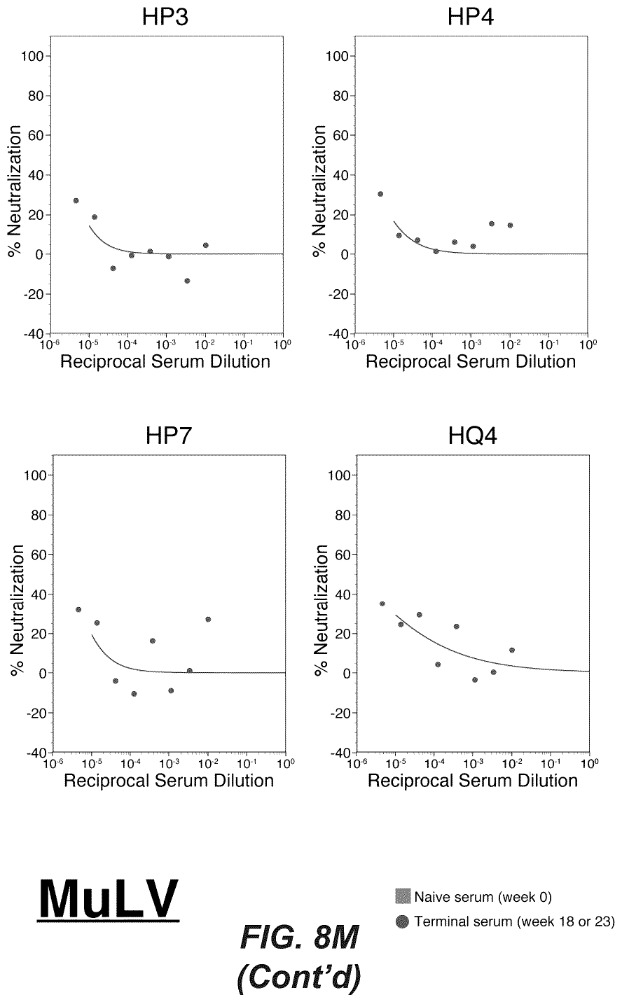

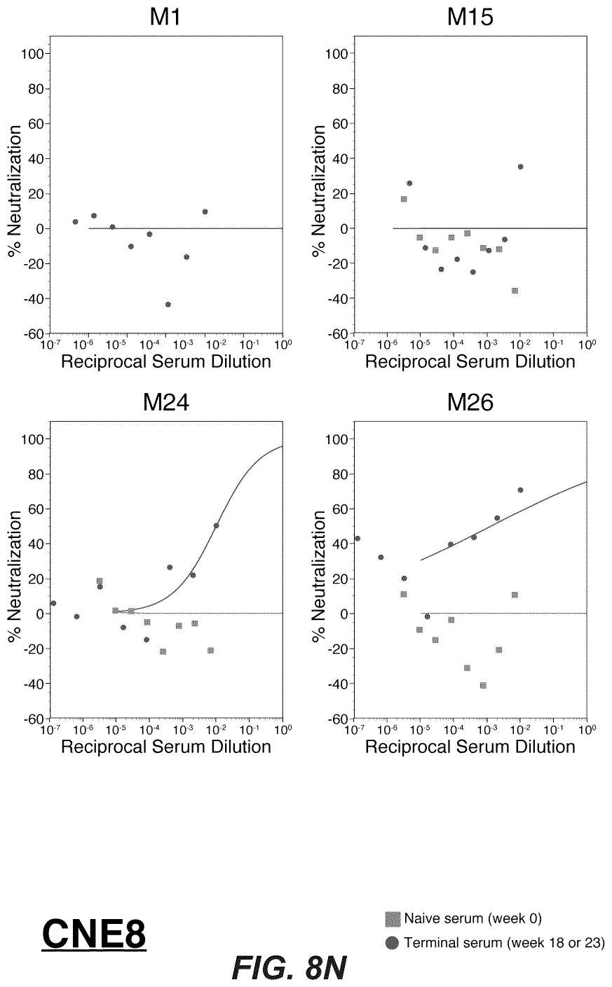

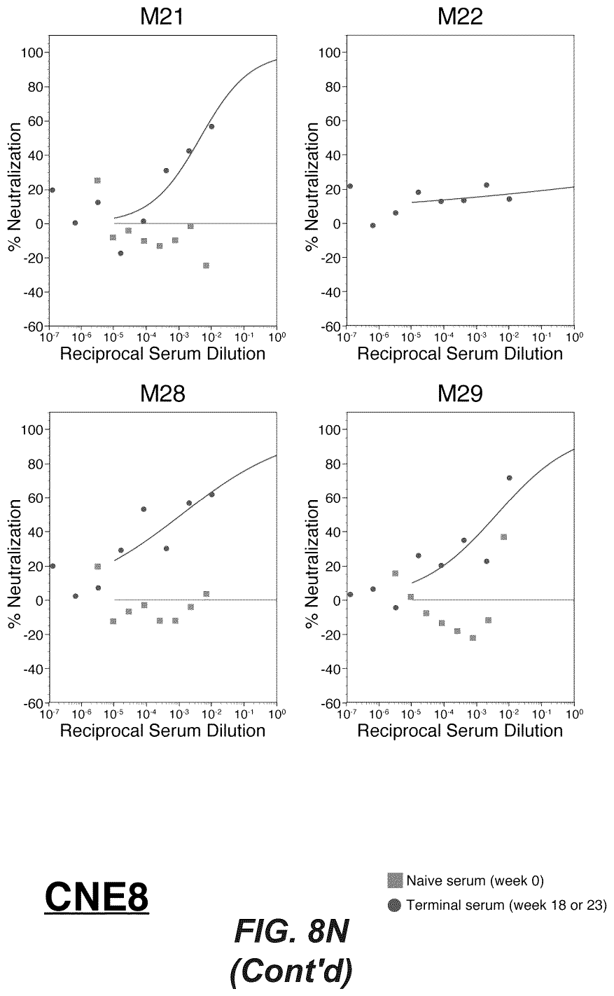

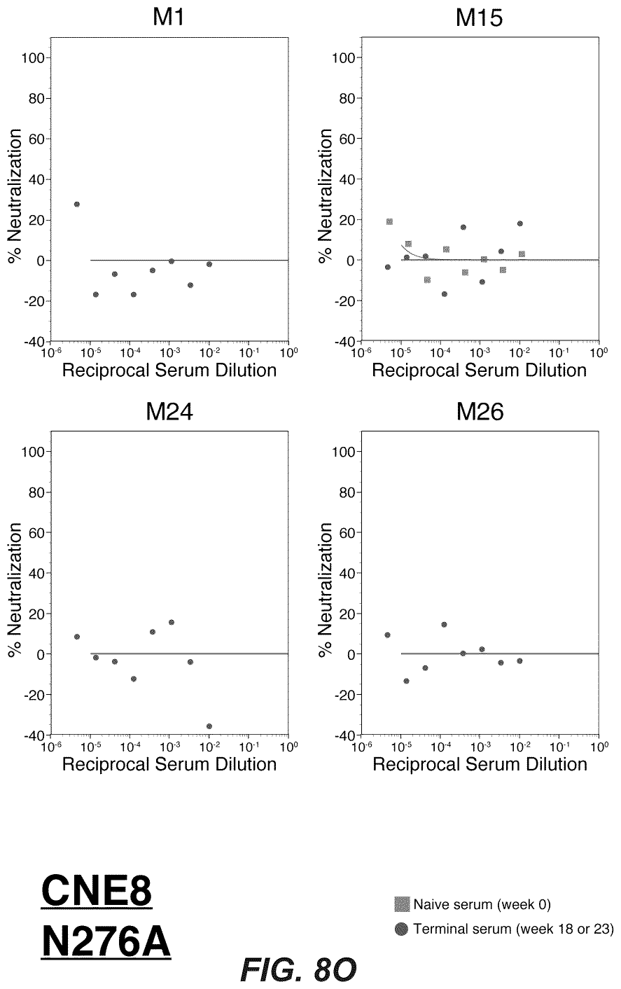

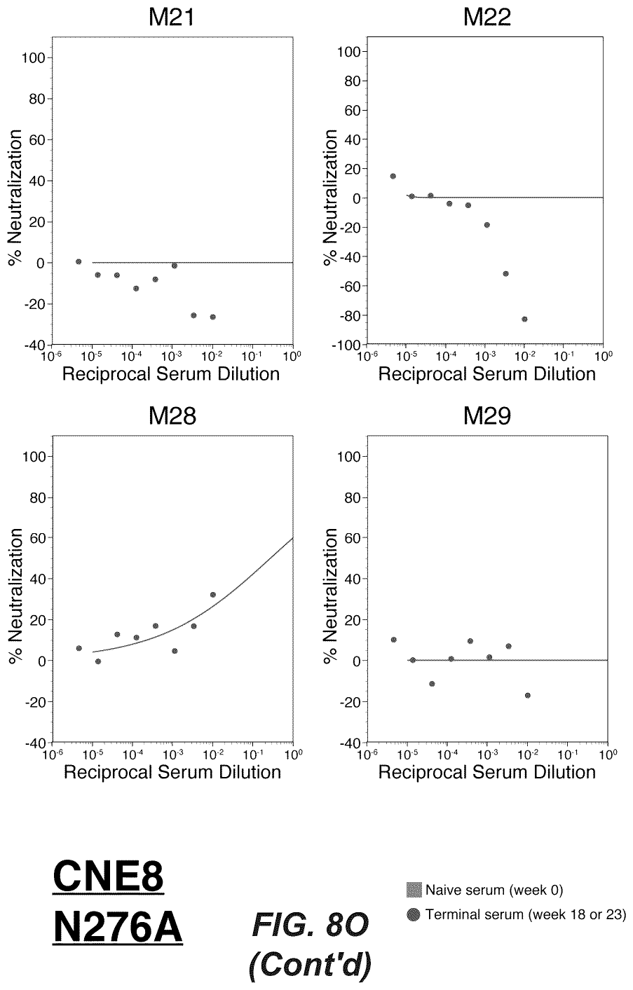

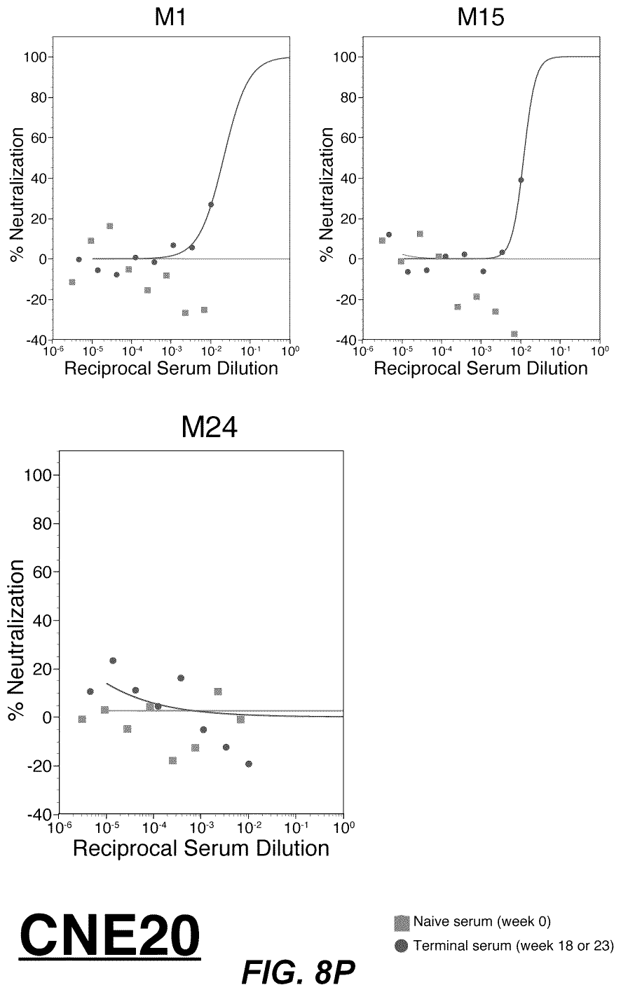

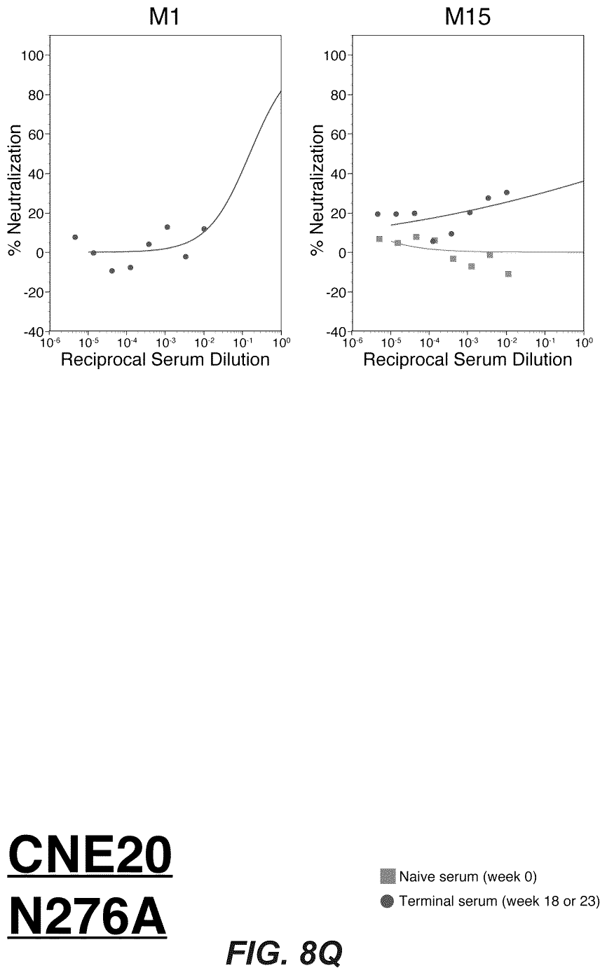

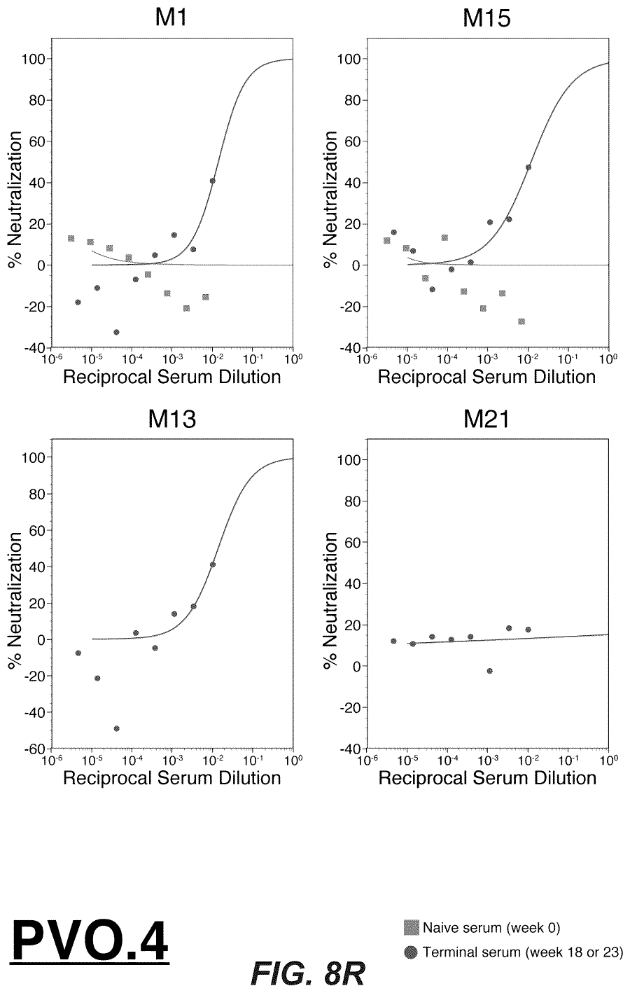

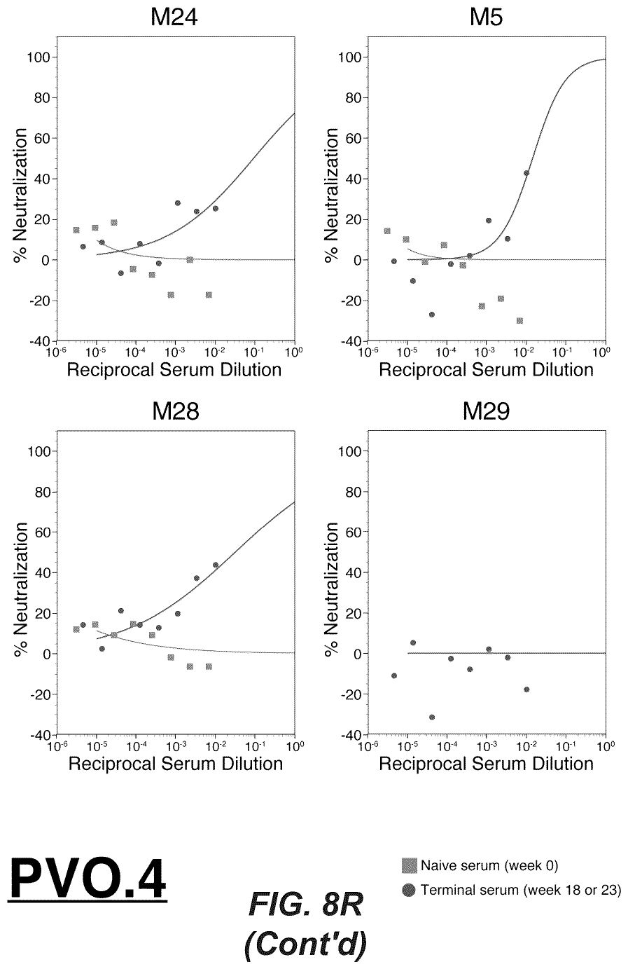

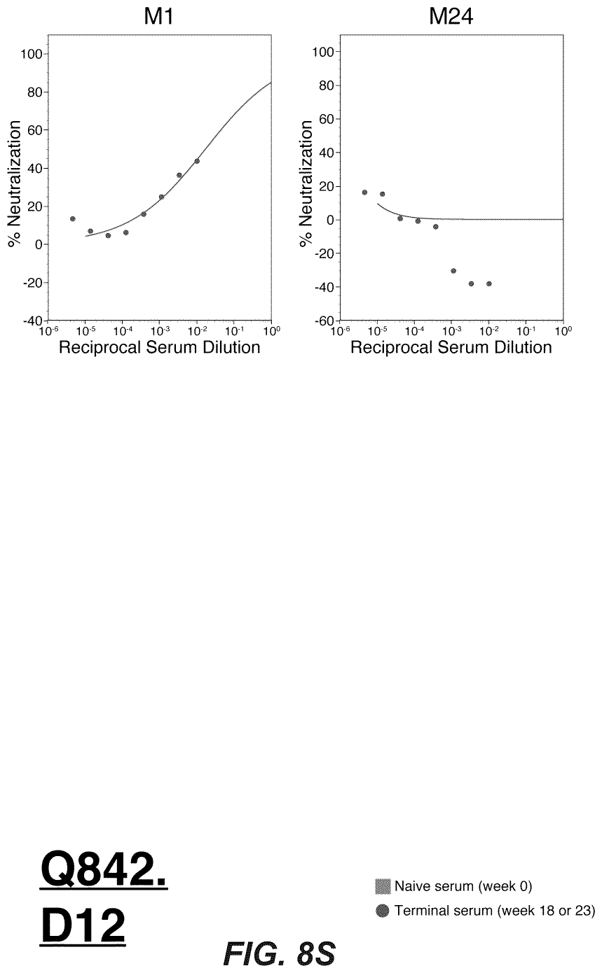

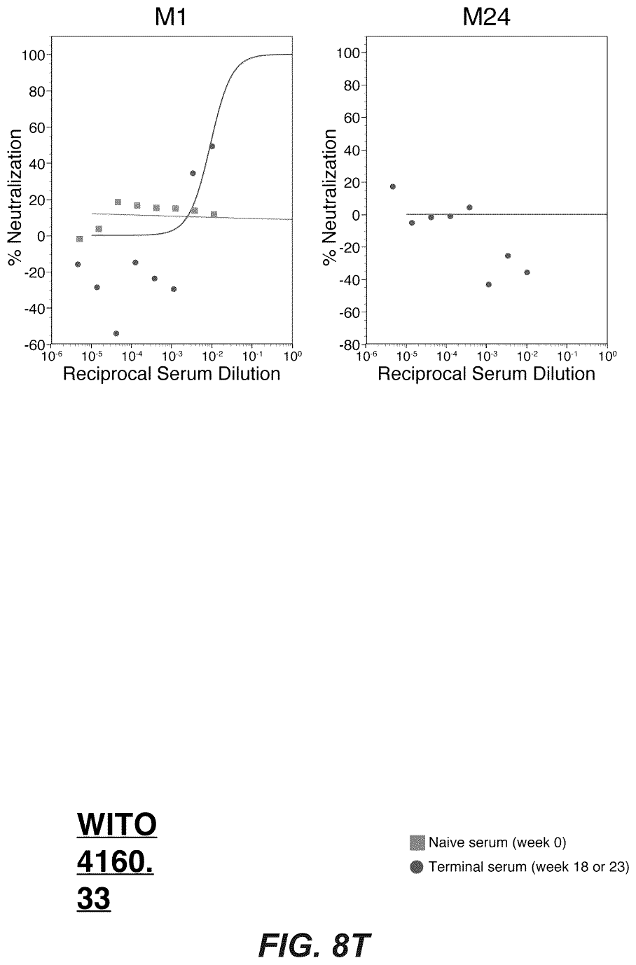

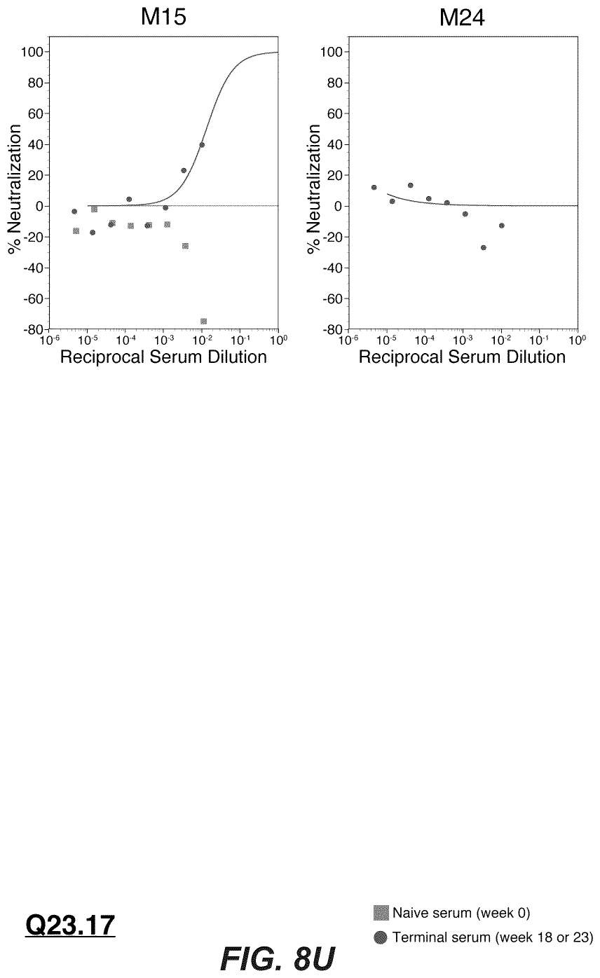

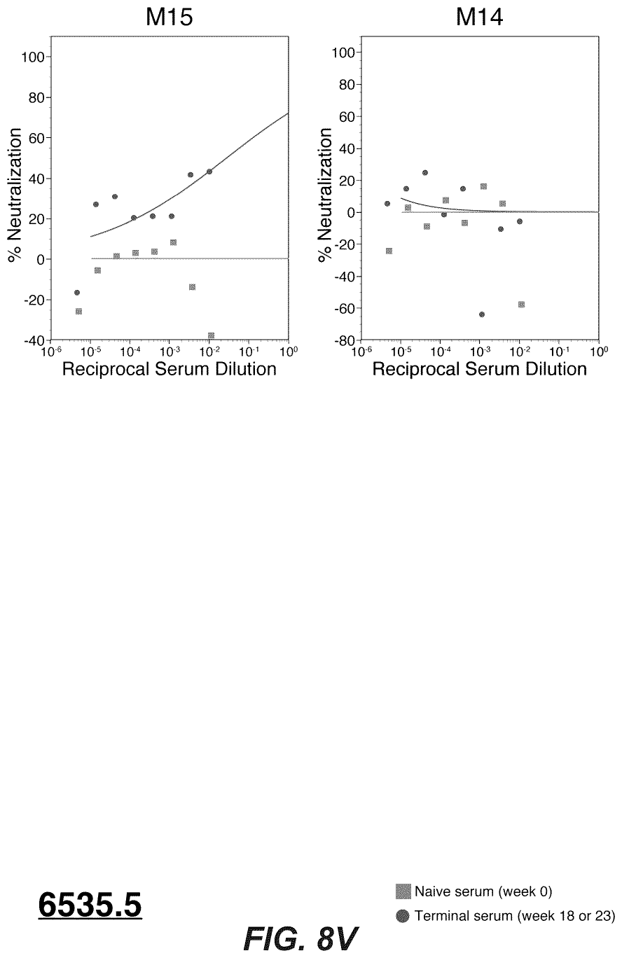

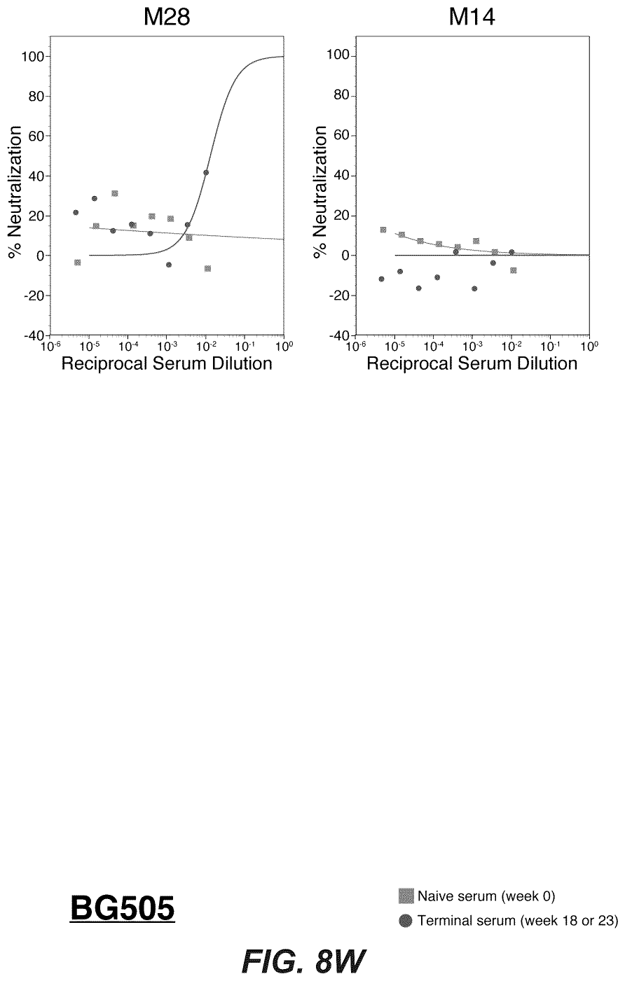

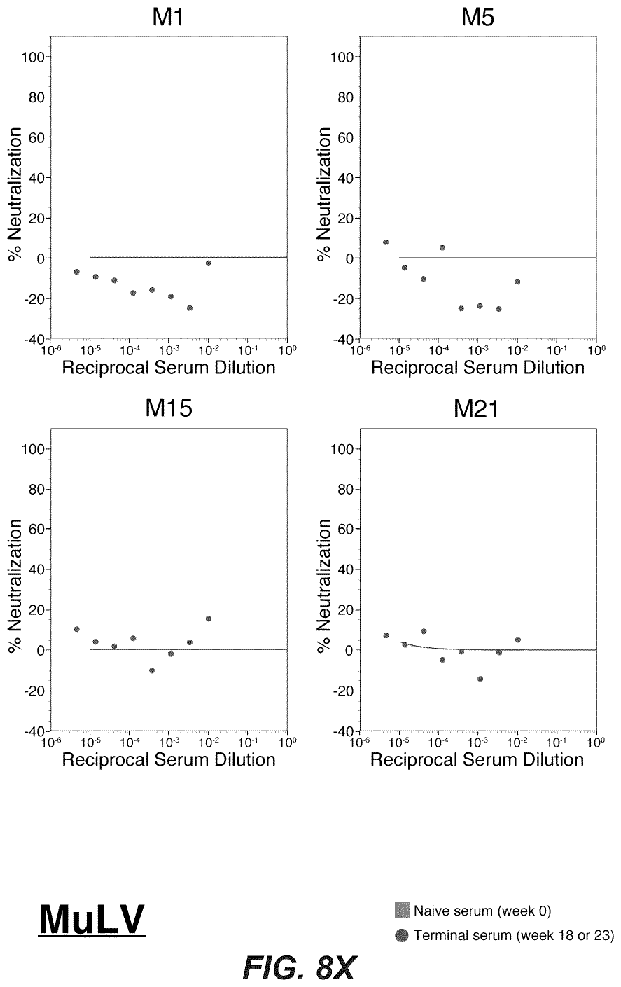

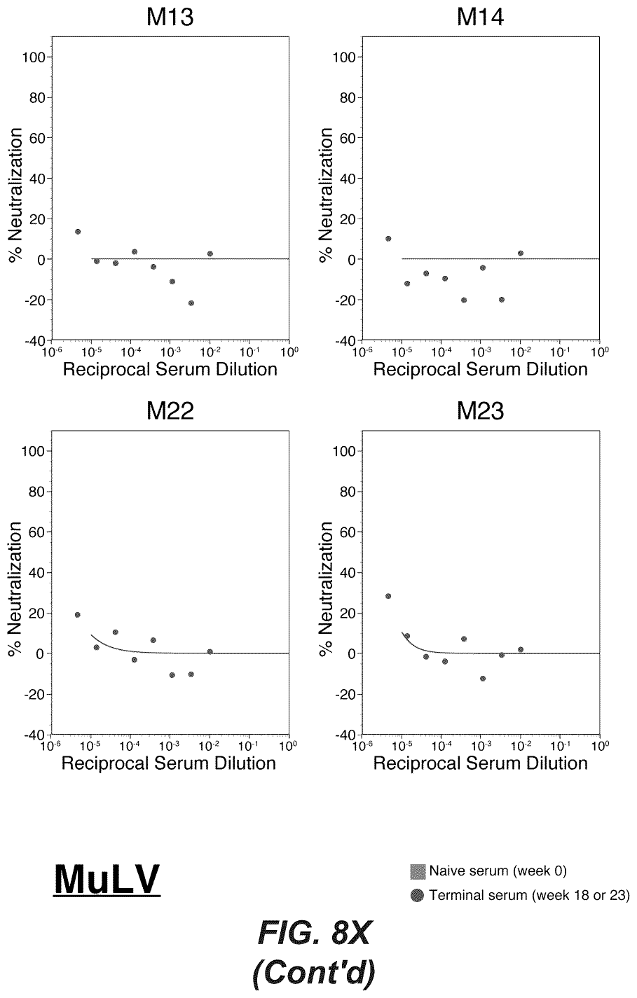

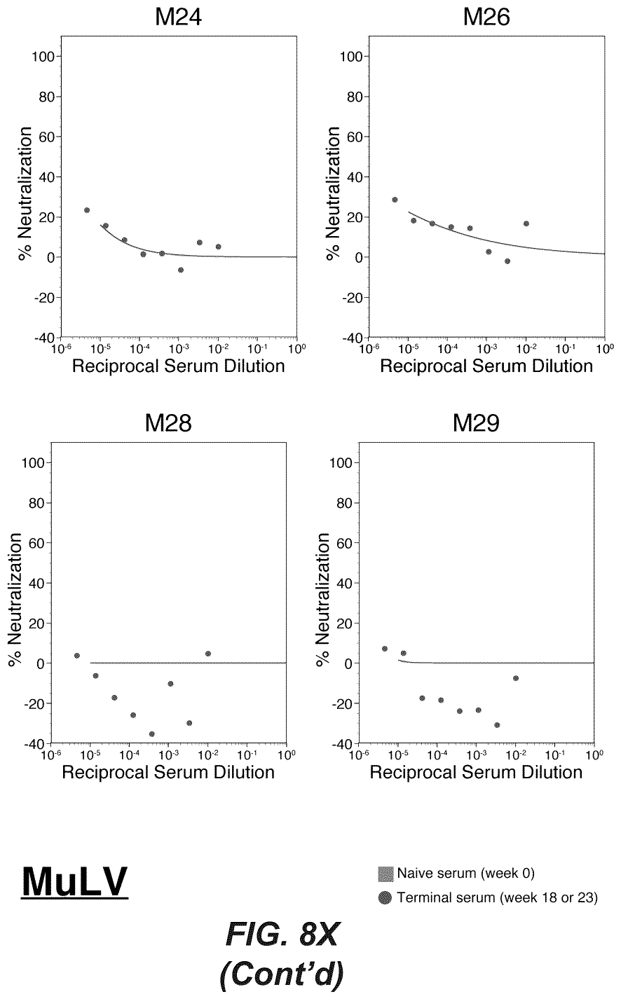

The IOMAgl mice were primed using mi3 nanoparticles coupled with the SOSIP version of the immunogen with the highest affinity to IOMA iGL (FIG. 2A, IGT2-mi3) adjuvanted with the SMNP adjuvant, and binding was compared by ELISA to IGT2 and a CD4bs knockout mutant IGT2 (CD4bs KO: G366R/D368R/D279N/A281T). Priming the IOMAgl mice with IGT2-mi3 elicited only weak responses to the priming and boosting (IGT1-mi3) immunogens (FIG. 2B-FIG. 2C). However, boosting with mi3 nanoparticles coupled with IGT1, which bound IOMA iGL with a lower affinity than IGT2 (FIG. 1C), increased the magnitude and specificity of the serum responses, as demonstrated by an increase in binding to IGT2 and IGT1 compared to IGT2- and IGT1-CD4bs KO (FIG. 2B-FIG. 2C). A comparable level of differential binding was preserved throughout the remaining immunizations (Group 1) following boosting with 426c degly2 D279N (degly2: removal of N460gp120 and N462gp120 PNGSs) followed by mosaic8-mi3, a nanoparticle coupled with 8 different wt SOSIPs chosen from a global HIV-1 reference panel used to screen bNAbs (Table 1). Serum binding also increased throughout the immunization regimen for 426c and 426c D279N, a mutation preferred by IOMA, compared to 426c-CD4bs KO (FIG. 2D). Terminal bleed sera showed binding to a panel of heterologous wt and N276A Env SOSIPs in (FIG. 2E-FIG. 2F) and when screened against a panel of IOMA-sensitive HIV-1 strains, 8 of 12 IOMA iGL knock-in animals neutralized up to 9 of 15 strains (FIG. 2G, FIG. 8A-FIG. 8M, Table 3). However, one of these mice (ET34) also neutralized the MuLV control virus, suggesting, without being bound by any particular theory, that the neutralization activity from this mouse is at least partially non-specific for HIV.

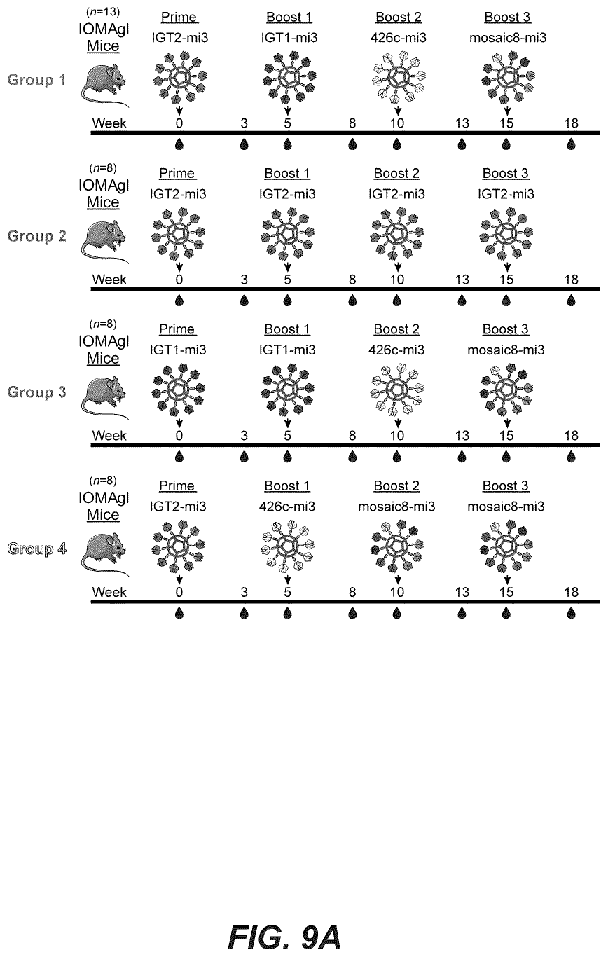

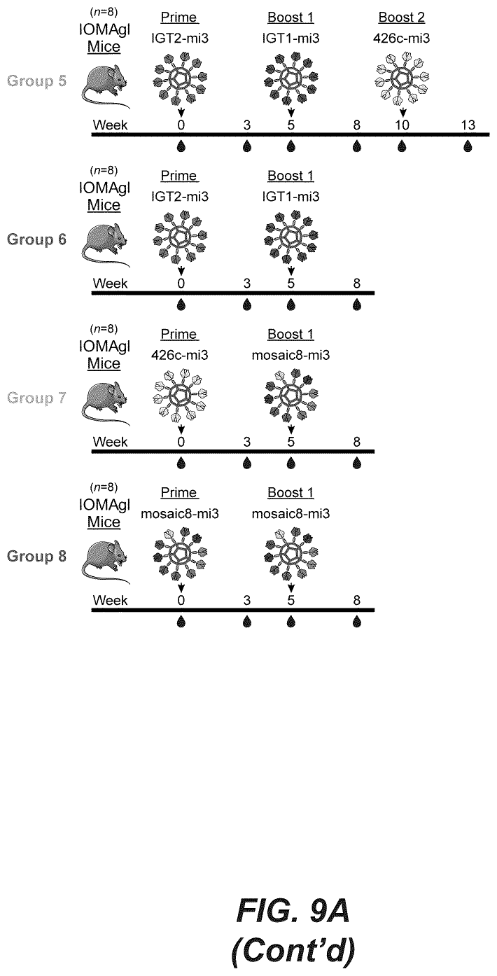

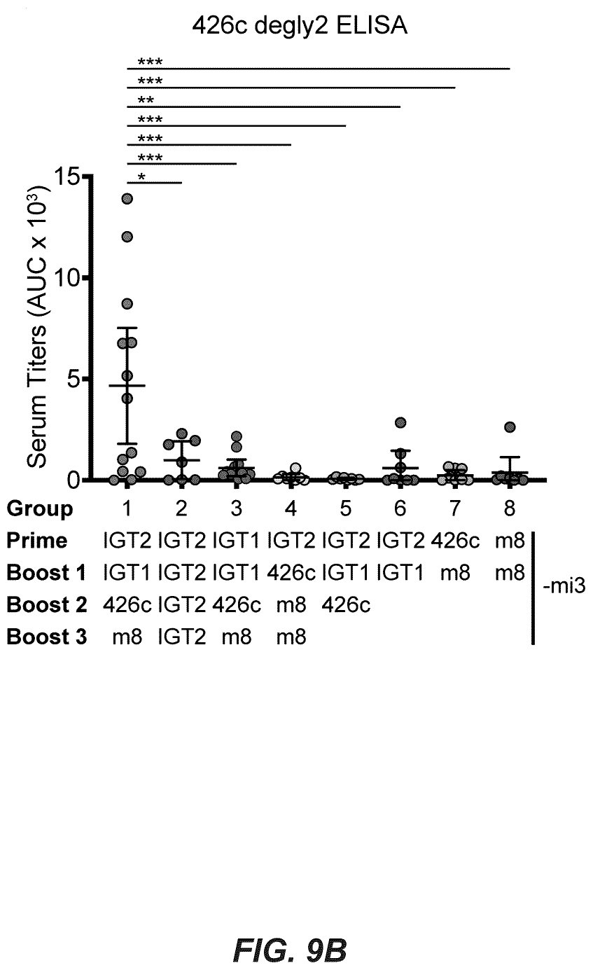

To determine whether a shorter immunization regimen could elicit heterologous neutralizing responses, 7 other immunization regimens were tested in IOMA iGL knock-in mice (FIG. 2H, FIG. 9A-FIG. 9B, groups 2-8). ELISA binding titers against 426c degly2 and 426c SOSIPs using serum from group 1, which was primed with IGT2-mi3 and sequentially boosted with IGT1-mi3, 426c degly2 D279N-mi3, and mosaic8-mi3, were significantly higher than binding titers from the other groups (p: <0.0001) (FIG. 2H, FIG. 9B). These results demonstrate the requirement for germline targeting through sequential immunization to induce IOMA-like antibodies.

bNAbs Isolated from IOMA iGL Knock-In Mice

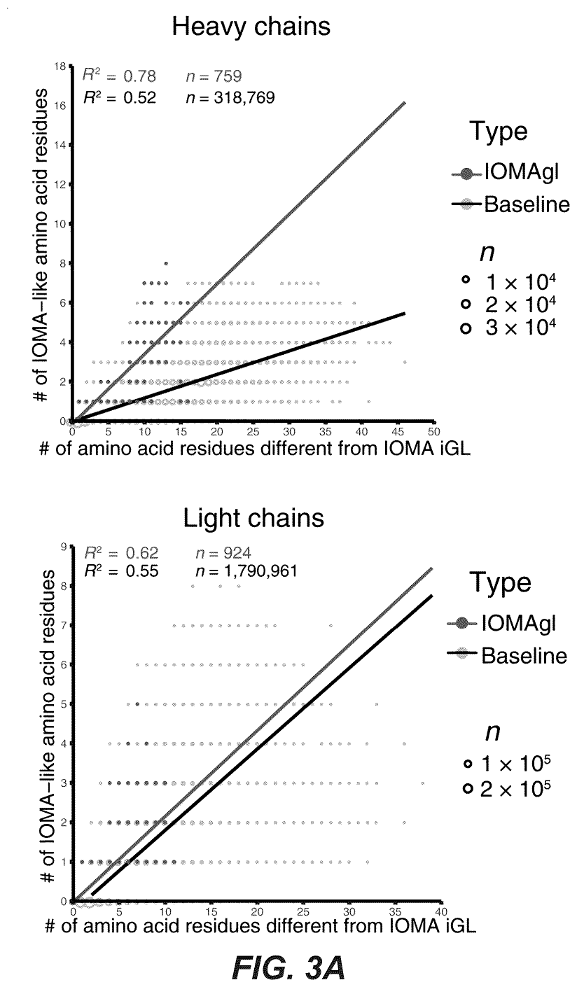

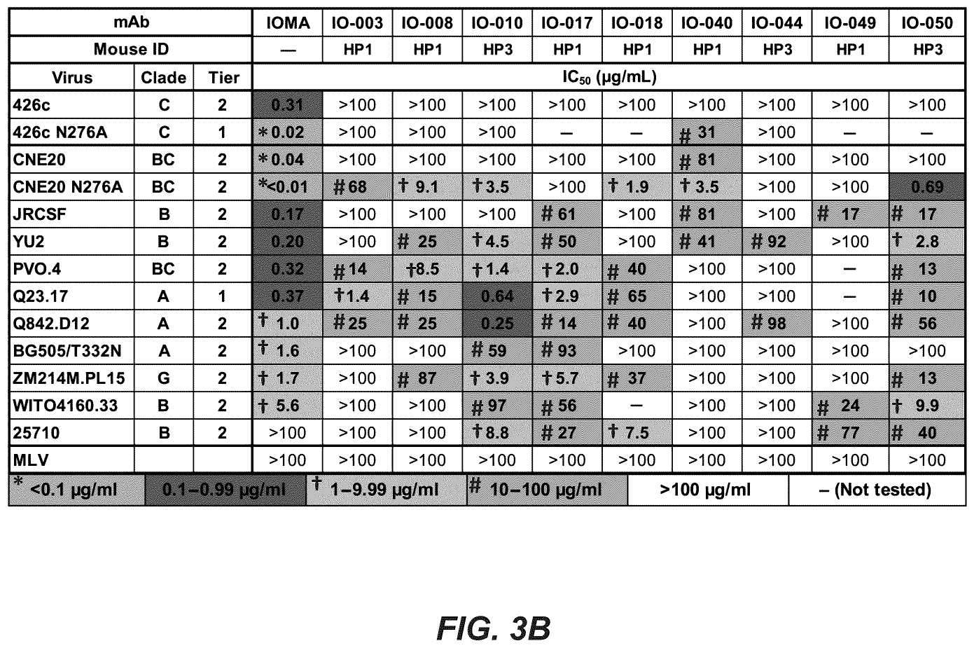

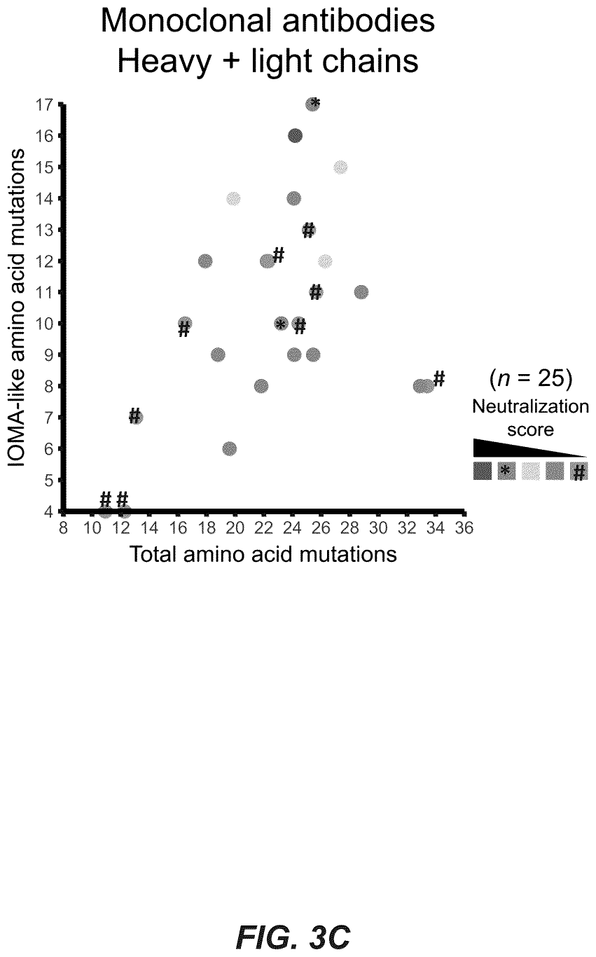

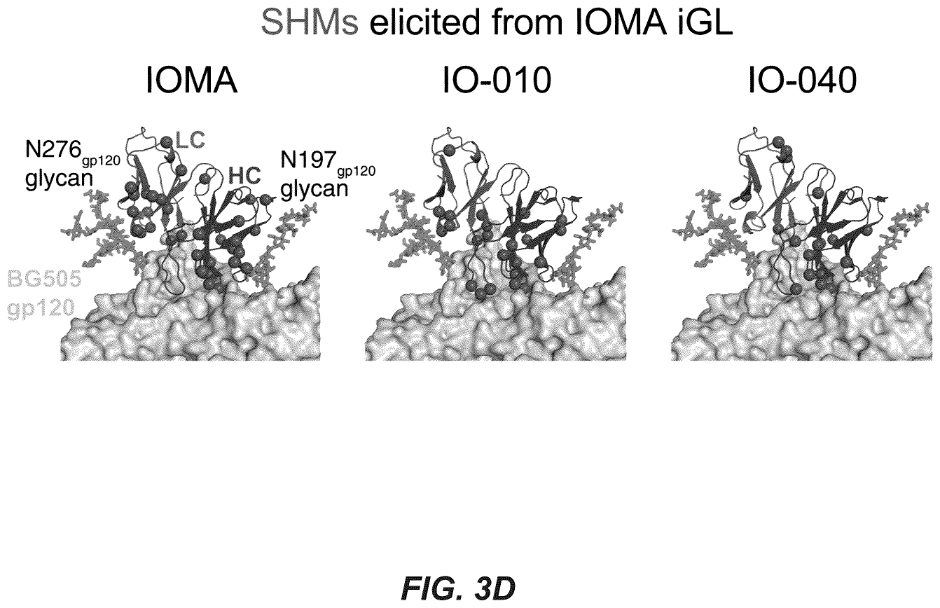

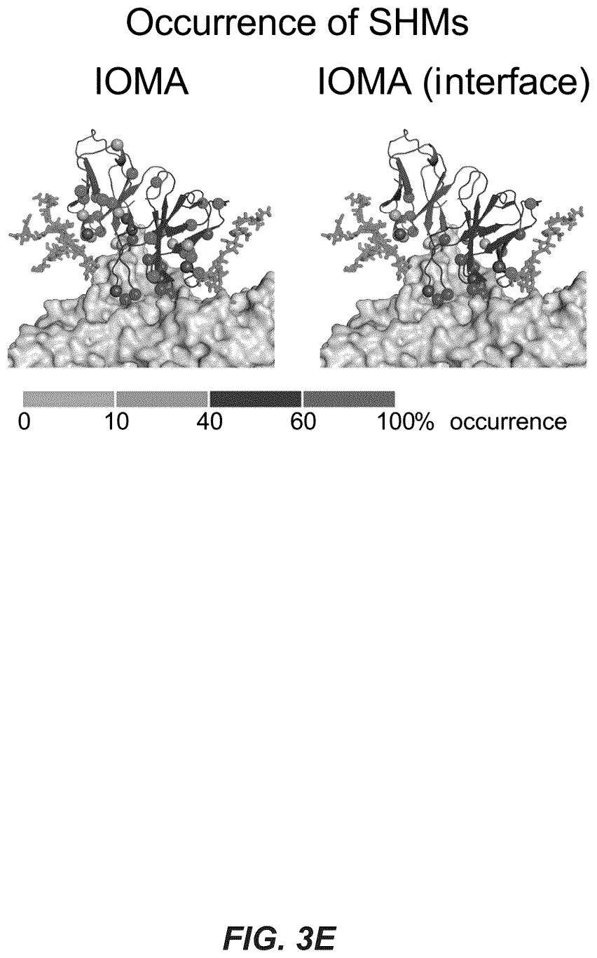

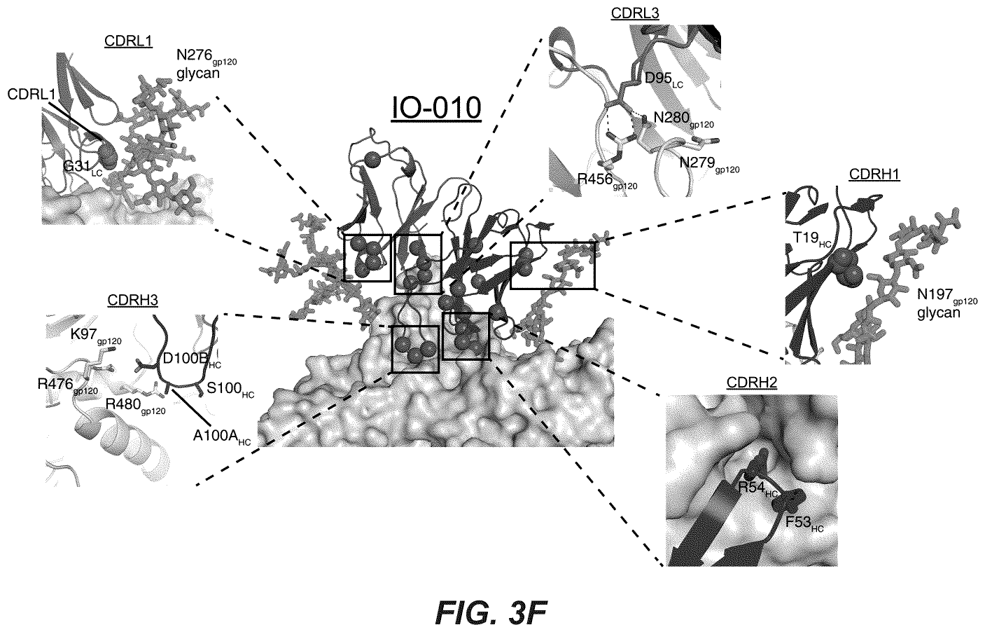

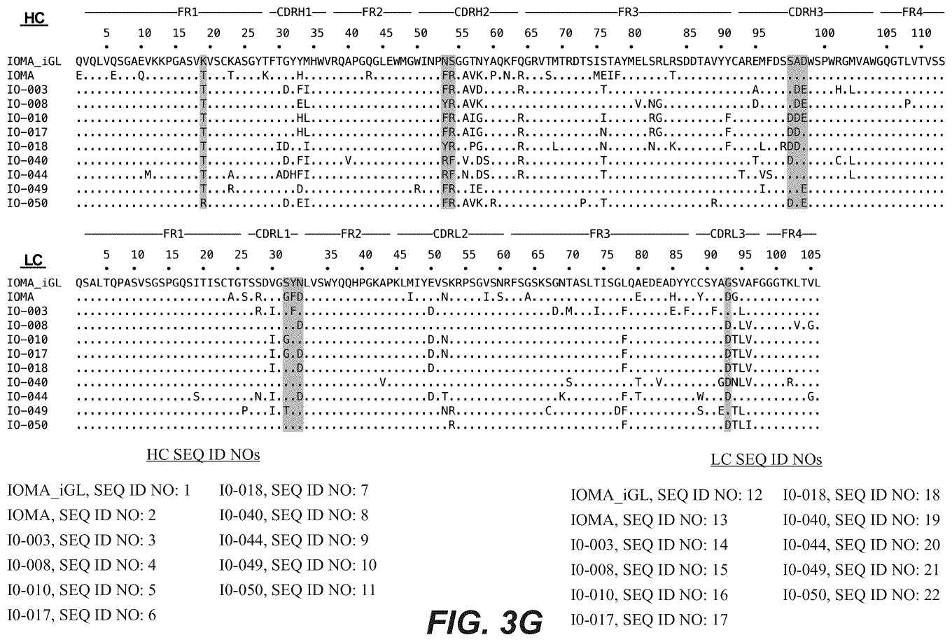

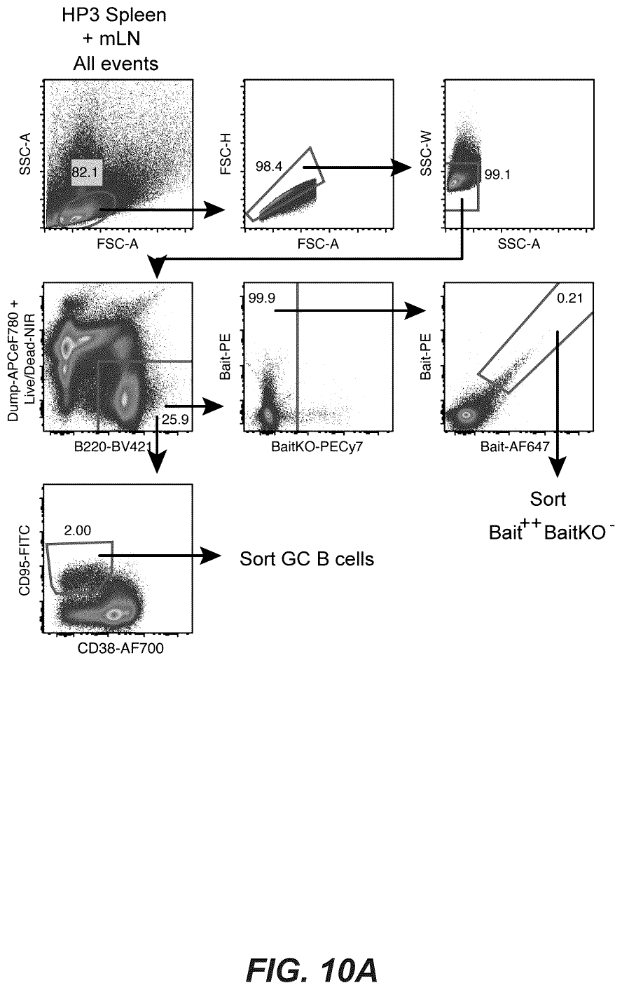

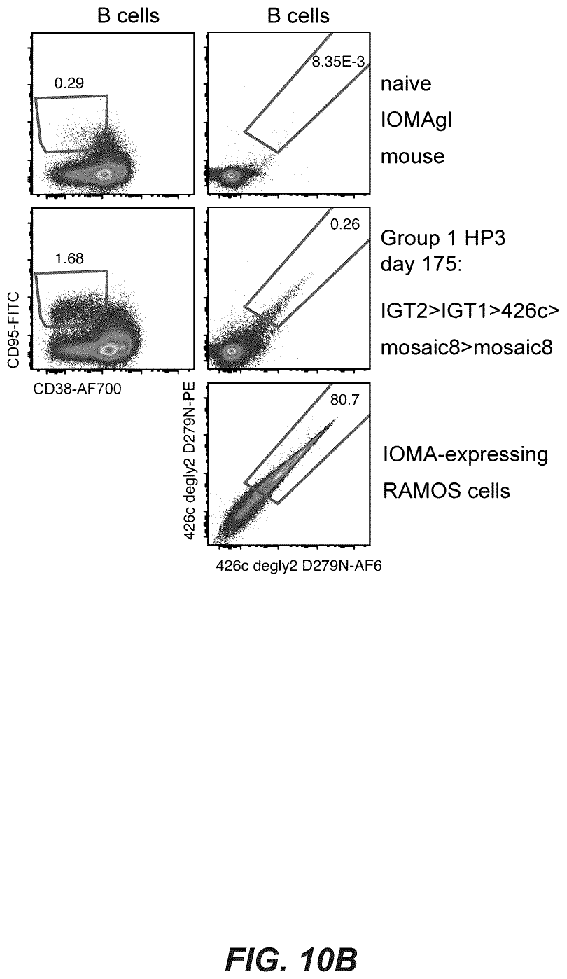

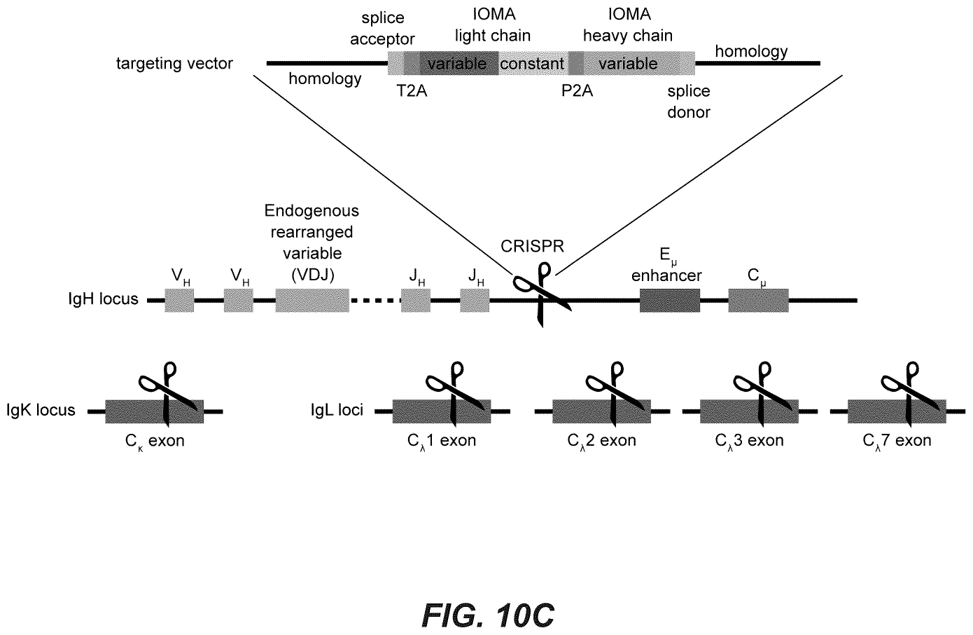

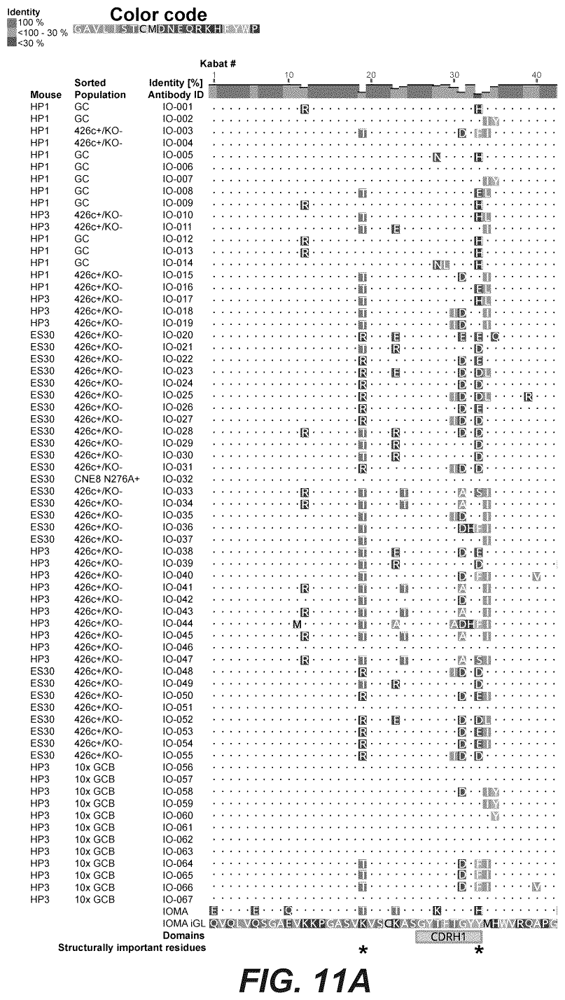

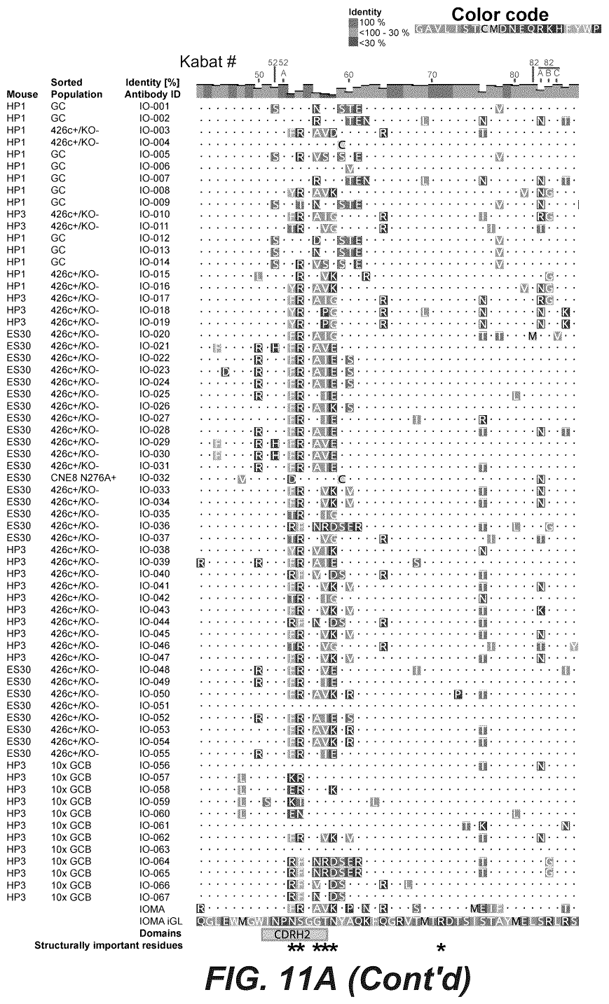

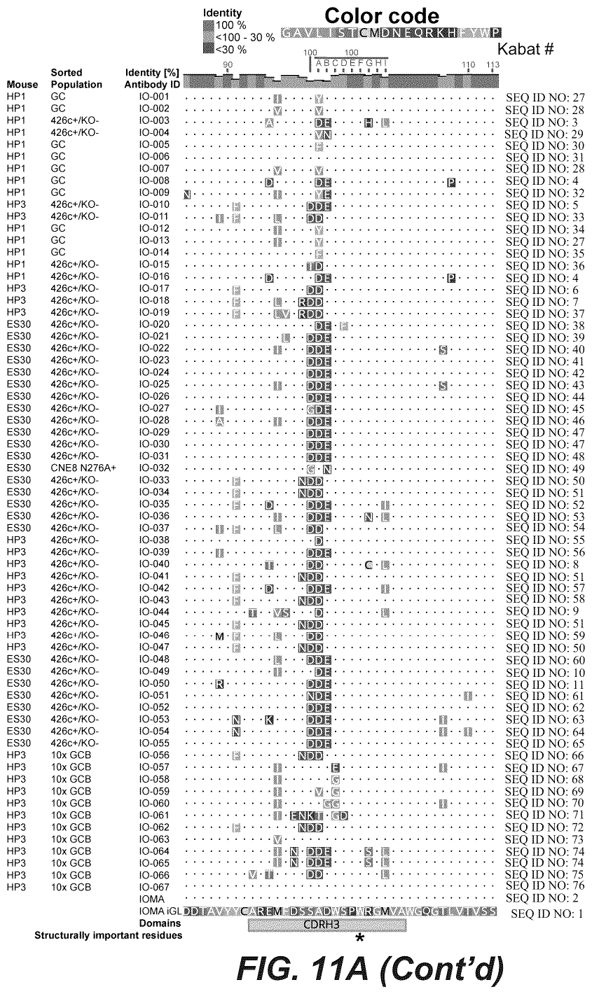

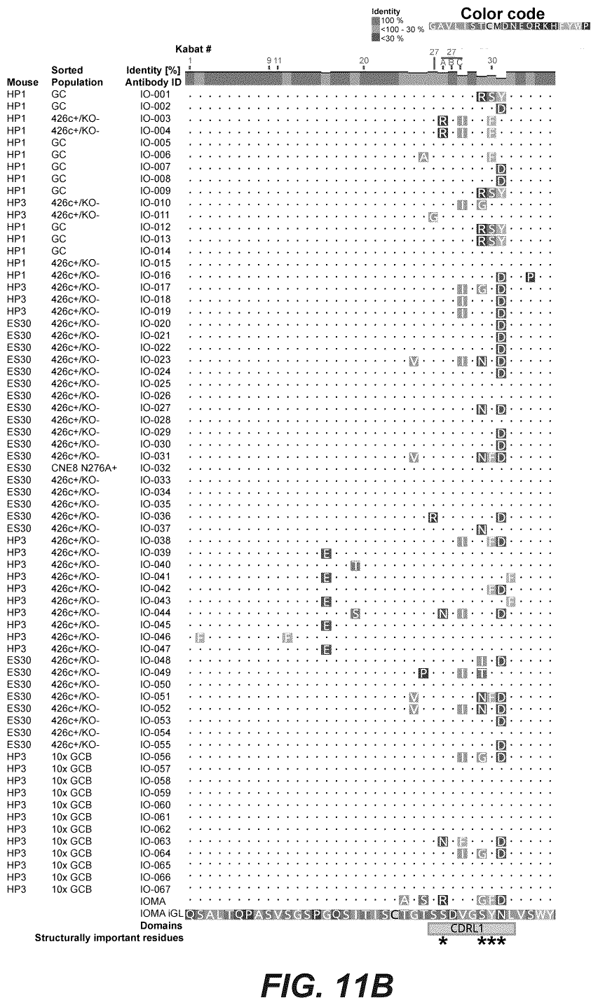

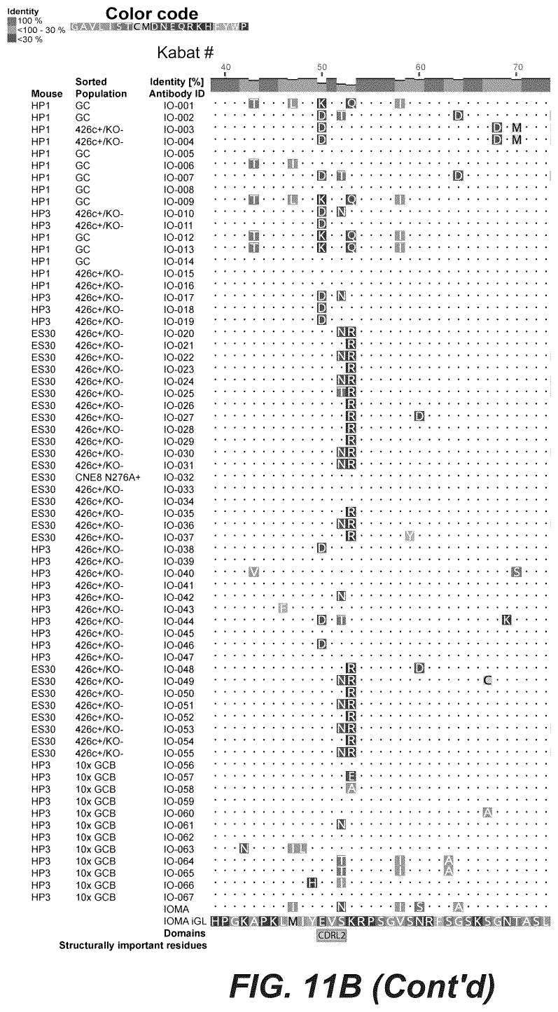

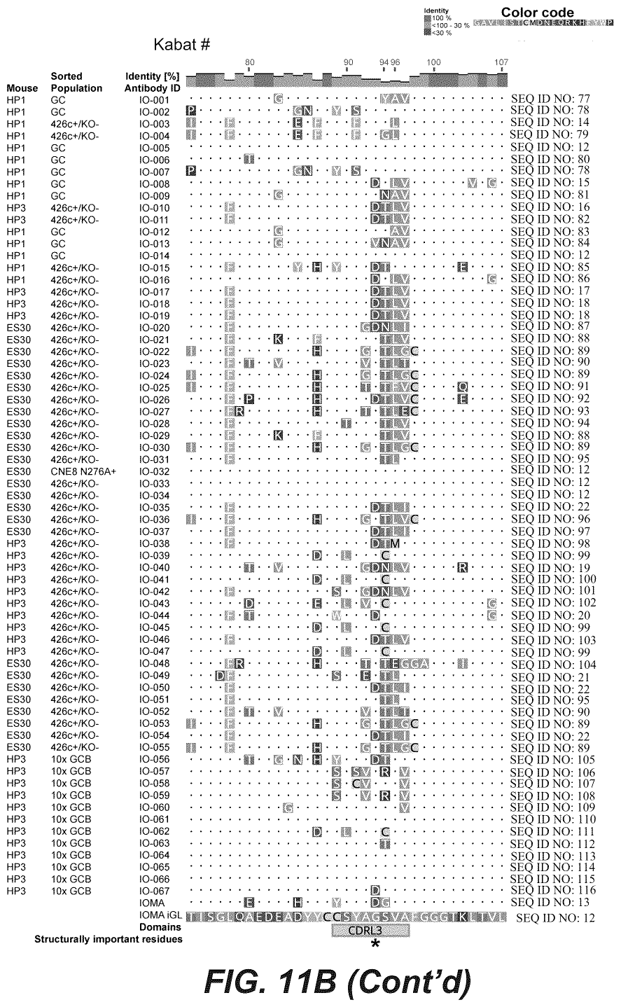

To analyze immunization-induced antibodies, B cells were isolated from spleen and mesenteric lymph nodes of three IOMA iGL knock-in mice of group 1 (ES30, HP1, and HP3) following the final boost (Week 18 or 23, FIG. 2G). Immunization-induced germinal center B cells were sorted or antigen-bait combinations of 426c degly2 D279N or CNE8 N276A together with 426c degly2 D279N-CD4bs KO (Table 1) were used to sort epitope-specific B cells (FIG. 10A-FIG. 10C). Among the identified HC and LC sequences, a correlation (R2=0.78 for HCs and R2=0.62 for LCs) was noted between the total number of V region amino acid mutations and V region mutations with identical or chemical similarity to IOMA. This was compared to unbiased VH1-2*01 or VL2-23*02 sequences derived from peripheral blood of HIV-negative human donors which showed both a lower rate and correlation (R2=0.52 for HCs and R2=0.55 for LCs) of IOMA-like mutations indicating that the immunization regimen induced maturation of IOMA iGL towards IOMA, particularly of the heavy chain which constitutes the majority of contact surface between IOMA and Env-based immunogens (FIG. 3A). 55 paired sequences were selected for antibody production based on mutation load and similarity to mature IOMA (FIG. 11A-FIG. 11B). In addition, 10× Genomics VDJ analysis of germinal center B cells revealed 5207 paired HC and LC sequences, of which another 12 were chosen for recombinant antibody production (FIG. 10A-FIG. 12B).

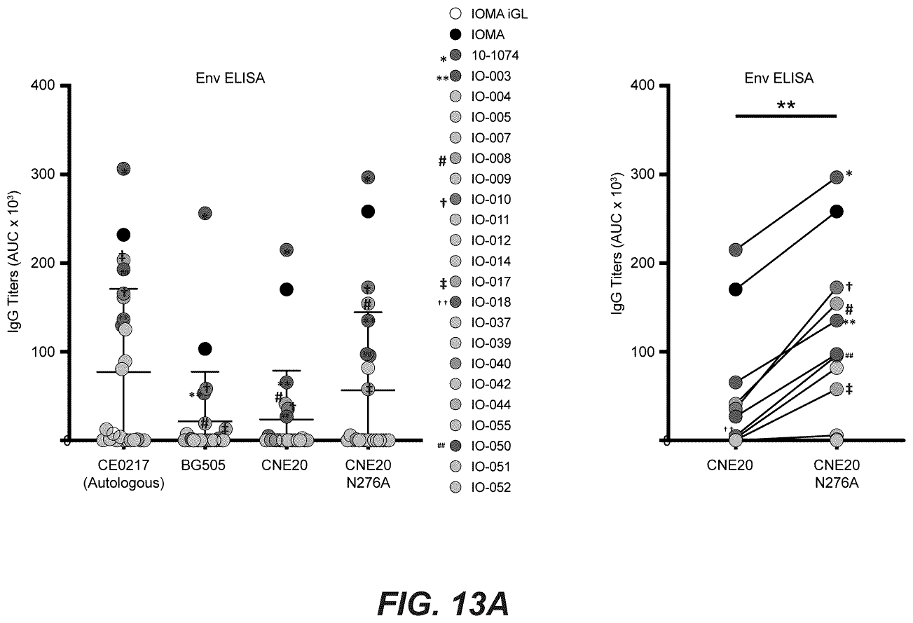

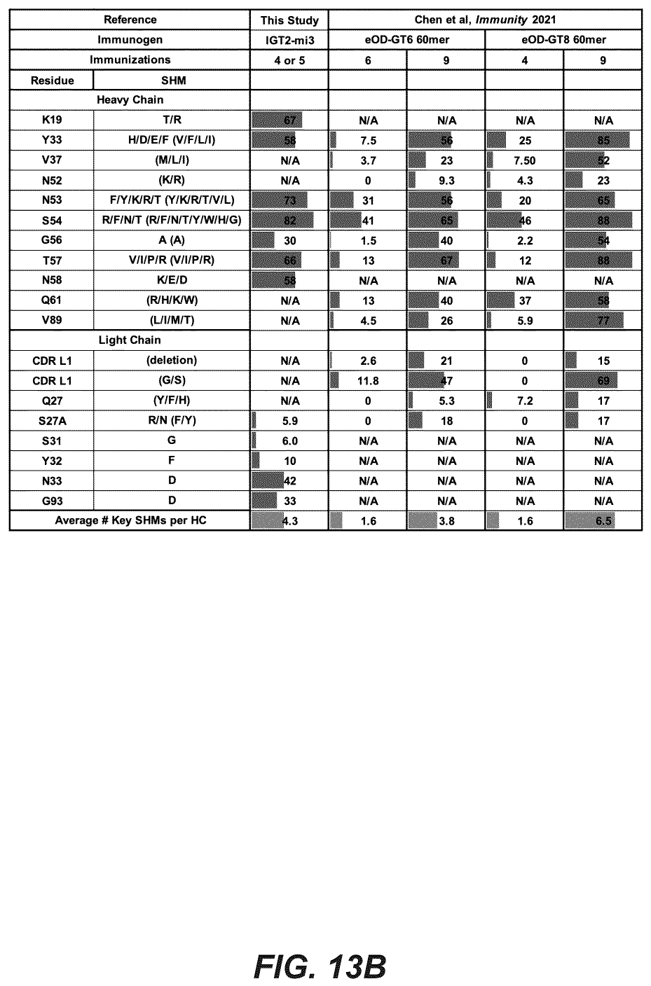

The selected monoclonal antibodies were tested for binding to a panel of heterologous Envs by ELISA (FIG. 13A). Isolated IOMA-like antibodies that demonstrated binding to the Envs were then evaluated in pseudotyped in vitro neutralization assays, and several exhibited similar neutralization potencies as mature IOMA on a small panel of heterologous HIV-1 strains. Some antibodies neutralized the tier 2 strain 25710, which IOMA does not neutralize, and IO-010 neutralized Q842.D12 better than IOMA (FIG. 3B). It was also noted that among the Env-binding monoclonal antibodies, stronger neutralization activity tended to occur with antibodies that shared a larger number of critical residues with IOMA (FIG. 3C).

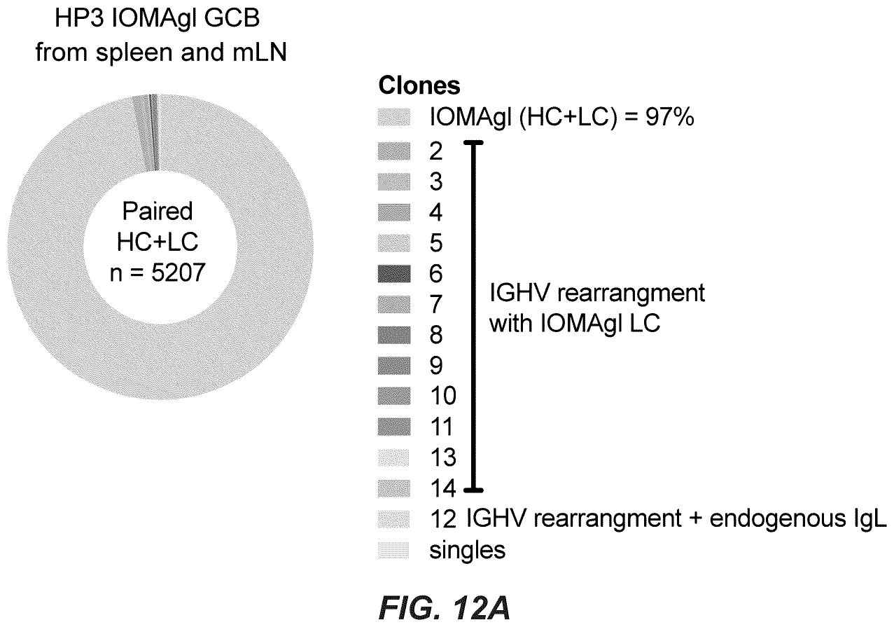

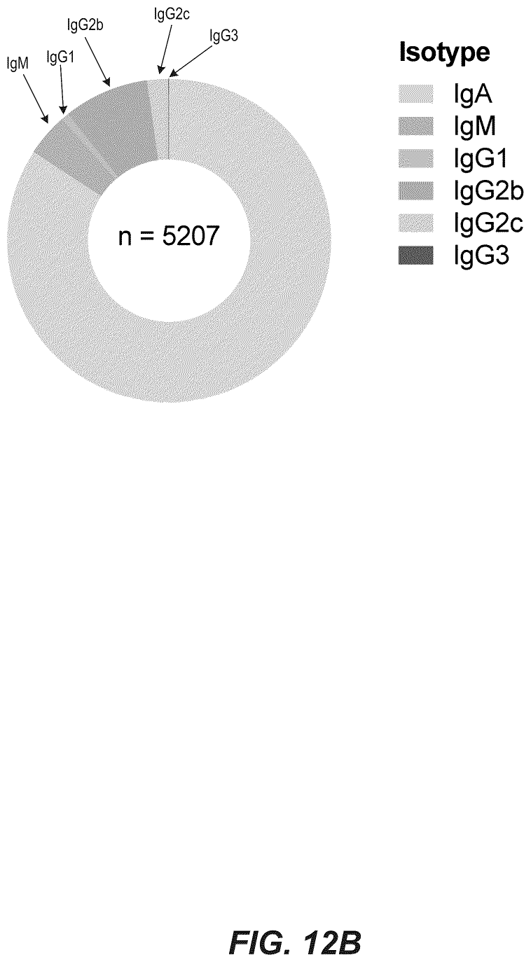

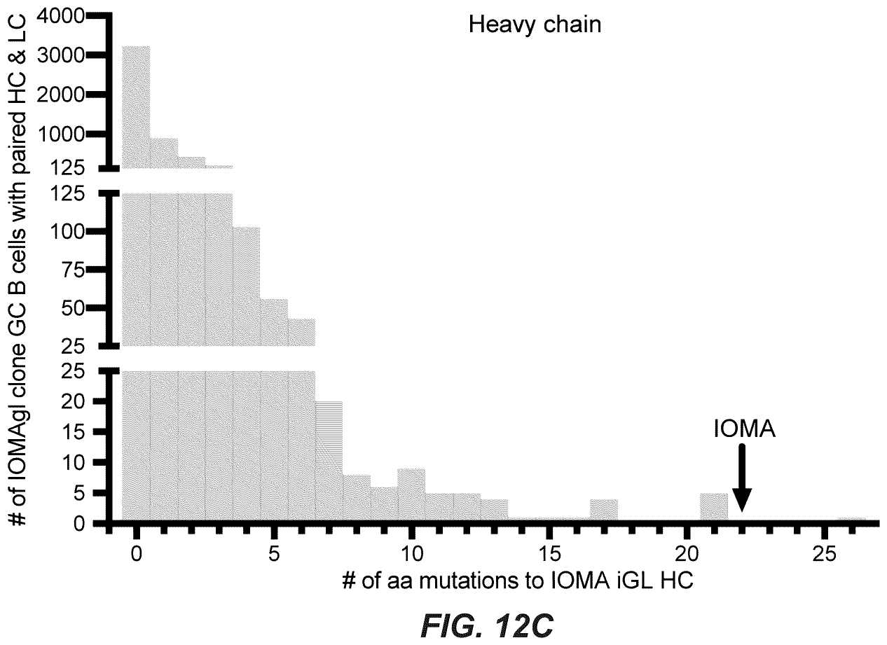

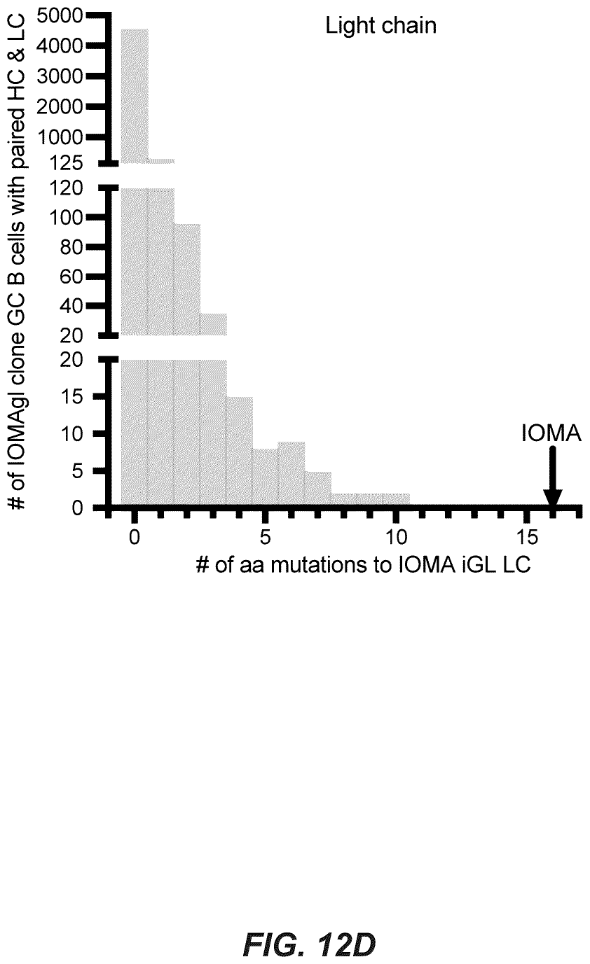

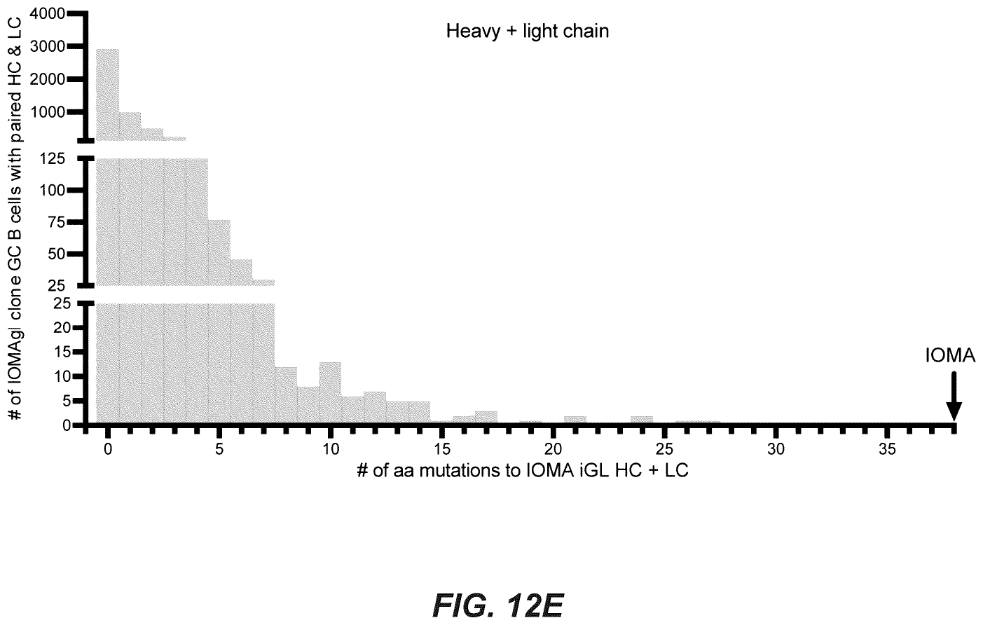

Two mature IOMA residues, CDRH2 residues F53HC and R54HC, interact with the CD4bs Phe43 binding pocket on gp120 and are critical for Env recognition (FIG. 3D-FIG. 3G, FIG. 11A-FIG. 11B). 29 of 67 clones chosen for antibody production (FIG. 11A-FIG. 11B) contained both mutations and another 15 contained R54HC, 5 of which were in combination with Y53HC, which is chemically similar to F53HC. N53FHC is a rare mutation that is found in only ˜0.13% of VH1-2*02-derived antibodies. In contrast, the immunization regimen disclosed herein elicited this mutation in ˜45% of antibodies, a ˜350-fold increase. S54R is elicited at slightly higher frequencies in VH1-2*02-derived antibodies (˜2.7%). However, the present immunization regimen elicited this mutation at an ˜24-fold higher rate compared to the random frequency of this mutation in VH1-2*02-derived antibodies (Table 4). In addition, the disclosed sequential immunization regimen selected for a negatively-charged DDE motif in CDRH3 (replacing the IOMA sequence of S100, A100A and D110B) in 23 of 63 sequences and another 27 sequences with at least 1 of the 3 mutations, which, without being bound by any particular theory, was likely selected for by a highly conserved patch of positively-charged residues found at the IOMA-contacting interface of the Envs used in the immunization regimen [K97_{gp120} (90% conserved), R476_{gp120} (R—64% conserved, R/K-98% conserved), and R480_{gp120} (99% conserved)] (FIG. 3D-FIG. 3G, FIG. 11A-FIG. 11B). To accommodate the N276_{gp120} glycan, IOMA acquired 3 mutations in CDRL1 (S29G_{LC}, Y30F_{LC}, N31D_{LC}). The group 1 immunization regimen elicited all 3 of these substitutions; however, none of the clones contained all these mutations. Of 63 antibodies 7 contained two and another 25 contained one of these mutations (FIG. 3D-FIG. 3E, FIG. 11A-FIG. 11B). Two of the most potent antibodies elicited by the immunization regimen, IO-010 and IO-017, acquired the S31G_{gp120} mutation, suggesting this mutation is more critical to accommodate the N276_{gp120} glycan (FIG. 3D-FIG. 3G) and generating antibody breadth and potency. While accommodation of the N276 glycan is critical for CD4bs bNAbs to develop breadth and potency, CD4bs bNAbs must also acquire mutations to better interact with the N197 glycan, such as K19T_{HC} in FRi. The immunization strategy elicited the K19T_{HC} mutation in 31 of 67 monoclonal antibodies (˜46%), which is ˜20-fold higher compared to the random frequency of this mutation in VH1-2*02-derived antibodies (Table 4). Within the CDRL3, VRC01-class bNAbs acquire a G96E_{LC} mutation that enables interactions with the CD4bs loop, while IOMA acquires a similar G95D_{LC} mutation. Once again, this mutation was elicited in 22 of 67 antibodies (˜33%) by the immunization regimen (FIG. 3D-FIG. 3G, FIG. 11A-FIG. 11B). An essential interaction of VRC01-class bNAbs involves the germline-encoded N58 residue in FR3_{HC}, which makes backbone contacts to the highly (˜95%) conserved R456_{gp120}. Due to a shift away from gp120 in CDRL2, IOMA acquires an N58K_{HC} substitution such that the longer lysine sidechain can access R456_{gp120}. The immunization regimen elicited substitutions at N58_{HC} to amino acids with longer sidechains in 39 of 67 (˜58%) antibodies and was mutated to N58K_{HC} in 17 of 67 (˜24%) antibodies, a ˜1.5-fold increase over the random frequency of the N58K_{HC} mutation (FIG. 3D-FIG. 3G, FIG. 11A-FIG. 111B, Table 4). The immunization regimen elicited additional IOMA-like mutations within CDRH2: G56A_{HC} (˜30%) and T57V_{HC} (˜31%), ˜3-fold and ˜74-fold increases over the random frequency in other VH1-2*02-derived antibodies (Table 4). The 10× Genomics VDJ analysis produced an unbiased view of the extent of SHM elicited in the germinal center over the course of the immunization regimen, which, excluding frame shifts, reached up to 26 amino acid mutations in the HC exceeding the number of mutations of the IOMA HC and up to 10 mutations in the LC (FIG. 12C-FIG. 12E).

Sera from Prime-Boosted Wt Mice Targeted the CD4bs and Displayed Heterologous Neutralizing Activity

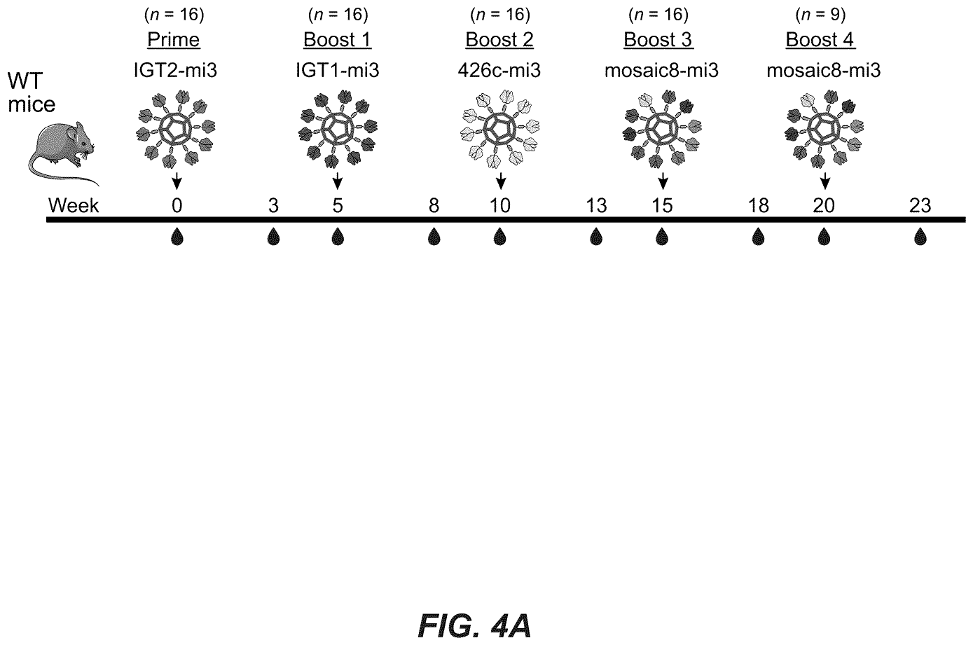

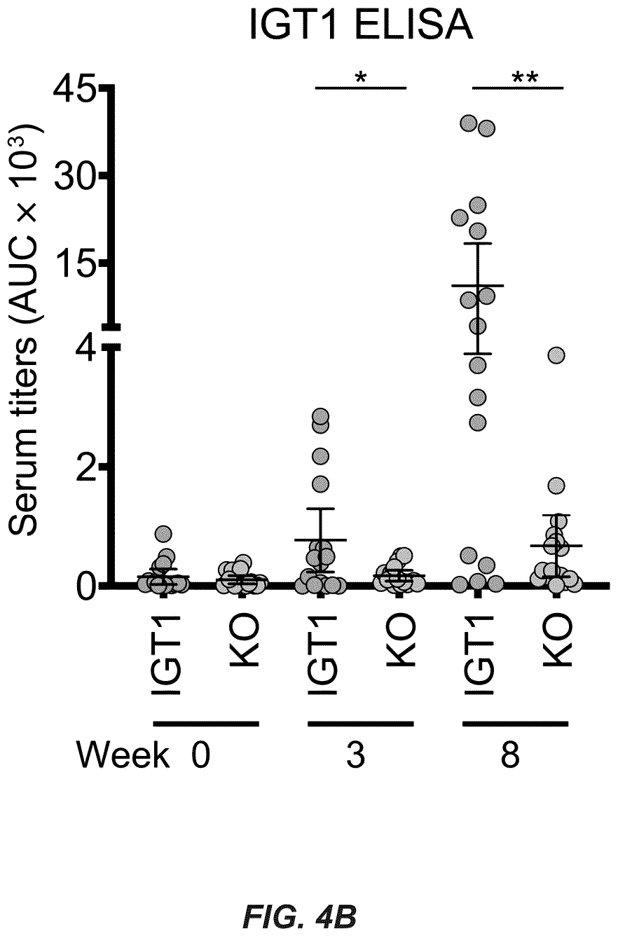

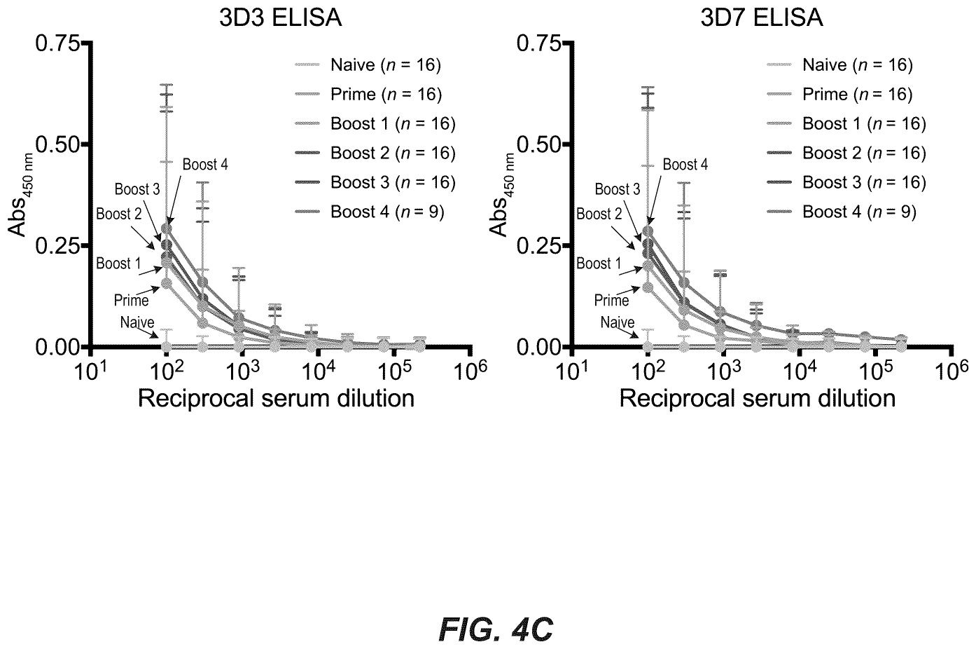

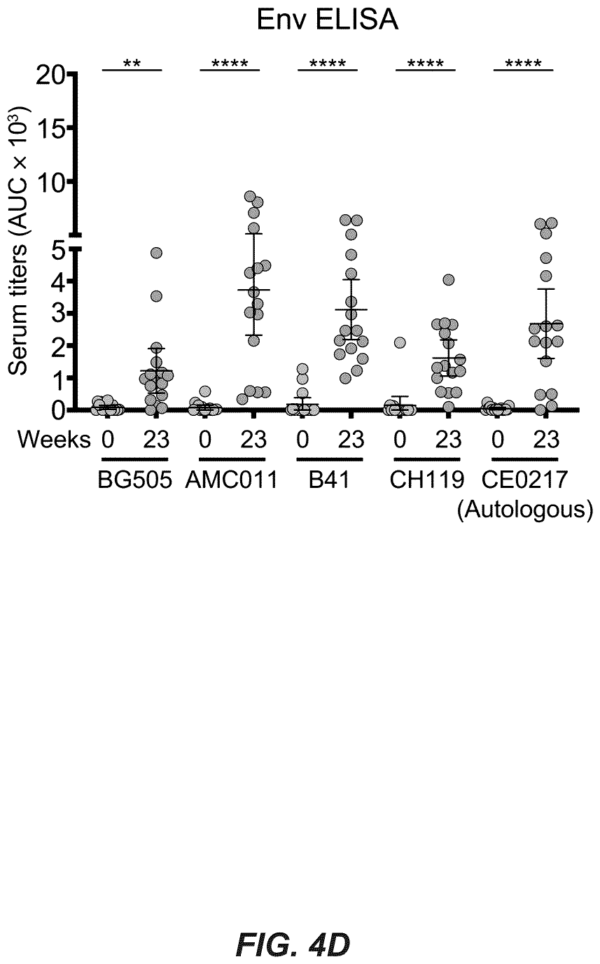

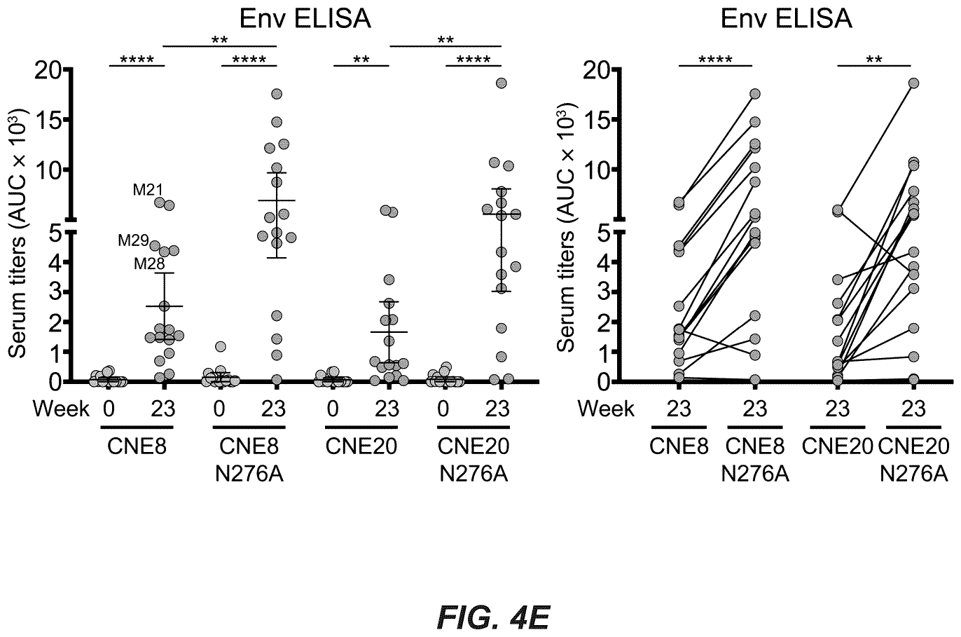

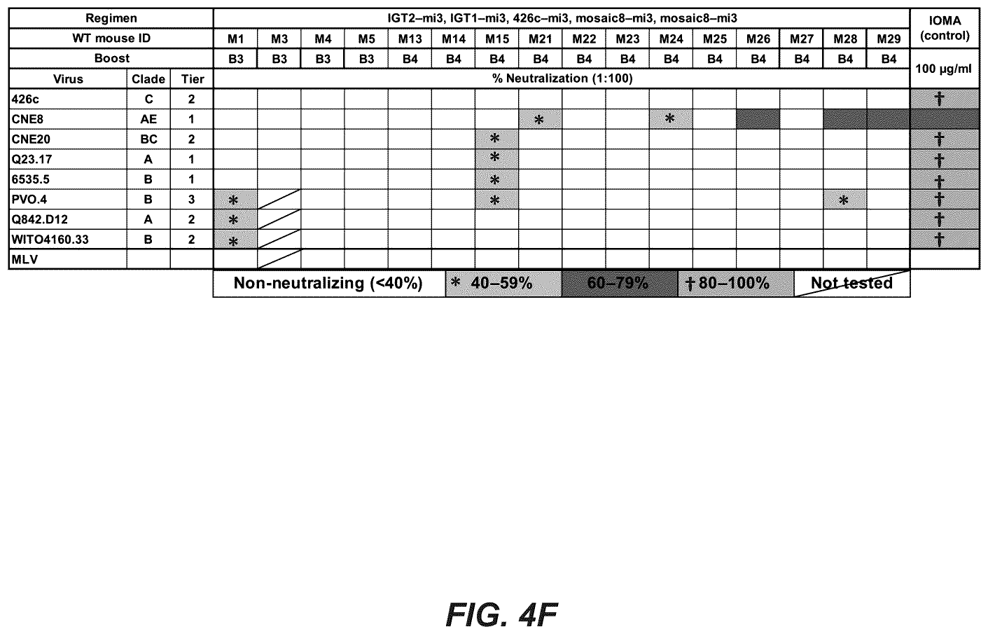

Next, the same immunization regimen was investigated in wt mice (FIG. 4A). Since IOMA does not have the same sequence requirements as VRC01-class bNAbs, it was hypothesized that a prime-boost with IGT2-IGT1 could induce IOMA-like antibodies (which can be defined as recognizing the CD4bs and including a normal-length CDRL3) in wt mice, even though these mice do not contain the VH1-2 germline gene segment. Priming with IGT2-mi3 in wt mice elicited strong serum binding responses that were CD4bs-specific (FIG. 4B, p≤0.05), compared to the IOMA iGL knock-in mice, which only responded robustly after boost with IGT1-mi3 (FIG. 2B-FIG. 2C). As in the IOMA iGL knock-in mice, the magnitude of these responses increased after boosting with IGT1-mi3, and importantly, a significant fraction of the response was still epitope-specific (p<0.001). To characterize antibodies in immunized serum, binding to anti-idiotypic monoclonal antibodies raised against IOMA Igl was measured. While naïve serum did not react with either of the anti-idiotypic antibodies, priming with IGT2-mi3 elicited serum responses that bound both anti-idiotypic antibodies, and boosting with IGT1-mi3 increased these responses (FIG. 4C). After further boosting with 426c-mi3 and mosaic8-mi3 (FIG. 4A), binding to heterologous wt Envs was measured. The immunization regimen elicited significantly increased binding responses to all 9 Envs (FIG. 4D-FIG. 4E, p≤0.05 to <0.001) in the majority of mice. Importantly, serum binding to CNE8 N276A_{gp120} and CNE20 N276A_{gp120} was significantly higher compared to CNE8 and CNE20 (p≤0.05), suggesting that these responses were at least partially specific to the CD4bs (FIG. 4E). Finally, neutralization activity against a panel of heterologous HIV-1 strains was evaluated and weak heterologous neutralization was detected in the serum of 7 of 16 wt animals (FIG. 4F, FIG. 8N-FIG. 8X, Table 5).

### Immunization of Rabbits and Rhesus Macaques Elicited CD4bs-Specific Responses

To evaluate the immunization regimen described herein in other wt animals with more potential relevance to humans, rabbits and rhesus macaques were immunized with IGT2-mi3 followed by IGT1-mi3 (FIG. 5A). For these experiments, only binding for antibody responses was assayed since heterologous neutralization was not achieved after a prime or a prime/single boost of a different HIV-1 immunogen in rabbits or non-human primates (NHPs). As with the wt mouse immunizations, the IGT2-mi3 immunization elicited robust responses that were partially epitope-specific as evaluated by comparing binding to IGT1 versus to IGT1-CD4bs KO (FIG. 5B). When boosted with IGT1-mi3, the responses showed significant increases in epitope specificity to the CD4bs in both rabbits and non-human primates (NHPs) (p<0.05) (FIG. 5B). In addition, post-prime and post-boost sera exhibited potent neutralization of pseudoviruses generated from the IGT2 and IGT1 immunogens (FIG. 5C). As provided above, neutralization of heterologous pseudoviruses was not evaluated since previous results using a different HIV-1 immunogen in rabbits and NHPs showed heterologous neutralization only after a second boost. The increase in epitope specificity and serum neutralization titers following boosting with IGT1 suggests that the presently disclosed immunization strategy is well optimized to elicit CD4bs antibody responses.

Described herein is an immunization regimen to elicit antibodies to the CD4bs epitope on HIV Env using engineered immunogens targeting IOMA-like CD4bs antibody precursors. A goal of the germline-targeting approach has been the induction of bNAbs at protective concentrations, but to date, no study has been able to accomplish this feat, although a study involving mRNA delivery of HIV-1 Env and gag genes reported reduced risk of SHIV infection in immunized NHPs. A previous study using a transgenic mouse expressing diverse VRC01 germline precursors demonstrated that priming with eOD-GT8 followed by sequential boosting with more native-like Envs elicited VRC01-like bNAbs. However, that study required 9 immunizations over 81 weeks to elicit VRC01-class antibodies with heterologous neutralization. By comparison, the method described herein elicited bNAbs with similar breadth and potency using only 4-5 immunizations in 18-23 weeks. In addition, sequence analysis of the monoclonal antibodies elicited in the IOMA iGL transgenic mice revealed that the presently disclosed immunization regimen was much more efficient at eliciting critical mutations required for bNAb development compared to the immunogens used in the attempts to elicit VRC01-class bNAbs. Finally, the neutralization profiles of monoclonal antibodies often correlated with serum neutralization from the mouse they were isolated from. For example, IO-010 and IO-017, which neutralized PVO.4 and Q23.17, were isolated from HP3 and HP1, whose serum also demonstrated neutralization activity against these strains (FIG. 2G and FIG. 8A-FIG. 8M).

Accommodation of the N276gp120 glycan is considered the major impediment to the elicitation of bNAbs targeting the CD4bs. To accommodate the N276 gp120 glycan, VRC01-class bNAbs require a 2-6 residue deletion or the selection of multiple glycines within CDRL1. IOMA requires simpler substitution of 4 residues in CDRL1 (S27ARLC, S29GLC, Y30FLC, N31DLC). These mutations were elicited in the disclosed immunization regimen, although no single clone contained all 4 of these residues. The two most potent monoclonal antibodies isolated from immunized iGL mice, 10-010 and 10-017, contained the S31G mutation, suggesting, without being bound by any particular theory, that this residue is most critical for accommodating the N276gp120 glycan in IOMA-like antibodies and to the development of bNAbs capable of potent heterologous neutralization. Although these antibodies were cloned from mice following the 4th or 5th immunization, sera from week 8 of the disclosed immunization regimen displayed significant binding to 426c Envs containing the N276gp120 glycan (FIG. 2D), suggesting these mutations were elicited following only two immunizations. In contrast, in the same study noted above, mutations within CDRL1 of VRC01 required to accommodate the N276gp120 glycan occurred only after the ninth immunization at 81 weeks (FIG. 13B). Additional mutations known to be important for binding to the CD4bs were also elicited earlier and at higher efficiencies in the present immunization regimen compared to previous studies (FIG. 13B). Importantly, no other reported vaccination regimen to elicit CD4bs antibodies has elicited all of the required SHMs to accommodate the N276gp120 glycan, making the results as described herein an important achievement for eliciting CD4bs bNAbs.

Since the CDRL1 of IOMA iGL was already in a helical conformation, the CDRL1 of the IOMA precursor cells selected by priming and boosting with IGT2 and IGT1 may have been, in some embodiments, in a conformation that allowed it to accommodate the N276gp120 glycan and therefore not required additional SHMs to accommodate the N276gp120 glycan introduced in the third immunization using 426c. Thus, in some embodiments, boosting with Envs that incorporate only high-mannose glycans at N276gp120 followed by boosting with Envs that only incorporate complex-type glycans at N276gp120 starting at the second or third immunizations may force IOMA precursor cells to adapt to more diverse and branching glycan moieties and acquire these critical SHMs. While the vaccine-elicited IOMA-like antibodies overall contained fewer SHMs and lower potencies compared to IOMA, the immunization described herein regimen took place over only ˜5 months. In contrast, PCIN63, the fastest known VH1-2-derived CD4bs bNAb to arise in a natural infection, first emerged ˜40 months post-infection and achieved breadth ˜64 months post-infection and only developed into the fully mature bNAb >70 months post-infection. Thus, the disclosed immunization regimen may, in some embodiments, result in higher levels of SHMs and increased antibody potency when analyzed over longer time courses or by providing additional boosting immunizations at intervals over a longer time period.

Utilizing the strategy developed herein in IOMA iGL knock-in mice, wt mice were immunized with the same immunization regimen (FIG. 4A). A prime-boost sequence with IGT2-mi3 (prime) and IGT1-mi3 (boost) elicited robust CD4bs-specific responses. Importantly, the antibodies elicited by these immunogens resembled IOMA based on binding to an anti-idiotypic antibody raised against IOMA iGL using previously described methods. Subsequent immunization with more native-like Envs, 426c degly2 and mosaic8, generated serum responses capable of neutralizing heterologous HIV strains. Importantly, serum neutralization correlated with ELISA binding titers; e.g., mice that elicited the highest serum binding titers against CNE8 (M21, M28, and M29) also elicited heterologous neutralizing activity against CNE8 pseudovirus. These results represent the first time CD4bs-specific responses and heterologous neutralization were elicited in wt mice, thereby setting a new standard by which to evaluate HIV immunogens in wt mice. Due to the success of our immunogens in wt mice, they were tested in additional animals with polyclonal antibody repertoires—rabbits and rhesus macaques. Once again, the priming immunogens elicited CD4bs-specific binding responses in both animal models, representing the first time a germline-targeting immunogen designed to target CD4bs Abs elicited epitope-specific responses in rabbits and rhesus macaques. In some embodiments, these experiments are complicated by the fact that NHPs do not contain the VH1-2 germline gene segment, which is required for eliciting VRC01-class bNAbs and potentially, without being bound by any particular theory, also IOMA-like Abs. In addition, bNAb activity would not be expected in terms of heterologous neutralization ˜6-10 weeks after a prime immunization since bNAbs require multiple years to arise in a natural infection. In fact, two previous studies immunizing NHPs with CD4bs-targeting immunogens, eOD-GT8 and CH505 M5.G458Y, did not report heterologous neutralization against wt viruses. Nevertheless, initial reports from a phase I clinical trial suggests a prime with eOD-GT8 can elicit VRC01-class bNAb precursor responses in healthy adult humans. Thus, a potential lack of heterologous neutralization in these immunization experiments does not suggest that an IOMA targeting strategy involving sequential immunization would not work in humans.

As a final boost, a mosaic8 nanoparticle presenting eight different wt Envs on the surface was used, with the intention of more efficiently selecting cross-reactive B cells and increasing neutralization breadth, a strategy that was employed to elicit cross-neutralizing responses to influenza or to zoonotic coronaviruses of potential pandemic interest. Indeed, serum isolated from both wt and transgenic mice after a mosaic8-mi3 boost bound to heterologous Envs in ELISAs and neutralized a panel of heterologous HIV pseudoviruses. Without being bound by any particular theory, the cross-neutralization may be due to boosting with mosaic8-mi3.

Although a previous study suggested using gp120 cores as an important intermediate immunization step, the approach disclosed herein resulted in heterologously-neutralizing antibodies using trimeric SOSIP-based Envs for all immunizations. This is an important distinction, since using trimeric Envs provides the additional benefit of simultaneous targeting of multiple bNAb epitopes. Indeed, a protective HIV-1 vaccine will, in some embodiments, require the elicitation of bNAbs to multiple epitopes to prevent escape from the host immune response during early infection to enable clearing of the virus. Thus, the presently disclosed immunogens provide a scaffold upon which to engineer other epitopes to initiate germline-targeting of additional bNAb precursors.

IOMA's relatively lower number of SHMs and normal-length CDRL3 suggest that eliciting IOMA-like bNAbs by vaccination can be easier to achieve, compared with eliciting VRC01-class bNAbs. Indeed, the fact the IOMA immunogens elicited CD4bs-specific responses in four animal models suggests that germline-targeting immunogens designed to elicit IOMA-like antibodies are an attractive route to generate an HIV-1 vaccine, which is supported by the disclosed engineered immunogens eliciting epitope-specific responses in wt animals and by a commonality of the mutations that were induced across individual transgenic mice. Furthermore, IOMA-like bNAbs have been isolated from multiple patients, suggesting an immunization regimen targeting this class of bNAbs may be universally effective in a global population. Although IOMA's neutralization breadth is smaller than that of other bNAbs, the fact that some vaccine-elicited IOMA-like antibodies neutralized strains that IOMA neutralizes less potently or does not neutralize at all suggests that polyclonal serum responses can be created that include individual antibodies with more breadth than IOMA. If elicited at sufficient levels, such antibodies may, in some embodiments, mediate protection from more strains than predicted by the original IOMA antibody. In some embodiments, this is an important property of an active vaccine, since clinical trials to evaluate protection from HIV-1 infection by passive administration of VRC01 in humans demonstrated a lack of protection from infection by HIV-1 strains against which the VRC01 exhibited weak in vitro potencies. Although polyclonal antibodies raised against the CD4bs may be more protective than a single administered monoclonal anti-CD4bs antibody, a successful HIV-1 vaccine will, in some embodiments, likely require broader and more potent responses to the CD4bs and other epitopes on HIV-1 Env. The results described herein provide new germline-targeting immunogens, demonstrating that IOMA-like precursors provide a new method to elicit CD4bs bNAbs to generate a protective HIV-1 vaccine.

### Materials and Methods

Study Design

The objective of this study was to determine whether IOMA-like Abs could be elicited through immunization and whether this process would be easier and more practical than regimens used to elicit other CD4bs Abs. To do this, a yeast display platform was established to screen gp120 libraries and select variants that bind to a germline IOMA precursor. The gp120s were then expressed in mammalian cells and validated biochemically and biophysically by SPR, ELISA, and SEC. Top candidate immunogens were then incorporated into immunization regimens and evaluated to determine whether they select B cells expressing IOMA-like precursors in transgenic and wildtype animal models. Animal immunizations and serum characterization were conducted by separate laboratories in a blinded manner. Furthermore, different labs were responsible for the immunizations of each animal—mice, rabbits, or NHPs. The size of animal cohorts, dosage, and time interval between immunizations were determined from previous experiments so that an optimal regimen could be used without the need for extensive testing. Experimental end points were determined on the basis of serum ELISA binding titers to wt Env proteins. The efficacy of the resulting immunization regimen was evaluated by serum ELISAs and serum neutralization assays. Monoclonal antibodies were isolated from transgenic animals demonstrating heterologous serum neutralization activity. These antibodies were further characterized in neutralization assays.

Antibody, Gp120, and Env Trimer Expression and Purification

Env immunogens were expressed as soluble SOSIP.664 native-like gp140 trimers as described. For SpyTagged trimers, either SpyTag (13 residues) or SpyTag003 (16 residues) was added to the C-terminus to allow formation of an irreversible isopeptide bond to SpyCatcher003 moieties. All soluble SOSIP Envs were expressed by transient transfection in HEK293-6E cells (National Research Council of Canada) or Expi293 cells (Life Technologies) and purified from transfected cell supernatants by 2G12 affinity chromatography. Soluble Envs were stored at 4° C. in 20 mM Tris pH 8.0, 150 mM sodium chloride (TBS) (untagged and AviTagged versions) or 20 mM sodium phosphate pH 7.5, 150 mM NaCl (PBS) (SpyTagged versions). Untagged gp120 proteins as cores with N/C termini and V1/V2/V3 loop truncations were also expressed by transient transfection of suspension-adapted HEK293-S cells. gp120s were purified using Ni-NTA affinity chromatography and Superdex 200 16/60 SEC. Proteins were stored in 20 mM Tris, pH 8.0, 150 mM sodium chloride.

The iGL sequences of IOMA was derived as described above. The iGL sequences of VRC01 and 3BNC60 were derived using previously described methods. The iGL of BG24, a VRC01-class bNAb with relatively few SHMs, was derived using previously described methods. IgGs were expressed by transient transfection in Expi293 cells or HEK293-6E cells and purified from cell supernatants using MabSelect SURE (Cytiva) columns followed by SEC purification using a 10/300 or 16/600 Superdex 200 (GE Healthcare) column equilibrated with PBS (20 mM sodium phosphate pH 7.4, 150 mM NaCl). His-tagged Fabs were prepared by transient transfection of truncated heavy chain genes encoding a C-terminal 6×-His tag with a light chain expression vector and purified from supernatants using a 5 mL HisTrap column (GE Healthcare) followed by SEC as described above.

ELISAs

Serum ELISAs were performed using randomly biotinylated SOSIP trimers using the EZ-Link NHS-PEG4-Biotin kit (Thermo Fisher Scientific) according to the manufacturer's guidelines. Based on the Pierce Biotin Quantitation kit (Thermo Fisher Scientific), the number of biotin molecules per protomer was estimated to be ˜1-4. Biotinylated SOSIP timers were immobilized on Streptavidin-coated 96-well plates (Thermo Fisher Scientific) at a concentration of 2-5 μg/mL in blocking buffer (1% BSA in TBS-T: 20 mM Tris pH 8.0, 150 mM NaCl, 0.1% Tween 20) for 1 h at RT. After washing plates in TBS-T, plates were incubated with a 3-fold concentration series of mouse, rabbit, or rhesus macaque serum at a top dilution of 1:100 in blocking buffer for 2-3 h at RT. After washing plates with TBS-T, HRP-conjugated goat anti-mouse Fc antibody (Southern Biotech, #1033-05) or HRP-conjugated goat anti-rabbit IgG Fc antibody (Abcam, ab98467) or HRP-conjugated goat anti-human multi-species IgG antibody (Southern Biotech, #2014-05) was added at a dilution of 1:8,000 in blocking buffer for 1 h at RT. After washing plates with TBS-T, 1-Step Ultra TMB substrate (Thermo Fisher Scientific) was added for ˜3 min. Reactions were quenched by addition of 1 N HCl and absorbance at 450 nm were analyzed using a plate reader (BioTek). ELISAs with gp120s and anti-idiotype monoclonal antibodies were performed as above except these proteins were immobilized directly onto high-binding 96-well assay plates (Costar) in 0.1 M sodium bicarbonate buffer (pH 9.8) at a concentration of 2-5 μg/mL in blocking buffer (1% BSA in TBS-T) for 2 h at RT. ELISAs with IgGs instead of serum were performed as above with a top IgG concentration of 100 μg/mL. All reported values represent the average of at least two independent experiments.

Preparation of SOSIP-Mi3 Nanoparticles

SpyCatcher003-mi3 particles were prepared by purification from BL21 (DE3)-RIPL E. coli (Agilent) transformed with a pET28a SpyCatcher003-mi3 gene (including an N-terminal 6×-His tag) as described. Briefly, cell pellets from transformed bacterial were lysed with a cell disruptor in the presence of 2.0 mM PMSF (Sigma). Lysates were spun at 21,000 g for 30 min, filtered with a 0.2 μm filter, and mi3 particles were isolated by ammonium sulfate precipitation followed by SEC purification using a HiLoad 16/600 Superdex 200 (GE Healthcare) column equilibrated with 25 mM Tris-HCl pH 8.0, 150 mM NaCl, 0.02% NaN3 (TBS). SpyCatcher003-mi3 particles were stored at 4° C. and used for conjugations for up to 1 month after filtering with a 0.2 μm filter and spinning for 30 min at 4° C. and 14,000 g.

Purified SpyCatcher003-mi3 was incubated with a 2-fold molar excess (SOSIP to mi3 subunit) of purified SpyTagged SOSIP (either a single SOSIP or an equimolar mixture of eight SOSIPs for making mosaic8 particles) overnight at RT in PBS. Conjugated SOSIP-mi3 particles were separated from free SOSIPs by SEC on a Superose 6 10/300 column (GE Healthcare) equilibrated with PBS. Fractions corresponding to conjugated mi3 particles were collected and analyzed by SDS-PAGE. Concentrations of conjugated mi3 particles were determined using the absorbance at 280 nm as measured on a Nanodrop spectrophotometer (Thermo Scientific).

Electron Microscopy of Sosip-Mi3 Nanoparticles

SOSIP-mi3 particles were characterized using negative stain electron microscopy (EM) to confirm stability and the presence of conjugated SOSIPs on the mi3 surface. Briefly, SOSIP-mi3 particles were diluted to 20 μg/mL in 20 mM Tris (pH 8.0), 150 mM NaCl and 3 μL of sample was applied onto freshly glow-discharged 300-mesh copper grids. Sample was incubated on the grid for 40 s and excess sample was then blotted away with filter paper (Whatman). 3 μL uranyl acetate was added for 40 s and excess stain was then blotted off with filter paper. Prepared grids were imaged on a Talos Arctica (ThermoFisher Scientific) transmission electron microscope at 200 keV using a Falcon III 4k×4k (ThermoFisher Scientific) direct electron detector at 13,500× magnification.

Mice

C57BL/6J and B6(Cg)-Tyrc-2J/J (B6 albino) mice were purchased from Jackson Laboratories. IghIOMAiGL and IgkIOMAiGL mice were generated with the Rockefeller University CRISPR and Genome Editing Center and Transgenic and Reproductive Technology Center in CY2.4 albino C57BL/6J-Tyrc-2J-derived embryonic stem cells. Chimeras were crossed to B6(Cg)-Tyrc-2J/J for germline transmission. IghIOMAiGL and IgkIOMAiGL mice carry the IGV(D)J genes encoding the IOMA iGL HC and LC respectively. IOMA iGL LC was targeted into the Igk locus deleting the endogenous mouse Igkj1 to Igkj5 gene segments. IOMA iGL HC was targeted into the Igh locus and deleting the endogenous mouse Ighd4-1 to Ighj4 gene segments thereby minimizing rearrangement of the locus (FIG. 7A-FIG. 7B). The constant regions of Igh and Igk remain of mouse origin. Mice were only crossed to C57BL/6J or B6(Cg)-Tyrc-2J/J or themselves and maintained at Rockefeller University and all experiments shown used double homozygous animals for IghIOMAiGL and IgkIOMAiGL abbreviated IOMAgl mice. These mice are available upon request. Mice were housed at a temperature of 22 C and humidity of 30-70% in a 12 h light/dark cycle with ad libitum access to food and water. Male and female mice aged 6-12 weeks at the start of the experiment were used throughout. All experiments were conducted with approval from the institutional review board and the institutional animal care and use committee at the Rockefeller University. Sample sizes were not calculated a priori. Given the nature of the comparisons, mice were not randomized into each experimental group and investigators were not blinded to group allocation. Instead, experimental groups were age- and sex-matched.

Animal Immunizations and Sampling

Mice were immunized intraperitoneally with 10 μg conjugated mi3-SOSIP in 100 μL PBS with 1 U SMNP adjuvant (kindly provided by Murillo Silva, Mariane B. Melo and Darrell J. Irvine, MIT). Serum samples were collected throughout the experiment by submandibular bleeding and animals were terminally bled under isoflurane anesthesia first submandibularly followed by cardiac puncture. Spleen and mesenteric lymph nodes were dissected, mashed though a 70 μm cell strainer and frozen in FCS with 10% DMSO in a gradual freezing (˜1° C./min) container, followed by transfer to liquid N2 for long-term storage.

Eight six-month-old New Zealand White rabbits (LabCorp) were used for immunizations. Rabbits were immunized subcutaneously with 50 μg of a SOSIP-mi3 in SMNP adjuvant (375 U/animal) as described. Serum samples were collected from rabbits at the time points indicated in FIG. 5A. Procedures in rabbits were approved by the Denver PA IACUC Committee.

Five rhesus macaques (Macaca mulatta) of Indian genetic origin were housed in a biosafety level 2 NIAID facility and cared for in accordance with Guide for Care and Use of Laboratory Animals Report number NIH 82-53 (Department of Health and Human Services, Bethesda, 1985). All animal procedures and experiments were performed according to protocols approved by the IACUC of NIAID, NIH. The NHPs used in this study did not express the MHC class I Mamu-A*01, Mamu-B*08 and Mamu-B*17 alleles. NHPs were immunized subcutaneously in the medial inner forelegs and hind legs (total of 4 sites per animal) with 200 μg of the indicated SOSIP-mi3 adjuvated in SMNP (375 U/animal). Immunizations and blood samples were obtained from naïve and immunized macaques at the time points indicated in FIG. 5A.

Flow Cytometry and Cell Sorting

Fresh bone marrow was flushed out of 1 femur and 1 tibia per mouse. Fresh mouse spleens were forced through a 70 μm mesh into FACS buffer (PBS containing 2% heat-inactivated FBS and 2 mM EDTA), and red blood cells of fresh spleens or bone marrow were lysed in ammonium-chloride-potassium buffer lysing buffer (Gibco) for 3 min. Frozen cells were thawed in a 37° C. water bath and immediately transferred to prewarmed mouse B cell medium consisting of RPMI-1640, supplemented with 10% heat-inactivated FBS, 10 mM HEPES, 1× antibiotic-antimycotic, 1 mM sodium pyruvate, 2 mM L-glutamine, and 53 μM 2-mercaptoethanol (all from Gibco).

Bait proteins were randomly conjugated to biotin and free biotin removed using EZ-Link Micro NHS-PEG₄-Biotinylation Kit (ThermoFisher #21955) according to the manufacturer's instructions.

Fluorophore conjugated bait and bait-KO antigen tetramers were prepared by mixing a 5 μg/mL solution of a single randomly-biotinylated bait protein with fluorophore-conjugated streptavidin (Table 7) at a 1:200 to 1:600 dilution in PBS for 30 min on ice. Conjugates were then mixed equivolumetrically.

RAMOS cells were harvested, washed in FACS buffer and stained with human FC-blocking reagent, biotinylated bait antigen-streptavidin tetramers (PE, AF647 and sometimes PECy7) and Zombie-NIR Live/Dead cell marker for 15 min before addition of anti-human antibodies to IgL-APC, IgK-BV421, IgM-FITC, and for some experiments, CD19-PECy7 (Table 7).

Mouse cells and controls (see below) were washed and resuspended in a solution of mouse Fc-receptor blocking antibody, fluorophore-conjugated antigen tetramers and Zombie-NIR Live/Dead cell marker for 15 min on ice. A mastermix of other antibodies was then added and cells stained for another 20 min on ice. Antibodies and reagents are listed in Table 7. All cells were analyzed on an LSRFortessa or cells were sorted on a FACS Aria III (both Becton Dickinson) using IOMA-expressing RAMOS cells as an antigen-binding positive control and splenocytes from naïve IOMAgl mice as negative controls (FIG. 10A-FIG. 10C). To derive absolute cell numbers, a master mix of AccuCheck counting beads (ThermoFisher #PCB100) in FACS buffer was prepared and 10⁴ beads/sample were added before acquisition. Absolute numbers of cells were calculated as shown in Equation 1 below:

$$\frac{[ count of acquired beads ]}{[ total beads per sample ]} [ fraction of organ used in stain ] \times \qquad ( 1 )$$

$$[ count of cell population ]$$

IOMA-expressing RAMOS cells were separated from unedited cells by sorting into RAMOS medium, and then washed and cultured as described above.

1838 single, mouse B cells from spleen and mesenteric lymph nodes of 3 IOMA iGL knock-in mice (ES30, HP1, and HP3) following the final boost (Week 18 or 23) were sorted into individual wells of a 96-well plate containing 5 μL of lysis buffer (TCL buffer (Qiagen, 1031576) with 1% of 2-β-mercaptoethanol). Plates were immediately frozen on dry ice and stored at −80° C. Singlet, live Zombie-NIR⁻ CD4⁻ CD8⁻ F4/80⁻ NK1.1⁻ CD 1b⁻ CD11c⁻ B220⁺ double Bait⁺ BaitKO⁻ lymphocytes were sorted unless GC B cells were sorted, which were gated as single, live Zombie-NIR⁻ CD4⁻ CD8⁻ F4/80⁻ NK1.1⁻ CD11b⁻ CD11c⁻ B220⁺ CD38⁻ FAS⁺ lymphocytes (See, FIG. 10A-FIG. 10C).

Mouse GC B cells for 10× Genomics single cell analysis were processed in PBS with 0.5% BSA instead of FACS buffer and 31,450 cells sorted into 5 μL of 0.05% BSA in PBS. Cells were spun down 400 g 6 min at 4° C. and volume adjusted to 22 μL before further processing.

Single Cell Antibody Cloning

Sequencing and cloning of mouse monoclonal antibodies from single cell-sorted B cells were performed as described with the modifications detailed in the supplementary materials.

Mutation Analysis

All HC and LC V(D)J sequences were translated and the CDR3 region was trimmed. The resulting V region was aligned against the IOMA iGL and IOMA using MAFFT. Indels were ignored for downstream analysis. All mismatches to IOMA iGL were counted as total mismatches (FIG. 3A). Only mismatches shared with IOMA mature when compared to IOMA iGL were used to assess chemical equivalence and calculate IOMA-like mutations (FIG. 3A). Chemical equivalence was as follows: Group 1: G/A/V/L/I; Group 2: S/T; Group 3: C/M; Group 4: D/N/E/Q; Group 5: R/K/H; Group 6: F/Y/W; Group 7: P. The baseline was calculated using extracted IGHV1-2, IGHJ5 (318,769) and IGLV2-23, IGLJ2 (1,790,961) sequences from healthy, HIV-negative donors and downloaded from cAb-Rep, a database of human shared BCR clonotypes available at https://cab-rep.c2b2.columbia.edu/.

3D neutralization plot shows the total number of V(D)J amino acid mutations (untrimmed) of each antibody vs the number of these mutations that are chemically equivalent to IOMA (FIG. 3C). Chemical equivalence defined as above.

In Vitro Neutralization Assays

Pseudovirus neutralization assays were conducted using methods known in the art, either in house (FIG. 2G, FIG. 3A, FIG. 4F, FIG. 5B) or at the Collaboration for AIDS Vaccine Discovery (CAVD) core neutralization facility (FIG. 6C). Monoclonal antibody IgGs were evaluated in duplicate with an 8-point, 3-fold dilution series starting at a top concentration of ˜100 μg/mL. All pseudovirus assays using monoclonal antibody IgGs were repeated at least twice for each value reported here. For polyclonal neutralizations, serum samples were heat inactivated at 56° C. for 30 min before being added to the neutralization assays, and then neutralization was evaluated in duplicate with an 8-point, 4-fold dilution series starting at a dilution of 1:60. The percent of neutralization at a 1:100 dilution (% 1:100) are reported for all serum samples. Tiers for viral strains were obtained from X. Brochet, M. P. Lefranc, V. Giudicelli, IMGT/V-QUEST: The highly customized and integrated system for IG and TR standardized V-J and V-D-J sequence analysis. Nucleic Acids Res. 36, W503-W508 (2008). Antibody neutralization score was calculated as shown in Equation 2 below:

$$[ neutralization score ] = {\sum }_{v = 1}^{n} \frac{5 - {log}_{10} ( {10}^{3} \times {[ {IC}_{50} ]}_{v} )}{n} \qquad ( 2 )$$

were n is number of different HIV pseudoviruses v tested for that antibody and [IC₅₀]ᵥ is the IC₅₀ of pseudovirus v in μg/mL.

Statistical Analysis

Comparisons between groups for ELISAs and neutralization assays were calculated using an unpaired or paired t-test in Prism 9.0 (Graphpad). Differences were considered significant when p values were less than 0.05. Exact p values are in the relevant figure at the top of the plot, with asterisks denoting level of significance (* denotes 0.01<p≤0.05, ** denotes 0.001<p≤0.01, *** denotes 0.0001<p≤0.001, and **** denotes p≤0.0001). Comparisons between total amino acid mutations and IOMA-like mutations in antibodies cloned from IOMA iGL mice (FIG. 3A-FIG. 3G) were performed using a Pearson correlation and R² values are presented.

Generation of Anti-Idiotypic Monoclonal Antibodies

Mice were injected three times with purified IOMA iGL. 3 days after the final injection spleens were harvested and used to generate hybridomas at the Fred Hutchinson Antibody Technology Center. Hybridoma supernatants were initially screened against IOMA iGL to identify antigen-specific hybridomas. Supernatants from positive wells were then screened against a panel of monoclonal antibodies that included IOMA, IOMA iGL, and inferred germlines of other anti-HIV-1 antibodies that served as isotype controls using a high throughput bead array. Two hybridomas of interest were identified; 3D3, which bound specifically to IOMA iGL, and 3D7, which bound to IOMA and IOMA iGL, which were subcloned from single cells. To produce recombinant anti-idiotypes, RNA was extracted from 1×10⁶ cells using the RNeasy kit (Qiagen), and the heavy and light chain sequences of the murine hybridomas were by obtained using the mouse Ig-primer set (69831; EMD Millipore) using methods known in the art. Sequences were codon optimized, cloned into pTT3-based IgG expression vectors with human constant regions using In-Fusion cloning (Clontech), expressed in 293 cells, and purified using Protein A chromatography.

X-Ray Crystallography

Crystallization screens for IOMA iGL Fab were performed using the sitting drop vapor diffusion method at room temperature (RT) by mixing 0.2 μL Fabs with 0.2 μL of reservoir solution (Hampton Research) using a TTP Labtech Mosquito automatic microliter pipetting robot. IOMA iGL Fab crystals were obtained in 20% (v/v) PEG 2000, 0.1 M Sodium Acetate (pH 4.6). Crystals were looped and cryopreserved in reservoir solution supplemented with 20% glycerol and flash frozen in liquid nitrogen.

The crystal structure of IOMA iGL Fab was solved with data sets. A 1.9 Å-resolution structure of IOMA—10-1074—BG505 was solved with a single data set collected at 100 K and 1 Å resolution on Beamline 12-2 at the Stanford Synchrotron Radiation Lightsource (SSRL) with a Pilatus 6M pixel detector (Dectris) that was indexed and integrated with iMosflm v7.4, and then merged with AILESS in the CCP4 software package v7.1.018. The structure was determined by molecular replacement using Phaser with one copy of IOMA Fab (PDB 5T3Z). Coordinates were refined with PHENIX v1.19.2-4158 with group B factor and TLS restraints. Manual rebuilding was performed iteratively with Coot v1.0.0. Data refinement statistics are shown in Table 2, with >98% of the residues in the favored region of the Ramachandran plot and <1% in the disallowed regions.

Cloning Yeast Libraries

Crystal structures of IOMA in complex with BG505 SOSIP.664 (PDB ID 5T3X and 5T3Z) were analyzed to determine mutations on gp120 that potentially could be beneficial for IOMA iGL binding. In addition, the crystal structure of IOMA iGL was modeled (PDB ID 7TQG) onto 426c.TM4ΔV1-3 (426c.TM4) gp120 (PDB ID 5FEC) and selected positions within gp120 that were predicted to be favorable for IOMA iGL binding. 426c.TM4ΔV1-3 (426c TM4) was chosen, an engineered clade C Env previously shown to activate B cell precursors of HIV-1 bNAbs targeting the CD4bs as the starting point for the library design.

Yeast libraries were generated using methods known in the art. Specifically, to generate the libraries of 426c gp120 variants, degenerate oligos were used in conjunction with an overlap assembly polymerase chain reaction (PCR) method. Overlapping primers for the PCR assembly reactions were designed using Primerize and shown in Table 6. NNK codons (where N=A/C/G/T and K=G/T) were utilized that encode for all 20 amino acids but decrease the chances of introducing a premature stop codon. Two different DNA fragments (426c library fragment 1 and 2) were synthesized first and then linearized in a final PCR step to generate the full-length 426c gp120 library used in yeast transformation. To obtain the full-length 426c gp120, a final PCR reaction was performed in which the PCR products of the 426c Library Fragment 1 and 2 were used as a template. Primers were used with overhangs complementary to the yeast display vector pCTCON-2 necessary for the homologous recombination in yeast. Library 2 was cloned in a similar manner as Library 1, but using a different set of primers as shown in Table 6 based on results from Library 1.

Yeast Transformation

The yeast display vector pCTCON-2 was used for cell surface display of the 426c gp120 proteins in Saccharomyces cerevisiae (S. cerevisiae) strain EBY100. A primary culture of 5 mL 2×YPD (40 g/L glucose, 20 g/L peptone, 20 g/L yeast extract) media was inoculated with a single S. cerevisiae EBY100 colony (freshly streaked on a YPD plate) and incubated overnight in a shaker at 30° C. and 250 rpm. 100 μL of the overnight yeast S. cerevisiae EBY100 cultures was transferred into 5 mL 2×YPD media and incubated overnight at 30° C., 250 rpm. The following day, 300 mL 2×YPD media was inoculated with the overnight precultures to an OD₆₀₀˜0.3 and was grown until an OD₆₀₀˜1.6. 3 mL of sterile filtered Tris/DTT (0.462 g 1,4-dithiothreitol in 3 mL 1 M Tris, pH 8.0) and 15 mL sterile filtered 2 M LiAc/TE (1.98 g LiAc in 10 mL of TE (10 mM Tris, 1 mM EDTA) was added and the culture incubated for 15 min at 30° C. and 250 rpm. Yeast cells were then pelleted at 3,500 g for 3 min and washed with 50 mL ice-cold sterile filtered NewE buffer (0.6 g Tris base, 91.09 g Sorbitol (1 M), 73.50 mg CaCl₂) in ddH₂O to a final volume of 500 mL, pH 7.5). After two additional wash steps, the pellet was re-suspended in 3 mL NewE buffer and 50 μg 426c library DNA insert and 10 μg pCTCON-2 vector (digested with NheI and BamHI) was added. 200 μL of this transformation mix was then aliquoted into pre-chilled 2 mm electroporation cuvettes (Bio-Rad) and electroporated at 1500 V with an average time constant of ˜4.5 ms using a Gene Pulser Xcell Electroporation System (Bio-Rad), which was repeated for the entire transformation mix. After electroporation, yeast cells were directly recovered with 2 mL 2×YPD media and transferred into 50 mL cold 2×YPD media (final volume up to 200 mL 2×YPD media) and grown for 1 h at 30° C. and 250 rpm. Serial dilutions of the freshly transformed yeast culture were plated on SDCAA (20 g/L glucose, 6.7 g/L Difco yeast nitrogen base, 1.4 g/L Yeast Synthetic Drop-out Medium Supplements without histidine, leucine, tryptophan and uracil, 20 mg/L uracil, 50 mg/L histidine, 100 mg/L leucine) agarose plates to test the viability and size of the library. After 1 h, the culture was removed and the cells were pelleted and resuspended in 500 mL SDCAA media+carbenicillin (100 μg/mL final concentration) and grown for two days at 30° C. and 250 rpm. To confirm the genetic diversity of the library, a yeast colony PCR was performed on the liquid culture and the PCR product was sequenced. Sequencing reactions were performed at Laragen Inc (Culver City, CA). The sequence data was analyzed using SeqMan Pro (DNASTAR, v13.02). After two days, cells were pelleted and glycerol stocks were made by suspending ˜10⁹ yeast cells in 1 mL of freezing buffer (0.335 g Yeast Nitrogen Base, 1 mL glycerol in 50 mL H₂O, sterilized by filtration). Aliquots were flash frozen in liquid nitrogen and stored at −80° C.

Magnetic-Activated Cell Sorting

Magnetic-activated cell sorting (MACS) was used to remove transformants containing stop codons. After growing up the freshly transformed cells for two days in SDCAA, cells were pelleted and induced at an OD₆₀₀˜1.0 in 100 mL SGCAA-carb (SDCAA prepared with 20 g/L galactose instead of glucose and supplemented with 100 μg/mL carbenicillin final concentration) for 20 h at 20° C. and 250 rpm. Yeast cells were washed 5 times with PBSF (PBS+0.1% bovine serum albumin (BSA)) and 10⁸ cells were incubated with 400 μL PBSF and 100 μL MACS™ anti-c-Myc MicroBeads (Miltenyi Biotec) for 45 min on a rotator at 4° C. Cells were then pelleted and resuspended in 5 mL PBSF and sorted using a MidiMACS Separator magnet (Miltenyi Biotec) in combination with an LS column (Miltenyi Biotec) equilibrated in PBSF. Isolated cells were then grown for 2 days in 100 mL SDCAA-carb at 30° C. and 250 rpm and then induced again with SGCAA-carb for 20 h at 20° C. and 250 rpm.

Yeast Flow Cytometry and Cell Sorting

To prepare the yeast library for FACS analysis, cells were pelleted at 3000 rpm for 2 min and washed 5 times with PBSF. Cells were then stained at a density of 10⁷ cells/mL with 1:500 anti-c-Myc antibody conjugated to AlexaFluor488 (Abcam, ab190026) and 1 μM IOMA iGL and incubated for 1-2 h on a rotator at 4° C. Cells were then washed twice with PBSF and resuspended in 200 μL PBSF with 1:1000 goat anti-human antibody conjugated to AlexaFluor647 (Abcam, ab190560, RRID:AB_2876372) and incubated for 30 min at 4° C. Cells were then analyzed on a MACSQuant Analyzer (Miltenyi Biotec) or sorted using an SY3200 cell sorter system (Sony). In either case, non-transformed yeast cells and single-stained transformed samples stained with either anti-c-Myc or IOMA iGL IgG were used to set the gates for analysis and collection. Cells that stained double-positive for both c-Myc and IOMA iGL were collected and grown in 5 mL SDCAA-carb for 1-2 days at 30° C. and 250 rpm and then transferred to 100 mL SDCAA-carb for an additional 1-2 days at 30° C. and 250 rpm. Cells were then pelleted and resuspended in H₂O and plated onto SDCAA-carb for 2-3 days at 30° C. After multiple iterative rounds of sorting (three rounds for Library 1 and seven rounds for Library 2), sequences were recovered by colony PCR and sequence confirmed (Laragen). Primers were used with specific complementary regions to enable ligation of the linear product into the expression vector pTT5 using the Gibson assembly method for protein production. After construction, plasmids were isolated from E. coli using the QIAprep Miniprep kit (Qiagen) and confirmed by Sanger sequencing (Laragen).

Generation of IOMA-Expressing RAMOS Cells by CRISPR Cas9 Gene Editing

A targeting vector was constructed using the NEB Hifi DNA assembly kit to clone a gBlock (IDT) into pUCmu. The gBlock (IDT) contained ˜0.5 kb homology arms to the human IgH locus which flanked an expression cassette consisting of the Cp splice acceptor, the entire IOMA LC gene, a furin-GSG-P2A sequence followed by the IOMA HC Leader-VDJ and the JH4 splice donor based on previously-described designs (FIG. 10C). Vectors were maxi-prepped (Machery-Nagle) for transfection.

RAMOS (RA 1) cells were purchased from ATCC (CRL-1596) and maintained in RPMI-1640 supplemented with 10 FCS, 1× antibiotic/antimycotic, 2 mM glutamine, 1 mM sodium pyruvate, 10 mM HEPES and 55 μM β-mercaptoethanol. Before transfection, cells were harvested, washed once in PBS and resuspended at 6×107 cells/mL in Neon kit buffer T (ThermoFisher). Three ribonucleoprotein complexes (RNPs) were prepared using 3 different sgRNAs. AGGCATCGGAAAATCCACAG (SEQ ID NO: 255) was used to target the IgH locus in the intron 3′ of IGHJ6 to integrate the sequence flanked by the appropriate homology arms from the targeting vector; CTGGGAGTTACCCGATTGGA (SEQ ID NO: 256) was used to ablate the human IGKC exon and CACGCATGAAGGGAGCACCG (SEQ ID NO: 257) was used to ablate all functional IGLC genes (IgLC1, IGL2, IGLC3 and IGLC7). Complexes were prepared by mixing 1.875 μL of 100 μM sgRNA with 1 μL of 61 μM Cas9 (all IDT) for a molar ratio of ˜3:1 followed by incubation for 20 min at RT. IGH:IGK:IGL RNPs were then mixed at a 2:1:1 v/v/v ratio. 2.6 μg targeting vector (at 4 mg/mL) were mixed with 1.5 μL IGH:IGK:IGL RNP mix and 11 μL RAMOS cells in buffer T. 10 μL of the final mix were transfected in a 10 μL Neon tip in a Neon device at 1350 V 30 ms 1 pulse. Cells were immediately transferred into 50 μL RAMOS medium without 1× antibiotic/antimycotic in a 48-well plate and 2 h later 450 μL full RAMOS medium was added. Cells were then cultured as before. Edited IOMA-expressing cells were bulk sorted by flow cytometry as live, singlet, CD19+, RC1 antigenhi, IgL+, IgK+IgM+(Table 7) and cultured as before. IOMA-expression was further verified by staining with 426c-, CNE8- and CNE20-derived SOSIPs and 426c-CD4bs-KO proteins to show specificity.

10× Genomics Single Cell Processing and Next Generation V(D)J Sequencing

Cells were counted in the final injection volume, and 18,000 cells loaded onto a Chromium Controller (10× Genomics). Single-cell RNA-seq libraries were prepared using the Chromium Single Cell 5 v2 Reagent Kit (PN-1000265) according to manufacturer's protocol. Chromium Single Cell Mouse BCR Amplification Kit (PN-1000255) was used for VDJ cDNA amplification. After QC, 5′ expression and VDJ Libraries were pooled 1:1 and sequenced on an Illumina NOVAseq S1 flowcell at the Rockefeller University Genomics Core.

Computational Analyses of V(D)J Sequences Derived from IOMAgl Mice by Next Generation Sequencing

The single-cell V(D)J assembly was carried out by Cell Ranger 6.0.1. A customized reference was created by adding the knocked-in IOMA iGL V(D)J genes to the mouse GRCm38 V(D)J reference so Cell Ranger could recognize and assemble the human/mouse chimera transcripts. Contigs associated with a valid cell barcode according to Cell Ranger were selected for downstream processing using seqtk version 1.3-r106 (https://github.com/lh3/seqtk).

IgBlast standalone version 1.14 was used to annotate the immunoglobulin sequences based on a custom database with mouse and human V(D)J genes. Productive IG sequences with more than 20 reads of coverage and with any identified isotype were selected for downstream processing. Unexpectedly, although the IgBlast algorithm identified the V and J genes for 8010 LC sequences, it failed to annotate the CDR3, and consequently, the information regarding their functionality was missing. 7782 (97.15%) sequences corresponding to the knock-in LC were extracted and submitted to IMGT/V-QUEST, which successfully identified the CDR3 and provided the productivity information.

Cell barcodes associated with sequences coded by different V genes for either HC or LC were considered doublets and were subsequently removed from downstream analysis. HCs and LCs derived from the same cell were paired, and clones were assigned using our IgPipeline (https://github.com/stratust/igpipeline/tree/igpipeline2_timepoint_v2).

Single Cell Antibody Cloning

The following modifications were applied to the described protocol from Viant C. et al. Sequencing, cloning, and antigen binding analysis of monoclonal antibodies isolated from single mouse B cells. STAR Protoc. 2021 Mar. 15; 2(2):100389. doi: 10.1016/j.xpro.2021.100389. PMID: 33778783; PMCID: PMC7985702. Briefly, single cell RNA in 96-well plates was purified using magnetic beads (RNAClean XP, Beckman Coulter, Cat #A63987). RNA was eluted from the magnetic beads with 11 μL of a solution containing 14.5 ng/L of random primers (Invitrogen, Cat #48190011), 0.5% of Igepal Ca-630 (type NP-40, 10% in dH₂O, MP Biomedicals, Cat #198596) and 0.6 U/μL of RNase inhibitor (Promega, Cat #N2615) in nuclease-free water (Qiagen, Cat #129117), and incubated at 65° C. for 3 min. cDNA was synthesized by reverse transcription (SuperScript™ III Reverse Transcriptase 10,000 U, Invitrogen, Cat #18080-044). cDNA was stored at −80° C. or used for antibody gene amplification by nested polymerase chain reaction (PCR) after addition of 10 μL of nuclease-free water.

Mouse antibody genes were amplified using HotstarTaq DNA polymerase (Qiagen Cat #203209) with the primer sets specific for the Igh^{IOMAiGL} and Igk^{IOMAiGL} transgenes. Primer sequences and reaction mixes are provided in Table 8. Thermocycler conditions were as follows for annealing (° C.)/elongation (s)/number of cycles: PCR1 (IgG, IgM and IgK): 51/55/50; PCR2 (IgG and IgM): 54/55/50; PCR2 (IgK): 50/55/50.

PCR products of antibody HC and LC genes were purified and Sanger-sequenced (Genewiz) and *ab1 files analyzed using our IgPipeline (https://github.com/stratust/igpipeline/tree/igpipeline2_timepoint_v2). V(D)J sequences were ordered as eBlocks (IDT) with short homologies for Gibson assembly and cloned into human IgG1 or human IgL2 expression vectors using the NEB Hifi DNA Assembly mix (NEB, Cat #E2621L). Plasmid sequences were verified by Sanger sequencing (Genewiz).

SPR Binding Studies

All SPR measurements were performed on a Biacore T200 (GE Healthcare) at 20° C. in HBS-EP+(GE Healthcare) running buffer. IgGs were directly immobilized onto a CM5 chip (GE Healthcare) to ˜3000 resonance units (RUs) using primary amine chemistry. A concentration series of monomeric gp120 core constructs (IGT2, IGT1, 426c TM4) were injected over the flow cells at increasing concentrations (top concentrations ranging from 600 μM to 10 μM) at a flow rate of 60 μL/min for 60 s and allowed to dissociate for 300 s. Regeneration of flow cells was achieved by injecting one pulse each of 10 mM glycine pH 2.0 at a flow rate of 90 μL/min. Kinetic analyses were used after subtraction of reference curves to derive on/off rates (kₐ/k_{d}) and binding constants (K_{D}s) using a 1:1 binding model with or without bulk refractive index change (RI) correction as appropriate (Biacore T200 Evaluation software v3.0). Reported affinities represent the average of two independent experiments. SPR experiments that were not used to derive binding affinities or kinetic constants were done using a single high concentration (1 μM) to qualitatively determine binding versus no binding.

Analysis Software

Unless stated otherwise, Geneious Prime 2021.2.2, MacVector 18.2.0 and DNAStar SeqMan Pro 17.1.1 were used for sequence analysis and graphs were created using R language. Flow cytometry data were processed using Mac versions of FlowJo 10.7.2. and GraphPad Prism 9.3 and Microsoft Excel for Mac 16.54 were used for data analysis. Structural figures were made using PyMOL (Schrödinger, LLC) or ChimeraX. V(D)J gene assignments of NHP and murine antibodies were done using IMGT/V-QUEST. Sequence alignments were done using Clustal Omega.

TABLE 1
AMINO ACID SEQUENCES FOR HIV ENVS AND ANTIBODIES USED IN THIS
STUDY

| Protein | | SEQ ID |
|---|---|---|
| Name | Sequence | NO: |
| IGT2 gp120 | VWKEAKTTLFCASDAKAYEKECHNVWATHACVPTDPNPQEVVLENVTENFNMWKNDMVDQMQ | 117 |
| | EDVISIWDQCLKPCVKLTNTSTLTQACPKVTFDPIPIHYCAPAGYAILKCNNKTENGKGPCN | |
| | NVSTVQCTHGIKPVVSTQLLINGSLAEEEIVIRSKNLRNNAKIIIVQLNKSVEIVCTRPNNG | |
| | GSGSGGDIRQAYCNISGRNWSEAVNQVKKKLKEHFPHKNISFQSSSGGDLEITTHSENCGGE | |
| | FFYCNTSGLENDTISNATIMLPCRIKQIINMWQEPGKAIYAPPIKGNITCKSDITGLLLLRD | |
| | GGNALRPTEIFRPSGGDMRDNWRSELYKYKVVEIKPLHHHHHH | |
| IGT1 gp120 | VWKEAKTTLFCASDAKAYEKECHNVWATHACVPTDPNPQEVVLENVTENFNMWKNDMVDQMQ | 118 |
| | EDVISIWDQCLKPCVKLTNTSTLTQACPKVTFDPIPIHYCAPAGYAILKCNNKTENGKGPCN | |
| | NVSTVQCTHGIKPVVSTQLLINGSLAEEEIVIRSKNLRNNAKIIIVQLNKSVEIVCTRPNNG | |
| | GSGSGGDIRQAYCNISGRNWSEAVNQVKKKLKEHFPHKNISFQSSSGGDLEITTHSENCGGE | |
| | FFYCNTSGLENDTISNATIMLPCRIKQIINMWQEPGKAIYAPPIKGNITCKSDITGLLLLRD | |
| | GGNSQRETEIFRPSGGDMRDNWRSELYKYKVVEIKPLHHHHHH | |
| 426c.TM4 | VWKEAKTTLFCASDAKAYEKECHNVWATHACVPTDPNPQEVVLENVTENFNMWKNDMVDQMQ | 119 |
| gp120 | EDVISIWDQCLKPCVKLTNTSTLTQACPKVTFDPIPIHYCAPAGYAILKCNNKTENGKGPCN | |
| | NVSTVQCTHGIKPVVSTQLLINGSLAEEEIVIRSKNLRDNAKIIIVQLNKSVEIVCTRPNNG | |
| | GSGSGGDIRQAYCNISGRNWSEAVNQVKKKLKEHFPHKNISFQSSSGGDLEITTHSFNCGGE | |
| | FFYCNTSGLENDTISNATIMLPCRIKQIINMWQEVGKAIYAPPIKGNITCKSDITGLLLLRD | |
| | GGDTTDNTEIFRPSGGDMRDNWRSELYKYKVVEIKPLHHHHHH | |
| IGT2 | GSNLWVTVYYGVPVWKEAKTTLFCASDAKAYEKEVHNVWATHACVPTDPNPQEVVLENVTEN | 120 |
| SOSIP | FNMWKNDMVDQMQEDVISIWDQSLKPCVKLTPLCVTLNCTNVNVTSNSTNVNSSSTDNTTLG | |
| | EIKNCSFDITTEIRDKTRKEYALFYRLDIVPLDNSSNPNSSNTYRLINCNTSTCTQACPKVT | |
| | FDPIPIHYCAPAGYAILKCNNKTFNGKGPCNNVSTVQCTHGIKPVVSTQLLINGSLAEEEIV | |
| | IRSKNLRNNAKIIIVQLNKSVEIVCTRPNNNTRRSIRIGPGQTFYATDIIGDIRQAYCNISG | |
| | RNWSEAVNQVKKKLKEHFPHKNISFQSSSGGDLEITTHSFNCGGEFFYCNTSGLENDTISNA | |
| | TIMLPCRIKQIINMWQEPGKCIYAPPIKGNITCKSDITGLLLLRDGGNALRPTEIFRPSGGD | |
| | MRDNWRSELYKYKVVKIEPLGVAPTRCKRRVVGRRRRRRAVGIGAVFLGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALD | |
| IGT1 | GSNLWVTVYYGVPVWKEAKTTLFCASDAKAYEKEVHNVWATHACVPTDPNPQEVVLENVTEN | 121 |
| SOSIP | FNMWKNDMVDQMQEDVISIWDQSLKPCVKLTPLCVTLNCTNVNVTSNSTNVNSSSTDNTTLG | |
| | EIKNCSFDITTEIRDKTRKEYALFYRLDIVPLDNSSNPNSSNTYRLINCNTSTCTQACPKVT | |
| | FDPIPIHYCAPAGYAILKCNNKTFNGKGPCNNVSTVQCTHGIKPVVSTQLLINGSLAEEEIV | |
| | IRSKNLRNNAKIIIVQLNKSVEIVCTRPNNNTRRSIRIGPGQTFYATDIIGDIRQAYCNISG | |
| | RNWSEAVNQVKKKLKEHFPHKNISFQSSSGGDLEITTHSFNCGGEFFYCNTSGLENDTISNA | |
| | TIMLPCRIKQIINMWQEPGKCIYAPPIKGNITCKSDITGLLLLRDGGNSQRETEIFRPSGGD | |
| | MRDNWRSELYKYKVVKIEPLGVAPTRCKRRVVGRRRRRRAVGIGAVFLGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALD | |
| 426c | RNWSEAVNQVKKKLKEHFPHKNISFQSSSGGDLEITTHSFNCGGEFFYCNTSGLENDTISNA | 122 |
| SQSIP | AENLWVTVYYGVPVWKEAKTTLFCASDAKAYEKEVHNVWATHACVPTDPNPQEVVLENVTEN | |
| | FNMWKNDMVDQMQEDVISIWDQSLKPCVKLTPLCVTLNCTNVNVTSNSTNVNSSSTDNTTLG | |
| | EIKNCSFDITTEIRDKTRKEYALFYRLDIVPLDNSSNPNSSNTYRLINCNTSTCTQACPKVT | |
| | FDPIPIHYCAPAGYAILKCNNKTFNGKGPCNNVSTVQCTHGIKPVVSTQLLINGSLAEEEIV | |
| | IRSKNLSDNAKIIIVQLNKSVEIVCTRPNNNTRRSIRIGPGQTFYATDIIGDIRQAYCNISG | |
| | TIMLPCRIKQIINMWQEVGKCIYAPPIKGNITCKSDITGLLLLRDGGNTTNNTEIFRPGGGD | |
| | MRDNWRSELYKYKVVKIEPLGVAPTRCKRRVVGRRRRRRAVGIGAVFLGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALD | |
| 426c | AENLWVTVYYGVPVWKEAKTTLFCASDAKAYEKEVHNVWATHACVPTDPNPQEVVLENVTEN | 123 |
| D279N | FNMWKNDMVDQMQEDVISIWDQSLKPCVKLTPLCVTLNCTNVNVTSNSTNVNSSSTDNTTLG | |
| SOSIP | EIKNCSFDITTEIRDKTRKEYALFYRLDIVPLDNSSNPNSSNTYRLINCNTSTCTQACPKVT | |
| | FDPIPIHYCAPAGYAILKCNNKTFNGKGPCNNVSTVQCTHGIKPVVSTQLLINGSLAEEEIV | |
| | IRSKNLSNNAKIIIVQLNKSVEIVCTRPNNNTRRSIRIGPGQTFYATDIIGDIRQAYCNISG | |
| | RNWSEAVNQVKKKLKEHFPHKNISFQSSSGGDLEITTHSFNCGGEFFYCNTSGLENDTISNA | |
| | TIMLPCRIKQIINMWQEVGKCIYAPPIKGNITCKSDITGLLLLRDGGNTTNNTEIFRPGGGD | |
| | MRDNWRSELYKYKVVKIEPLGVAPTRCKRRVVGRRRRRRAVGIGAVFLGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALD | |
| 426c degly2 | AENLWVTVYYGVPVWKEAKTTLFCASDAKAYEKEVHNVWATHACVPTDPNPQEVVLENVTEN | 124 |
| SOSIP | FNMWKNDMVDQMQEDVISIWDQSLKPCVKLTPLCVTLNCTNVNVTSNSTNVNSSSTDNTTLG | |
| | EIKNCSFDITTEIRDKTRKEYALFYRLDIVPLDNSSNPNSSNTYRLINCNTSTCTQACPKVT | |
| | FDPIPIHYCAPAGYAILKCNNKTFNGKGPCNNVSTVQCTHGIKPVVSTQLLINGSLAEEEIV | |
| | IRSKNLTDNAKIIIVQLNKSVEIVCTRPNNNTRRSIRIGPGQTFYATDIIGDIRQAYCNISG | |
| | RNWSEAVNQVKKKLKEHFPHKNISFQSSSGGDLEITTHSFNCGGEFFYCNTSGLENDTISNA | |
| | TIMLPCRIKQIINMWQEVGKCIYAPPIKGNITCKSDITGLLLLRDGGNTANNAEIFRPGGGD | |
| | MRDNWRSELYKYKVVKIEPLGVAPTRCKRRVVGRRRRRRAVGIGAVFLGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALD | |
| 426c degly2 | IRSKNLTNNAKIIIVQLNKSVEIVCTRPNNNTRRSIRIGPGQTFYATDIIGDIRQAYCNISG | 125 |
| D279N SOSIP | AENLWVTVYYGVPVWKEAKTTLFCASDAKAYEKEVHNVWATHACVPTDPNPQEVVLENVTEN | |
| | FNMWKNDMVDQMQEDVISIWDQSLKPCVKLTPLCVTLNCTNVNVTSNSTNVNSSSTDNTTLG | |
| | EIKNCSFDITTEIRDKTRKEYALFYRLDIVPLDNSSNPNSSNTYRLINCNTSTCTQACPKVT | |
| | FDPIPIHYCAPAGYAILKCNNKTFNGKGPCNNVSTVQCTHGIKPVVSTQLLINGSLAEEEIV | |
| | RNWSEAVNQVKKKLKEHFPHKNISFQSSSGGDLEITTHSFNCGGEFFYCNTSGLENDTISNA | |
| | TIMLPCRIKQIINMWQEVGKCIYAPPIKGNITCKSDITGLLLLRDGGNTANNAEIFRPGGGD | |
| | MRDNWRSELYKYKVVKIEPLGVAPTRCKRRVVGRRRRRRAVGIGAVELGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALD | |
| 426c degly3 | AENLWVTVYYGVPVWKEAKTTLFCASDAKAYEKEVHNVWATHACVPTDPNPQEVVLENVTEN | 126 |
| SOSIP | FNMWKNDMVDQMQEDVISIWDQSLKPCVKLTPLCVTLNCTNVNVTSNSTNVNSSSTDNTTLG | |
| | EIKNCSFDITTEIRDKTRKEYALFYRLDIVPLDNSSNPNSSNTYRLINCNTSTCTQACPKVT | |
| | FDPIPIHYCAPAGYAILKCNNKTFNGKGPCNNVSTVQCTHGIKPVVSTQLLINGSLAEEEIV | |
| | IRSKALTDNAKIIIVQLNKSVEIVCTRPNNNTRRSIRIGPGQTFYATDIIGDIRQAYCNISG | |
| | RNWSEAVNQVKKKLKEHFPHKNISFQSSSGGDLEITTHSFNCGGEFFYCNTSGLENDTISNA | |
| | TIMLPCRIKQIINMWQEVGKCIYAPPIKGNITCKSDITGLLLLRDGGNTANNAEIFRPGGGD | |
| | MRDNWRSELYKYKVVKIEPLGVAPTRCKRRVVGRRRRRRAVGIGAVELGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALD | |
| BG505.v4.1- | VQFNTPVQINCTRPNNNTRKSIRIGPGQWFYATGDIIGDIRQAHCNVSKATWNETLGKVVKQ | 127 |
| GT1 | AENLWVTVYYGVPVWKDAETTLFCASDAKAYETKKHNVWATHACVPTDPNPQEIHLENVTEE | |
| SOSIP | FNMWKNNMVEQMHTDIISLWDQSLKPCVKLTPLCVTLQCTNVTNAITDDMRGELKNCSENMT | |
| | TELRDKRQKVHALFYKLDIVPINENQNTSYRLINCNTAAITQACPKVSFEPIPIHYCAPAGE | |
| | AILKCKDKKFNGTGPCPSVSTVQCTHGIKPVVSTQLLINGSLAEEEVMIRSEDIRNNAKNIL | |
| | LRKHFGNNTIIRFANSSGGDLEVTTHSFNCGGEFFYCDTSGLENSTWISNTSVQGSNSTGSN | |
| | DSITLPCRIKQIINMWQRIGQAMYAPPIQGVIRCVSNITGLILTRDGGSTDSTTETFRPSGG | |
| | DMRDNWRSELYKYKVVKIEPLGVAPTRCKRRVVGRRRRRRAVGIGAVFLGFLGAAGSTMGAA | |
| | SMTLTVQARNLLSGIVQQQSNLLRAPEAQQHLLKLTVWGIKQLQARVLAVERYLRDQQLLGI | |
| | WGCSGKLICCTNVPWNSSWSNRNLSEIWDNMTWLQWDKEISNYTQIIYGLLEESQNQQEKNE | |
| | QDLLALD | |
| eOD-GT8 | DTITLPCRPAPPPHCSSNITGLILTRQGGYSNANTVIFRPSGGDWRDIARCQIAGTVVSTQL | 128 |
| | FLNGSLAEEEVVIRSEDWRDNAKSICVQLATSVEIACTGAGHCAISRAKWANTLKQIASKLR | |
| | EQYGAKTIIFKPSSGGDPEFVNHSFNCGGEFFYCASTQLFASTWFASTGTGTK | |
| IGT2 | GSNLWVTVYYGVPVWKEAKTTLFCASDAKAYEKEVHNVWATHACVPTDPNPQEVVLENVTEN | 129 |
| SOSIP | FNMWKNDMVDQMQEDVISIWDQSLKPCVKLTPLCVTLNCTNVNVTSNSTNVNSSSTDNTTLG | |
| Spy Tag | EIKNCSFDITTEIRDKTRKEYALFYRLDIVPLDNSSNPNSSNTYRLINCNTSTCTQACPKVT | |
| | FDPIPIHYCAPAGYAILKCNNKTFNGKGPCNNVSTVQCTHGIKPVVSTQLLINGSLAEEEIV | |
| | IRSKNLRNNAKIIIVQLNKSVEIVCTRPNNNTRRSIRIGPGQTFYATDIIGDIRQAYCNISG | |
| | RNWSEAVNQVKKKLKEHFPHKNISFQSSSGGDLEITTHSFNCGGEFFYCNTSGLENDTISNA | |
| | TIMLPCRIKQIINMWQEPGKCIYAPPIKGNITCKSDITGLLLLRDGGNALRPTEIFRPSGGD | |
| | MRDNWRSELYKYKVVKIEPLGVAPTRCKRRVVGRRRRRRAVGIGAVELGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALDGGGGSGGGSGGGSGSGAHIVMVDAYKPTK | |
| IGT1 | GSNLWVTVYYGVPVWKEAKTTLFCASDAKAYEKEVHNVWATHACVPTDPNPQEVVLENVTEN | 130 |
| SOSIP | FNMWKNDMVDQMQEDVISIWDQSLKPCVKLTPLCVTLNCTNVNVTSNSTNVNSSSTDNTTLG | |
| Spy Tag | EIKNCSFDITTEIRDKTRKEYALFYRLDIVPLDNSSNPNSSNTYRLINCNTSTCTQACPKVT | |
| | FDPIPIHYCAPAGYAILKCNNKTFNGKGPCNNVSTVQCTHGIKPVVSTQLLINGSLAEEEIV | |
| | IRSKNLRNNAKIIIVQLNKSVEIVCTRPNNNTRRSIRIGPGQTFYATDIIGDIRQAYCNISG | |
| | RNWSEAVNQVKKKLKEHFPHKNISFQSSSGGDLEITTHSFNCGGEFFYCNTSGLENDTISNA | |
| | TIMLPCRIKQIINMWQEPGKCIYAPPIKGNITCKSDITGLLLLRDGGNSQRETEIFRPSGGD | |
| | MRDNWRSELYKYKVVKIEPLGVAPTRCKRRVVGRRRRRRAVGIGAVELGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALDGGGGSGGGSGGGSGSGAHIVMVDAYKPTK | |
| 426c degly2 | AENLWVTVYYGVPVWKEAKTTLFCASDAKAYEKEVHNVWATHACVPTDPNPQEVVLENVTEN | 131 |
| D279N | FNMWKNDMVDQMQEDVISIWDQSLKPCVKLTPLCVTLNCTNVNVTSNSTNVNSSSTDNTTLG | |
| SOSIP | EIKNCSFDITTEIRDKTRKEYALFYRLDIVPLDNSSNPNSSNTYRLINCNTSTCTQACPKVT | |
| Spy Tag | FDPIPIHYCAPAGYAILKCNNKTFNGKGPCNNVSTVQCTHGIKPVVSTQLLINGSLAEEEIV | |
| | IRSKNLTNNAKIIIVQLNKSVEIVCTRPNNNTRRSIRIGPGQTFYATDIIGDIRQAYCNISG | |
| | RNWSEAVNQVKKKLKEHFPHKNISFQSSSGGDLEITTHSFNCGGEFFYCNTSGLENDTISNA | |
| | TIMLPCRIKQIINMWQEVGKCIYAPPIKGNITCKSDITGLLLLRDGGNTANNAEIFRPGGGD | |
| | MRDNWRSELYKYKVVKIEPLGVAPTRCKRRVVGRRRRRRAVGIGAVELGFLGAAGSTMGAAS | |
| | MTLTVQARNLLSGIVQQQSNLLRAPEAQQHLLKLTVWGIKQLQARVLAVERYLRDQQLLGIW | |
| | GCSGKLICCTNVPWNSSWSNRNLSEIWDNMTWLQWDKEISNYTQIIYGLLEESQNQQEKNEQ | |
| | DLLALDGGGGSGGGSGGGSGSGRGVPHIVMVDAYKRYK | |
| 398F1 | AENLWVTVYYGVPVWKDAETTLFCASDAKAYHTEVHNVWATHACVPTDPNPQEINLENVTEE | 132 |
| SOSIP | FNMWKNKMVEQMHTDIISLWDQSLKPCVQLTPLCVTLDCQYNVTNINSTSDMAREINNCSYN | |
| Spy Tag | ITTELRDREQKVYSLFYRSDIVQMNSDNSSKYRLINCNTSACKQACPKVTFEPIPIHYCAPA | |
| | GFAILKCKDKEFNGTGPCKNVSTVQCTHGIKPVVSTQLLINGSLAEEKVMIRSENITDNAKN | |
| | IIVQFKEPVKINCTRPNNNTRKSVRIGPGQTFYATGEIIGDIRQAHCNVSKAHWENTLQEVA | |
| | NQLKLMIHSNKTIIFANSSGGDLEITTHSFNCGGEFFYCYTSGLFNYTENDTSTNSTESKSN | |
| | DTITLQCRIKQIINMWQRAGQCVYAPPIPGIIRCESNITGLILTRDGGNNNSNTNETFRPGG | |
| | GDMRDNWRSELYRYKVVKIEPLGVAPTRCKRRVVGRRRRRRAVGIGAVSLGFLGAAGSTMGA | |
| | ASMTLTVQARNLLSGIVQQQSNLLRAPEPQQHLLKDTHWGIKQLQARVLAVEHYLRDQQLLG | |
| | IWGCSGKLICCTNVPWNSSWSNRNLSEIWDNMTWLQWDKEISNYTQIIYGLLEESQNQQEKN | |
| | EQDLLALDGGGGSGGGSGGGSGSGAHIVMVDAYKPTK | |
| BJOX2000 | AENLWVTVYYGVPVWKEATTTLFCASDAKAYDTEVHNVWATHACVPTDPDPQEMFLENVTEN | 133 |
| SOSIP | FNMWKNNMVDQMHEDVISLWDQSLKPCVKLTPLCVTLECKNVNSSSSDTKNGTDPEMKNCSF | |
| SpyTag | NATTELRDRKQKVYALFYKLDIVPLNEKNSSEYRLINCNTSTCTQACPKVTFDPIPIHYCTP | |
| | AGYAILKCNDEKFNGTGPCSNVSTVQCTHGIKPVVSTQLLINGSLAEKGIVIRSENLTNNVK | |
| | TIIVHLNQSVEILCIRPNNNTRKSIRIGPGQTFYATGEIIGDIRQAHCNISGKVWNETLQRV | |
| | GEKLAEYFPNKTIKFNSSSGGDLEITTHSFNCGGEFFYCNTSKLENGTENGTYMPNVTEGNS | |
| | TISIPCRIKQIINMWQKVGRCMYAPPIEGNITCKSKITGLLLERDGGPENDTEIFRPGGGDM | |
| | RNNWRSELYKYKVVEIKPLGVAPTRCKRRVVGRRRRRRAVGIGAVSLGFLGAAGSTMGAASM | |
| | TLTVQARNLLSGIVQQQSNLLRAPEPQQHLLKDTHWGIKQLQARVLAVEHYLRDQQLLGIWG | |
| | CSGKLICCTNVPWNSSWSNRNLSEIWDNMTWLQWDKEISNYTQIIYGLLEESQNQQEKNEQD | |
| | LLALDGGGGSGGGSGGGSGSGAHIVMVDAYKPTK | |
| CE1176 | AENLWVTVYYGVPVWKEAKTTLFCASDAKAYEKEVHNVWATHACVPTDPNPQEMVLENVTEN | 134 |
| SOSIP | FNMWKNDMVDQMHEDVISLWDQSLKPCVKLTPLCVTLTCTNTTVSNGSSNSNANFEEMKNCS | |
| Spy Tag | FNATTEIKDKKKNEYALFYKLDIVPLNNSSGKYRLINCNTSACAQACPKVTFEPIPIHYCAP | |
| | AGYAILKCNNKTFNGTGPCNNVSTVQCTHGIKPVVSTQLLINGSLAEKEIIIRSENLTNNAK | |
| | TIIIHFNESVGIVCTRPSNNTRKSIRIGPGQTFYATGDIIGDIRQAHCNVSKQNWNRTLQQV | |
| | GRKLAEHFPNRNITFNHSSGGDLEITTHSFNCRGEFFYCNTSGLENGTYHPNGTYNETAVNS | |
| | SDTITLQCRIKQIINMWQEVGRCMYAPPIAGNITCNSTITGLLLTRDGGINQTGEEIFRPGG | |
| | GDMRDNWRNELYKYKVVEIKPLGVAPTRCKRRVVGRRRRRRAVGIGAVSLGFLGAAGSTMGA | |
| | ASMTLTVQARNLLSGIVQQQSNLLRAPEPQQHLLKDTHWGIKQLQARVLAVEHYLRDQQLLG | |
| | IWGCSGKLICCTNVPWNSSWSNRNLSEIWDNMTWLQWDKEISNYTQIIYGLLEESQNQQEKN | |
| | EQDLLALDGGGGSGGGSGGGSGSGAHIVMVDAYKPTK | |
| CE0217 | AENLWVTVYYGVPVWREAKTTLFCASDAKAYEREVHNVWATHACVPTDPNPQERVLENVTEN | 135 |
| SOSIP | FNMWKNNMVDQMHEDIISLWDESLKPCIKLTPLCVTLNCGNAIVNESTIEGMKNCSFNVTTE | |
| Spy Tag | LKDKKKKEYALFYKLDVVPLNGENNNSNSKNFSEYRLINCNTSTCTQACPKVSFDPIPIHYC | |
| | APAGFAILKCNNETFNGTGPCNNVSTVQCTHGIKPVVSTQLLINGSLAEKEIIIRSENLTNN | |
| | AKIIIVHLNNPVKIICTRPGNNTRKSMRIGPGQTFYATGDIIGDIRRAYCNISEKTWYDTLK | |
| | NVSDKFQEHFPNASIEFKPSAGGDLEITTHSFNCRGEFFYCDTSELENGTYNNSTYNSSNNI | |
| | TLQCKIKQIINMWQGVGRCMYAPPIAGNITCESNITGLLLTRDGGNNKSTPETFRPGGGDMR | |
| | DNWRSELYKYKVVEIKPLGVAPTRCKRRVVGRRRRRRAVGIGAVSLGFLGAAGSTMGAASMT | |
| | LTVQARNLLSGIVQQQSNLLRAPEPQQHLLKDTHWGIKQLQARVLAVEHYLRDQQLLGIWGC | |
| | SGKLICCTNVPWNSSWSNRNLSEIWDNMTWLQWDKEISNYTQIIYGLLEESQNQQEKNEQDL | |
| | LALDGGGGSGGGSGGGSGSGAHIVMVDAYKPTK | |
| CNE55 | AENLWVTVYYGVPVWRDADTTLFCASDAKAHETEVHNVWATHACVPTDPNPQEIHLVNVTEN | 136 |
| SOSIP | FNMWKNKMVEQMQEDVISLWDESLKPCVKLTPLCVTLNCTTANTNETKNNTTDDNIKDEMKN | |
| Spy Tag | CTFNMTTEIRDKKQRVSALFYKLDIVPIDDSKNNSEYRLINCNTSVCKQACPKVSFDPIPIH | |
| | YCTPAGYVILKCNDKNFNGTGPCKNVSSVQCTHGIKPVVSTQLLINGSLAEEEIIIRSENLT | |
| | DNAKNIIVHLNKSVEINCTRPSNNTRTSVRIGPGQVFYRTGDITGDIRKAYCNISGTEWNKT | |
| | LTQVAEKLKEHFNKTIVYQPPSGGDLEITMHHFNCRGEFFYCNTTQLFNNSVGNSTIKLPCR | |
| | IKQIINMWQGVGQCMYAPPISGAINCLSNITGILLTRDGGGNNRSNETFRPGGGNIKDNWRS | |
| | ELYKYKVVEIEPLGVAPTRCKRRVVGRRRRRRAVGIGAVSLGFLGAAGSTMGAASMTLTVQA | |
| | RNLLSGIVQQQSNLLRAPEPQQHLLKDTHWGIKQLQARVLAVEHYLRDQQLLGIWGCSGKLI | |
| | CCTNVPWNSSWSNRNLSEIWDNMTWLQWDKEISNYTQIIYGLLEESQNQQEKNEQDLLALDG | |
| | GGGSGGGSGGGSGSGAHIVMVDAYKPTK | |
| Tro11 | AENLWVTVYYGVPVWKDASTTLFCASDAKAYDTEVHNVWATHACVPTDPNPQEVVLGNVTEN | 137 |
| ISOSIP | FNMWKNNMVDQMHEDIISLWDQSLKPCVKLTPLCVTLNCTDNITNTNTNSSKNSSTHSYNNS | |
| Spy Tag | LEGEMKNCSFNITAGIRDKVKKEYALFYKLDVVPIEEDKDTNKTTYRLRSCNTSVCTQACPK | |
| | VTFEPIPIHYCAPAGFAILKCNDKKFNGTGPCTNVSTVQCTHGIRPVVSTQLLINGSLAEEE | |
| | VVIRSENFTNNAKTIIVQLNESIAINCTRPNNNTRRSIHIGPGRAFYATGDIIGDIRQAHCN | |
| | ISRTEWNSTLRQIVTKLREQLGDPNKTIIFNQSSGGDTEITMHSFNCGGEFFYCNTTKLENS | |
| | TWNGNNTTESDSTGENITLPCRIKQIINLWQEVGKCMYAPPIKGQISCSSNITGLLLTRDGG | |
| | NNNSSGPETFRPGGGNMKDNWRSELYKYKVIKIEPLGVAPTRCKRRVVGRRRRRRAVGIGAV | |
| | SLGFLGAAGSTMGAASMTLTVQARNLLSGIVQQQSNLLRAPEPQQHLLKDTHWGIKQLQARV | |
| | LAVEHYLRDQQLLGIWGCSGKLICCTNVPWNSSWSNRNLSEIWDNMTWLQWDKEISNYTQII | |
| | YGLLEESQNQQEKNEQDLLALDGGGGSGGGSGGGSGSGAHIVMVDAYKPTK | |
| X1632 | AENLWVTVYYGVPVWEDADTTLFCASDAKAYSTESHNVWATHACVPTDPNPQEIYLENVTED | 138 |
| SOSIP | FNMWENNMVEQMQEDIISLWDESLKPCVKLTPLCVTLTCTNVTNVTDSVGTNSRLKGYKEEL | |
| Spy Tag | KNCSFNTTTEIRDKKKQEYALFYKLDIVPINDNSNNSNGYRLINCNVSTCKQACPKVSEDPI | |
| | PIHYCAPAGFAILKCRDKEFNGTGTCRNVSTVQCTHGIKPVVSTQLLINGSLAEGDIVIRSE | |
| | NITDNAKTIIVHLNKTVSITCTRPNNNTRKSIRIGPGQALYATGAIIGDTRQAHCNISGSEW | |
| | YEMIQNVKNKLNETFKKNITENPSSGGDLEITTHSFNCRGEFFYCNTSELENSSHLENGSTL | |
| | STNGTITLPCRIKQIVRMWQRVGQCMYAPPIAGNITCRSNITGLLLTRDGGTNKDTNEAETF | |
| | RPGGGDMRDNWRSELYKYKVVKIKPLGVAPTRCKRRVVGRRRRRRAVGIGAVSLGELGAAGS | |
| | TMGAASMTLTVQARNLLSGIVQQQSNLLRAPEPQQHLLKDTHWGIKQLQARVLAVEHYLRDQ | |
| | QLLGIWGCSGKLICCTNVPWNSSWSNRNLSEIWDNMTWLQWDKEISNYTQIIYGLLEESQNQ | |
| | QEKNEQDLLALDGGGGSGGGSGGGSGSGAHIVMVDAYKPTK | |
| X2278 | AENLWVTVYYGVPVWKEATTTLFCASEAKAYDTEVHNIWATHACVPTDPNPQEMELKNVTEN | 139 |
| SOSIP | FNMWKNNMVEQMHQDIISLWDQSLKPCVKLTPLCVTLDCTNINSTNSTNNTSSNSKMEETIG | |
| Spy Tag | VIKNCSFNVTTNIRDKVKKENALFYSLDLVSIGNSNTSYRLISCNTSICTQACPKVSEDPIP | |
| | IHYCAPAGFAILKCRDKKFNGTGPCRNVSSVQCTHGIRPVVSTQLLINGSLAEEEIVIRSAN | |
| | LTDNAKTIIIQLNETIQINCTRPNNNTRRSIPIGPGRTFYATGDIIGDIRKAYCNISATKWN | |
| | NTLRQIAEKLREKENKTIIFNQSSGGDPEVVRHTFNCGGEFFYCNSSQLENSTWYSNGTSNG | |
| | GLNNSANITLPCRIKQIINLWQEVGKCMYAPPIKGVINCLSNITGIILTRDGGENNGTTETF | |
| | RPGGGDMRDNWRSELYKYKVVKIEPLGVAPTRCKRRVVGRRRRRRAVGIGAVSLGELGAAGS | |
| | TMGAASMTLTVQARNLLSGIVQQQSNLLRAPEPQQHLLKDTHWGIKQLQARVLAVEHYLRDQ | |
| | QLLGIWGCSGKLICCTNVPWNSSWSNRNLSEIWDNMTWLQWDKEISNYTQIIYGLLEESQNQ | |
| | QEKNEQDLLALDGGGGSGGGSGGGSGSGAHIVMVDAYKPTK | |
| BG505 | NLWVTVYYGVPVWKDAETTLFCASDAKAYETEKHNVWATHACVPTDPNPQEIHLENVTEEFN | 140 |
| SOSIP | MWKNNMVEQMHTDIISLWDQSLKPCVKLTPLCVTLQCTNVINNITDDMRGELKNCSENMTTE | |
| | LRDKKQKVYSLFYRLDVVQINENQGNRSNNSNKEYRLINCNTSAITQACPKVSFEPIPIHYC | |
| | APAGFAILKCKDKKFNGTGPCPSVSTVQCTHGIKPVVSTQLLINGSLAEEEVMIRSENITNN | |
| | AKNILVQFNTPVQINCTRPNNNTRKSIRIGPGQAFYATGDIIGDIRQAHCNVSKATWNETLG | |
| | KVVKQLRKHFGNNTIIRFANSSGGDLEVTTHSFNCGGEFFYCNTSGLENSTWISNTSVQGSN | |
| | STGSNDSITLPCRIKQIINMWQRIGQAMYAPPIQGVIRCVSNITGLILTRDGGSTNSTTETF | |
| | RPGGGDMRDNWRSELYKYKVVKIEPLGVAPTRCKRRVVGRRRRRRAVGIGAVSLGFLGAAGS | |
| | TMGAASMTLTVQARNLLSGIVQQQSNLLRAPEPQQHLLKDTHWGIKQLQARVLAVEHYLRDQ | |
| | QLLGIWGCSGKLICCTNVPWNSSWSNRNLSEIWDNMTWLQWDKEISNYTQIIYGLLEESQNQ | |
| | QEKNEQDLLALD | |
| AMC011 | AEQLWVTVYYGVPVWKEATTTLFCASDARAYDTEVRNVWATHCCVPTDPNPQEVVLENVTEN | 141 |
| SOSIP | FNMWKNNMVEQMHEDIISLWDQSLKPCVKLTPLCVTLNCTDLRNATNTNATNTTSSSRGTME | |
| | GGEIKNCSFNITTSMRDKVQKEYALFYKLDVVPIKNDNTSYRLISCNTSVITQACPKVSFEP | |
| | IPIHYCAPAGFAILKCNNKTFNGTGPCTNVSTVQCTHGIRPVVSTQLLINGSLAEEEVVIRS | |
| | ANFTDNAKIIIVQLNKSVEINCTRPNNNTRKSIHIGPGRWFYTTGEIIGDIRQAHCNISGTK | |
| | WNDTLKQIVVKLKEQFGNKTIVFNHSSGGDPEIVMHSFNCGGEFFYCNSTQLFNSTWNDTTG | |
| | SNYTGTIVLPCRIKQIVNMWQEVGKAMYAPPIKGQIRCSSNITGLILIRDGGKNRSENTEIF | |
| | RPGGGDMRDNWRSELYKYKVVKIEPLGIAPTKCKRRVVQRRRRRRAVGIGAVELGELGAAGS | |
| | TMGAASMTLTVQARQLLSGIVQQQNNLLRAPECQQHLLKLTVWGIKQLQARVLAVERYLKDQ | |
| | QLLGIWGCSGKLICCTAVPWNTSWSNKSYNQIWNNMTWMEWEREIDNYTSLIYTLIEDSQNQ | |
| | QEKNEQELLELD | |
| B41 SOSIP | AAKKWVTVYYGVPVWKEATTTLFCASDAKAYDTEVHNVWATHACVPTDPNPQEIVLGNVTEN | 142 |
| | FNMWKNNMVEQMHEDIISLWDQSLKPCVKLTPLCVTLNCNNVNTNNTNNSTNATISDWEKME | |
| | TGEMKNCSFNVTTSIRDKIKKEYALFYKLDVVPLENKNNINNTNITNYRLINCNTSVITQAC | |
| | PKVSFEPIPIHYCAPAGFAILKCNSKTFNGSGPCTNVSTVQCTHGIRPVVSTQLLINGSLAE | |
| | EEIVIRSENITDNAKTIIVQLNEAVEINCTRPNNNTRKSIHIGPGRWFYATGDIIGNIRQAH | |
| | CNISKARWNETLGQIVAKLEEQFPNKTIIFNHSSGGDPEIVTHSFNCGGEFFYCNTTPLENS | |
| | TWNNTRTDDYPTGGEQNITLQCRIKQIINMWQGVGKAMYAPPIRGQIRCSSNITGLLLTRDG | |
| | GRDQNGTETFRPGGGNMRDNWRSELYKYKVVKIEPLGIAPTACKRRVVQRRRRRRAVGLGAF | |
| | ILGFLGAAGSTMGAASMALTVQARLLLSGIVQQQNNLLRAPEAQQHMLQLTVWGIKQLQARV | |
| | LAVERYLRDQQLLGIWGCSGKIICCTNVPWNDSWSNKTINEIWDNMTWMQWEKEIDNYTQHI | |
| | YTLLEVSQIQQEKNEQELLELD | |
| CH119 | AENLWVTVYYGVPVWKEATTTLFCASDAKAYDTEVHNVWATHACVPTDPSPQELVLENVTEN\| | 143 |
| SOSIP | FNMWKNEMVNQMHEDVISLWDQSLKPCVKLTPLCVTLECSKVSNNETDKYNGTEEMKNCSEN | |
| | ATTVVRDRQQKVYALFYRLDIVPLTEKNSSENSSKYYRLINCNTSACTQACPKVSFEPIPIH | |
| | YCTPAGYAILKCNDKTFNGTGPCHNVSTVQCTHGIKPVVSTQLLINGSLAEGEIIIRSENLT | |
| | NNVKTILVHLNQSVEIVCTRPNNNTRKSIRIGPGQTFYATGDIIGDIRQAHCNISKWHETLK | |
| | RVSEKLAEHFPNKTINFTSSSGGDLEITTHSFTCRGEFFYCNTSGLFNSTYMPNGTYLHGDT | |
| | NSNSSITIPCRIKQIINMWQEVGRCMYAPPIEGNITCKSNITGLLLVRDGGTESNNTETNNT | |
| | EIFRPGGGDMRDNWRSELYKYKVVEIKPLGVAPTRCKRRVVGRRRRRRAVGIGAVSLGFLGA | |
| | AGSTMGAASMTLTVQARNLLSGIVQQQSNLLRAPEPQQHLLKDTHWGIKQLQARVLAVEHYL | |
| | RDQQLLGIWGCSGKLICCTNVPWNSSWSNRNLSEIWDNMTWLQWDKEISNYTQIIYGLLEES | |
| | QNQQEKNEQDLLALD | |
| CE0217 | AENLWVTVYYGVPVWREAKTTLFCASDAKAYEREVHNVWATHACVPTDPNPQERVLENVTEN | 144 |
| SOSIP | FNMWKNNMVDQMHEDIISLWDESLKPCIKLTPLCVTLNCGNAIVNESTIEGMKNCSFNVTTE | |
| | LKDKKKKEYALFYKLDVVPLNGENNNSNSKNFSEYRLINCNTSTCTQACPKVSEDPIPIHYC | |
| | APAGFAILKCNNETFNGTGPCNNVSTVQCTHGIKPVVSTQLLINGSLAEKEIIIRSENLINN | |
| | AKIIIVHLNNPVKIICTRPGNNTRKSMRIGPGQTFYATGDIIGDIRRAYCNISEKTWYDTLK | |
| | NVSDKFQEHFPNASIEFKPSAGGDLEITTHSFNCRGEFFYCDTSELFNGTYNNSTYNSSNNI | |
| | TLQCKIKQIINMWQGVGRCMYAPPIAGNITCESNITGLLLTRDGGNNKSTPETFRPGGGDMR | |
| | DNWRSELYKYKVVEIKPLGVAPTRCKRRVVGRRRRRRAVGIGAVSLGFLGAAGSTMGAASMT | |
| | LTVQARNLLSGIVQQQSNLLRAPEPQQHLLKDTHWGIKQLQARVLAVEHYLRDQQLLGIWGC | |
| | SGKLICCTNVPWNSSWSNRNLSEIWDNMTWLQWDKEISNYTQIIYGLLEESQNQQEKNEQDL | |
| | LALD | |
| CNE8 | AENLWVTVYYGVPVWRDADTTLFCASDAKAYDTEVHNVWATHACVPTDPNPQEIHLENVTEN | 145 |
| SOSIP | FNMWKNKMAEQMQEDVISLWDESLKPCVQLTPLCVTLNCTNANLNATVNASTTIGNITDEVR | |
| | NCSFNTTTELRDKKQNVYALFYKLDIVPINNNSEYRLINCNTSVCKQACPKVSEDPIPIHYC | |
| | APAGYAILRCNDKNFNGTGPCKNVSSVQCTHGIKPVVSTQLLINGSLAEDEIIIRSENLTDN | |
| | VKTIIVHLNKSVEINCTRPSNNTRTSVRIGPGQVFYRTGDIIGDIRKAYCNISRTKWHETLK | |
| | QVATKLREHENKTIIFQPPSGGDIEITMHHFNCRGEFFYCNTTKLENSTWGENTTMEGHNDT | |
| | IVLPCRIKQIVNMWQGVGQCMYAPPIRGSINCVSNITGILLTRDGGTNMSNETFRPGGGNIK | |
| | DNWRSELYKYKVVEIEPLGVAPTRCKRRVVGRRRRRRAVGIGAVSLGFLGAAGSTMGAASMT | |
| | LTVQARNLLSGIVQQQSNLLRAPEPQQHLLKDTHWGIKQLQARVLAVEHYLRDQQLLGIWGC | |
| | SGKLICCTNVPWNSSWSNRNLSEIWDNMTWLQWDKEISNYTQIIYGLLEESQNQQEKNEQDL | |
| | LALD | |
| CNE8 | AENLWVTVYYGVPVWRDADTTLFCASDAKAYDTEVHNVWATHACVPTDPNPQEIHLENVTEN | 146 |
| N276A | FNMWKNKMAEQMQEDVISLWDESLKPCVQLTPLCVTLNCTNANLNATVNASTTIGNITDEVR | |
| SOSIP | NCSFNTTTELRDKKQNVYALFYKLDIVPINNNSEYRLINCNTSVCKQACPKVSFDPIPIHYC | |
| | APAGYAILRCNDKNFNGTGPCKNVSSVQCTHGIKPVVSTQLLINGSLAEDEIIIRSEALTDN | |
| | VKTIIVHLNKSVEINCTRPSNNTRTSVRIGPGQVFYRTGDIIGDIRKAYCNISRTKWHETLK | |
| | QVATKLREHFNKTIIFQPPSGGDIEITMHHFNCRGEFFYCNTTKLFNSTWGENTTMEGHNDT | |
| | IVLPCRIKQIVNMWQGVGQCMYAPPIRGSINCVSNITGILLTRDGGTNMSNETFRPGGGNIK | |
| | DNWRSELYKYKVVEIEPLGVAPTRCKRRVVGRRRRRRAVGIGAVSLGFLGAAGSTMGAASMT | |
| | LTVQARNLLSGIVQQQSNLLRAPEPQQHLLKDTHWGIKQLQARVLAVEHYLRDQQLLGIWGC | |
| | SGKLICCTNVPWNSSWSNRNLSEIWDNMTWLQWDKEISNYTQIIYGLLEESQNQQEKNEQDL | |
| | LALD | |
| CNE20 | NLWVTVYYGVPVWKEATTTLFCASDAKAYDTEVHNVWATHACVPTDPNPHELVLENVTENEN | 147 |
| SOSIP | MWKNEMVNQMHEDVISLWDQSLKPCVKLTPLCVTLECGNITTRKESMTEMKNCSFNATTVVK | |
| | DRKQTVYALFYKLDIVPLSGKNSSGYYRLINCNTSACTQACPKVNEDPIPIHYCTPAGYAIL | |
| | KCNDKTFNGTGPCHNVSTVQCTHGIKPVISTQLLLNGSLAEGEIVIRSENLTNNAKIIIVHL | |
| | NQTVEIVCTRPGNNTRKSIRIGPGQTFYATGEIIGNIRQAHCNISENQWHKTLQNVSKKLAE | |
| | HFQNKTITFASSSGGDLEITTHSFNCRGEFFYCNTSGLENGTYMSNNTEGNSSSIITIPCRI | |
| | KQIINMWQEVGRCIYAPPIEGNITCKSNITGLLLERDGGTESNDTEIFRPGGGDMRNNWRSE | |
| | LYKYKVVEIKPLGVAPTRCKRRVVGRRRRRRAVGIGAVSLGELGAAGSTMGAASMTLTVQAR | |
| | NLLSGIVQQQSNLLRAPEPQQHLLKDTHWGIKQLQARVLAVEHYLRDQQLLGIWGCSGKLIC | |
| | CTNVPWNSSWSNRNLSEIWDNMTWLQWDKEISNYTQIIYGLLEESQNQQEKNEQDLLALD | |
| CNE20 | NLWVTVYYGVPVWKEATTTLFCASDAKAYDTEVHNVWATHACVPTDPNPHELVLENVTENEN | 148 |
| N276A | MWKNEMVNQMHEDVISLWDQSLKPCVKLTPLCVTLECGNITTRKESMTEMKNCSFNATTVVK | |
| SOSIP | DRKQTVYALFYKLDIVPLSGKNSSGYYRLINCNTSACTQACPKVNFDPIPIHYCTPAGYAIL | |
| | KCNDKTFNGTGPCHNVSTVQCTHGIKPVISTQLLINGSLAEGEIVIRSEALTNNAKIIIVHL | |
| | NQTVEIVCTRPGNNTRKSIRIGPGQTFYATGEIIGNIRQAHCNISENQWHKTLQNVSKKLAE | |
| | HFQNKTITFASSSGGDLEITTHSFNCRGEFFYCNTSGLFNGTYMSNNTEGNSSSIITIPCRI | |
| | KQIINMWQEVGRCIYAPPIEGNITCKSNITGLLLERDGGTESNDTEIFRPGGGDMRNNWRSE | |
| | LYKYKVVEIKPLGVAPTRCKRRVVGRRRRRRAVGIGAVSLGFLGAAGSTMGAASMTLTVQAR | |
| | NLLSGIVQQQSNLLRAPEPQQHLLKDTHWGIKQLQARVLAVEHYLRDQQLLGIWGCSGKLIC | |
| | CTNVPWNSSWSNRNLSEIWDNMTWLQWDKEISNYTQIIYGLLEESQNQQEKNEQDLLALD | |
| IOMA HC | EVQLVESGAQVKKPGASVTVSCTASGYKFTGYHMHWVRQAPGRGLEWMGWINPERGAVKYPQ | 149 |
| Fab | NFRGRVSMTRDTSMEIFYMELSRLTSDDTAVYYCAREMEDSSADWSPWRGMVAWGQGTLVTV | |
| | SSASTKGPSVFPLAPSSKSTSGGTAALGCLVKDYFPEPVTVSWNSGALTSGVHTFPAVLQSS | |
| | GLYSLSSVVTVPSSSLGTQTYICNVNHKPSNTKVDKRVEPKSCDKT | |
| IOMA HC | EVQLVESGAQVKKPGASVTVSCTASGYKFTGYHMHWVRQAPGRGLEWMGWINPFRGAVKYPQ | 150 |
| | NFRGRVSMTRDTSMEIFYMELSRLTSDDTAVYYCAREMEDSSADWSPWRGMVAWGQGTLVTV | |
| | SSASTKGPSVFPLAPSSKSTSGGTAALGCLVKDYFPEPVTVSWNSGALTSGVHTFPAVLQSS | |
| | GLYSLSSVVTVPSSSLGTQTYICNVNHKPSNTKVDKRVEPKSCDKTHTCPPCPAPELLGGPS | |
| | VFLFPPKPKDTLMISRTPEVTCVVVDVSHEDPEVKENWYVDGVEVHNAKTKPREEQYNSTYR | |
| | VVSVLTVLHQDWLNGKEYKCKVSNKALPAPIEKTISKAKGQPREPQVYTLPPSREEMTKNQV | |
| | SLTCLVKGFYPSDIAVEWESNGQPENNYKTTPPVLDSDGSFFLYSKLTVDKSRWQQGNVFSC | |
| | SVMHEALHNHYTQKSLSLSPGK | |
| IOMA LC | QSALTQPASVSGSPGQSITISCAGSSRDVGGFDLVSWYQQHPGKAPKLIIYEVNKRPSGISS | 151 |
| | RFSASKSGNTASLTISGLQEEDEAHYYCYSYADGVAFGGGTKLTVLGQPKAAPSVTLFPPSS | |
| | EELQANKATLVCLISDFYPGAVTVAWKADSSPVKAGVETTTPSKQSNNKYAASSYLSLTPEQ | |
| | WKSHRSYSCQVTHEGSTVEKTVAPTECS | |
| IOMA iGL | QVQLVQSGAEVKKPGASVKVSCKASGYTFTGYYMHWVRQAPGQGLEWMGWINPNSGGTNYAQ | 152 |
| HC Fab | KFQGRVTMTRDTSISTAYMELSRLRSDDTAVYYCARDFTSSYDSSGYYHEGYWGQGTLVTVS | |
| | SASTKGPSVFPLAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKT | |
| IOMA iGL | QVQLVQSGAEVKKPGASVKVSCKASGYTFTGYYMHWVRQAPGQGLEWMGWINPNSGGTNYAQ | 153 |
| HC | KFQGRVTMTRDTSISTAYMELSRLRSDDTAVYYCARDFTSSYDSSGYYHEGYWGQGTLVTVS | |
| | SASTKGPSVFPLAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCPAPELLGGPSV | |
| | FLFPPKPKDTLMISRTPEVTCVVVDVSHEDPEVKENWYVDGVEVHNAKTKPREEQYNSTYRV | |
| | VSVLTVLHQDWLNGKEYKCKVSNKALPAPIEKTISKAKGQPREPQVYTLPPSREEMTKNQVS | |
| | LTCLVKGFYPSDIAVEWESNGQPENNYKTTPPVLDSDGSFFLYSKLTVDKSRWQQGNVESCS | |
| | VMHEALHNHYTQKSLSLSPGK | |
| IOMA iGL | QSALTQPASVSGSPGQSITISCTGTSSDVGSYNLVSWYQQHPGKAPKLMIYEVSKRPSGVSN | 154 |
| LC | RFSGSKSGNTASLTISGLQAEDEADYYCCSYAGSVAFGGGTKLTVLGQPKAAPSVTLFPPSS | |
| | EELQANKATLVCLISDFYPGAVTVAWKADSSPVKAGVETTTPSKQSNNKYAASSYLSLTPEQ | |
| | WKSHRSYSCQVTHEGSTVEKTVAPTECS | |
| VRC01 iGL | SSVVTVPSSSLGTQTYICNVNHKPSNTKVDKRVEPKSCDKTHTCPPCPAPELLGGPSVFLFP | 155 |
| HC | QVQLVQSGAEVKKPGASVKVSCKASGYTFTGYYMHWVRQAPGQGLEWMGWINPNSGGTNYAQ | |
| | KFQGRVTMTRDTSISTAYMELSRLRSDDTAVYYCARGKNSDYNWDFQHWGQGTLVTVSSAST | |
| | KGPSVFPLAPSSKSTSGGTAALGCLVKDYFPEPVTVSWNSGALTSGVHTFPAVLQSSGLYSL | |
| | PKPKDTLMISRTPEVTCVVVDVSHEDPEVKFNWYVDGVEVHNAKTKPREEQYNSTYRVVSVL | |
| | TVLHQDWLNGKEYKCKVSNKALPAPIEKTISKAKGQPREPQVYTLPPSREEMTKNQVSLTCL | |
| | VKGFYPSDIAVEWESNGQPENNYKTTPPVLDSDGSFFLYSKLTVDKSRWQQGNVFSCSVMHE | |
| | ALHNHYTQKSLSLSPGK | |
| VRC01 iGL | SGSGSGTDFTLTISSLEPEDFAVYYCQQYEFFGQGTKLEIKRTVAAPSVFIFPPSDEQLKSG | 156 |
| LC | EIVLTQSPATLSLSPGERATLSCRASQSVSSYLAWYQQKPGQAPRLLIYDASNRATGIPARF | |
| | TASVVCLLNNFYPREAKVQWKVDNALQSGNSQESVTEQDSKDSTYSLSSTLTLSKADYEKHK | |
| | VYACEVTHQGLSSPVTKSENRGEC | |
| 3BNC60 | QVQLVQSGAEVKKPGASVKVSCKASGYTFTGYYMHWVRQAPGQGLEWMGWINPNSGGTNYAQ | 157 |
| iGL HC | KFQGRVTMTRDTSISTAYMELSRLRSDDTAVYYCARERSDFWDFDLWGRGTLVTVSSASTKG | |
| | PSVFPLAPSSKSTSGGTAALGCLVKDYFPEPVTVSWNSGALTSGVHTFPAVLQSSGLYSLSS | |
| | VVTVPSSSLGTQTYICNVNHKPSNTKVDKRVEPKSCDKTHTCPPCPAPELLGGPSVFLFPPK | |
| | PKDTLMISRTPEVTCVVVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVFSCSVMHEAL | |
| | HNHYTQKSLSLSPGK | |
| 3BNC60 | DIQMTQSPSSLSASVGDRVTITCQASQDISNYLNWYQQKPGKAPKLLIYDASNLETGVPSRF | 158 |
| iGL LC | SGSGSGTDFTFTISSLQPEDIATYYCQQYEFIGPGTKVDIKRTVAAPSVFIFPPSDEQLKSG | |
| | TASVVCLLNNFYPREAKVQWKVDNALQSGNSQESVTEQDSKDSTYSLSSTLTLSKADYEKHK | |
| | VYACEVTHQGLSSPVTKSENRGEC | |
| BG24 iGL | QVQLVQSGAEVKKPGASVKVSCKASGYTFTGYYMHWVRQAPGQGLEWMGWINPNSGGTNYAQ | 159 |
| HC | KFQGRVTMTRDTSISTAYMELSRLRSDDTAVYYCATQLELDSSAGYAFDIWGQGTMVTVSSA | |
| | STKGPSVFPLAPSSKSTSGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTCPPCPAPELLGGPSVEL | |
| | FPPKPKDTLMISRTPEVTCVVVDVSHEDPEVKENWYVDGVEVHNAKTKPREEQYNSTYRVVS | |
| | VLTVLHQDWLNGKEYKCKVSNKALPAPIEKTISKAKGQPREPQVYTLPPSREEMTKNQVSLT | |
| | CLVKGFYPSDIAVEWESNGQPENNYKTTPPVLDSDGSFFLYSKLTVDKSRWQQGNVFSCSVM | |
| | HEALHNHYTQKSLSLSPGK | |
| BG24 iGL | QSALTQPRSVSGSPGQSVTISCTGTSSDVGGYNYVSWYQQHPGKAPKLMIYDVSKRPSGVPD | 160 |
| LC | RFSGSKSGNTASLTISGLQAEDEADYYCSSYEYFGGGTKLTVLSQPKAAPSVTLFPPSSEEL | |
| | QANKATLVCLISDFYPGAVTVAWKADSSPVKAGVETTTPSKQSNNKYAASSYLSLTPEQWKS | |
| | HRSYSCQVTHEGSTVEKTVAPTECS | |
| HXB2CG | MRVKEKYQHLWRWGWRWGTMLLGMLMICSATEKLWVTVYYGVPVWKEATTTLFCASDAKAYD | 268 |
| Env | TEVHNVWATHACVPTDPNPQEVVLVNVTENFNMWKNDMVEQMHEDIISLWDQSLKPCVKLTP | |
| (gp160) | LCVSLKCTDLKNDTNTNSSSGRMIMEKGEIKNCSFNISTSIRGKVQKEYAFFYKLDIIPIDN | |
| | DTTSYKLTSCNTSVITQACPKVSFEPIPIHYCAPAGFAILKCNNKTENGTGPCTNVSTVQCT | |
| | HGIRPVVSTQLLINGSLAEEEVVIRSVNFTDNAKTIIVQLNTSVEINCTRPNNNTRKRIRIQ | |
| | RGPGRAFVTIGKIGNMRQAHCNISRAKWNNTLKQIASKLREQFGNNKTIIFKQSSGGDPEIV | |
| | THSFNCGGEFFYCNSTQLFNSTWFNSTWSTEGSNNTEGSDTITLPCRIKQIINMWQKVGKAM | |
| | YAPPISGQIRCSSNITGLLLTRDGGNSNNESEIFRPGGGDMRDNWRSELYKYKVVKIEPLGV | |
| | APTKAKRRVVQREKRAVGIGALFLGFLGAAGSTMGAASMTLTVQARQLLSGIVQQQNNLLRA | |
| | IEAQQHLLQLTVWGIKQLQARILAVERYLKDQQLLGIWGCSGKLICTTAVPWNASWSNKSLE | |
| | QIWNHTTWMEWDREINNYTSLIHSLIEESQNQQEKNEQELLELDKWASLWNWENITNWLWYI | |
| | KLFIMIVGGLVGLRIVFAVLSIVNRVRQGYSPLSFQTHLPTPRGPDRPEGIEEEGGERDRDR | |
| | SIRLVNGSLALIWDDLRSLCLFSYHRLRDLLLIVTRIVELLGRRGWEALKYWWNLLQYWSQE | |
| | LKNSAVSLLNATAIAVAEGTDRVIEVVQGACRAIRHIPRRIRQGLERILL | |

TABLE 2

| X-RAY DATA COLLECTION FOR | | |
|---|---|---|
| IOMA IGL FAB CRYSTALS | | |
| Space group | P 2₁ 2₁ 2₁ | |
| Cell dimensions | | |
| a, b, c (Å) | 57.7, 66.7, 166.3 | |
| α, β, γ (*) | 90, 90, 90 | |
| Resolution (Å) | 38.6-2.07 | (2.15-2.07)^{a} |
| R _{merge} | 0.08 | (0.58) |
| R ₚᵢₘ | 0.05 | (0.36) |
| I/σ(I) | 9.7 | (2.5) |
| CC _{1/2} | 0.99 | (0.92) |
| Completeness (%) | 99 | (99) |
| Redundancy | 6.3 | (6.6) |
| Refinement | | |
| Resolution (Å) | 38.6-2.07 | |
| No. reflections | 39,372 | |
| R_{work}/R_{free} | 0.224/0.257 | |
| No. atoms | | |
| Protein | 3,241 | |
| Ligand/ion | N/A | |
| B factors (Å²) | | |
| Protein | 46.7 | |
| Ligand/ion | N/A | |
| R.m.s. deviations | | |

| | |
|---|---|
| Bond lengths (Å) | 0.008 |
| Bond angles (°) | 1.00 |

^{a}Values in parentheses are for the highest-resolution shell.

TABLE 3A
SERUM NEUTRALIZATION DATA
FOR IOMA IGL TRANSGENIC MICE

| | | | ES30 | | ES32 | | ES34 | |
|---|---|---|---|---|---|---|---|---|
| | | | B3 | | B3 | | B3 | |
| | | | (week 18) | | (week 18) | | (week 18) | |
| | | | | % | | % | | % |
| Virus | Clade | Tier | ID₅₀ | 1:100 | ID₅₀ | 1:100 | ID₅₀ | 1:100 |
| 426c | C | 2 | — | — | — | — | — | — |
| 25710 | B | 2 | — | — | — | — | — | — |
| CNE8 | AE | 1 | <100 | 0 | <100 | 0 | <100 | 0 |
| CNE8 | AE | 1 | 463 | 71 | <100 | 0 | <100 | 0 |
| N276A | | | | | | | | |
| CNE20 | BC | 2 | <100 | 49 | <100 | 0 | <100 | 0 |
| CNE20 | BC | 2 | 14,922 | 95 | <100 | 0 | <100 | 0 |
| N276A | | | | | | | | |
| JRCSF | B | 2 | 136 | 65 | <100 | 0 | <100 | 0 |
| Q23.17 | A | 1 | 100 | 51 | <100 | 0 | <100 | 0 |
| YU2 | B | 2 | 571 | 86 | <100 | 0 | <100 | 0 |
| BG505 | A | 2 | — | — | — | — | — | — |
| T332N | | | | | | | | |
| 6535.5 | B | 1 | — | — | — | — | — | — |
| 3415_V1_C1 | A | 2 | — | — | — | — | — | — |
| CAAN5342.A2 | B | 2 | — | — | — | — | — | — |
| PVO.4 | B | 3 | 113 | 52 | <100 | 0 | <100 | 0 |
| Q842.D12 | A | 2 | — | — | — | — | — | — |
| RHPA4259.7 | B | 2 | — | — | — | — | — | — |
| WITO4160.33 | B | 2 | <100 | 0 | <100 | 0 | <100 | 0 |
| ZM214M.PL15 | C | 2 | — | — | — | — | — | — |
| MuLV | | | <100 | 0 | <100 | 0 | <100 | 0 |

TABLE 3B
SERUM NEUTRALIZATION DATA
FOR IOMA IGL TRANSGENIC MICE

| | | ES37 | | | ET33 | | ET34 | |
|---|---|---|---|---|---|---|---|---|
| | | B3 (week 18) | | | B3 (week 18) | | B3 (week 18) | |
| Virus | Clade | Tier | ID₅₀ | % 1:100 | ID₅₀ | % 1:100 | ID₅₀ | % 1:100 |
| 426c | C | 2 | — | — | <100 | 0 | <100 | 0 |
| 25710 | B | 2 | — | — | <100 | 0 | <100 | 0 |
| CNE8 | AE | 1 | <100 | 0 | <100 | 0 | 104 | 54 |
| CNE8 N276A | AE | 1 | <100 | 0 | <100 | 0 | <100 | 40 |
| CNE20 | BC | 2 | <100 | 0 | <100 | 0 | <100 | 0 |
| CNE20 N276A | BC | 2 | <100 | 0 | <100 | 0 | <100 | 36 |
| JRCSF | B | 2 | <100 | 0 | <100 | 0 | <100 | 0 |
| Q23.17 | A | 1 | <100 | 0 | <100 | 0 | <100 | 26 |
| YU2 | B | 2 | <100 | 0 | <100 | 0 | 112 | 56 |
| BG505 T332N | A | 2 | — | — | <100 | 0 | <100 | 0 |
| 6535.5 | B | 1 | — | — | <100 | 0 | 104 | 53 |
| 3415_V1_C1 | A | 2 | — | — | 154 | 53 | 102 | 59 |
| CAAN5342.A2 | B | 2 | — | — | <100 | 0 | <100 | 41 |
| PVO.4 | B | 3 | <100 | 0 | <100 | 22 | <100 | 57 |
| Q842.D12 | A | 2 | — | — | <100 | 0 | <100 | 0 |
| RHPA4259.7 | B | 2 | — | — | <100 | 0 | <100 | 43 |
| WITO4160.33 | B | 2 | <100 | 0 | <100 | 0 | <100 | 45 |
| ZM214M.PL15 | C | 2 | — | — | <100 | 0 | <100 | 0 |
| MuLV | | | <100 | 0 | <100 | 0 | 162 | 65 |

TABLE 3C
SERUM NEUTRALIZATION DATA FOR
IOMA IGL TRANSGENIC MICE

| | | | HP1 | | HP2 | | HP3 | |
|---|---|---|---|---|---|---|---|---|
| | | | B4 | | B4 | | B4 | |
| | | | (week 23) | | (week 23) | | (week 23) | |
| | | | | % | | % | | % |
| Virus | Clade | Tier | ID₅₀ | 1:100 | ID₅₀ | 1:100 | ID₅₀ | 1:100 |
| 426c | C | 2 | <100 | 0 | <100 | 0 | <100 | 0 |
| 25710 | B | 2 | <100 | 0 | <100 | 0 | <100 | 0 |
| CNE8 | AE | 1 | <100 | 0 | <100 | 0 | <100 | 0 |
| CNE8 N276A | AE | 1 | <100 | 26 | <100 | 0 | <100 | 0 |
| CNE20 | BC | 2 | <100 | 23 | <100 | 0 | <100 | 40 |
| CNE20 N276A | BC | 2 | 338 | 83 | 610 | 79 | 903 | 88 |
| JRCSF | B | 2 | <100 | 0 | <100 | 0 | <100 | 0 |
| Q23.17 | A | 1 | 120 | 57 | <100 | 30 | <100 | 28 |
| YU2 | B | 2 | <100 | 0 | <100 | 0 | <100 | 0 |
| BG505 T332N | A | 2 | <100 | 0 | <100 | 0 | <100 | 0 |
| 6535.5 | B | 1 | 165 | 67 | <100 | 0 | <100 | 0 |
| 3415_V1_C1 | A | 2 | <100 | 0 | <100 | 40 | <100 | 0 |
| CAAN5342.A2 | B | 2 | <100 | 0 | <100 | 0 | <100 | 0 |
| PVO.4 | B | 3 | <100 | 43 | <100 | 45 | <100 | 12 |
| Q842.D12 | A | 2 | <100 | 0 | <100 | 0 | <100 | 0 |
| RHPA4259.7 | B | 2 | <100 | 0 | <100 | 0 | <100 | 0 |
| WITO4160.33 | B | 2 | <100 | 28 | 145 | 53 | <100 | 0 |
| ZM214M.PL15 | C | 2 | <100 | 0 | <100 | 0 | <100 | 0 |
| MuLV | | | <100 | 0 | <100 | 0 | <100 | 0 |

TABLE 3D
SERUM NEUTRALIZATION DATA FOR IOMA IGL TRANSGENIC MICE

| | | HP4 | | | HP6 | | HP7 | | HQ4 | |
|---|---|---|---|---|---|---|---|---|---|---|
| | | B4 | | | B4 | | B4 | | B4 | |
| | | (week 23) | | | (week 23) | | (week 23) | | (week 23) | |
| | | | | % | | % | | % | | % |
| Virus | Clade | Tier | ID₅₀ | 1:100 | ID₅₀ | 1:100 | ID₅₀ | 1:100 | ID₅₀ | 1:100 |
| 426c | C | 2 | <100 | 0 | — | — | <100 | 0 | <100 | 0 |
| 25710 | B | 2 | <100 | 0 | — | — | <100 | 0 | <100 | 0 |
| CNE8 | AE | 1 | <100 | 0 | — | — | <100 | 0 | <100 | 0 |
| CNE8 | AE | 1 | <100 | 12 | — | — | <100 | 0 | <100 | 0 |
| N276A | | | | | | | | | | |
| CNE20 | BC | 2 | <100 | 0 | — | — | <100 | 0 | <100 | 0 |
| CNE20 | BC | 2 | <100 | 16 | — | — | 2,017 | 98 | <100 | 0 |
| N276A | | | | | | | | | | |
| JRCSF | B | 2 | <100 | 0 | — | — | <100 | 0 | <100 | 0 |
| Q23.17 | A | 1 | <100 | 0 | — | — | <100 | 0 | <100 | 0 |
| YU2 | B | 2 | <100 | 0 | — | — | <100 | 0 | <100 | 0 |
| BG505 | A | 2 | <100 | 0 | — | — | <100 | 0 | <100 | 0 |
| T332N | | | | | | | | | | |
| 6535.5 | B | 1 | <100 | 0 | | | <100 | 0 | <100 | 0 |
| 3415_V1_C1 | A | 2 | <100 | 0 | — | — | <100 | 0 | 108 | 50 |
| CAAN5342A2 | B | 2 | <100 | 0 | — | — | <100 | 0 | <100 | 0 |
| PVO.4 | B | 3 | <100 | 35 | — | — | 115 | 50 | <100 | 0 |
| Q842.D12 | A | 2 | <100 | 0 | — | — | <100 | 0 | <100 | 0 |
| RHPA4259.7 | B | 2 | <100 | 0 | — | — | <100 | 0 | <100 | 0 |
| WITO4160.33 | B | 2 | <100 | 48 | — | — | <100 | 40 | <100 | 0 |
| ZM214M.PL15 | C | 2 | <100 | 0 | — | — | <100 | 0 | <100 | 0 |
| MuLV | | | <100 | 0 | — | — | <100 | 0 | <100 | 0 |

TABLE 4A
MUTATIONAL ANALYSIS OF ANTIBODIES
ISOLATED FROM IOMA IGL TRANSGENIC MICE

| | | | | # of IGT2- | | | | |
|---|---|---|---|---|---|---|---|---|
| VH1-2*02 | | | | induced | | | | |
| amino | VH | amino acid | Random | mAbs with | IGT2 | IOMA | Critical | VRC01 |
| acid | Position | substitution | Frequency | this SHM | Freq. | Substitution | Interaction | Substitution |
| Q | 1 | | — | 67 | 100.0 | E | | |
| | | E | 0.0 | 0 | 0.0 | | | |
| V | 2 | | | 67 | 100.0 | V | | |
| Q | 3 | | | 67 | 100.0 | Q | | |
| L | 4 | | | 67 | 100.0 | L | | |
| V | 5 | | | 67 | 100.0 | V | | |
| Q | 6 | | | 67 | 100.0 | E | | |
| | | E | 0.1 | 0 | 0.0 | | | |
| S | 7 | | | 67 | 100.0 | S | | |
| G | 8 | | | 67 | 100.0 | G | | |
| A | 9 | | | 67 | 100.0 | A | | G |
| E | 10 | | | 67 | 100.0 | Q | | Q |
| | | Q | 0.2 | 0 | 0.0 | | | |
| V | 11 | | | 66 | 98.5 | V | | M |
| | | M | 3.3 | 1 | 1.5 | | | |
| K | 12 | | | 56 | 83.6 | K | | |
| | | R | 6.9 | 11 | 16.4 | | | |
| K | 13 | | | 67 | 100.0 | K | | |
| P | 14 | | | 67 | 100.0 | P | | |
| G | 15 | | | 67 | 100.0 | G | | E |
| A | 16 | | | 67 | 100.0 | A | | |
| S | 17 | | | 67 | 100.0 | S | | |
| V | 18 | | | 67 | 100.0 | V | | M |
| K | 19 | | | 22 | 32.8 | T | YES | R |
| | | T | 2.3 | 31 | 46.3 | | | |
| | | R | 9.6 | 14 | 20.9 | | | |
| V | 20 | | | 67 | 100.0 | V | | I |
| S | 21 | | | 67 | 100.0 | S | | |
| C | 22 | | | 67 | 100.0 | C | | |
| K | 23 | | | 55 | 82.1 | T | | R |
| | | A | 0.2 | 1 | 1.5 | | | |
| | | T | 2.3 | 0 | 0.0 | | | |
| | | E | 3.3 | 5 | 7.5 | | | |
| | | R | 4.6 | 6 | 9.0 | | | |
| A | 24 | | | 61 | 91.0 | A | | |
| | | T | 13.5 | 6 | 9.0 | | | |
| S | 25 | | | 67 | 100.0 | S | | |
| G | 26 | | | 67 | 100.0 | G | | |
| Y | 27 | | | 67 | 100.0 | Y | | |
| T | 28 | | | 65 | 97.0 | K | | E |
| | | K | 0.6 | 0 | 0.0 | | | |
| | | N | 2.0 | 2 | 3.0 | | | |
| F | 29 | | | 66 | 98.5 | F | | |
| | | L | 3.8 | 1 | 1.5 | | | |
| T | 30 | | | 58 | 86.6 | T | | I |
| | | A | 1.7 | 1 | 1.5 | | | |
| | | I | 7.2 | 8 | 11.9 | | | |
| G | 31 | | | 32 | 47.8 | G | | D |
| | | E | 0.8 | 1 | 1.5 | | | |
| | | A | 11.7 | 6 | 9.0 | | | |
| | | D | 34.3 | 28 | 41.8 | | | |
| Y | 32 | | | 65 | 97.0 | Y | | C |
| | | H | 8.5 | 2 | 3.0 | | | |
| Y | 33 | | | 27 | 40.3 | H | YES | T |
| | | E | 0.1 | 14 | 20.9 | | | |
| | | D | 0.6 | 9 | 13.4 | | | |
| | | S | 1.9 | 2 | 3.0 | | | |
| | | H | 4.5 | 8 | 11.9 | | | |
| | | F | 8.4 | 7 | 10.4 | | | |
| M | 34 | | | 33 | 49.3 | M | | L |
| | | L | 13.8 | 7 | 10.4 | | | |
| | | I | 48.4 | 27 | 40.3 | | | |
| H | 35 | | | 61 | 91.0 | H | | N |
| | | Q | 2.2 | 1 | 1.5 | | | |
| | | Y | 3.5 | 5 | 7.5 | | | |
| W | 36 | | | 67 | 100.0 | W | | |
| V | 37 | | | 67 | 100.0 | V | | I |
| R | 38 | | | 67 | 100.0 | R | | |
| Q | 39 | | | 66 | 98.5 | Q | | L |
| | | R | 1.1 | 1 | 1.5 | | | |
| A | 40 | | | 65 | 97.0 | A | | |
| | | V | 2.0 | 2 | 3.0 | | | |
| P | 41 | | | 67 | 100.0 | P | | |
| G | 42 | | | 67 | 100.0 | G | | |
| Q | 43 | | | 66 | 98.5 | R | | K |
| | | R | 1.5 | 1 | 1.5 | | | |
| G | 44 | | | 67 | 100.0 | G | | R |
| L | 45 | | | 64 | 95.5 | L | | P |
| | | F | 1.9 | 3 | 4.5 | | | |
| E | 46 | | | 66 | 98.5 | E | | |
| | | D | 0.5 | 1 | 1.5 | | | |
| W | 47 | | | 67 | 100.0 | W | | |
| M | 48 | | | 62 | 92.5 | M | | |
| | | L | 4.9 | 4 | 6.0 | | | |
| | | V | 6.4 | 1 | 1.5 | | | |
| G | 49 | | | 67 | 100.0 | G | | |
| W | 50 | | | 52 | 77.6 | W | | |
| | | R | 0.0 | 14 | 20.9 | | | |
| | | L | 0.8 | 1 | 1.5 | | | |
| I | 51 | | | 66 | 98.5 | I | | L |
| | | S | 0.5 | 1 | 1.5 | | | |
| N | 52 | | | 58 | 86.6 | N | | K |
| | | H | 2.2 | 3 | 4.5 | | | |
| | | S | 4.4 | 6 | 9.0 | | | |
| P | (52A) | | | 67 | 100.0 | P | | |
| N | 53 | | | 15 | 22.4 | F | YES | R |
| | | F | 0.1 | 30 | 44.8 | | | |
| | | E | 1.0 | 2 | 3.0 | | | |
| | | T | 1.4 | 5 | 7.5 | | | |
| | | R | 1.8 | 7 | 10.4 | | | |
| | | Y | 3.5 | 5 | 7.5 | | | |
| | | D | 8.0 | 1 | 1.5 | | | |
| | | K | 13.5 | 2 | 3.0 | | | |
| S | 54 | | | 12 | 17.9 | R | YES | G |
| | | F | 0.2 | 7 | 10.4 | | | |
| | | R | 2.7 | 45 | 67.2 | | | |
| | | N | 11.9 | 1 | 1.5 | | | |
| | | T | 14.8 | 2 | 3.0 | | | |
| G | 55 | | | 67 | 100.0 | G | | |
| G | 56 | | | 31 | 46.3 | A | YES | A |
| | | R | 0.9 | 2 | 3.0 | | | |
| | | N | 0.9 | 8 | 11.9 | | | |
| | | V | 6.3 | 5 | 7.5 | | | |
| | | A | 11.3 | 20 | 29.9 | | | |
| | | D | 22.4 | 1 | 1.5 | | | |
| T | 57 | | | 21 | 31.3 | V | YES | V |
| | | V | 0.4 | 21 | 31.3 | | | |
| | | R | 0.9 | 3 | 4.5 | | | |
| | | P | 1.3 | 2 | 3.0 | | | |
| | | I | 1.4 | 18 | 26.9 | | | |
| | | S | 1.9 | 2 | 3.0 | | | |
| N | 58 | | | 18 | 26.9 | K | YES | |
| | | G | 0.7 | 10 | 14.9 | | | |
| | | E | 1.4 | 15 | 22.4 | | | |
| | | D | 7.2 | 8 | 11.9 | | | |
| | | K | 16.5 | 16 | 23.9 | | | |
| Y | 59 | | | 52 | 77.6 | Y | | |
| | | C | 0.7 | 2 | 3.0 | | | |
| | | S | 5.6 | 13 | 19.4 | | | |
| A | 60 | | | 42 | 62.7 | P | | |
| | | R | 0.1 | 3 | 4.5 | | | |
| | | E | 1.6 | 3 | 4.5 | | | |
| | | T | 1.9 | 6 | 9.0 | | | |
| | | P | 2.1 | 0 | 0.0 | | | |
| | | V | 2.2 | 8 | 11.9 | | | |
| | | S | 3.0 | 5 | 7.5 | | | |
| Q | 61 | | | 56 | 83.6 | Q | | R |
| | | R | 2.8 | 3 | 4.5 | | | |
| | | E | 4.3 | 8 | 11.9 | | | |
| K | 62 | | | 64 | 95.5 | N | | P |
| | | R | 7.5 | 1 | 1.5 | | | |
| | | N | 9.4 | 2 | 3.0 | | | |
| F | 63 | | | 66 | 98.5 | F | | L |
| | | L | 2.4 | 1 | 1.5 | | | |
| Q | 64 | | | 56 | 83.6 | R | | |
| | | R | 4.8 | 11 | 16.4 | | | |
| G | 65 | | | 67 | 100.0 | G | | |
| R | 66 | | | 67 | 100.0 | R | | |
| V | 67 | | | 66 | 98.5 | V | | |
| | | L | 2.3 | 1 | 1.5 | | | |
| T | 68 | | | 64 | 95.5 | S | | |
| | | I | 2.4 | 2 | 3.0 | | | |
| | | S | 4.4 | 1 | 1.5 | | | |
| M | 69 | | | 64 | 95.5 | M | | |
| | | L | 12.3 | 3 | 4.5 | | | |
| T | 70 | | | 67 | 100.0 | T | | |
| R | 71 | | | 67 | 100.0 | R | YES | |
| D | 72 | | | 67 | 100.0 | D | | |
| T | 73 | | | 66 | 98.5 | T | | V |
| | | P | 0.8 | 1 | 1.5 | | | |
| S | 74 | | | 66 | 98.5 | S | | Y |
| | | T | 0.7 | 1 | 1.5 | | | |
| I | 75 | | | 67 | 100.0 | M | | S |
| | | M | 1.5 | 0 | 0.0 | | | |
| S | 76 | | | 37 | 55.2 | E | | D |
| | | E | 0.1 | 0 | 0.0 | | | |
| | | I | 0.3 | 1 | 1.5 | | | |
| | | K | 0.6 | 1 | 1.5 | | | |
| | | R | 3.1 | 1 | 1.5 | | | |
| | | T | 13.3 | 20 | 29.9 | | | |
| | | N | 18.0 | 7 | 10.4 | | | |
| T | 77 | | | 64 | 95.5 | I | | |
| | | I | 0.6 | 3 | 4.5 | | | |
| A | 78 | | | 60 | 89.6 | F | | |
| | | F | 0.5 | 0 | 0.0 | | | |
| | | T | 2.1 | 1 | 1.5 | | | |
| | | V | 15.3 | 6 | 9.0 | | | |
| Y | 79 | | | 67 | 100.0 | Y | | F |
| M | 80 | | | 64 | 95.5 | M | | L |
| | | L | 9.1 | 3 | 4.5 | | | |
| E | 81 | | | 65 | 97.0 | E | | |
| | | V | 0.4 | 2 | 3.0 | | | |
| L | 82 | | | 66 | 98.5 | L | | |
| | | M | 2.3 | 1 | 1.5 | | | |
| S | (82A) | | | 45 | 67.2 | S | | R |
| | | K | 1.2 | 1 | 1.5 | | | |
| | | R | 8.1 | 2 | 3.0 | | | |
| | | N | 8.9 | 16 | 23.9 | | | |
| | | T | 12.8 | 3 | 4.5 | | | |
| R | (82B) | | | 59 | 88.1 | R | | S |
| | | G | 13.4 | 8 | 11.9 | | | |
| L | (82C) | | | 66 | 98.5 | L | | |
| | | V | 0.9 | 1 | 1.5 | | | |
| R | 83 | | | 60 | 89.6 | T | | T |
| | | N | 0.7 | 1 | 1.5 | | | |
| | | I | 1.7 | 1 | 1.5 | | | |
| | | K | 7.9 | 2 | 3.0 | | | |
| | | T | 29.6 | 3 | 4.5 | | | |
| S | 84 | | | 66 | 98.5 | S | | V |
| | | Y | 2.3 | 1 | 1.5 | | | |
| D | 85 | | | 66 | 98.5 | D | | |
| | | N | 1.0 | 1 | 1.5 | | | |
| D | 86 | | | 67 | 100.0 | D | | |
| T | 87 | | | 67 | 100.0 | T | | |
| A | 88 | | | 67 | 100.0 | A | | |
| V | 89 | | | 60 | 89.6 | V | | |
| | | R | 0.1 | 1 | 1.5 | | | |
| | | A | 0.3 | 1 | 1.5 | | | |
| | | M | 4.6 | 1 | 1.5 | | | |
| | | I | 10.8 | 4 | 6.0 | | | |
| Y | 90 | | | 67 | 100.0 | Y | | |
| Y | 91 | | | 48 | 71.6 | Y | | F |
| | | N | 0.0 | 2 | 3.0 | | | |
| | | F | 14.6 | 17 | 25.4 | | | |
| C | 92 | | | 67 | 100.0 | C | | |
| A | 93 | | | 65 | 97.0 | A | | T |
| | | T | 3.5 | 1 | 1.5 | | | |
| | | V | 5.1 | 1 | 1.5 | | | |
| R | 94 | | | 67 | 100.0 | R | | |

TABLE 4B
MUTATIONAL ANALYSIS OF ANTIBODIES ISOLATED
FROM IOMA IGL TRANSGENIC MICE

| VL2- | | | | | | | |
|---|---|---|---|---|---|---|---|
| 23*02 | | | | # of IGT2 | | | |
| amino | VL | amino acid | Random | induced mAbs | IGT2 | IOMA | Critical |
| acid | Position | substitution | Frequency | with this SHM | Frequency | Substitution | Interaction |
| O | 1 | | | 67 | 100.0 | Q | |
| S | 2 | | | 66 | 98.5 | S | |
| | | F | 0.1 | 1 | 1.5 | | |
| A | 3 | | | 67 | 100.0 | A | |
| L | 4 | | | 67 | 100.0 | L | |
| T | 5 | | | 67 | 100.0 | T | |
| Q | 6 | | | 67 | 100.0 | Q | |
| P | 7 | | | 67 | 100.0 | P | |
| A | 8 | | | 67 | 100.0 | A | |
| S | 9 | | | 67 | 100.0 | S | |
| V | 11 | | | 67 | 100.0 | V | |
| S | 12 | | | 66 | 98.5 | S | |
| | | F | 0.1 | 1 | 1.5 | | |
| G | 13 | | | 67 | 100.0 | G | |
| S | 14 | | | 67 | 100.0 | S | |
| P | 15 | | | 67 | 100.0 | P | |
| G | 16 | | | 62 | 92.5 | G | |
| | | E | 0.1 | 5 | 7.5 | | |
| O | 17 | | | 67 | 100.0 | Q | |
| S | 18 | | | 67 | 100.0 | S | |
| I | 19 | | | 65 | 97.0 | I | |
| | | S | 0.1 | 1 | 1.5 | | |
| | | T | 0.1 | 1 | 1.5 | | |
| T | 20 | | | 67 | 100.0 | T | |
| I | 21 | | | 67 | 100.0 | I | |
| S | 22 | | | 67 | 100.0 | S | |
| C | 23 | | | 67 | 100.0 | C | |
| T | 24 | | | 67 | 100.0 | A | |
| | | A | 2.3 | 0 | 0.0 | | |
| G | 25 | | | 63 | 94.0 | G | |
| | | V | 0.1 | 4 | 6.0 | | |
| T | 26 | | | 65 | 97.0 | S | |
| | | P | 0.6 | 1 | 1.5 | | |
| | | A | 2.6 | 1 | 1.5 | | |
| | | S | 7.2 | 1 | 1.5 | | |
| S | 27 | | | 65 | 97.0 | S | |
| | | G | 2.2 | 1 | 1.5 | | |
| | | R | 2.5 | 1 | 1.5 | | |
| S | (27A) | | | 63 | 94.0 | R | YES |
| | | R | 2.4 | 2 | 3.0 | | |
| | | N | 6.1 | 2 | 3.0 | | |
| D | (27B) | | | 67 | 100.0 | D | |
| V | (27C) | | | 53 | 79.1 | V | |
| | | F | 1.9 | 1 | 1.5 | | |
| | | I | 17.0 | 13 | 19.4 | | |
| G | 28 | | | 67 | 100.0 | G | |
| S | 29 | | | 51 | 76.1 | G | YES |
| | | I | 2.0 | 1 | 1.5 | | |
| | | R | 3.2 | 4 | 6.0 | | |
| | | G | 5.3 | 4 | 6.0 | | |
| | | T | 14. | 1 | 1.5 | | |
| | | N | 14.5 | 6 | 9.0 | | |
| Y | 30 | | | 56 | 83.6 | F | YES |
| | | S | 3.0 | 4 | 6.0 | | |
| | | F | 4.1 | 7 | 10.4 | | |
| N | 31 | | | 35 | 52.2 | D | YES |
| | | Y | 1.3 | 4 | 6.0 | | |
| | | D | 10.2 | 28 | 41.8 | | |
| L | 32 | | | 65 | 97.0 | L | |
| | | F | 8.2 | 2 | 3.0 | | |
| V | 33 | | | 67 | 100.0 | V | |
| S | 34 | | | 66 | 98.5 | S | |
| | | P | 0.0 | 1 | 1.5 | | |
| W | 35 | | | 67 | 100.0 | W | |
| Y | 36 | | | 67 | 100.0 | Y | |
| Q | 37 | | | 67 | 100.0 | Q | |
| Q | 38 | | | 67 | 100.0 | Q | |
| H | 39 | | | 67 | 100.0 | H | |
| P | 40 | | | 67 | 100.0 | P | |
| G | 41 | | | 67 | 100.0 | G | |
| K | 42 | | | 66 | 98.5 | K | |
| | | N | 0.7 | 1 | 1.5 | | |
| A | 43 | | | 61 | 91.0 | A | |
| | | T | 0.8 | 5 | 7.5 | | |
| | | V | 7.6 | 1 | 1.5 | | |
| P | 44 | | | 67 | 100.0 | P | |
| K | 45 | | | 67 | 100.0 | K | |
| L | 46 | | | 66 | 98.5 | L | |
| | | F | 2.4 | 1 | 1.5 | | |
| M | 47 | | | 63 | 94.0 | I | |
| | | L | 10.7 | 2 | 3.0 | | |
| | | I | 34.4 | 2 | 3.0 | | |
| I | 48 | | | 66 | 98.5 | I | |
| | | L | 2.6 | 1 | 1.5 | | |
| Y | 49 | | | 66 | 98.5 | Y | |
| | | H | 1.8 | 1 | 1.5 | | |
| E | 50 | | | 51 | 76.1 | E | |
| | | K | 0.3 | 4 | 6.0 | | |
| | | D | 6.5 | 12 | 17.9 | | |
| V | 51 | | | 67 | 100.0 | V | |
| S | 52 | | | 46 | 68.7 | N | |
| | | I | 3.2 | 1 | 1.5 | | |
| | | N | 19.8 | 14 | 20.9 | | |
| | | T | 29.2 | 6 | 9.0 | | |
| K | 53 | | | 38 | 56.7 | K | |
| | | A | 0.2 | 1 | 1.5 | | |
| | | R | 3.8 | 23 | 34.3 | | |
| | | Q | 4.9 | 4 | 6.0 | | |
| | | E | 7.1 | 1 | 1.5 | | |
| R | 54 | | | 67 | 100.0 | R | |
| P | 55 | | | 67 | 100.0 | P | |
| S | 56 | | | 67 | 100.0 | S | |
| G | 57 | | | 67 | 100.0 | G | |
| V | 58 | | | 61 | 91.0 | I | |
| | | I | 10.9 | 6 | 9.0 | | |
| S | 59 | | | 66 | 98.5 | S | |
| | | Y | 0.0 | 1 | 1.5 | | |
| N | 60 | | | 65 | 97.0 | S | |
| | | S | 7.3 | 1 | 1.5 | | |
| | | D | 17.3 | 1 | 1.5 | | |
| R | 61 | | | 67 | 100.0 | R | |
| F | 62 | | | 67 | 100.0 | F | |
| S | 63 | | | 65 | 97.0 | S | |
| | | A | 0.4 | 2 | 3.0 | | |
| G | 64 | | | 65 | 97.0 | A | |
| | | D | 0.1 | 1 | 1.5 | | |
| | | A | 5.3 | 1 | 1.5 | | |
| S | 65 | | | 67 | 100.0 | S | |
| K | 66 | | | 67 | 100.0 | K | |
| S | 67 | | | 65 | 97.0 | S | |
| | | C | 0.0 | 1 | 1.5 | | |
| | | A | 0.5 | 1 | 1.5 | | |
| G | 68 | | | 65 | 97.0 | G | |
| | | D | 3.3 | 2 | 3.0 | | |
| N | 69 | | | 66 | 98.5 | N | |
| | | K | 0.8 | 1 | 1.5 | | |
| T | 70 | | | 64 | 95.5 | T | |
| | | M | 0.8 | 2 | 3.0 | | |
| | | S | 0.8 | 1 | 1.5 | | |
| A | 71 | | | 67 | 100.0 | A | |
| S | 72 | | | 67 | 100.0 | S | |
| L | 73 | | | 67 | 100.0 | L | |
| T | 74 | | | 56 | 83.6 | T | |
| | | P | 0.0 | 2 | 3.0 | | |
| | | I | 0.5 | 9 | 13.4 | | |
| I | 75 | | | 67 | 100.0 | I | |
| S | 76 | | | 67 | 100.0 | S | |
| G | 77 | | | 66 | 98.5 | G | |
| | | D | 0.5 | 1 | 1.5 | | |
| L | 78 | | | 32 | 47.8 | L | |
| | | F | 0.0 | 35 | 52.2 | | |
| Q | 79 | | | 65 | 97.0 | Q | |
| | | R | 2.5 | 2 | 3.0 | | |
| A | 80 | | | 59 | 88.1 | E | |
| | | E | 0.1 | 0 | 0.0 | | |
| | | D | 0.3 | 1 | 1.5 | | |
| | | T | 4.9 | 6 | 9.0 | | |
| | | P | 5.1 | 1 | 1.5 | | |
| E | 81 | | | 67 | 100.0 | E | |
| D | 82 | | | 67 | 100.0 | D | |
| E | 83 | | | 57 | 85.1 | E | |
| | | V | 0.0 | 3 | 4.5 | | |
| | | K | 0.1 | 2 | 3.0 | | |
| | | G | 0.2 | 5 | 7.5 | | |
| A | 84 | | | 66 | 98.5 | A | |
| | | G | 4.2 | 1 | 1.5 | | |
| D | 85 | | | 61 | 91.0 | H | |
| | | G | 0.2 | 2 | 3.0 | | |
| | | Y | 0.9 | 1 | 1.5 | | |
| | | N | 2.1 | 1 | 1.5 | | |
| | | H | 2.6 | 0 | 0.0 | | |
| | | E | 4.1 | 2 | 3.0 | | |
| Y | 86 | | | 65 | 97.0 | Y | |
| | | N | | 2 | 3.0 | | |
| Y | 87 | | | 45 | 67.2 | Y | |
| | | E | 0.0 | 1 | 1.5 | | |
| | | D | 0.0 | 5 | 7.5 | | |
| | | H | 4.3 | 12 | 17.9 | | |
| | | F | 6.2 | 4 | 6.0 | | |
| C | 88 | | | 67 | 100.0 | C | |
| C | 89 | | | 57 | 85.1 | Y | |
| | | W | 0.8 | 1 | 1.5 | | |
| | | Y | 1.8 | 4 | 6.0 | | |
| | | S | 10.3 | 5 | 7.5 | | |
| S | 90 | | | 60 | 89.6 | S | |
| | | L | 0.7 | 6 | 9.0 | | |
| | | T | 1.0 | 1 | 1.5 | | |
| Y | 96 | | | 61 | 91.0 | Y | |
| | | C | 0.7 | 1 | 1.5 | | |
| | | S | 2.1 | 3 | 4.5 | | |
| | | F | 7.4 | 2 | 3.0 | | |
| A | 97 | | | 48 | 71.6 | A | |
| | | E | 1.4 | 1 | 1.5 | | |
| | | T | 5.8 | 3 | 4.5 | | |
| | | G | 7.2 | 9 | 13.4 | | |
| | | V | 8.8 | 6 | 9.0 | | |

TABLE 5A
SERUM NEUTRALIZATION IN WILDTYPE MICE

| | | | M1 | | M3 | | M4 | | M5 | |
|---|---|---|---|---|---|---|---|---|---|---|
| | | | B3 (week 18) | | B3 (week 18) | | B3 (week 18) | | B3 (week 18) | |
| | | | | % | | % | | % | | % |
| Virus | Clade | Tier | ID₅₀ | 1:100 | ID₅₀ | 1:100 | ID₅₀ | 1:100 | ID₅₀ | 1:100 |
| 426c | C | 2 | <100 | 0 | <100 | 0 | <100 | 0 | <100 | 0 |
| 25710 | B | 2 | <100 | 0 | <100 | 0 | <100 | 0 | <100 | 0 |
| CNE8 | AE | 1 | <100 | 0 | <100 | 0 | <100 | 0 | <100 | 0 |
| CNE8 N276A | AE | 1 | <100 | 0 | <100 | 0 | <100 | 0 | <100 | 0 |
| CNE20 | BC | 2 | <100 | 0 | <100 | 0 | <100 | 0 | <100 | 0 |
| CNE20 N276A | BC | 2 | <100 | 0 | <100 | 0 | <100 | 0 | <100 | 0 |
| JRCSF | B | 2 | <100 | 0 | <100 | 0 | <100 | 0 | <100 | 0 |
| Q23.17 | A | 1 | <100 | 0 | <100 | 0 | <100 | 0 | <100 | 0 |
| YU2 | B | 2 | <100 | 0 | <100 | 0 | <100 | 0 | <100 | 0 |
| BG505 T332N | A | 2 | <100 | 0 | <100 | 0 | <100 | 0 | <100 | 0 |
| 6535.5 | B | 1 | <100 | 0 | <100 | 0 | <100 | 0 | <100 | 0 |
| 3415_V1_C1 | A | 2 | <100 | 0 | <100 | 0 | <100 | 0 | <100 | 0 |
| CAAN5342.A2 | B | 2 | <100 | 0 | — | — | <100 | 0 | <100 | 0 |
| PVO.4 | B | 3 | 113 | 57 | — | — | <100 | 0 | <100 | 0 |
| Q842.D12 | A | 2 | <100 | 43 | — | — | <100 | 0 | <100 | 0 |
| RHPA4259.7 | B | 2 | <100 | 0 | — | — | <100 | 0 | <100 | 0 |
| WITO4160.33 | B | 2 | <100 | 47 | — | — | <100 | 0 | <100 | 0 |
| ZM214M.PL15 | C | 2 | <100 | 0 | — | — | <100 | 0 | <100 | 0 |
| MuLV | | | <100 | 0 | — | — | <100 | 0 | <100 | 0 |

TABLE 5B
SERUM NEUTRALIZATION IN WILDTYPE MICE

| | | M13 | | | M14 | | M15 | | M21 | |
|---|---|---|---|---|---|---|---|---|---|---|
| | | B3 (week 18) | | | B3 (week 18) | | B3 (week 18) | | B4 (week 23) | |
| | | | | % | | % | | % | | % |
| Virus | Clade | Tier | ID₅₀ | 1:100 | ID₅₀ | 1:100 | ID₅₀ | 1:100 | ID₅₀ | 1:100 |
| 426c | C | 2 | <100 | 0 | <100 | 0 | <100 | 0 | <100 | 0 |
| 25710 | B | 2 | <100 | 0 | <100 | 0 | <100 | 0 | <100 | 0 |
| CNE8 | AE | 1 | <100 | 0 | <100 | 0 | <100 | 0 | 225 | 56 |
| CNE8 N276A | AE | 1 | <100 | 0 | <100 | 0 | <100 | 0 | <100 | 0 |
| CNE20 | BC | 2 | <100 | 0 | <100 | 0 | <100 | 40 | <100 | 0 |
| CNE20 N276A | BC | 2 | <100 | 0 | <100 | 0 | <100 | 0 | <100 | 0 |
| JRCSF | B | 2 | <100 | 0 | <100 | 0 | <100 | 0 | <100 | 0 |
| Q23.17 | A | 1 | <100 | 0 | <100 | 0 | <100 | 40 | <100 | 0 |
| YU2 | B | 2 | <100 | 0 | <100 | 0 | <100 | 0 | <100 | 0 |
| BG505 T332N | A | 2 | <100 | 0 | <100 | 0 | <100 | 0 | <100 | 0 |
| 6535.5 | B | 1 | <100 | 0 | <100 | 0 | <100 | 47 | <100 | 0 |
| 3415_V1_C1 | A | 2 | <100 | 0 | <100 | 0 | <100 | 0 | <100 | 0 |
| CAAN5342.A2 | B | 2 | <100 | 0 | <100 | 0 | <100 | 0 | <100 | 0 |
| PVO.4 | B | 3 | <100 | 41 | <100 | 0 | 145 | 58 | <100 | 0 |
| Q842.D12 | A | 2 | <100 | 0 | <100 | 0 | <100 | 0 | <100 | 0 |
| RHP A4259.7 | B | 2 | <100 | 0 | <100 | 0 | <100 | 0 | <100 | 0 |
| WITO4160.33 | B | 2 | <100 | 0 | <100 | 0 | <100 | 0 | <100 | 0 |
| ZM214M.PL15 | C | 2 | <100 | 0 | <100 | 0 | <100 | 0 | <100 | 0 |
| MuLV | | | <100 | 0 | <100 | 0 | <100 | 0 | <100 | 0 |

TABLE 5C
SERUM NEUTRALIZATION IN WILDTYPE MICE

| | | | M22 | | M23 | | M24 | | M25 | |
|---|---|---|---|---|---|---|---|---|---|---|
| | | | B4 (week 23) | | B4 (week 23) | | B4 (week 23) | | B4 (week 23) | |
| | | | | % | | % | | % | | % |
| Virus | Clade | Tier | ID₅₀ | 1:100 | ID₅₀ | 1:100 | ID₅₀ | 1:100 | ID₅₀ | 1:100 |
| 426c | C | 2 | <100 | 0 | <100 | 0 | <100 | 0 | <100 | 0 |
| 25710 | B | 2 | <100 | 0 | <100 | 0 | <100 | 0 | <100 | 0 |
| CNE8 | AE | 1 | <100 | 0 | <100 | 0 | 100 | 50 | <100 | 0 |
| CNE8 N276A | AE | 1 | <100 | 0 | <100 | 0 | <100 | 0 | <100 | 0 |
| CNE20 | BC | 2 | <100 | 0 | <100 | 0 | <100 | 0 | <100 | 0 |
| CNE20 N276A | BC | 2 | <100 | 0 | <100 | 0 | <100 | 0 | <100 | 0 |
| JRCSF | B | 2 | <100 | 0 | <100 | 0 | <100 | 0 | <100 | 0 |
| Q23.17 | A | 1 | <100 | 0 | <100 | 0 | <100 | 0 | <100 | 0 |
| YU2 | B | 2 | <100 | 0 | <100 | 0 | <100 | 0 | <100 | 0 |
| BG505 T332N | A | 2 | <100 | 0 | <100 | 0 | <100 | 0 | <100 | 0 |
| 6535.5 | B | 1 | <100 | 0 | <100 | 0 | <100 | 0 | <100 | 0 |
| 3415_V1_C1 | A | 2 | <100 | 0 | <100 | 0 | <100 | 0 | <100 | 0 |
| CAAN5342.A2 | B | 2 | <100 | 0 | <100 | 0 | <100 | 0 | <100 | 0 |
| PVO.4 | B | 3 | <100 | 0 | <100 | 0 | <100 | 0 | <100 | 0 |
| Q842.D12 | A | 2 | <100 | 0 | <100 | 0 | <100 | 0 | <100 | 0 |
| RHP A4259.7 | B | 2 | <100 | 0 | <100 | 0 | <100 | 0 | <100 | 0 |
| WITO4160.33 | B | 2 | <100 | 0 | <100 | 0 | <100 | 0 | <100 | 0 |
| ZM214M.PL15 | C | 2 | <100 | 0 | <100 | 0 | <100 | 0 | <100 | 0 |
| MuLV | | | <100 | 0 | <100 | 0 | <100 | 0 | <100 | 0 |

TABLE 5D
SERUM NEUTRALIZATION IN WILDTYPE MICE

| | | M26 | | | M27 | | M28 | | M29 | |
|---|---|---|---|---|---|---|---|---|---|---|
| | | B4 (week 23) | | | B4 (week 23) | | B4 (week 23) | | B4 (week 23) | |
| | | | | % | | % | | % | | % |
| Virus | Clade | Tier | ID₅₀ | 1:100 | ID₅₀ | 1:100 | ID₅₀ | 1:100 | ID₅₀ | 1:100 |
| 426c | C | 2 | <100 | 0 | <100 | 0 | <100 | 0 | <100 | 0 |
| 25710 | B | 2 | <100 | 0 | <100 | 0 | <100 | 0 | <100 | 0 |
| CNE8 | AE | 1 | 729 | 70 | <100 | 0 | 84 | 61 | 242 | 71 |
| CNE8 N276A | AE | 1 | <100 | 0 | <100 | 0 | <100 | 0 | <100 | 0 |
| CNE20 | BC | 2 | <100 | 0 | <100 | 0 | <100 | 0 | <100 | 0 |
| CNE20 N276A | BC | 2 | <100 | 0 | <100 | 0 | <100 | 0 | <100 | 0 |
| JRCSF | B | 2 | <100 | 0 | <100 | 0 | <100 | 0 | <100 | 0 |
| Q23.17 | A | 1 | <100 | 0 | <100 | 0 | <100 | 0 | <100 | 0 |
| YU2 | B | 2 | <100 | 0 | <100 | 0 | <100 | 0 | <100 | 0 |
| BG505 T332N | A | 2 | <100 | 0 | <100 | 0 | <100 | 41 | <100 | 0 |
| 6535.5 | B | 1 | <100 | 0 | <100 | 0 | <100 | 0 | <100 | 0 |
| 3415_V1_C1 | A | 2 | <100 | 0 | <100 | 0 | <100 | 0 | <100 | 0 |
| CAAN5342.A2 | B | 2 | <100 | 0 | <100 | 0 | <100 | 0 | <100 | 0 |
| PVO.4 | B | 3 | <100 | 0 | <100 | 0 | <100 | 43 | <100 | 0 |
| Q842.D12 | A | 2 | <100 | 0 | <100 | 0 | <100 | 0 | <100 | 0 |
| RHPA4259.7 | B | 2 | <100 | 0 | <100 | 0 | <100 | 0 | <100 | 0 |
| WITO4160.33 | B | 2 | <100 | 0 | <100 | 0 | <100 | 0 | <100 | 0 |
| ZM214M.PL15 | C | 2 | <100 | 0 | <100 | 0 | <100 | 0 | <100 | 0 |
| MuLV | | | <100 | 0 | <100 | 0 | <100 | 0 | <100 | 0 |

TABLE 6
SERUM NEUTRALIZATION IN WILDTYPE MICE

| | | | SEQ ID |
|---|---|---|---|
| Oligo Name | Fragment | Sequence | NO: |
| 426c Library | 1 | GTCTGGAAAGAGGCTAAGACCACACTG | 161 |
| 1 For | | | |
| 426c Library | 1 | CAGGTTTTTTGATCTGATCACAATCTCTTC | 162 |
| 1 Rev | | | |
| 426c Library | 2 | GAAGAGATTGTGATCAGATCAAAAAACCTGNNKAACA | 163 |
| 1-1 For | | ATGCCAAGATCATTATCGTGC | |
| 426c Library | 2 | ATCTCCACACTCTTATTCAGCTGCACGATAATGATCTT | 164 |
| 1-2 Rev | | GGCATT | |
| 426c Library | 2 | AGCTGAATAAGAGTGTGGAGATCGTCTGCACACGACC | 165 |
| 1-3 For | | TAACA | |
| 426c Library | 2 | GCCTGCCGAATATCTCCCCCAGATCCGCTGCCGCCATT | 166 |
| 1-4 Rev | | GTTAGGTCGTGTGCAGACG | |
| 426c Library | 2 | GGAGATATTCGGCAGGCTTATTGTAACATCAGTGGCAG | 167 |
| 1-5 For | | AAATTGGTCAGAAGCCGTGAA | |
| 426c Library | 2 | TGGGGGAAGTGCTCTTTCAGCTTTTTCTTGACCTGGTTC | 168 |
| 1-6 Rev | | ACGGCTTCTGACCAATTT | |
| 426c Library | 2 | AAAGAGCACTTCCCCCATAAGAATATTAGCTTTCAGTC | 169 |
| 1-7 For | | TAGTTCAGGCGGGGAC | |
| 426c Library | 2 | TCGCCTCCGCAGTTGAAGGAGTGTGTGGTGATTTCCAG | 170 |
| 1-8 Rev | | GTCCCCGCCTGAACTA | |
| 426c Library | 2 | ACTGCGGAGGCGAGTTCTTTTACTGTAATACATCCGGC | 171 |
| 1-9 For | | CTGTTTAACG | |
| 426c Library | 2 | CCGGCAAGGCAGCATGATTGTGGCATTAGAAATGGTA | 172 |
| 1-10 Rev | | TCGTTAAACAGGCCGGATGTA | |
| 426c Library | 2 | GCTGCCTTGCCGGATCAAGCAGATTATCAACATGTGGC | 173 |
| 1-11 For | | AGGAA | |
| 426c Library | 2 | TGCCCTTGATGGGTGGTGCATAGATAGCCTTTCCMNNT | 174 |
| 1-12 Rev | | TCCTGCCACATGTTGATAATC | |
| 426c Library | 2 | CCACCCATCAAGGGCAATATCACCTGTAAGAGTGACA | 175 |
| 1-13 For | | TTACAGGGCTGCTGCTGCTGAGA | |
| 426c Library | 2 | GCCGGAAAATCTCGGTmnnmnnmnnmnnmnnTCCCCCATC | 176 |
| 1-14 Rev | | TCTCAGCAGCAGCAGCC | |
| 426c Library | 2 | ACCGAGATTTTCCGGCCTAGCGGAGGAGACATGCGAG | 177 |
| 1-15 For | | ATAATTGGCGGTCTGAACTG | |
| 426c Library | 2 | GGATCCCAGAGGCTTGATCTCGACCACCTTATATTTGT | 178 |
| 1-16 Rev | | ACAGTTCAGACCGCCAATTA | |
| 426c Library | 3 | CTGGTGGAGGCGGTAGCGGAGGCGGAGGGTCGGCTAG | 179 |
| 1 For | | CGTCTGGAAAGAGGCTAAGACCA | |
| 426c Library | 3 | TTACAAGTCCTCTTCAGAAATAAGCTTTTGTTCGGATC | 180 |
| 1 Rev | | CCAGAGGCTTGATCTCGACCAC | |
| 426c Library | 1 | GTCTGGAAAGAGGCTAAGACCACACTG | 181 |
| 2 For | | | |
| 426c Library | 1 | CAGGTTTTTTGATCTGATCACAATCTCTTC | 182 |
| 2 Rev | | | |
| 426c Library | 2 | GAAGAGATTGTGATCAGATCAAAAAACCTGNNKAACA | 183 |
| 2-1 For | | ATGCCAAGATCATTATCGTGC | |
| 426c Library | 2 | ATCTCCACACTCTTATTCAGCTGCACGATAATGATCTT | 184 |
| 2-2 Rev | | GGCATT | |
| 426c Library | 2 | AGCTGAATAAGAGTGTGGAGATCGTCTGCACACGACC | 185 |
| 2-3 For | | TAACA | |
| 426c Library | 2 | GCCTGCCGAATATCTCCCCCAGATCCGCTGCCGCCATT | 186 |
| 2-4 Rev | | GTTAGGTCGTGTGCAGACG | |
| 426c Library | 2 | GGAGATATTCGGCAGGCTTATTGTAACATCAGTGGCAG | 187 |
| 2-5 For | | AAATTGGTCAGAAGCCGTGAA | |
| 426c Library | 2 | TGGGGGAAGTGCTCTTTCAGCTTTTTCTTGACCTGGTTC | 188 |
| 2-6 Rev | | ACGGCTTCTGACCAATTT | |
| 426c Library | 2 | AAAGAGCACTTCCCCCATAAGAATATTAGCTTTCAGTC | 189 |
| 2-7 For | | TAGTTCAGGCGGGGAC | |
| 426c Library | 2 | TCGCCTCCGCAGTTGAAGGAGTGTGTGGTGATTTCCAG | 190 |
| 2-8 Rev | | GTCCCCGCCTGAACTA | |
| 426c Library | 2 | ACTGCGGAGGCGAGTTCTTTTACTGTAATACATCCGGC | 191 |
| 2-9 For | | CTGTTTAACG | |
| 426c Library | 2 | CCGGCAAGGCAGCATGATTGTGGCATTAGAAATGGTA | 192 |
| 2-10 Rev | | TCGTTAAACAGGCCGGATGTA | |
| 426c Library | 2 | GCTGCCTTGCCGGATCAAGCAGATTATCAACATGTGGC | 193 |
| 2-11 For | | AGGAA | |
| 426c Library | 2 | TGCCCTTGATGGGTGGTGCATAGATAGCCTTTCCMNNT | 194 |
| 2-12 Rev | | TCCTGCCACATGTTGATAATC | |
| 426c Library | 2 | CCACCCATCAAGGGCAATATCACCTGTAAGAGTGACA | 195 |
| 2-13 For | | TTACAGGGCTGCTGCTGCTGAGA | |
| 426c Library | 2 | GCCGGAAAATCTCGGTmnnmnnmnnmnnmnnTCCCCCATC | 196 |
| 2-14 Rev | | TCTCAGCAGCAGCAGCC | |
| 426c Library | 2 | ACCGAGATTTTCCGGCCTAGCGGAGGAGACATGCGAG | 197 |
| 2-15 For | | ATAATTGGCGGTCTGAACTG | |
| 426c Library | 2 | GGATCCCAGAGGCTTGATCTCGACCACCTTATATTTGT | 198 |
| 2-16 Rev | | ACAGTTCAGACCGCCAATTA | |
| 426c Library | 3 | CTGGTGGAGGCGGTAGCGGAGGCGGAGGGTCGGCTAG | 199 |
| 2 For | | CGTCTGGAAAGAGGCTAAGACCA | |
| 426c Library | 3 | TTACAAGTCCTCTTCAGAAATAAGCTTTTGTTCGGATC | 200 |
| 2 Rev | | CCAGAGGCTTGATCTCGACCAC | |

TABLE 7
FLOW CYTOMETRIC REAGENTS

| | Target | Antibody | Company/ | | |
|---|---|---|---|---|---|
| Reagent | species | clone | Source | Cat.# | RRID |
| CD16/32 | mouse | 2.4G2 | BD Biosciences | 553142 | AB_394657 |
| CD4-APCeF780 | mouse | RM4-5 | Thermo Fisher | 47-0042-82 | AB_1272183 |
| CD8a-APCeF780 | mouse | 53-6.7 | Thermo Fisher | 47-0081-82 | AB_1272185 |
| NK1.1-APCeF780 | mouse | PK136 | Thermo Fisher | 47-5941-82 | AB_2735070 |
| F4/80-APCeF780 | mouse | BM8 | Thermo Fisher | 47-4801-82 | AB_2735036 |
| Ly-6G/C (Gr1)- | mouse | RB6-8C5 | Thermo Fisher | 47-5931-82 | AB_1518804 |
| APCeF780 | | | | | |
| CD11b-APCeF780 | mouse | M1/70 | Thermo Fisher | 47-0112-82 | AB_1603193 |
| CD11c-APCeF780 | mouse | N418 | Thermo Fisher | 47-0114-82 | AB_1548652 |
| CD93-APC | mouse | AA4.1 | Thermo Fisher | 17-5892-82 | AB_469466 |
| TER-119-APCCy0 | mouse | TER-119 | BD Pharmingen | 560509 | AB_1645230 |
| CD95 (FAS)-FITC | mouse | SA367H8 | BioLegend | 152606 | AB_2632901 |
| CD38-AF700 | mouse | 90 | Thermo Fisher | 56-0381-82 | AB_657740 |
| CD45R/B220-BV421 | mouse/ | RA3-6B2 | BD Horizon | 562922 | AB_2737894 |
| | human | | | | |
| CD45R/B220-BV605 | mouse/ | RA3-6B2 | BioLegend | 103244 | AB_2563312 |
| | human | | | | |
| IgD-BV786 | mouse | 11-26c.2a | BD Horizon | 563618 | AB_2738322 |
| CD19-PECy7 | mouse | 6D5 | BioLegend | 115520 | AB_313655 |
| CD2-PE | mouse | RM2-5 | BioLegend | 100108 | AB_2073690 |
| CD23-PE | mouse | B3B4 | BioLegend | 101607 | AB_312832 |
| Ig light chain lambda- | mouse | RML-42 | BioLegend | 407306 | AB_961363 |
| APC | | | | | |
| Ig light chain kappa- | mouse | 187.1 | BD Horizon | 562888 | AB_2737867 |
| BV421 | | | | | |
| CD21/CD35 | mouse | 7G6 | BD Horizon | 562756 | AB_2737772 |
| IgM Fab-FITC | mouse | polyclonal | Jackson | 115-097-020 | AB_2338618 |
| | | | Immunoresearch | | |
| Zombie NIR | N/A* | N/A | BioLegend | 423105 | N/A |
| Streptavidin-PE | N/A | N/A | BD Pharmingen | 554061 | AB_10053328 |
| Streptavidin-AF647 | N/A | N/A | BioLegend | 405237 | N/A |
| Streptavidin-PECy7 | N/A | N/A | BioLegend | 405206 | N/A |
| RC1-biotin | N/A | N/A | in house | N/A | N/A |
| CNE8 N276A-biotin | N/A | N/A | in house | N/A | N/A |
| 426c degly2 D279N- | N/A | N/A | in house | N/A | N/A |
| biotin | | | | | |
| 426c degly2 D279N | N/A | N/A | in house | N/A | N/A |
| CD4bs-KO -biotin | | | | | |
| Human Fc Block | human | N/A | BD Horizon | 564220 | AB_2869554 |
| Ig light chain lambda- | human | MHL38 | BioLegend | 316610 | AB_493629 |
| APC | | | | | |
| CD19-PECy7 | human | SJ25C1 | BioLegend | 363012 | AB_2564203 |
| IgM-FITC | human | MHM88 | BioLegend | 314506 | AB_493009 |
| Ig light chain kappa- | human | MHK-49 | BioLegend | 316518 | AB_2561581 |
| BV421 | | | | | |

*N/A not applicable

TABLE 8A
SINGLE CELL ANTIBODY CLONING REACTION CONDITIONS (PCR1 IgH
PRIMERS)

| | Primer sequence | SEQ ID NO: |
|---|---|---|
| HH_1FL (forward, leader) | CCATGGGATGGTCATGTATCA | 258 |
| HH_1RG (reverse, IgG) | GGACAGGGATCCAGAGTTCC | 259 |
| HH_1RM (reverse, IgM) | CCCATGGCCACCAGATTCTT | 260 |

TABLE 8B
SINGLE CELL ANTIBODY CLONING REACTION
CONDITIONS (PCR1 IgH PCR MASTERMIX)

| Reagent | Volume/plate (μL) | Concentration |
|---|---|---|
| nuclease free water | 3328 | |
| 10 × buffer | 384 | 1× |
| dNTP (25 mM) | 48 | 0.3 mM |
| 5′ forward Primer | HC 15; LC 19 | HC 0.25 μM; |
| (50 μM) | | LC 0.25 μM |
| 3′ reverse Primer | HC 23 | HC 0.30 μM; |
| (50 μM) | (IgG/IgM 1:1); | LC 0.25 μM |
| | LC 19 | |
| HotStart DNA | 42 | 0.055 U/μL |
| Polymerase | | |
| (5 U/μL) | | |
| total | 3840 | |

TABLE 8D
SINGLE CELL ANTIBODY CLONING REACTION
CONDITIONS (PCR1 IgK PCR MASTERMIX)

| Reagent | Volume/plate (μL) | Concentration |
|---|---|---|
| nuclease free water | 3328 | |
| 10 × buffer | 384 | 1× |
| dNTP (25 mM) | 48 | 0.3 mM |
| 5′ forward Primer | HC 15; LC 19 | HC 0.25 μM; |
| (50 μM) | | LC 0.25 μM |
| 3′ reverse Primer | HC 23 | HC 0.30 μM; |
| (50 μM) | (IgG/IgM 1:1); | LC 0.25 μM |
| | LC 19 | |
| HotStart DNA | 42 | 0.055 U/μL |
| Polymerase | | |
| (5 U/μL) | | |
| total | 3840 | |

TABLE 8C
SINGLE CELL ANTIBODY CLONING REACTION CONDITIONS (PCR1 IgK
PRIMERS)

| | Primer sequence | SEQ ID NO: |
|---|---|---|
| HH_1FL (forward, leader) | CCATGGGATGGTCATGTATCA | 258 |
| HH_1RK (reverse, IgK) | GACTGAGGCACCTCCAGATG | 260 |
| HH_1RM (reverse, IgM) | CCCATGGCCACCAGATTCTT | 262 |

TABLE 8E
SINGLE CELL ANTIBODY CLONING REACTION CONDITIONS (PCR2 IgH
PRIMERS

| | Primer sequence | SEQ ID NO: |
|---|---|---|
| HH_2FL (forward, leader) | GTAGCAACTGCAACCGGTGTACATTCT | 263 |
| HH_2RG (reverse, IgG) | GCTCAGGGAARTAGCCCTTGAC | 264 |
| HH_2RM (reverse, IgM) | AGGGGGAAGACATTTGGGAAGGAC | 265 |

TABLE 8F
SINGLE CELL ANTIBODY CLONING REACTION
CONDITIONS (PCR2 IgH PCR MASTERMIX)

| Reagent | Volume/plate (μL) | Concentration |
|---|---|---|
| nuclease free water | 2536 | |
| loading buffer* | 800 | |
| 10 × buffer | 384 | 1× |
| dNTP (25 mM) | 48 | 0.3 mM |
| 5′ forward Primer | HC 12; LC 15 | HC 0.16 μM; |
| (50 μM) | | LC 0.2 μM |
| 3′ reverse Primer | HC 18 | HC 0.23 μM; |
| (50 μM) | (IgG/IgM 1:1); | LC 0.2 μM |
| | LC 15 | |
| HotStart DNA | 42 | 0.055 U/μL |
| Polymerase | | |
| (5 U/μL) | | |

*loading buffer: 40% (w/v) sucrose in nuclease free water with cresol red added to dark red color.

TABLE 8G
SINGLE CELL ANTIBODY CLONING REACTION CONDITIONS (PCR2 IgK
PRIMERS

| | Primer sequence | SEQ ID NO: |
|---|---|---|
| HH_2FL (forward, leader) | GTAGCAACTGCAACCGGTGTACATTCT | 263 |
| HH_2RK (reverse, IgK) | AACTGCTCACTGGATGGTGG | 266 |

TABLE 8H
SINGLE CELL ANTIBODY CLONING REACTION
CONDITIONS (PCR2 IgK PCR MASTERMIX)

| Reagent | Volume/plate (μL) | Concentration |
|---|---|---|
| nuclease free water | 2536 | |
| loading buffer* | 800 | |
| 10 × buffer | 384 | 1× |
| dNTP (25 mM) | 48 | 0.3 mM |
| 5′ forward Primer | HC 12; LC 15 | HC 0.16 μM; |
| (50 μM) | | LC 0.2 μM |
| 3′ reverse Primer | HC 18 | HC 0.23 μM; |
| (50 μM) | (IgG/IgM 1:1); | LC 0.2 μM |
| | LC 15 | |
| HotStart DNA | 42 | 0.055 U/μL |
| Polymerase | | |
| (5 U/μL) | | |

*loading buffer: 40% (w/v) sucrose in nuclease free water with cresol red added to dark red color.

TABLE 9
EXEMPLARY CDRH SEQUENCES OF DISCLOSED ANTIBODIES

| | | SEQ ID | | SEQ ID | | SEQ ID |
|---|---|---|---|---|---|---|
| Antibody | CDRH1 | NO: | CDRH2 | NO: | CDRH3 | NO: |
| IOMA | FTGYY | 201 | INPNSGGTNY | 211 | AREMFDSSADWSP | 221 |
| iGL | MHWV | | AQKFQ | | WRGMVAW | |
| IOMA | FTGYH | 202 | INPFRGAVKY | 212 | AREMFDSSADWSP | 221 |
| | MHWV | | PQNFR | | WRGMVAW | |
| IO-003 | FTDYFI | 203 | INPFRGAVDY | 213 | ARAMFDSSDEWSP | 222 |
| | HWV | | AQKFR | | WHGLVAW | |
| IO-008 | FTGYEL | 204 | INPYRGAVKY | 214 | ARDMFDSSDEWSP | 223 |
| | HWV | | AQKFQ | | WRGMVAW | |
| IO-010 | FTGYHL | 205 | INPFRGAIGYA | 215 | AREMFDSDDEWSP | 224 |
| | HWV | | QKFR | | WRGMVAW | |
| IO-017 | FTGYHL | 205 | INPFRGAIGYA | 215 | AREMFDSDDDWSP | 225 |
| | HWV | | QKFR | | WRGMVAW | |
| IO-018 | TFIDYYI | 206 | INPYRGGPGY | 216 | ARELFDRDDDWSP | 226 |
| | HWV | | AQKFR | | WRGMVAW | |
| IO-040 | TFTDYF | 207 | INPRFGVTDSA | 217 | ARTMFDSDDDWSP | 227 |
| | IHWV | | QKFR | | WCGLVAW | |
| IO-044 | TFADHF | 208 | INPRFGNTDSA | 218 | TREVSDSSDDWSPW | 228 |
| | IHWV | | QKFR | | RGLVAW | |
| IO-049 | TFTGYD | 209 | INPFRGGIEYA | 219 | AREIFDSSDEWSPW | 229 |
| | MHWV | | QKFQ | | RGMVAW | |
| IO-050 | TFTDYE | 210 | INPFRGAVKY | 220 | AREMFDSDDEWSP | 224 |
| | IHWV | | RQKFQ | | WRGMVAW | |

TABLE 10
EXEMPLARY CDRL SEQUENCES OF DISCLOSED ANTIBODIES

| | | SEQ | | SEQ | | SEQ |
|---|---|---|---|---|---|---|
| Antibody | CDRL1 | ID NO: | CDRL2 | ID NO: | CDRL3 | ID NO: |
| IOMA | SSDVGSYN | 230 | LMIYEVSKRPSGVSN | 239 | CSYAGSVAF | 246 |
| iGL | | | | | | |
| IOMA | SRDVGGFD | 231 | LIIYEVNKRPSGISS | 240 | YSYADGVAF | 247 |
| IO-003 | SRDIGSFN | 232 | LMIYDVSKRPSGVSN | 241 | CSFAGSLAF | 248 |
| IO-008 | SSDVGSYD | 233 | LMIYEVSKRPSGVSN | 239 | CSYADSLVF | 249 |
| IO-010 | SSDIGGYN | 234 | LMIYDVNKRPSGVSN | 242 | CSYADTLVF | 250 |
| IO-017 | SSDIGGYD | 235 | LMIYDVNKRPSGVSN | 242 | CSYADTLVF | 250 |
| IO-018 | SSDIGSYD | 236 | LMIYDVSKRPSGVSN | 241 | CSYADTLVF | 250 |
| IO-040 | SSDVGSYN | 230 | LMIYEVSKRPSGVSN | 239 | CSYGDNLVF | 251 |
| IO-044 | SNDIGSYD | 237 | LMIYDVTKRPSGVSN | 243 | WSYADSVAF | 252 |
| IO-049 | SSDIGTYN | 238 | LMIYEVNRRPSGVSN | 244 | SSYEGTLAF | 253 |
| IO-050 | SSDVGSYN | 230 | LMIYEVSRRPSGVSN | 245 | CSYADTLIF | 254 |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An isolated polypeptide comprising an amino acid sequence that is at least 90% identical to the sequence of SEQ ID NO: 119, wherein the isolated polypeptide comprises at least an amino acid mutation in a position corresponding to D279, V430, D460, T461, T462, D463, or N464 of SEQ ID NO: 119.

2.-4. (canceled)

5. The isolated polypeptide of claim 1, wherein the isolated polypeptide comprises the sequence of SEQ ID NO: 118.

6.-8. (canceled)

9. The isolated polypeptide of claim 1, wherein the isolated polypeptide comprises the sequence of SEQ ID NO: 117.

10.-11. (canceled)

12. The isolated polypeptide of claim 1, wherein the isolated polypeptide binds to a neutralizing antibody with an affinity of about 30 μM or less, wherein the neutralizing antibody has specificity for a CD4 binding site of an HIV Env protein.

13.-22. (canceled)

23. The isolated polypeptide of claim 1, comprising an amino acid sequence that is at least 90% identical to the sequence of SEQ ID NO: 121 or SEQ ID NO: 120; or comprising the sequence of SEQ ID NO: 121 or SEQ ID NO: 120.

24.-30. (canceled)

31. The isolated polypeptide of claim 1, comprising an amino acid sequence that is at least 90% identical to the sequence of SEQ ID NO: 130 or SEQ ID NO: 129; or comprising the sequence of SEQ ID NO: 130 or SEQ ID NO: 129.

32.-40. (canceled)

41. A vaccine composition, comprising a carrier associated with a plurality of human immunodeficiency virus (HIV) immunogens, wherein the carrier is a monovalent or a multivalent carrier; and wherein the plurality of HIV immunogens comprise an isolated polypeptide comprising an amino acid sequence that is at least 90% identical to the sequence of SEQ ID NO: 129 or SEQ ID NO: 130.

42.-47. (canceled)

48. The vaccine composition of claim 41, wherein the carrier comprises a self-assembling nanoparticle; and optionally the self-assembling nanoparticle is an i301 nanoparticle or a variant thereof, or a mi3 nanoparticle or a variant thereof.

49. The vaccine composition of claim 41, comprising a plurality of particle-forming proteins, and optionally one or more of the plurality of particle-forming proteins comprise a 2-dehydro-3-deoxy-phosphogluconate (KDPG) aldolase or a variant thereof.

50. The vaccine composition of claim 49, wherein an HIV immunogen of the plurality of HIV immunogens is attached to a particle-forming protein of the plurality of particle-forming proteins, and optionally the HIV immunogen of the plurality of HIV immunogens is attached to the particle-forming protein of the plurality of particle-forming proteins through a Spytag/SpyCatcher binding pair; and further optionally, wherein the HIV immunogen of the plurality of HIV immunogens comprises a Spytag at the C-terminus of the HIV immunogen and the particle-forming protein of a plurality of particle-forming proteins comprises a SpyCatcher at the N-terminus of the particle-forming protein and/or wherein the HIV immunogen of the plurality of HIV immunogens comprises an Env protein or portion thereof, and the Env protein or portion thereof comprises a Spytag at the C-terminus of the Env protein or portion thereof and the particle-forming protein of a plurality of particle-forming proteins comprises a SpyCatcher at the N-terminus of the particle-forming protein.

51. (canceled)

52. The vaccine composition of claim 41, further comprising an adjuvant; optionally, wherein the adjuvant is selected from the group consisting of: saponin/MPLA nanoparticles (SMNP), aluminum hydroxide, alhydrogel, AddaVax, MF59, ASO3, Freund's adjuvant, Montanide ISA51, CpG, Poly I:C, glucopyranosyl lipid A, flagellin, resiquimod, and a combination thereof.

53.-64. (canceled)

65. A method for treating or preventing an HIV infection, or treating or preventing a disease or disorder caused by an HIV infection in a subject in need thereof, comprising: administering to the subject one or more vaccine compositions, thereby treating or preventing the HIV infection or the disease or disorder caused by the HIV infection in the subject, wherein the method comprises administering to the subject
(a) a first vaccine composition comprising a carrier associated with a plurality of human immunodeficiency virus (HIV) immunogens, wherein the plurality of HIV immunogens comprise an isolated polypeptide comprising an amino acid sequence that is at least 90% identical to the sequence of SEQ ID NO: 129;
(b) a second vaccine composition comprising a carrier associated with a plurality of human immunodeficiency virus (HIV) immunogens, wherein the plurality of HIV immunogens comprise an isolated polypeptide comprising an amino acid sequence that is at least 90% identical to the sequence SEQ ID NO: 130;
(c) a third vaccine composition comprising a carrier associated with a plurality of human immunodeficiency virus (HIV) immunogens, wherein the plurality of HIV immunogens comprise an isolated polypeptide comprising an amino acid sequence that is at least 90% identical to the sequence SEQ ID NO: 131; and/or
(d) a fourth and fifth vaccine composition each comprising a carrier associated with a plurality of human immunodeficiency virus (HIV) immunogens, comprising three, four, five, six, seven, or eight HIV immunogens, wherein each of the three, four, five, six, seven, or eight of HIV immunogens is derived from an HIV variant different from the other.

66.-67. (canceled)

68. The method of claim 65, wherein administering the one or more vaccine compositions induces a polyclonal serum response in the subject, induces broadly neutralizing responses in the subject against one or more HIV variants, and/or boosts a neutralizing antibody response in the subject.

69.-71. (canceled)

72. The method of claim 65, wherein administration of the second vaccine composition to the subject occurs about two, three, four, or five weeks after administration of the first vaccine composition to the subject.

73. (canceled)

74. The method of claim 65, wherein administration of the third vaccine composition to the subject occurs about two, three, four, or five weeks after administration of the second vaccine composition to the subject; wherein administration of the fourth vaccine composition to the subject occurs about two, three, four, or five weeks after administration of the third vaccine composition to the subject; and/or wherein administration of the fifth vaccine composition to the subject occurs about two, three, four, or five weeks after administration of the fourth vaccine composition to the subject.

75. The method of claim 68, wherein the neutralizing antibody response in the subject is characterized by at least a 2-fold increase in neutralizing titer following administration of the second vaccine composition as determined by a pseudo-virus neutralization assay; optionally, as compared to the subject prior to or after administration of the first vaccine composition to the subject.

76. The method of claim 68, wherein administration of the first and second vaccine compositions results in at least a 2-fold increase in the number of antibodies from the serum of the subject capable of specifically binding to a CD4 binding site epitope of an Env protein; optionally as compared to the subject prior to or after administration of the first vaccine composition.

77. (canceled)

78. The method of claim 68, wherein the neutralizing antibody response in the subject is characterized by neutralization of two or more pseudo-viruses comprising an Env protein or portion thereof, each of an HIV variant different from one another, by the sera of the subject, following administration of the fifth vaccine composition; wherein neutralization is defined as having a percent neutralization of about 40% or more at a serum dilution of about 1:100, as measured by a pseudo-virus neutralization assay and/or wherein administration of the first, second, third, fourth, and fifth vaccine compositions results in at least a 0.5-fold increase in the number of antibodies from the serum of the subject capable of specifically binding to a CD4 binding site epitope of an Env protein; optionally as compared to the subject prior to or after administration of the first vaccine composition.

79.-83. (canceled)

84. The method of claim 65, wherein administering the first vaccine composition is a prime and administration of the second, third, fourth and fifth vaccine compositions are each a boost and/or wherein the administration comprises intravenous, intraperitoneal or subcutaneous administration.

85.-98. (canceled)

99. The method of claim 65, wherein the plurality of HIV immunogens each comprise (1) an amino acid sequence having at least 65%, 70%, 75%, 80%, 85%, 90%, 95%, 98%, or 99% sequence identity to an amino acid sequence selected from the group consisting of SEQ ID NOs: 132-139; or (2) an amino acid sequence selected from the group consisting of SEQ ID NOs: 132-139.


SEQUENCE LISTING (268 sequences)

SEQ ID NO: 1 - protein, 126 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREMFDSSADWSPWRGMVAWGQGTLVTVSS

SEQ ID NO: 2 - protein, 126 aa, synthetic construct
EVQLVESGAQVKKPGASVTVSCTASGYKFTGYHMHWVRQAPGRGLEWMGWINPFRGAVKYPQNFRGRVSMTRDTSMEIFYMELSRLTSDDTAVYYCAREMFDSSADWSPWRGMVAWGQGTLVTVSS

SEQ ID NO: 3 - protein, 126 aa, synthetic construct
QVQLVQSGAEVKKPGASVTVSCKASGYTFTDYFIHWVRQAPGQGLEWMGWINPFRGAVDYAQKFRGRVTMTRDTSITTAYMELSRLRSDDTAVYYCARAMFDSSDEWSPWHGLVAWGQGTLVTVSS

SEQ ID NO: 4 - protein, 126 aa, synthetic construct
QVQLVQSGAEVKKPGASVTVSCKASGYTFTGYELHWVRQAPGQGLEWMGWINPYRGAVKYAQKFQGRVTMTRDTSISTAYMVLNGLRSDDTAVYYCARDMFDSSDEWSPWRGMVAWGQGTPVTVSS

SEQ ID NO: 5 - protein, 126 aa, synthetic construct
QVQLVQSGAEVKKPGASVTVSCKASGYTFTGYHLHWVRQAPGQGLEWMGWINPFRGAIGYAQKFRGRVTMTRDTSIITAYMELRGLRSDDTAVYFCAREMFDSDDEWSPWRGMVAWGQGTLVTVSS

SEQ ID NO: 6 - protein, 126 aa, synthetic construct
QVQLVQSGAEVKKPGASVTVSCKASGYTFTGYHLHWVRQAPGQGLEWMGWINPFRGAIGYAQKFRGRVTMTRDTSINTAYMELRGLRSDDTAVYFCAREMFDSDDDWSPWRGMVAWGQGTLVTVSS

SEQ ID NO: 7 - protein, 126 aa, synthetic construct
QVQLVQSGAEVKKPGASVTVSCKASGYTFIDYYIHWVRQAPGQGLEWMGWINPYRGGPGYAQKFRGRVTLTRDTSINTAYMELNRLKSDDTAVYFCARELFDRDDDWSPWRGMVAWGQGTLVTVSS

SEQ ID NO: 8 - protein, 126 aa, synthetic construct
QVQLVQSGAEVKKPGASVTVSCKASGYTFTDYFIHWVRQVPGQGLEWMGWINPRFGVTDSAQKFRGRVTMTRDTSITTAYMELSRLRSDDTAVYYCARTMFDSDDDWSPWCGLVAWGQGTLVTVSS

SEQ ID NO: 9 - protein, 126 aa, synthetic construct
QVQLVQSGAEMKKPGASVTVSCAASGYTFADHFIHWVRQAPGQGLEWMGWINPRFGNTDSAQKFRGRVTMTRDTSITTAYMELSRLRSDDTAVYYCTREVSDSSDDWSPWRGLVAWGQGTLVTVSS

SEQ ID NO: 10 - protein, 126 aa, synthetic construct
QVQLVQSGAEVKKPGASVTVSCRASGYTFTGYDMHWVRQAPGQGLEWMGRINPFRGGIEYAQKFQGRVTMTRDTSISTAYMELSRLRSDDTAVYYCAREIFDSSDEWSPWRGMVAWGQGTLVTVSS

SEQ ID NO: 11 - protein, 126 aa, synthetic construct
QVQLVQSGAEVKKPGASVRVSCKASGYTFTDYEIHWVRQAPGQGLEWMGWINPFRGAVKYRQKFQGRVTMTRDPSITTAYMELSRLRSDDTARYYCAREMFDSDDEWSPWRGMVAWGQGTLVTVSS

SEQ ID NO: 12 - protein, 108 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCCSYAGSVAFGGGTKLTVL

SEQ ID NO: 13 - protein, 108 aa, synthetic construct
QSALTQPASVSGSPGQSITISCAGSSRDVGGFDLVSWYQQHPGKAPKLIIYEVNKRPSGISSRFSASKSGNTASLTISGLQEEDEAHYYCYSYADGVAFGGGTKLTVL

SEQ ID NO: 14 - protein, 108 aa, synthetic construct
QSALTQPASVSGSPGQSITISCTGTSRDIGSFNLVSWYQQHPGKAPKLMIYDVSKRPSGVSNRFSGSKSDNMASLIISGFQAEDEAEYFCCSFAGSLAFGGGTKLTVL

SEQ ID NO: 15 - protein, 108 aa, synthetic construct
QSALTQPASVSGSPGQSITISCTGTSSDVGSYDLVSWYQQHPGKAPKLMIYEVSKRPSGVSNRFSGSKSGNTASLTISGLQAEDEADYYCCSYADSLVFGGGTKVTGL

SEQ ID NO: 16 - protein, 108 aa, synthetic construct
QSALTQPASVSGSPGQSITISCTGTSSDIGGYNLVSWYQQHPGKAPKLMIYDVNKRPSGVSNRFSGSKSGNTASLTISGFQAEDEADYYCCSYADTLVFGGGTKLTVL

SEQ ID NO: 17 - protein, 108 aa, synthetic construct
QSALTQPASVSGSPGQSITISCTGTSSDIGGYDLVSWYQQHPGKAPKLMIYDVNKRPSGVSNRFSGSKSGNTASLTISGFQAEDEADYYCCSYADTLVFGGGTKLTVL

SEQ ID NO: 18 - protein, 108 aa, synthetic construct
QSALTQPASVSGSPGQSITISCTGTSSDIGSYDLVSWYQQHPGKAPKLMIYDVSKRPSGVSNRFSGSKSGNTASLTISGFQAEDEADYYCCSYADTLVFGGGTKLTVL

SEQ ID NO: 19 - protein, 108 aa, synthetic construct
QSALTQPASVSGSPGQSTTISCTGTSSDVGSYNLVSWYQQHPGKVPKLMIYEVSKRPSGVSNRFSGSKSGNSASLTISGLQTEDVADYYCCSYGDNLVFGGGTRLTVL

SEQ ID NO: 20 - protein, 108 aa, synthetic construct
QSALTQPASVSGSPGQSSTISCTGTSNDIGSYDLVSWYQQHPGKAPKLMIYDVTKRPSGVSNRFSGSKSGKTASLTISGFQTEDEADYYCWSYADSVAFGGGTKLTGL

SEQ ID NO: 21 - protein, 108 aa, synthetic construct
QSALTQPASVSGSPGQSITISCTGPSSDIGTYNLVSWYQQHPGKAPKLMIYEVNRRPSGVSNRFSGSKCGNTASLTISDFQAEDEADYYCSSYEGTLAFGGGTKLTVL

SEQ ID NO: 22 - protein, 108 aa, synthetic construct
QSALTQPASVSGSPGQSITISCTGTSSDVGSYNLVSWYQQHPGKAPKLMIYEVSRRPSGVSNRFSGSKSGNTASLTISGFQAEDEADYYCCSYADTLIFGGGTKLTVL

SEQ ID NO: 23 - protein, 98 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 24 - protein, 99 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCCSYAGSSTF

SEQ ID NO: 25 - protein, 95 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSS

SEQ ID NO: 26 - protein, 106 aa, synthetic construct
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTV

SEQ ID NO: 27 - protein, 126 aa, synthetic construct
QVQLVQSGAEVRKPGASVKVSCKASGYTFTGYHMHWVRQAPGQGLEWMGWISPNSGNTNSTEKFQGRVTMTRDTSISTVYMELSRLRSDDTAVYYCAREIFDSSYDWSPWRGMVAWGQGTLVTVSS

SEQ ID NO: 28 - protein, 126 aa, synthetic construct
QVQLVQSGAEVKKPGASVKVSCKASGYTFTGYYIYWVRQAPGQGLEWMGWINPNSGRTNYTENFQGRVTLTRDTSINTAYMELNRLTSDDTAVYYCAREVFDSSVDWSPWRGMVAWGQGTLVTVSS

SEQ ID NO: 29 - protein, 126 aa, synthetic construct
QVQLVQSGAEVKKPGASVKVSCKASGYTFTGYYMHWVRQAPGQGLEWMGWINPNSGGTNCAQKFQGRVTMTRDTSISTAYMELSRLRSDDTAVYYCAREMFDSSVNWSPWRGMVAWGQGTLVTVSS

SEQ ID NO: 30 - protein, 126 aa, synthetic construct
QVQLVQSGAEVKKPGASVKVSCKASGYNFTGYHMHWVRQAPGQGLEWMGWISPNRGVSNSAEKFQGRVTMTRDTSISTVYMELSRLRSDDTAVYYCAREMFDSSFDWSPWRGMVAWGQGTLVTVSS

SEQ ID NO: 31 - protein, 126 aa, synthetic construct
QVQLVQSGAEVKKPGASVKVSCKASGYTFTGYYMHWVRQAPGQGLEWMGWINPNSGGTNYVQKFQGRVTMTRDTSISTAYMELSRLRSDDTAVYYCAREMFDSSADWSPWRGMVAWGQGTLVTVSS

SEQ ID NO: 32 - protein, 126 aa, synthetic construct
QVQLVQSGAEVRKPGASVKVSCKASGYTFTGYHMHWVRQAPGQGLEWMGWISPNTGNTNSTEKFQGRVTMTRDTSISTVYMELNRLRSNDTAVYYCAREIFDSSYEWSPWRGMVAWGQGTLVTVSS

SEQ ID NO: 33 - protein, 126 aa, synthetic construct
QVQLVQSGAEVKKPGASVTVSCEASGYTFTGYYIHWVRQAPGQGLEWMGWINPTRGGVGYAQKFRGRVTMTRDTSISIAYMELTRLRSDDTAIYFCARELFDSDDDWSPWRGMVAWGQGTLVTVSS

SEQ ID NO: 34 - protein, 126 aa, synthetic construct
QVQLVQSGAEVRKPGASVKVSCKASGYTFTGYHMHWVRQAPGQGLEWMGWISPNSGDTNSTEKFQGRVTMTRDTSISTVYMELSRLRSDDTAVYYCAREIFDSSYDWSPWRGMVAWGQGTLVTVSS

SEQ ID NO: 35 - protein, 126 aa, synthetic construct
QVQLVQSGAEVKKPGASVKVSCKASGYNLTGYHMHWVRQAPGQGLEWMGWISPNRGVSNSAEKFQGRVTMTRDTSISTVYMELSRLRSDDTAVYYCAREMFDSSFDWSPWRGMVAWGQGTLVTVSS

SEQ ID NO: 36 - protein, 126 aa, synthetic construct
QVQLVQSGAEVKKPGASVTVSCKASGYTFTDYYIHWVRQAPGQGLEWMGLINPNRGGVKYAQRFQGRVTMTRDTSISTAYMELSGLRSDDTAVYYCAREMFDSTDDWSPWRGMVAWGQGTLVTVSS

SEQ ID NO: 37 - protein, 126 aa, synthetic construct
QVQLVQSGAEVKKPGASVTVSCKASGYTFIDYYIHWVRQAPGQGLEWMGWINPYRGGPGYAQKFRGRVTMTRDTSINTAYMELNRLKSDDTAVYFCARELVDRDDDWSPWRGMVAWGQGTLVTVSS

SEQ ID NO: 38 - protein, 126 aa, synthetic construct
QVQLVQSGAEVKKPGASVRVSCEASGYTFTEYEMQWVRQAPGQGLEWMGWINPFRGAIGYAQKFQGRVTMTRDTSITTTYMEMSRVRSDDTAVYYCAREMFDSSDEWFPWRGMVAWGQGTLVTVSS

SEQ ID NO: 39 - protein, 126 aa, synthetic construct
QVQLVQSGAEVKKPGASVTVSCRASGYTFTGYDMHWVRQAPGQGFEWMGRIHPFRGAVEYAQKFQGRVTMTRDTSISTAYMELSRLRSDDTAVYYCAREMLDSDDEWSPWRGMVAWGQGTLVTVSS

SEQ ID NO: 40 - protein, 126 aa, synthetic construct
QVQLVQSGAEVKKPGASVRVSCKASGYTFTDYEMHWVRQAPGQGLEWMGRINPFRGAIEYSQKFQGRVTMTRDTSISTAYMELSRLRSDDTAVYYCAREIFDSDDEWSPWRGMVAWGQGSLVTVSS

SEQ ID NO: 41 - protein, 126 aa, synthetic construct
QVQLVQSGAEVKKPGASVRVSCEASGYTFTDYDLHWVRQAPGQGLDWMGRINPFRGAIEYSQKFQGRVTMTRDTSISTAYMELSRLRSDDTAVYYCAREMFDSDDEWSPWRGMVAWGQGTLVTVSS

SEQ ID NO: 42 - protein, 126 aa, synthetic construct
QVQLVQSGAEVKKPGASVRVSCKASGYTFTDYDMHWVRQAPGQGLEWMGRINPFRGAIEYSQKFQGRVTMTRDTSISTAYMELSRLRSDDTAVYYCAREMFDSDDEWSPWRGMVAWGQGTLVTVSS

SEQ ID NO: 43 - protein, 126 aa, synthetic construct
QVQLVQSGAEVKKPGASVRVSCKASGYTFIDYDLHWVRRAPGQGLEWMGRINPFRGGIEYAQKFQGRVTMTRDTSISTAYLELSRLRSDDTAVYYCAREIFDSDDEWSPWRGMVAWGQGSLVTVSS

SEQ ID NO: 44 - protein, 126 aa, synthetic construct
QVQLVQSGAEVKKPGASVRVSCKASGYTFTDYEMHWVRQAPGQGLEWMGWINPFRGAIKYSQKFQGRVTMTRDTSISTAYMELSRLRSDDTAVYYCAREMFDSDDEWSPWRGMVAWGQGTLVTVSS

SEQ ID NO: 45 - protein, 126 aa, synthetic construct
QVQLVQSGAEVKKPGASVRVSCKASGYTFIDYDMHWVRQAPGQGLEWMGWINPFRGGIEYAQKFQGRVIMTRDTSIRTAYMELSRLRSDDTAIYYCAREMFDSGDEWSPWRGMVAWGQGTLVTVSS

SEQ ID NO: 46 - protein, 126 aa, synthetic construct
QVQLVQSGAEVRKPGASVTVSCRASGYTFTDYDMHWVRQAPGQGLEWMGRINPFRGAIEYAQKFQGRVTMTRDTSITTAYMELNRLTSDDTAAYYCAREIFDSDDEWSPWRGMVAWGQGTLVTVSS

SEQ ID NO: 47 - protein, 126 aa, synthetic construct
QVQLVQSGAEVKKPGASVTVSCRASGYTFTGYDMHWVRQAPGQGFEWMGRIHPFRGAVEYAQKFQGRVTMTRDTSISTAYMELSRLRSDDTAVYYCAREMFDSDDEWSPWRGMVAWGQGTLVTVSS

SEQ ID NO: 48 - protein, 126 aa, synthetic construct
QVQLVQSGAEVKKPGASVRVSCKASGYTFIDYDMHWVRQAPGQGLEWMGRINPFRGAIEYAQKFQGRVTMTRDTSITTAYMELSRLRSDDTAVYYCAREMFDSDDEWSPWRGMVAWGQGTLVTVSS

SEQ ID NO: 49 - protein, 126 aa, synthetic construct
QVQLVQSGAEVKKPGASVKVSCKASGYTFTGYYMHWVRQAPGQGLEWVGWINPDSGGTNCAQKFQGRVTMTRDTSISTAYMELNRLRSDDTAVYYCAREMFDSGANWSPWRGMVAWGQGTLVTVSS

SEQ ID NO: 50 - protein, 126 aa, synthetic construct
QVQLVQSGAEVRKPGASVTVSCKTSGYTFTAYSIHWVRQAPGQGLEWMGWINPFRGGVKYVQKFQGRVTMTRDTSITTAYMELNRLRSDDTAVYFCAREMFDNDDDWSPWRGMVAWGQGTLVTVSS

SEQ ID NO: 51 - protein, 126 aa, synthetic construct
QVQLVQSGAEVRKPGASVTVSCKTSGYTFTAYYIHWVRQAPGQGLEWMGWINPFRGGVKYVQKFQGRVTMTRDTSITTAYMELNRLRSDDTAVYFCAREMFDNDDDWSPWRGMVAWGQGTLVTVSS

SEQ ID NO: 52 - protein, 126 aa, synthetic construct
QVQLVQSGAEVKKPGASVTVSCKASGYTFIDYYIHWVRQAPGQGLEWMGWINPTRGGIGYAQKFQGRVTMTRDTSISTAYMELSRLRSDDTAVYFCARDMFDSDDEWSPWRGIVAWGQGTLVTVSS

SEQ ID NO: 53 - protein, 126 aa, synthetic construct
QVQLVQSGAEVKKPGASVTVSCKASGYTFTDHFIHWVRQAPGQGLEWMGWINPRFGNRDSERKFQGRVTMTRDTSITTAYLELSGLRSDDTAVYYCAREIFDSDDEWSPWNGLVAWGQGTLVTVSS

SEQ ID NO: 54 - protein, 126 aa, synthetic construct
QVQLVQSGAEVKKPGASVTVSCKASGYTFTGYYIHWVRQAPGQGLEWMGWINPTRGGVGYAQKFRGRVTMTRDTSISIAYMELTRLRSDDTAIYFCARELFDSDDDWSPWRGMVAWGQGTLVTVSS

SEQ ID NO: 55 - protein, 126 aa, synthetic construct
QVQLVQSGAEVKKPGASVTVSCEASGYTFTDYEMHWVRQAPGQGLEWMGWINPYRGVIKYAQKFQGRVTMTRDTSINTAYMELSRLRSDDTAVYYCAREMFDSSDDWSPWRGMVAWGQGTLVTVSS

SEQ ID NO: 56 - protein, 126 aa, synthetic construct
QVQLVQSGAEVKKPGASVTVSCRASGYTFTGYDMHWVRQAPGRGLEWMGRINPFRGAIEYAQKFQGRVSMTRDTSISTAYMELSRLRSDDTAIYYCAREMFDSDDEWSPWRGMVAWGQGTLVTVSS

SEQ ID NO: 57 - protein, 126 aa, synthetic construct
QVQLVQSGAEVKKPGASVTVSCKASGYTFTDYYIHWVRQAPGQGLEWMGWINPTRGGIGYAQKFQGRVTMTRDTSINTAYMELSRLRSDDTAVYFCARDMFDSDDEWSPWRGIVAWGQGTLVTVSS

SEQ ID NO: 58 - protein, 126 aa, synthetic construct
QVQLVQSGAEVRKPGASVTVSCKTSGYTFTAYYIHWVRQAPGQGLEWMGWINPFRGGVKYVQKFQGRVTMTRDTSITTAYMELKRLRSDDTAVYFCAREMFDNDDDWSPWRGMVAWGQGTLVTVSS

SEQ ID NO: 59 - protein, 126 aa, synthetic construct
QVQLVQSGAEVKKPGASVKVSCKASGYTFTGYYMHWVRQAPGQGLEWMGWINPTRGGVGYAQKFRGRVTMTRDTSISIAYMELTRLRYDDTAMYFCARELFDSDDDWSPWRGMVAWGQGTLVTVSS

SEQ ID NO: 60 - protein, 126 aa, synthetic construct
QVQLVQSGAEVKKPGASVRVSCKASGYTFIDYDMHWVRQAPGQGLEWMGRINPFRGGVEYAQKFQGRVIMTRDTSISTAYMELSRLISDDTAVYYCARELFDSDDEWSPWRGMVAWGQGTLVTVSS

SEQ ID NO: 61 - protein, 126 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREMFDSNDEWSPWRGMVAWGQGTLVIVSS

SEQ ID NO: 62 - protein, 126 aa, synthetic construct
QVQLVQSGAEVKKPGASVRVSCEASGYTFTDYDLHWVRQAPGQGLEWMGRINPFRGAIEYSQKFQGRVTMTRDTSISTAYMELSRLRSDDTAVYYCAREMFDSDDEWSPWRGMVAWGQGTLVTVSS

SEQ ID NO: 63 - protein, 126 aa, synthetic construct
QVQLVQSGAEVKKPGASVRVSCKASGYTFTDYEIHWVRQAPGQGLEWMGWINPFRGAVKYRQKFQGRVTMTRDTSITTAYMELSRLRSDDTAVYNCARKMFDSDDEWSPWRGMVAWGQGILVTVSS

SEQ ID NO: 64 - protein, 126 aa, synthetic construct
QVQLVQSGAEVKKPGASVRVSCKASGYTFTDYEIHWVRQAPGQGLEWMGWINPFRGAVKYRQKFQGRVTMTRDTSITTAYMELSRLRSDDTAVYNCAREMFDSDDEWSPWRGMVAWGQGILVIVSS

SEQ ID NO: 65 - protein, 126 aa, synthetic construct
QVQLVQSGAEVKKPGASVRVSCKASGYTFIDYDMHWVRQAPGQGLEWMGRINPFRGGIEYAQKFQGRVTMTRDTSISTAYMELSRLRSDDTAVYYCAREMFDSDDEWSPWRGMVAWGQGTLVTVSS

SEQ ID NO: 66 - protein, 126 aa, synthetic construct
QVQLVQSGAEVKKPGASVKVSCKASGYTFTGYYMHWVRQAPGQGLEWMGWINPNSGGTNYAQKFQGRVTMTRDTSITTAYMELNRLRSDDTAVYFCAREMFDNDDDWSPWRGMVAWGQGTLVTVSS

SEQ ID NO: 67 - protein, 126 aa, synthetic construct
QVQLVQSGAEVKKPGASVKVSCKASGYTFTGYYMHWVRQAPGQGLEWLGWINPKRGGTNYAQKFQGRVTMTRDTSISTAYMELSRLRSDDTAVYYCAREIFDSSADESPWRGMVAWGQGILVTVSS

SEQ ID NO: 68 - protein, 126 aa, synthetic construct
QVQLVQSGAEVKKPGASVKVSCKASGYTFTDYYIYWVRQAPGQGLEWLGWINPERGGTKYAQKFQGRVTMTRDTSISTAYMELSRLRSDDTAVYYCAREIFDSSADGSPWRGMVAWGQGTLVTVSS

SEQ ID NO: 69 - protein, 126 aa, synthetic construct
QVQLVQSGAEVKKPGASVKVSCKASGYTFTGYYIYWVRQAPGQGLEWLGWSNPKTGGTNYAQKLQGRVTMTRDTSISTAYMELSRLRSDDTAVYYCAREIFDSSVDGSPWRGMVAWGQGTLVTVSS

SEQ ID NO: 70 - protein, 126 aa, synthetic construct
QVQLVQSGAEVKKPGASVKVSCKASGYTFTGYYMYWVRQAPGQGLEWLGWINPENGGTNYAQKFQGRVTMTRDTSISTAYLELSRLRSDDTAVYYCAREIFDSSAGGSPWRGMVAWGQGILVTVSS

SEQ ID NO: 71 - protein, 126 aa, synthetic construct
QVQLVQSGAEVKKPGASVKVSCKASGYTFTGYYMHWVRQAPGQGLEWMGWINPNSGGTNYAQKFQGRVTMTRDTTIKTAYMELSRLNSDDTAVYYCAREIFENKTDGDPWRGMVAWGQGTLVTVSS

SEQ ID NO: 72 - protein, 126 aa, synthetic construct
QVQLVQSGAEVKKPGASVKVSCKASGYTFTGYYMHWVRQAPGQGLEWMGWINPFRGGVKYVQKFQGRVTMTRDTSITTAYMELNRLRSDDTAVYFCAREMFDNDDDWSPWRGMVAWGQGTLVTVSS

SEQ ID NO: 73 - protein, 126 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREVFDSSADWSPWRGMVAWGQGTLVTVSS

SEQ ID NO: 74 - protein, 126 aa, synthetic construct
QVQLVQSGAEVKKPGASVTVSCKASGYTFTDYFIHWVRQAPGQGLEWMGWINPRFGNRDSERKFQGRVTMTRDTSITTAYMELSGLRSDDTAVYYCAREIFNSDDEWSPWSGLVAWGQGTLVTVSS

SEQ ID NO: 75 - protein, 126 aa, synthetic construct
QVQLVQSGAEVKKPGASVTVSCKASGYTFTDYFIHWVRQVPGQGLEWMGWINPRFGVTDSAQKFRGRLTMTRDTSISTAYMELSRLRSDDTAVYYCVRTMFDSDDDWSPWRGLVAWGQGTLVTVSS

SEQ ID NO: 76 - protein, 126 aa, synthetic construct
QVQLVQSGAEVKKPGASVKVSCKASGYTFTGYYMHWVRQAPGQGLEWMGWINPRFGNTDSAQKFQGRVTMTRDTSISTAYMELSRLRSDDTAVYYCAREMFDSSADWSPWRGMVAWGQGTLVTVSS

SEQ ID NO: 77 - protein, 108 aa, synthetic construct
QSALTQPASVSGSPGQSITISCTGTSSDVGRSYLVSWYQQHPGKTPKLLIYKVSQRPSGISNRFSGSKSGNTASLTISGLQAEDGADYYCCSYAGYAVFGGGTKLTVL

SEQ ID NO: 78 - protein, 108 aa, synthetic construct
QSALTQPASVSGSPGQSITISCTGTSSDVGSYDLVSWYQQHPGKAPKLMIYDVTKRPSGVSNRFSDSKSGNTASLPISGLQAEDEAGNYCYSSAGSVAFGGGTKLTVL

SEQ ID NO: 79 - protein, 108 aa, synthetic construct
QSALTQPASVSGSPGQSITISCTGTSRDIGSFNLVSWYQQHPGKAPKLMIYDVSKRPSGVSNRFSGSKSDNMASLIISGFQAEDEAEYFCCSFAGGLAFGGGTKLTVL

SEQ ID NO: 80 - protein, 108 aa, synthetic construct
QSALTQPASVSGSPGQSITISCTGASSDVGSFNLVSWYQQHPGKTPKLIIYEVSKRPSGVSNRFSGSKSGNTASLTISGLQTEDEADYYCCSYAGSVAFGGGTKLTVL

SEQ ID NO: 81 - protein, 108 aa, synthetic construct
QSALTQPASVSGSPGQSITISCTGTSSDVGRSYLVSWYQQHPGKTPKLLIYKVSQRPSGISNRFSGSKSGNTASLTISGLQAEDGADYYCCSYAGNAVFGGGTKLTVL

SEQ ID NO: 82 - protein, 108 aa, synthetic construct
QSALTQPASVSGSPGQSITISCTGTGSDVGSYNLVSWYQQHPGKAPKLMIYDVSKRPSGVSNRFSGSKSGNTASLTISGFQAEDEADYYCCSYADTLVFGGGTKLTVL

SEQ ID NO: 83 - protein, 108 aa, synthetic construct
QSALTQPASVSGSPGQSITISCTGTSSDVGRSYLVSWYQQHPGKTPKLMIYKVSQRPSGISNRFSGSKSGNTASLTISGLQAEDGADYYCCSYAGSAVFGGGTKLTVL

SEQ ID NO: 84 - protein, 108 aa, synthetic construct
QSALTQPASVSGSPGQSITISCTGTSSDVGRSYLVSWYQQHPGKTPKLMIYKVSQRPSGISNRFSGSKSGNTASLTISGLQAEDGADYYCCSYAVNAVFGGGTKLTVL

SEQ ID NO: 85 - protein, 108 aa, synthetic construct
QSALTQPASVSGSPGQSITISCTGTSSDVGSYNLVSWYQQHPGKAPKLMIYEVSKRPSGVSNRFSGSKSGNTASLTISGFQAEDEAYYHCYSYADTVAFGGGTELTVL

SEQ ID NO: 86 - protein, 108 aa, synthetic construct
QSALTQPASVSGSPGQSITISCTGTSSDVGSYDLVPWYQQHPGKAPKLMIYEVSKRPSGVSNRFSGSKSGNTASLTISGLQAEDEADYYCCSYADSLVFGGGTKLTGL

SEQ ID NO: 87 - protein, 108 aa, synthetic construct
QSALTQPASVSGSPGQSITISCTGTSSDVGSYDLVSWYQQHPGKAPKLMIYEVNRRPSGVSNRFSGSKSGNTASLTISGFQAEDEADYYCCSYGDNLIFGGGTKLTVL

SEQ ID NO: 88 - protein, 108 aa, synthetic construct
QSALTQPASVSGSPGQSITISCTGTSSDVGSYDLVSWYQQHPGKAPKLMIYEVSRRPSGVSNRFSGSKSGNTASLTISGFQAEDKADYFCCSYAGTLVFGGGTKLTVL

SEQ ID NO: 89 - protein, 108 aa, synthetic construct
QSALTQPASVSGSPGQSITISCTGTSSDVGSYDLVSWYQQHPGKAPKLMIYEVNRRPSGVSNRFSGSKSGNTASLIISGFQAEDEADYHCCSYGGTLGCGGGTKLTVL

SEQ ID NO: 90 - protein, 108 aa, synthetic construct
QSALTQPASVSGSPGQSITISCTVTSSDIGNYDLVSWYQQHPGKAPKLMIYEVSRRPSGVSNRFSGSKSGNTASLTISGFQTEDVADYYCCSYVGTLTFGGGTKLTVL

SEQ ID NO: 91 - protein, 108 aa, synthetic construct
QSALTQPASVSGSPGQSITISCTGTSSDVGSYNLVSWYQQHPGKAPKLMIYEVTRRPSGVSNRFSGSKSGNTASLIISGFQAEDEADYHCCSYTGTFVCGGGTQLTVL

SEQ ID NO: 92 - protein, 108 aa, synthetic construct
QSALTQPASVSGSPGQSITISCTGTSSDVGSYNLVSWYQQHPGKAPKLMIYEVSRRPSGVSNRFSGSKSGNTASLTISGFQPEDEADYHCCSYADTLVCGGGTELTVL

SEQ ID NO: 93 - protein, 108 aa, synthetic construct
QSALTQPASVSGSPGQSITISCTGTSSDVGNYDLVSWYQQHPGKAPKLMIYEVSRRPSGVSDRFSGSKSGNTASLTISGFRAEDEADYHCCSYTGTLECGGGTKLTVL

SEQ ID NO: 94 - protein, 108 aa, synthetic construct
QSALTQPASVSGSPGQSITISCTGTSSDVGSYNLVSWYQQHPGKAPKLMIYEVSRRPSGVSNRFSGSKSGNTASLTISGFQAEDEADYYCCTYAGTLVFGGGTKLTVL

SEQ ID NO: 95 - protein, 108 aa, synthetic construct
QSALTQPASVSGSPGQSITISCTVTSSDVGNFDLVSWYQQHPGKAPKLMIYEVNRRPSGVSNRFSGSKSGNTASLTISGFQAEDEADYYCCSYAGTLAFGGGTKLTVL

SEQ ID NO: 96 - protein, 108 aa, synthetic construct
QSALTQPASVSGSPGQSITISCTGTRSDVGSYDLVSWYQQHPGKAPKLMIYEVNRRPSGVSNRFSGSKSGNTASLIISGFQAEDEADYHCCSYGGTLVCGGGTKLTVL

SEQ ID NO: 97 - protein, 108 aa, synthetic construct
QSALTQPASVSGSPGQSITISCTGTSSDVGNYNLVSWYQQHPGKAPKLMIYEVSRRPSGVYNRFSGSKSGNTASLTISGFQAEDEADYYCCSYADTLIFGGGTKLTVL

SEQ ID NO: 98 - protein, 108 aa, synthetic construct
QSALTQPASVSGSPGQSITISCTGTSSDIGSFDLVSWYQQHPGKAPKLMIYDVSKRPSGVSNRFSGSKSGNTASLTISGFQAEDEADYYCCSYADTMAFGGGTKLTVL

SEQ ID NO: 99 - protein, 108 aa, synthetic construct
QSALTQPASVSGSPEQSITISCTGTSSDVGSYNLVSWYQQHPGKAPKLMIYEVSKRPSGVSNRFSGSKSGNTASLTISGLQAEDEADYDCCLYAGCVAFGGGTKLTVL

SEQ ID NO: 100 - protein, 108 aa, synthetic construct
QSALTQPASVSGSPEQSITISCTGTSSDVGSYNFVSWYQQHPGKAPKLMIYEVSKRPSGVSNRFSGSKSGNTASLTISGLQAEDEADYDCCLYAGCVAFGGGTKLTVL

SEQ ID NO: 101 - protein, 108 aa, synthetic construct
QSALTQPASVSGSPGQSITISCTGTSSDVGSFDLVSWYQQHPGKAPKLMIYEVNKRPSGVSNRFSGSKSGNTASLTISGFQAEDEADYYCSSYGDNLVFGGGTKLTVL

SEQ ID NO: 102 - protein, 108 aa, synthetic construct
QSALTQPASVSGSPEQSITISCTGTSSDVGSYNFVSWYQQHPGKAPKFMIYEVSKRPSGVSNRFSGSKSGNTASLTISGLQDEDEADYECCLYVGCVAFGGGTKLTGL

SEQ ID NO: 103 - protein, 108 aa, synthetic construct
QFALTQPASVFGSPGQSITISCTGTSSDVGSYNLVSWYQQHPGKAPKLMIYDVSKRPSGVSNRFSGSKSGNTASLTISGFQAEDEADYYCCSYADTLVFGGGTKLTVL

SEQ ID NO: 104 - protein, 108 aa, synthetic construct
QSALTQPASVSGSPGQSITISCTGTSSDVGIYDLVSWYQQHPGKAPKLMIYEVSRRPSGVSDRFSGSKSGNTASLTISGFRAEDEADYHCCSYTGTEGGAGGTILTVL

SEQ ID NO: 105 - protein, 108 aa, synthetic construct
QSALTQPASVSGSPGQSITISCTGTSSDIGGYDLVSWYQQHPGKAPKLMIYEVSKRPSGVSNRFSGSKSGNTASLTISGLQTEDGANYHCYSYADTVAFGGGTKLTVL

SEQ ID NO: 106 - protein, 108 aa, synthetic construct
QSALTQPASVSGSPGQSITISCTGTSSDVGSYNLVSWYQQHPGKAPKLMIYEVSERPSGVSNRFSGSKSGNTASLTISGLQAEDEADYYCSSSVGRVVFGGGTKLTVL

SEQ ID NO: 107 - protein, 108 aa, synthetic construct
QSALTQPASVSGSPGQSITISCTGTSSDVGSYNLVSWYQQHPGKAPKLMIYEVSARPSGVSNRFSGSKSGNTASLTISGLQAEDEADYYCSSCVGSVVFGGGTKLTVL

SEQ ID NO: 108 - protein, 108 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCSSYVGRVVFGGGTKLTVL

SEQ ID NO: 109 - protein, 108 aa, synthetic construct
QSALTQPASVSGSPGQSITISCTGTSSDVGSYNLVSWYQQHPGKAPKLMIYEVSKRPSGVSNRFSGSKAGNTASLTISGLQAEDEGDYYCCSYAGSVVFGGGTKLTVL

SEQ ID NO: 110 - protein, 108 aa, synthetic construct
QSALTQPASVSGSPGQSITISCTGTSSDVGSYNLVSWYQQHPGKAPKLMIYEVNKRPSGVSNRFSGSKSGNTASLTISGLQAEDEADYYCCSYAGSVAFGGGTKLTVL

SEQ ID NO: 111 - protein, 108 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYDCCLYAGCVAFGGGTKLTVL

SEQ ID NO: 112 - protein, 108 aa, synthetic construct
QSALTQPASVSGSPGQSITISCTGTSNDFGSYDLVSWYQQHPGNAPKLILYEVSKRPSGVSNRFSGSKSGNTASLTISGLQAEDEADYYCCSYAGTVAFGGGTKLTVL

SEQ ID NO: 113 - protein, 108 aa, synthetic construct
QSALTQPASVSGSPGQSITISCTGTSSDIGGYDLVSWYQQHPGKAPKLMIYEVTKRPSGISNRFAGSKSGNTASLTISGLQAEDEADYYCCSYAGSVAFGGGTKLTVL

SEQ ID NO: 114 - protein, 108 aa, synthetic construct
QSALTQPASVSGSPGQSITISCTGTSSDVGSYNLVSWYQQHPGKAPKLMIYEVTKRPSGISNRFAGSKSGNTASLTISGLQAEDEADYYCCSYAGSVAFGGGTKLTVL

SEQ ID NO: 115 - protein, 108 aa, synthetic construct
QSALTQPASVSGSPGQSITISCTGTSSDVGSYNLVSWYQQHPGKAPKLMIHEVIKRPSGVSNRFSGSKSGNTASLTISGLQAEDEADYYCCSYAGSVAFGGGTKLTVL

SEQ ID NO: 116 - protein, 108 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCCSYADSVAFGGGTKLTVL

SEQ ID NO: 117 - protein, 353 aa, synthetic construct
VWKEAKTTLFCASDAKAYEKECHNVWATHACVPTDPNPQEVVLENVTENFNMWKNDMVDQMQEDVISIWDQCLKPCVKLTNTSTLTQACPKVTFDPIPIHYCAPAGYAILKCNNKTFNGKGPCNNVSTVQCTHGIKPVVSTQLLLNGSLAEEEIVIRSKNLRNNAKIIIVQLNKSVEIVCTRPNNGGSGSGGDIRQAYCNISGRNWSEAVNQVKKKLKEHFPHKNISFQSSSGGDLEITTHSFNCGGEFFYCNTSGLFNDTISNATIMLPCRIKQIINMWQEPGKAIYAPPIKGNITCKSDITGLLLLRDGGNALRPTEIFRPSGGDMRDNWRSELYKYKVVEIKPLHHHHHH

SEQ ID NO: 118 - protein, 353 aa, synthetic construct
VWKEAKTTLFCASDAKAYEKECHNVWATHACVPTDPNPQEVVLENVTENFNMWKNDMVDQMQEDVISIWDQCLKPCVKLTNTSTLTQACPKVTFDPIPIHYCAPAGYAILKCNNKTFNGKGPCNNVSTVQCTHGIKPVVSTQLLLNGSLAEEEIVIRSKNLRNNAKIIIVQLNKSVEIVCTRPNNGGSGSGGDIRQAYCNISGRNWSEAVNQVKKKLKEHFPHKNISFQSSSGGDLEITTHSFNCGGEFFYCNTSGLFNDTISNATIMLPCRIKQIINMWQEPGKAIYAPPIKGNITCKSDITGLLLLRDGGNSQRETEIFRPSGGDMRDNWRSELYKYKVVEIKPLHHHHHH

SEQ ID NO: 119 - protein, 353 aa, synthetic construct
VWKEAKTTLFCASDAKAYEKECHNVWATHACVPTDPNPQEVVLENVTENFNMWKNDMVDQMQEDVISIWDQCLKPCVKLTNTSTLTQACPKVTFDPIPIHYCAPAGYAILKCNNKTFNGKGPCNNVSTVQCTHGIKPVVSTQLLLNGSLAEEEIVIRSKNLRDNAKIIIVQLNKSVEIVCTRPNNGGSGSGGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPSGGDMRDNWRSELYKYKVVEIKPLHHHHHH

SEQ ID NO: 120 - protein, 626 aa, synthetic construct
GSNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKNLRNNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEPGKCIYAPPIKGNITCKSDITGLLLLRDGGNALRPTEIFRPSGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 121 - protein, 626 aa, synthetic construct
GSNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKNLRNNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEPGKCIYAPPIKGNITCKSDITGLLLLRDGGNSQRETEIFRPSGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 122 - protein, 626 aa, synthetic construct
AENLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKNLSDNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKCIYAPPIKGNITCKSDITGLLLLRDGGNTTNNTEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 123 - protein, 626 aa, synthetic construct
AENLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKNLSNNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKCIYAPPIKGNITCKSDITGLLLLRDGGNTTNNTEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 124 - protein, 626 aa, synthetic construct
AENLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKNLTDNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKCIYAPPIKGNITCKSDITGLLLLRDGGNTANNAEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 125 - protein, 626 aa, synthetic construct
AENLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKNLTNNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKCIYAPPIKGNITCKSDITGLLLLRDGGNTANNAEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 126 - protein, 626 aa, synthetic construct
AENLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKALTDNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKCIYAPPIKGNITCKSDITGLLLLRDGGNTANNAEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 127 - protein, 627 aa, synthetic construct
AENLWVTVYYGVPVWKDAETTLFCASDAKAYETKKHNVWATHACVPTDPNPQEIHLENVTEEFNMWKNNMVEQMHTDIISLWDQSLKPCVKLTPLCVTLQCTNVTNAITDDMRGELKNCSFNMTTELRDKRQKVHALFYKLDIVPINENQNTSYRLINCNTAAITQACPKVSFEPIPIHYCAPAGFAILKCKDKKFNGTGPCPSVSTVQCTHGIKPVVSTQLLLNGSLAEEEVMIRSEDIRNNAKNILVQFNTPVQINCTRPNNNTRKSIRIGPGQWFYATGDIIGDIRQAHCNVSKATWNETLGKVVKQLRKHFGNNTIIRFANSSGGDLEVTTHSFNCGGEFFYCDTSGLFNSTWISNTSVQGSNSTGSNDSITLPCRIKQIINMWQRIGQAMYAPPIQGVIRCVSNITGLILTRDGGSTDSTTETFRPSGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 128 - protein, 177 aa, synthetic construct
DTITLPCRPAPPPHCSSNITGLILTRQGGYSNANTVIFRPSGGDWRDIARCQIAGTVVSTQLFLNGSLAEEEVVIRSEDWRDNAKSICVQLATSVEIACTGAGHCAISRAKWANTLKQIASKLREQYGAKTIIFKPSSGGDPEFVNHSFNCGGEFFYCASTQLFASTWFASTGTGTK

SEQ ID NO: 129 - protein, 655 aa, synthetic construct
GSNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKNLRNNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEPGKCIYAPPIKGNITCKSDITGLLLLRDGGNALRPTEIFRPSGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 130 - protein, 655 aa, synthetic construct
GSNLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKNLRNNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEPGKCIYAPPIKGNITCKSDITGLLLLRDGGNSQRETEIFRPSGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 131 - protein, 658 aa, synthetic construct
AENLWVTVYYGVPVWKEAKTTLFCASDAKAYEKEVHNVWATHACVPTDPNPQEVVLENVTENFNMWKNDMVDQMQEDVISIWDQSLKPCVKLTPLCVTLNCTNVNVTSNSTNVNSSSTDNTTLGEIKNCSFDITTEIRDKTRKEYALFYRLDIVPLDNSSNPNSSNTYRLINCNTSTCTQACPKVTFDPIPIHYCAPAGYAILKCNNKTFNGKGPCNNVSTVQCTHGIKPVVSTQLLLNGSLAEEEIVIRSKNLTNNAKIIIVQLNKSVEIVCTRPNNNTRRSIRIGPGQTFYATDIIGDIRQAYCNISGRNWSEAVNQVKKKLKEHFPHKNISFQSSSGGDLEITTHSFNCGGEFFYCNTSGLFNDTISNATIMLPCRIKQIINMWQEVGKCIYAPPIKGNITCKSDITGLLLLRDGGNTANNAEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGRGVPHIVMVDAYKRYK

SEQ ID NO: 132 - protein, 657 aa, synthetic construct
AENLWVTVYYGVPVWKDAETTLFCASDAKAYHTEVHNVWATHACVPTDPNPQEINLENVTEEFNMWKNKMVEQMHTDIISLWDQSLKPCVQLTPLCVTLDCQYNVTNINSTSDMAREINNCSYNITTELRDREQKVYSLFYRSDIVQMNSDNSSKYRLINCNTSACKQACPKVTFEPIPIHYCAPAGFAILKCKDKEFNGTGPCKNVSTVQCTHGIKPVVSTQLLLNGSLAEEKVMIRSENITDNAKNIIVQFKEPVKINCTRPNNNTRKSVRIGPGQTFYATGEIIGDIRQAHCNVSKAHWENTLQEVANQLKLMIHSNKTIIFANSSGGDLEITTHSFNCGGEFFYCYTSGLFNYTFNDTSTNSTESKSNDTITLQCRIKQIINMWQRAGQCVYAPPIPGIIRCESNITGLILTRDGGNNNSNTNETFRPGGGDMRDNWRSELYRYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 133 - protein, 654 aa, synthetic construct
AENLWVTVYYGVPVWKEATTTLFCASDAKAYDTEVHNVWATHACVPTDPDPQEMFLENVTENFNMWKNNMVDQMHEDVISLWDQSLKPCVKLTPLCVTLECKNVNSSSSDTKNGTDPEMKNCSFNATTELRDRKQKVYALFYKLDIVPLNEKNSSEYRLINCNTSTCTQACPKVTFDPIPIHYCTPAGYAILKCNDEKFNGTGPCSNVSTVQCTHGIKPVVSTQLLLNGSLAEKGIVIRSENLTNNVKTIIVHLNQSVEILCIRPNNNTRKSIRIGPGQTFYATGEIIGDIRQAHCNISGKVWNETLQRVGEKLAEYFPNKTIKFNSSSGGDLEITTHSFNCGGEFFYCNTSKLFNGTFNGTYMPNVTEGNSTISIPCRIKQIINMWQKVGRCMYAPPIEGNITCKSKITGLLLERDGGPENDTEIFRPGGGDMRNNWRSELYKYKVVEIKPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 134 - protein, 657 aa, synthetic construct
AENLWVTVYYGVPVWKEAKTTLFCASDAKAYEKEVHNVWATHACVPTDPNPQEMVLENVTENFNMWKNDMVDQMHEDVISLWDQSLKPCVKLTPLCVTLTCTNTTVSNGSSNSNANFEEMKNCSFNATTEIKDKKKNEYALFYKLDIVPLNNSSGKYRLINCNTSACAQACPKVTFEPIPIHYCAPAGYAILKCNNKTFNGTGPCNNVSTVQCTHGIKPVVSTQLLLNGSLAEKEIIIRSENLTNNAKTIIIHFNESVGIVCTRPSNNTRKSIRIGPGQTFYATGDIIGDIRQAHCNVSKQNWNRTLQQVGRKLAEHFPNRNITFNHSSGGDLEITTHSFNCRGEFFYCNTSGLFNGTYHPNGTYNETAVNSSDTITLQCRIKQIINMWQEVGRCMYAPPIAGNITCNSTITGLLLTRDGGINQTGEEIFRPGGGDMRDNWRNELYKYKVVEIKPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 135 - protein, 653 aa, synthetic construct
AENLWVTVYYGVPVWREAKTTLFCASDAKAYEREVHNVWATHACVPTDPNPQERVLENVTENFNMWKNNMVDQMHEDIISLWDESLKPCIKLTPLCVTLNCGNAIVNESTIEGMKNCSFNVTTELKDKKKKEYALFYKLDVVPLNGENNNSNSKNFSEYRLINCNTSTCTQACPKVSFDPIPIHYCAPAGFAILKCNNETFNGTGPCNNVSTVQCTHGIKPVVSTQLLLNGSLAEKEIIIRSENLTNNAKIIIVHLNNPVKIICTRPGNNTRKSMRIGPGQTFYATGDIIGDIRRAYCNISEKTWYDTLKNVSDKFQEHFPNASIEFKPSAGGDLEITTHSFNCRGEFFYCDTSELFNGTYNNSTYNSSNNITLQCKIKQIINMWQGVGRCMYAPPIAGNITCESNITGLLLTRDGGNNKSTPETFRPGGGDMRDNWRSELYKYKVVEIKPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 136 - protein, 648 aa, synthetic construct
AENLWVTVYYGVPVWRDADTTLFCASDAKAHETEVHNVWATHACVPTDPNPQEIHLVNVTENFNMWKNKMVEQMQEDVISLWDESLKPCVKLTPLCVTLNCTTANTNETKNNTTDDNIKDEMKNCTFNMTTEIRDKKQRVSALFYKLDIVPIDDSKNNSEYRLINCNTSVCKQACPKVSFDPIPIHYCTPAGYVILKCNDKNFNGTGPCKNVSSVQCTHGIKPVVSTQLLLNGSLAEEEIIIRSENLTDNAKNIIVHLNKSVEINCTRPSNNTRTSVRIGPGQVFYRTGDITGDIRKAYCNISGTEWNKTLTQVAEKLKEHFNKTIVYQPPSGGDLEITMHHFNCRGEFFYCNTTQLFNNSVGNSTIKLPCRIKQIINMWQGVGQCMYAPPISGAINCLSNITGILLTRDGGGNNRSNETFRPGGGNIKDNWRSELYKYKVVEIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 137 - protein, 671 aa, synthetic construct
AENLWVTVYYGVPVWKDASTTLFCASDAKAYDTEVHNVWATHACVPTDPNPQEVVLGNVTENFNMWKNNMVDQMHEDIISLWDQSLKPCVKLTPLCVTLNCTDNITNTNTNSSKNSSTHSYNNSLEGEMKNCSFNITAGIRDKVKKEYALFYKLDVVPIEEDKDTNKTTYRLRSCNTSVCTQACPKVTFEPIPIHYCAPAGFAILKCNDKKFNGTGPCTNVSTVQCTHGIRPVVSTQLLLNGSLAEEEVVIRSENFTNNAKTIIVQLNESIAINCTRPNNNTRRSIHIGPGRAFYATGDIIGDIRQAHCNISRTEWNSTLRQIVTKLREQLGDPNKTIIFNQSSGGDTEITMHSFNCGGEFFYCNTTKLFNSTWNGNNTTESDSTGENITLPCRIKQIINLWQEVGKCMYAPPIKGQISCSSNITGLLLTRDGGNNNSSGPETFRPGGGNMKDNWRSELYKYKVIKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 138 - protein, 661 aa, synthetic construct
AENLWVTVYYGVPVWEDADTTLFCASDAKAYSTESHNVWATHACVPTDPNPQEIYLENVTEDFNMWENNMVEQMQEDIISLWDESLKPCVKLTPLCVTLTCTNVTNVTDSVGTNSRLKGYKEELKNCSFNTTTEIRDKKKQEYALFYKLDIVPINDNSNNSNGYRLINCNVSTCKQACPKVSFDPIPIHYCAPAGFAILKCRDKEFNGTGTCRNVSTVQCTHGIKPVVSTQLLLNGSLAEGDIVIRSENITDNAKTIIVHLNKTVSITCTRPNNNTRKSIRIGPGQALYATGAIIGDTRQAHCNISGSEWYEMIQNVKNKLNETFKKNITFNPSSGGDLEITTHSFNCRGEFFYCNTSELFNSSHLFNGSTLSTNGTITLPCRIKQIVRMWQRVGQCMYAPPIAGNITCRSNITGLLLTRDGGTNKDTNEAETFRPGGGDMRDNWRSELYKYKVVKIKPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 139 - protein, 661 aa, synthetic construct
AENLWVTVYYGVPVWKEATTTLFCASEAKAYDTEVHNIWATHACVPTDPNPQEMELKNVTENFNMWKNNMVEQMHQDIISLWDQSLKPCVKLTPLCVTLDCTNINSTNSTNNTSSNSKMEETIGVIKNCSFNVTTNIRDKVKKENALFYSLDLVSIGNSNTSYRLISCNTSICTQACPKVSFDPIPIHYCAPAGFAILKCRDKKFNGTGPCRNVSSVQCTHGIRPVVSTQLLLNGSLAEEEIVIRSANLTDNAKTIIIQLNETIQINCTRPNNNTRRSIPIGPGRTFYATGDIIGDIRKAYCNISATKWNNTLRQIAEKLREKFNKTIIFNQSSGGDPEVVRHTFNCGGEFFYCNSSQLFNSTWYSNGTSNGGLNNSANITLPCRIKQIINLWQEVGKCMYAPPIKGVINCLSNITGIILTRDGGENNGTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGGSGGGSGGGSGSGAHIVMVDAYKPTK

SEQ ID NO: 140 - protein, 632 aa, synthetic construct
NLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 141 - protein, 632 aa, synthetic construct
AEQLWVTVYYGVPVWKEATTTLFCASDARAYDTEVRNVWATHCCVPTDPNPQEVVLENVTENFNMWKNNMVEQMHEDIISLWDQSLKPCVKLTPLCVTLNCTDLRNATNTNATNTTSSSRGTMEGGEIKNCSFNITTSMRDKVQKEYALFYKLDVVPIKNDNTSYRLISCNTSVITQACPKVSFEPIPIHYCAPAGFAILKCNNKTFNGTGPCTNVSTVQCTHGIRPVVSTQLLLNGSLAEEEVVIRSANFTDNAKIIIVQLNKSVEINCTRPNNNTRKSIHIGPGRWFYTTGEIIGDIRQAHCNISGTKWNDTLKQIVVKLKEQFGNKTIVFNHSSGGDPEIVMHSFNCGGEFFYCNSTQLFNSTWNDTTGSNYTGTIVLPCRIKQIVNMWQEVGKAMYAPPIKGQIRCSSNITGLILIRDGGKNRSENTEIFRPGGGDMRDNWRSELYKYKVVKIEPLGIAPTKCKRRVVQRRRRRRAVGIGAVFLGFLGAAGSTMGAASMTLTVQARQLLSGIVQQQNNLLRAPECQQHLLKLTVWGIKQLQARVLAVERYLKDQQLLGIWGCSGKLICCTAVPWNTSWSNKSYNQIWNNMTWMEWEREIDNYTSLIYTLIEDSQNQQEKNEQELLELD

SEQ ID NO: 142 - protein, 642 aa, synthetic construct
AAKKWVTVYYGVPVWKEATTTLFCASDAKAYDTEVHNVWATHACVPTDPNPQEIVLGNVTENFNMWKNNMVEQMHEDIISLWDQSLKPCVKLTPLCVTLNCNNVNTNNTNNSTNATISDWEKMETGEMKNCSFNVTTSIRDKIKKEYALFYKLDVVPLENKNNINNTNITNYRLINCNTSVITQACPKVSFEPIPIHYCAPAGFAILKCNSKTFNGSGPCTNVSTVQCTHGIRPVVSTQLLLNGSLAEEEIVIRSENITDNAKTIIVQLNEAVEINCTRPNNNTRKSIHIGPGRWFYATGDIIGNIRQAHCNISKARWNETLGQIVAKLEEQFPNKTIIFNHSSGGDPEIVTHSFNCGGEFFYCNTTPLFNSTWNNTRTDDYPTGGEQNITLQCRIKQIINMWQGVGKAMYAPPIRGQIRCSSNITGLLLTRDGGRDQNGTETFRPGGGNMRDNWRSELYKYKVVKIEPLGIAPTACKRRVVQRRRRRRAVGLGAFILGFLGAAGSTMGAASMALTVQARLLLSGIVQQQNNLLRAPEAQQHMLQLTVWGIKQLQARVLAVERYLRDQQLLGIWGCSGKIICCTNVPWNDSWSNKTINEIWDNMTWMQWEKEIDNYTQHIYTLLEVSQIQQEKNEQELLELD

SEQ ID NO: 143 - protein, 635 aa, synthetic construct
AENLWVTVYYGVPVWKEATTTLFCASDAKAYDTEVHNVWATHACVPTDPSPQELVLENVTENFNMWKNEMVNQMHEDVISLWDQSLKPCVKLTPLCVTLECSKVSNNETDKYNGTEEMKNCSFNATTVVRDRQQKVYALFYRLDIVPLTEKNSSENSSKYYRLINCNTSACTQACPKVSFEPIPIHYCTPAGYAILKCNDKTFNGTGPCHNVSTVQCTHGIKPVVSTQLLLNGSLAEGEIIIRSENLTNNVKTILVHLNQSVEIVCTRPNNNTRKSIRIGPGQTFYATGDIIGDIRQAHCNISKWHETLKRVSEKLAEHFPNKTINFTSSSGGDLEITTHSFTCRGEFFYCNTSGLFNSTYMPNGTYLHGDTNSNSSITIPCRIKQIINMWQEVGRCMYAPPIEGNITCKSNITGLLLVRDGGTESNNTETNNTEIFRPGGGDMRDNWRSELYKYKVVEIKPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 144 - protein, 624 aa, synthetic construct
AENLWVTVYYGVPVWREAKTTLFCASDAKAYEREVHNVWATHACVPTDPNPQERVLENVTENFNMWKNNMVDQMHEDIISLWDESLKPCIKLTPLCVTLNCGNAIVNESTIEGMKNCSFNVTTELKDKKKKEYALFYKLDVVPLNGENNNSNSKNFSEYRLINCNTSTCTQACPKVSFDPIPIHYCAPAGFAILKCNNETFNGTGPCNNVSTVQCTHGIKPVVSTQLLLNGSLAEKEIIIRSENLTNNAKIIIVHLNNPVKIICTRPGNNTRKSMRIGPGQTFYATGDIIGDIRRAYCNISEKTWYDTLKNVSDKFQEHFPNASIEFKPSAGGDLEITTHSFNCRGEFFYCDTSELFNGTYNNSTYNSSNNITLQCKIKQIINMWQGVGRCMYAPPIAGNITCESNITGLLLTRDGGNNKSTPETFRPGGGDMRDNWRSELYKYKVVEIKPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 145 - protein, 624 aa, synthetic construct
AENLWVTVYYGVPVWRDADTTLFCASDAKAYDTEVHNVWATHACVPTDPNPQEIHLENVTENFNMWKNKMAEQMQEDVISLWDESLKPCVQLTPLCVTLNCTNANLNATVNASTTIGNITDEVRNCSFNTTTELRDKKQNVYALFYKLDIVPINNNSEYRLINCNTSVCKQACPKVSFDPIPIHYCAPAGYAILRCNDKNFNGTGPCKNVSSVQCTHGIKPVVSTQLLLNGSLAEDEIIIRSENLTDNVKTIIVHLNKSVEINCTRPSNNTRTSVRIGPGQVFYRTGDIIGDIRKAYCNISRTKWHETLKQVATKLREHFNKTIIFQPPSGGDIEITMHHFNCRGEFFYCNTTKLFNSTWGENTTMEGHNDTIVLPCRIKQIVNMWQGVGQCMYAPPIRGSINCVSNITGILLTRDGGTNMSNETFRPGGGNIKDNWRSELYKYKVVEIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 146 - protein, 624 aa, synthetic construct
AENLWVTVYYGVPVWRDADTTLFCASDAKAYDTEVHNVWATHACVPTDPNPQEIHLENVTENFNMWKNKMAEQMQEDVISLWDESLKPCVQLTPLCVTLNCTNANLNATVNASTTIGNITDEVRNCSFNTTTELRDKKQNVYALFYKLDIVPINNNSEYRLINCNTSVCKQACPKVSFDPIPIHYCAPAGYAILRCNDKNFNGTGPCKNVSSVQCTHGIKPVVSTQLLLNGSLAEDEIIIRSEALTDNVKTIIVHLNKSVEINCTRPSNNTRTSVRIGPGQVFYRTGDIIGDIRKAYCNISRTKWHETLKQVATKLREHFNKTIIFQPPSGGDIEITMHHFNCRGEFFYCNTTKLFNSTWGENTTMEGHNDTIVLPCRIKQIVNMWQGVGQCMYAPPIRGSINCVSNITGILLTRDGGTNMSNETFRPGGGNIKDNWRSELYKYKVVEIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 147 - protein, 618 aa, synthetic construct
NLWVTVYYGVPVWKEATTTLFCASDAKAYDTEVHNVWATHACVPTDPNPHELVLENVTENFNMWKNEMVNQMHEDVISLWDQSLKPCVKLTPLCVTLECGNITTRKESMTEMKNCSFNATTVVKDRKQTVYALFYKLDIVPLSGKNSSGYYRLINCNTSACTQACPKVNFDPIPIHYCTPAGYAILKCNDKTFNGTGPCHNVSTVQCTHGIKPVISTQLLLNGSLAEGEIVIRSENLTNNAKIIIVHLNQTVEIVCTRPGNNTRKSIRIGPGQTFYATGEIIGNIRQAHCNISENQWHKTLQNVSKKLAEHFQNKTITFASSSGGDLEITTHSFNCRGEFFYCNTSGLFNGTYMSNNTEGNSSSIITIPCRIKQIINMWQEVGRCIYAPPIEGNITCKSNITGLLLERDGGTESNDTEIFRPGGGDMRNNWRSELYKYKVVEIKPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 148 - protein, 618 aa, synthetic construct
NLWVTVYYGVPVWKEATTTLFCASDAKAYDTEVHNVWATHACVPTDPNPHELVLENVTENFNMWKNEMVNQMHEDVISLWDQSLKPCVKLTPLCVTLECGNITTRKESMTEMKNCSFNATTVVKDRKQTVYALFYKLDIVPLSGKNSSGYYRLINCNTSACTQACPKVNFDPIPIHYCTPAGYAILKCNDKTFNGTGPCHNVSTVQCTHGIKPVISTQLLLNGSLAEGEIVIRSEALTNNAKIIIVHLNQTVEIVCTRPGNNTRKSIRIGPGQTFYATGEIIGNIRQAHCNISENQWHKTLQNVSKKLAEHFQNKTITFASSSGGDLEITTHSFNCRGEFFYCNTSGLFNGTYMSNNTEGNSSSIITIPCRIKQIINMWQEVGRCIYAPPIEGNITCKSNITGLLLERDGGTESNDTEIFRPGGGDMRNNWRSELYKYKVVEIKPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 149 - protein, 232 aa, synthetic construct
EVQLVESGAQVKKPGASVTVSCTASGYKFTGYHMHWVRQAPGRGLEWMGWINPFRGAVKYPQNFRGRVSMTRDTSMEIFYMELSRLTSDDTAVYYCAREMFDSSADWSPWRGMVAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKT

SEQ ID NO: 150 - protein, 456 aa, synthetic construct
EVQLVESGAQVKKPGASVTVSCTASGYKFTGYHMHWVRQAPGRGLEWMGWINPFRGAVKYPQNFRGRVSMTRDTSMEIFYMELSRLTSDDTAVYYCAREMFDSSADWSPWRGMVAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 214 aa, synthetic construct
QSALTQPASVSGSPGQSITISCAGSSRDVGGFDLVSWYQQHPGKAPKLIIYEVNKRPSGISSRFSASKSGNTASLTISGLQEEDEAHYYCYSYADGVAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 152 - protein, 231 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDFTSSYDSSGYYHEGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKT

SEQ ID NO: 153 - protein, 455 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDFTSSYDSSGYYHEGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 214 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCCSYAGSVAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 155 - protein, 451 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGKNSDYNWDFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 210 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQYEFFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 449 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARERSDFWDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 210 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYEFIGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 453 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCATQLELDSSAGYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 211 aa, synthetic construct
QSALTQPRSVSGSPGQSVTISCTGTSSDVGGYNYVSWYQQHPGKAPKLMIYDVSKRPSGVPDRFSGSKSGNTASLTISGLQAEDEADYYCSSYEYFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 161 - DNA, 27 bp, synthetic construct
  source 1..27: mol_type = other DNA
gtctggaaagaggctaagaccacactg

SEQ ID NO: 162 - DNA, 30 bp, synthetic construct
  source 1..30: mol_type = other DNA
caggttttttgatctgatcacaatctcttc

SEQ ID NO: 163 - DNA, 58 bp, synthetic construct
  source 1..58: mol_type = other DNA
gaagagattgtgatcagatcaaaaaacctgnnkaacaatgccaagatcattatcgtgc

SEQ ID NO: 164 - DNA, 44 bp, synthetic construct
  source 1..44: mol_type = other DNA
atctccacactcttattcagctgcacgataatgatcttggcatt

SEQ ID NO: 165 - DNA, 42 bp, synthetic construct
  source 1..42: mol_type = other DNA
agctgaataagagtgtggagatcgtctgcacacgacctaaca

SEQ ID NO: 166 - DNA, 57 bp, synthetic construct
  source 1..57: mol_type = other DNA
gcctgccgaatatctcccccagatccgctgccgccattgttaggtcgtgtgcagacg

SEQ ID NO: 167 - DNA, 59 bp, synthetic construct
  source 1..59: mol_type = other DNA
ggagatattcggcaggcttattgtaacatcagtggcagaaattggtcagaagccgtgaa

SEQ ID NO: 168 - DNA, 57 bp, synthetic construct
  source 1..57: mol_type = other DNA
tgggggaagtgctctttcagctttttcttgacctggttcacggcttctgaccaattt

SEQ ID NO: 169 - DNA, 54 bp, synthetic construct
  source 1..54: mol_type = other DNA
aaagagcacttcccccataagaatattagctttcagtctagttcaggcggggac

SEQ ID NO: 170 - DNA, 54 bp, synthetic construct
  source 1..54: mol_type = other DNA
tcgcctccgcagttgaaggagtgtgtggtgatttccaggtccccgcctgaacta

SEQ ID NO: 171 - DNA, 48 bp, synthetic construct
  source 1..48: mol_type = other DNA
actgcggaggcgagttcttttactgtaatacatccggcctgtttaacg

SEQ ID NO: 172 - DNA, 58 bp, synthetic construct
  source 1..58: mol_type = other DNA
ccggcaaggcagcatgattgtggcattagaaatggtatcgttaaacaggccggatgta

SEQ ID NO: 173 - DNA, 43 bp, synthetic construct
  source 1..43: mol_type = other DNA
gctgccttgccggatcaagcagattatcaacatgtggcaggaa

SEQ ID NO: 174 - DNA, 59 bp, synthetic construct
  source 1..59: mol_type = other DNA
tgcccttgatgggtggtgcatagatagcctttccmnnttcctgccacatgttgataatc

SEQ ID NO: 175 - DNA, 60 bp, synthetic construct
  source 1..60: mol_type = other DNA
ccacccatcaagggcaatatcacctgtaagagtgacattacagggctgctgctgctgaga

SEQ ID NO: 176 - DNA, 57 bp, synthetic construct
  source 1..57: mol_type = other DNA
gccggaaaatctcggtmnnmnnmnnmnnmnntcccccatctctcagcagcagcagcc

SEQ ID NO: 177 - DNA, 57 bp, synthetic construct
  source 1..57: mol_type = other DNA
accgagattttccggcctagcggaggagacatgcgagataattggcggtctgaactg

SEQ ID NO: 178 - DNA, 58 bp, synthetic construct
  source 1..58: mol_type = other DNA
ggatcccagaggcttgatctcgaccaccttatatttgtacagttcagaccgccaatta

SEQ ID NO: 179 - DNA, 60 bp, synthetic construct
  source 1..60: mol_type = other DNA
ctggtggaggcggtagcggaggcggagggtcggctagcgtctggaaagaggctaagacca

SEQ ID NO: 180 - DNA, 60 bp, synthetic construct
  source 1..60: mol_type = other DNA
ttacaagtcctcttcagaaataagcttttgttcggatcccagaggcttgatctcgaccac

SEQ ID NO: 181 - DNA, 27 bp, synthetic construct
  source 1..27: mol_type = other DNA
gtctggaaagaggctaagaccacactg

SEQ ID NO: 182 - DNA, 30 bp, synthetic construct; identical to SEQ ID NO: 162
  source 1..30: mol_type = other DNA

SEQ ID NO: 183 - DNA, 58 bp, synthetic construct; identical to SEQ ID NO: 163
  source 1..58: mol_type = other DNA

SEQ ID NO: 184 - DNA, 44 bp, synthetic construct; identical to SEQ ID NO: 164
  source 1..44: mol_type = other DNA

SEQ ID NO: 185 - DNA, 42 bp, synthetic construct; identical to SEQ ID NO: 165
  source 1..42: mol_type = other DNA

SEQ ID NO: 186 - DNA, 57 bp, synthetic construct; identical to SEQ ID NO: 166
  source 1..57: mol_type = other DNA

SEQ ID NO: 187 - DNA, 59 bp, synthetic construct; identical to SEQ ID NO: 167
  source 1..59: mol_type = other DNA

SEQ ID NO: 188 - DNA, 57 bp, synthetic construct; identical to SEQ ID NO: 168
  source 1..57: mol_type = other DNA

SEQ ID NO: 189 - DNA, 54 bp, synthetic construct; identical to SEQ ID NO: 169
  source 1..54: mol_type = other DNA

SEQ ID NO: 190 - DNA, 54 bp, synthetic construct; identical to SEQ ID NO: 170
  source 1..54: mol_type = other DNA

SEQ ID NO: 191 - DNA, 48 bp, synthetic construct; identical to SEQ ID NO: 171
  source 1..48: mol_type = other DNA

SEQ ID NO: 192 - DNA, 58 bp, synthetic construct; identical to SEQ ID NO: 172
  source 1..58: mol_type = other DNA

SEQ ID NO: 193 - DNA, 43 bp, synthetic construct; identical to SEQ ID NO: 173
  source 1..43: mol_type = other DNA

SEQ ID NO: 194 - DNA, 59 bp, synthetic construct; identical to SEQ ID NO: 174
  source 1..59: mol_type = other DNA

SEQ ID NO: 195 - DNA, 60 bp, synthetic construct; identical to SEQ ID NO: 175
  source 1..60: mol_type = other DNA

SEQ ID NO: 196 - DNA, 57 bp, synthetic construct; identical to SEQ ID NO: 176
  source 1..57: mol_type = other DNA

SEQ ID NO: 197 - DNA, 57 bp, synthetic construct; identical to SEQ ID NO: 177
  source 1..57: mol_type = other DNA

SEQ ID NO: 198 - DNA, 58 bp, synthetic construct; identical to SEQ ID NO: 178
  source 1..58: mol_type = other DNA

SEQ ID NO: 199 - DNA, 60 bp, synthetic construct; identical to SEQ ID NO: 179
  source 1..60: mol_type = other DNA

SEQ ID NO: 200 - DNA, 60 bp, synthetic construct; identical to SEQ ID NO: 180
  source 1..60: mol_type = other DNA

SEQ ID NO: 201 - protein, 9 aa, synthetic construct
FTGYYMHWV

SEQ ID NO: 202 - protein, 9 aa, synthetic construct
FTGYHMHWV

SEQ ID NO: 203 - protein, 9 aa, synthetic construct
FTDYFIHWV

SEQ ID NO: 204 - protein, 9 aa, synthetic construct
FTGYELHWV

SEQ ID NO: 205 - protein, 9 aa, synthetic construct
FTGYHLHWV

SEQ ID NO: 206 - protein, 10 aa, synthetic construct
TFIDYYIHWV

SEQ ID NO: 207 - protein, 10 aa, synthetic construct
TFTDYFIHWV

SEQ ID NO: 208 - protein, 10 aa, synthetic construct
TFADHFIHWV

SEQ ID NO: 209 - protein, 10 aa, synthetic construct
TFTGYDMHWV

SEQ ID NO: 210 - protein, 10 aa, synthetic construct
TFTDYEIHWV

SEQ ID NO: 211 - protein, 15 aa, synthetic construct
INPNSGGTNYAQKFQ

SEQ ID NO: 212 - protein, 15 aa, synthetic construct
INPFRGAVKYPQNFR

SEQ ID NO: 213 - protein, 15 aa, synthetic construct
INPFRGAVDYAQKFR

SEQ ID NO: 214 - protein, 15 aa, synthetic construct
INPYRGAVKYAQKFQ

SEQ ID NO: 215 - protein, 15 aa, synthetic construct
INPFRGAIGYAQKFR

SEQ ID NO: 216 - protein, 15 aa, synthetic construct
INPYRGGPGYAQKFR

SEQ ID NO: 217 - protein, 15 aa, synthetic construct
INPRFGVTDSAQKFR

SEQ ID NO: 218 - protein, 15 aa, synthetic construct
INPRFGNTDSAQKFR

SEQ ID NO: 219 - protein, 15 aa, synthetic construct
INPFRGGIEYAQKFQ

SEQ ID NO: 220 - protein, 15 aa, synthetic construct
INPFRGAVKYRQKFQ

SEQ ID NO: 221 - protein, 20 aa, synthetic construct
AREMFDSSADWSPWRGMVAW

SEQ ID NO: 222 - protein, 20 aa, synthetic construct
ARAMFDSSDEWSPWHGLVAW

SEQ ID NO: 223 - protein, 20 aa, synthetic construct
ARDMFDSSDEWSPWRGMVAW

SEQ ID NO: 224 - protein, 20 aa, synthetic construct
AREMFDSDDEWSPWRGMVAW

SEQ ID NO: 225 - protein, 20 aa, synthetic construct
AREMFDSDDDWSPWRGMVAW

SEQ ID NO: 226 - protein, 20 aa, synthetic construct
ARELFDRDDDWSPWRGMVAW

SEQ ID NO: 227 - protein, 20 aa, synthetic construct
ARTMFDSDDDWSPWCGLVAW

SEQ ID NO: 228 - protein, 20 aa, synthetic construct
TREVSDSSDDWSPWRGLVAW

SEQ ID NO: 229 - protein, 20 aa, synthetic construct
AREIFDSSDEWSPWRGMVAW

SEQ ID NO: 230 - protein, 8 aa, synthetic construct
SSDVGSYN

SEQ ID NO: 231 - protein, 8 aa, synthetic construct
SRDVGGFD

SEQ ID NO: 232 - protein, 8 aa, synthetic construct
SRDIGSFN

SEQ ID NO: 233 - protein, 8 aa, synthetic construct
SSDVGSYD

SEQ ID NO: 234 - protein, 8 aa, synthetic construct
SSDIGGYN

SEQ ID NO: 235 - protein, 8 aa, synthetic construct
SSDIGGYD

SEQ ID NO: 236 - protein, 8 aa, synthetic construct
SSDIGSYD

SEQ ID NO: 237 - protein, 8 aa, synthetic construct
SNDIGSYD

SEQ ID NO: 238 - protein, 8 aa, synthetic construct
SSDIGTYN

SEQ ID NO: 239 - protein, 15 aa, synthetic construct
LMIYEVSKRPSGVSN

SEQ ID NO: 240 - protein, 15 aa, synthetic construct
LIIYEVNKRPSGISS

SEQ ID NO: 241 - protein, 15 aa, synthetic construct
LMIYDVSKRPSGVSN

SEQ ID NO: 242 - protein, 15 aa, synthetic construct
LMIYDVNKRPSGVSN

SEQ ID NO: 243 - protein, 15 aa, synthetic construct
LMIYDVTKRPSGVSN

SEQ ID NO: 244 - protein, 15 aa, synthetic construct
LMIYEVNRRPSGVSN

SEQ ID NO: 245 - protein, 15 aa, synthetic construct
LMIYEVSRRPSGVSN

SEQ ID NO: 246 - protein, 9 aa, synthetic construct
CSYAGSVAF

SEQ ID NO: 247 - protein, 9 aa, synthetic construct
YSYADGVAF

SEQ ID NO: 248 - protein, 9 aa, synthetic construct
CSFAGSLAF

SEQ ID NO: 249 - protein, 9 aa, synthetic construct
CSYADSLVF

SEQ ID NO: 250 - protein, 9 aa, synthetic construct
CSYADTLVF

SEQ ID NO: 251 - protein, 9 aa, synthetic construct
CSYGDNLVF

SEQ ID NO: 252 - protein, 9 aa, synthetic construct
WSYADSVAF

SEQ ID NO: 253 - protein, 9 aa, synthetic construct
SSYEGTLAF

SEQ ID NO: 254 - protein, 9 aa, synthetic construct
CSYADTLIF

SEQ ID NO: 255 - RNA, 20 nt, synthetic construct
  source 1..20: mol_type = other RNA
aggcatcggaaaatccacag

SEQ ID NO: 256 - RNA, 20 nt, synthetic construct
  source 1..20: mol_type = other RNA
ctgggagttacccgattgga

SEQ ID NO: 257 - RNA, 20 nt, synthetic construct
  source 1..20: mol_type = other RNA
cacgcatgaagggagcaccg

SEQ ID NO: 258 - DNA, 21 bp, synthetic construct
  source 1..21: mol_type = other DNA
ccatgggatggtcatgtatca

SEQ ID NO: 259 - DNA, 20 bp, synthetic construct
  source 1..20: mol_type = other DNA
ggacagggatccagagttcc

SEQ ID NO: 260 - DNA, 20 bp, synthetic construct
  source 1..20: mol_type = other DNA
cccatggccaccagattctt

SEQ ID NO: 261 - DNA, 20 bp, synthetic construct
  source 1..20: mol_type = other DNA
gactgaggcacctccagatg

SEQ ID NO: 262 - DNA, 20 bp, synthetic construct
  source 1..20: mol_type = other DNA
cccatggccaccagattctt

SEQ ID NO: 263 - DNA, 27 bp, synthetic construct
  source 1..27: mol_type = other DNA
gtagcaactgcaaccggtgtacattct

SEQ ID NO: 264 - DNA, 22 bp, synthetic construct
  source 1..22: mol_type = other DNA
gctcagggaartagcccttgac

SEQ ID NO: 265 - DNA, 24 bp, synthetic construct
  source 1..24: mol_type = other DNA
agggggaagacatttgggaaggac

SEQ ID NO: 266 - DNA, 20 bp, synthetic construct
  source 1..20: mol_type = other DNA
aactgctcactggatggtgg

SEQ ID NO: 267 - protein, 121 aa, synthetic construct
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSS

SEQ ID NO: 268 - protein, 856 aa, synthetic construct
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL